SEQUENCE LISTING (61 sequences)

SEQ ID NO: 1 - protein, 662 aa, Homo sapiens
QEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 2 - DNA, 2076 bp, Homo sapiens
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgctccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccgcggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggggcaggtgaccgtggcctgcgaggagggctggaccctgactggctgcagcgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccag

SEQ ID NO: 3 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 4 - protein, 447 aa, Artificial Sequence
  Alirocumab heavy chain
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 220 aa, Artificial Sequence
  Alirocumab light chain
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 444 aa, Artificial Sequence
  Bococizumab heavy chain
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 214 aa, Artificial Sequence
  Bococizumab light chain
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 114 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYGMDVWGQGTTVTVSS

SEQ ID NO: 9 - protein, 115 aa, Homo sapiens
QIQLVQSGAEVKKPGASVKVSCKASGYPLTSYGISWVRQAPGQGLEWMGWISAYNGNTNYAQKVQGSVTMTTDTSTSTVYMELRSLRSDDTAVYYCARGYGMDVWGQGTTVTVSS

SEQ ID NO: 10 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 11 - protein, 115 aa, Homo sapiens; identical to SEQ ID NO: 10

SEQ ID NO: 12 - protein, 115 aa, Homo sapiens; identical to SEQ ID NO: 10

SEQ ID NO: 13 - protein, 115 aa, Homo sapiens
QVQLVQSGAEVKKPGASVKVSCKASGYTLTSYGISWVRQAPGQGLEWMGWISFYNGNTNYAQKVQGRVTMTTDTSTSTVYMELRSLRSDDTAVYYCARGYGMDVWGQGTTVTVSS

SEQ ID NO: 14 - protein, 115 aa, Homo sapiens
QVQLVQSGAEVKRPGASVKVSCKASGYTLTSYGISWVRQAPGQGLEWMGWISVYNGNTNYAQKVQGRVTMTTDTSTSTVYMELRSLSSDDTAVYYCARGYGMDVWGQGTTVTVSS

SEQ ID NO: 15 - protein, 115 aa, Homo sapiens
QIQLVQSGAEVKKPGASVKVSCKASGYTLTSYGISWVRQAPGQGLEWMGWISFYNGNTNYAQKVQGRVTMTTDTSTSTVYMELRSLRSDDTAVYFCARGYGMDVWGQGTTVTVSS

SEQ ID NO: 16 - protein, 115 aa, Homo sapiens
QVQLVQSGAEVKKPGASLKVSCKASGYSLTSYGISWVRQAPGQGLEWMGWISAYNGNTNYAQKVQGRVTMTTDTSTSTVYMEVRSLRSDDTAVYYCARGYGMDVWGQGTTVTVSS

SEQ ID NO: 17 - protein, 115 aa, Homo sapiens
QVQLVQSGAEVKKPGASVKVSCKASGYPLTSYGISWVRQAPGQGLEWMGWISAYNGNTNYAQKVQGRVTMTTDTSTSTVYMELRSLRSDDTAVYYCARGYGMDVWGQGTTVTVSS

SEQ ID NO: 18 - protein, 115 aa, Homo sapiens
QVQLVQSGAEVKKPGASVKVSCKASGYALTSYGISWVRQAPGQGLEWMGWISAYNGNTNYAQKVQGRVTMTTDTSTSTVYMELRSLRSDDTAVYYCARGYGMDVWGQGTTVTVSS

SEQ ID NO: 19 - protein, 115 aa, Homo sapiens
QVQLVQSGAEVKKPGASVKVSCKASGYSFTSYGISWVRQAPGQGLEWMGWVSAYNGNTNYAQKFQGRVTMTTDTSTSTAYMELRSLRSDDTAVYYCARGYVMDVWGQGTTVTVSS

SEQ ID NO: 20 - protein, 115 aa, Homo sapiens
QVQLVQSGAEVKKPGASVKVSCKASGYTFPSYGISWVRQAPGQGLEWMGWISAYNGNTNYAEKLQGRVTMTTDTSTSTAYMEVRSLRSDDTAVFYCARGYVMDVWGQGTTVTVSS

SEQ ID NO: 21 - protein, 113 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGYDYWGQGTLVTVSS

SEQ ID NO: 22 - protein, 115 aa, Homo sapiens
QVQLVQSGAEVKKPGASVKVSCKASGYTFTSYGISWVRQAPGQGLEWMGWISTYNGNTNYAQKVQGRVTMTTDTSTSTAYMELRSLRSDDTAVYYCARGYTRDYWGQGTLVTVSS

SEQ ID NO: 23 - protein, 116 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARNWGAFDVWGQGTMVTVSS

SEQ ID NO: 24 - protein, 119 aa, Homo sapiens
EVQLVESGGGLVQPGGSLRLSCAASGFTFSRYWMSWVRQAPGKGLEWVANIKHDGSEKYYVDSVKGRFTISRDNAKNSLYLQMNSLRAEDTAVYYCARESNWGFAFDVWGHGTMVTVSS

SEQ ID NO: 25 - protein, 116 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARNWGAFDIWGQGTMVTVSS

SEQ ID NO: 26 - protein, 119 aa, Homo sapiens
EVQLVESGGGLVQPGGSLRLSCVVSGFTFSSYWMSWVRQAPGKGLEWVANIKQDGSEKYYVDSVKGRFTISRDNAKNSLYLQMNSLRAEDTAVYYCARESNWGFAFDIWGQGTMVTVSS

SEQ ID NO: 27 - protein, 119 aa, Homo sapiens
EVQLVESGGGLVQPGGSLRLSCAASGLTFSNFWMSWVRQAPGKGLEWVANIKQDGSEKYYVDSVKGRFTISRDNAKNSLYLQMNSLRAEDTAVYSCTRESNWGFAFDIWGQGTMVTVSS

SEQ ID NO: 28 - protein, 123 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDYDFWSGYYTAFDVWGQGTMVTVSS

SEQ ID NO: 29 - protein, 123 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 30 - protein, 112 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKFDYWGQGTLVTVSS

SEQ ID NO: 31 - protein, 117 aa, Homo sapiens
EVQLLESGGGLVQPGGSLRLSCAASGFTFSSYAMSWVRQAPGKGLEWVSTISGSGGRTYYADSVKGRFTISRDNSKNTLYLQMNSLRAEDTAVYYCAKEVGSPFDYWGQGTLVTVSS

SEQ ID NO: 32 - protein, 117 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLMVYADYWGQGTLVTVSS

SEQ ID NO: 33 - protein, 121 aa, Homo sapiens
EVQLLESGGGLVQPGGSLRLSCAASGFTFSSYAMNWVRQAPGKGLEWVSTISGSGDNTYYADSVKGRFTISRDNSKNTLYLQMNSLRAEDTAVYYCAKKFVLMVYAMLDYWGQGTLVTVSS

SEQ ID NO: 34 - protein, 121 aa, Homo sapiens
EVQLLESGGGLVQPGGSLRLSCAASGFTFSSYAMNWVRQAPGKGLEWVSTISGSGGNTYYADSVKGRFTISRDNSKNTLYLQMNSLRAEDTAVYYCAKKFVLMVYAMLDYWGQGTLVTVSS

SEQ ID NO: 35 - protein, 116 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARYYYGMDVWGQGTTVTVSS

SEQ ID NO: 36 - protein, 123 aa, Homo sapiens
QVQLVESGGGVVQPGRSLRLSCAASGFTFSSYGMHWVRQAPGKGLEWVAVIWYDGSDKYYADSVKGRFTISRDNSKNTLYLQMNSLRAEDTAVYYCARETGPLKLYYYGMDVWGQGTTVTVSS

SEQ ID NO: 37 - protein, 116 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARIAAGMDVWGQGTTVTVSS

SEQ ID NO: 38 - protein, 122 aa, Homo sapiens
QVHLVESGGGVVQPGRSLRLSCAASGFTFNSFGMHWVRQAPGKGLEWVALIWSDGSDKYYADSVKGRFTISRDNSKNTLYLQMNSLRAEDTAVYYCARAIAALYYYYGMDVWGQGTTVTVSS

SEQ ID NO: 39 - protein, 122 aa, Homo sapiens
QVQLVESGGGVVQPGRSLRLSCAASGFTFSSFGMHWVRQAPGKGLEWVALIWNDGSNKYYADSVKGRFTISRDNSKNTLYLQMNSLRAEDTAVYYCARAIAALYYYYGMDVWGHGTTVTVSS

SEQ ID NO: 40 - protein, 122 aa, Homo sapiens
QVHLVESGGGVVQPGRSLRLSCAASGFTFSSFGMHWVRQAPGKGLEWVALIWNDGSNKYYADSVKGRFTISRDNSKNTLYLQMNSLRAEDTAVYYCARAIAALYYYYGMDVWGHGTTVTVSS

SEQ ID NO: 41 - protein, 122 aa, Homo sapiens
QVQLVESGGGVVQPGRSLRLSCAASGFTFSSFGMHWVRQAPGKGLEWVALIWNDGSNKYYADSVKGRFTISRDNSKNTLYLQMNSLRAEDTAVYYCARAIAALYYYYGMDVWGQGTTVTVSS

SEQ ID NO: 42 - protein, 122 aa, Homo sapiens
QVHLVESGGGVVQPGRSLRLSCAASGFTFNSFGMHWVRQAPGKGLEWVALIWSDGSDEYYADSVKGRFTISRDNSKNTLYLQMNSLRAEDTAVYYCARAIAALYYYYGMDVWGQGTTVTVSS

SEQ ID NO: 43 - protein, 122 aa, Homo sapiens; identical to SEQ ID NO: 41

SEQ ID NO: 44 - protein, 122 aa, Homo sapiens
QVQLVESGGGVVQPGRSLRLSCAASGFTFSSYGMHWVRQAPGKGLEWVAIIWYDGSNKYYADSVKGRFTISRDNSKNTLYLQMNSLRAEDTAVYYCARRGGLAARPGGMDVWGQGTTVTVSS

SEQ ID NO: 45 - protein, 122 aa, Homo sapiens
QVQLVESGGGVVQPGRSLRLSCAASGFTFSSYGMHWVRQAPGKGLECVAIIWYDGSNKYYADSVKGRFTISRDNSKNTLYLQMNSLRAEDTAVYYCARRGGLAARPGGMDVWGQGTTVTVSS

SEQ ID NO: 46 - protein, 122 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARGIAVAYYYYGMDVWGQGTTVTVSS

SEQ ID NO: 47 - protein, 122 aa, Homo sapiens
QVQLVESGGGVVQPGRSLRLSCAASGFTFRSYGMHWVRQAPGKGLEWVALIWHDGSNTYYVDSVKGRFTISRDNSKNTLYLQMNSLRAEDTAVYYCARGIAVAYYYYGMDVWGQGTTVTVSS

SEQ ID NO: 48 - protein, 117 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARYYYGMDVWGQGTTVTVSS

SEQ ID NO: 49 - protein, 122 aa, Homo sapiens
QVQLQESGPGLVKPSQTLSLTCTVSGGSISSSDYYWSWIRQHPGKGLEWIGYIYYSGSTYYNPSLKSRITISVDTSKNLFSLKLSSVTAADTAVYYCARGGVTTYYYAMDVWGQGTTVTVSS

SEQ ID NO: 50 - protein, 120 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCAREDTAMVYFDYWGQGTLVTVSS

SEQ ID NO: 51 - protein, 121 aa, Homo sapiens
QVQLQESGPGLVKPSQTLSLTCTVSGGSISSGGYYWSWIRQHPGKGLEWIGYIYNSGSTYYNPSLKSRVTISVDTSKNQFSLKLSSVTAADTAVYYCAREDTAMVPYFDYWGQGTLVTVSS

SEQ ID NO: 52 - protein, 115 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGQLVFDYWGQGTLVTVSS

SEQ ID NO: 53 - protein, 116 aa, Homo sapiens
QVQLQQWGAGLLKPSETLSLTCAVYGGSFSAYYWNWIRQPPGKGLEWIGEINHSGRTDYNPSLKSRVTISVDTSKKQFSLKLNSVTAADTAVYYCARGQLVPFDYWGQGTLVTVSS

SEQ ID NO: 54 - protein, 115 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARFDYWGQGTLVTVSS

SEQ ID NO: 55 - protein, 121 aa, Homo sapiens
QVQLQQSGPGLVKPSQTLSLTCAISGDSVSSNSAAWNWIRQSPSRGLEWLGRTYYRSKWYKNYSVSVKSRITINPDTSKNQFSLQLNSVTPGDTAVYYCARGGPTAAFDYWGQGTLVTVSS

SEQ ID NO: 56 - protein, 441 aa, Homo sapiens
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - protein, 215 aa, Homo sapiens
ESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 58 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 61 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2020/0368350 A1
(43) Pub. Date: Nov. 26, 2020

(54) COMBINED THERAPIES FOR ATHEROSCLEROSIS, INCLUDING ATHEROSCLEROTIC CARDIOVASCULAR DISEASE

(71) Applicant: Amgen Inc., Thousand Oaks, CA (US)

(72) Inventors: Ransi Mudalinayake SOMARATNE, Orinda, CA (US); Robert Andrew Donald SCOTT, Highland Park, IL (US); Scott WASSERMAN, Newbury Park, CA (US); Narimon HONARPOUR, Pacific Palisades, CA (US); Stephen NICHOLLS, Hyde Park, South Australia (AU)

(21) Appl. No.: 16/348,653

(22) PCT Filed: Nov. 13, 2017
(86) PCT No.: PCT/US2017/061346

§ 371 Date: May 9, 2019

Related U.S. Application Data
(60) Provisional application No. 62/421,685, filed on Nov. 14, 2016.
(60) Provisional application No. 62/471,874, filed on Mar. 15, 2017.
(60) Provisional application No. 62/515,117, filed on Jun. 5, 2017.
(60) Provisional application No. 62/581,244, filed on Nov. 3, 2017.
(60) Provisional application No. 62/584,600, filed on Nov. 10, 2017.

(51) Int. Cl. A61K 39/395; A61K 31/40; A61K 31/505; A61K 31/366; A61K 31/235; A61K 31/405; A61K 31/47; A61P 9/10

(52) U.S. Cl. CPC A61K 39/3955; A61K 31/40; A61K 31/505; A61P 9/10; A61K 31/235; A61K 31/405; A61K 31/47; A61K 31/366

(57) ABSTRACT

Provided herein are combinations of therapies that provide for the treatment, including regression, of atherosclerosis and/or improvement of cardiovascular outcomes. Generally described, this includes a first, non-PCSK9 LDL-C lowering agent (such as a statin or other non-PCSK9 LDL-C lowering therapy), combined with a second, PCSK9 inhibitor therapy (such as a PCSK9 antibody or anti-RNA). The application of both therapies, at adequately elevated levels so as to reduce the LDL-C level of the subject to very low levels, for an adequate period of time, has been determined to provide an added benefit of further protection from atherosclerosis and improve a subject's cardiovascular outcomes.

### RELATED APPLICATIONS

This application claims priority to U.S. Provisional Application Ser. No. 62/421,685, filed Nov. 14, 2016, Ser. No. 62/471,874, filed Mar. 15, 2017, Ser. No. 62/515,117, filed Jun. 5, 2017, Ser. No. 62/581,244, filed Nov. 3, 2017, and Ser. No. 62/584,600, filed Nov. 10, 2017, each of which is hereby incorporated by reference in their entireties.

### SEQUENCE LISTING AND TABLES IN ELECTRONIC FORMAT

The present application is being filed along with a Sequence Listing in electronic format. The Sequence Listing is provided as a file entitled APMOL018WO.TXT, last saved Nov. 13, 2017, created on Nov. 8, 2017, which is 88,325 bytes in size. The information in the electronic format of the Sequence Listing is incorporated herein by reference in its entirety.

### BACKGROUND

### Field

The present invention relates to combined therapies for the treatment of atherosclerosis, including atherosclerotic cardiovascular disease.

### Description of the Related Art

There are a number and variety of LDL lowering therapies available in cholesterol management that have been developed over the last couple of decades. These compounds, and methods of using these compounds, have been found to be effective at lowering LDL-C levels in various subjects to various levels.

### SUMMARY

In some embodiments, a method of treating coronary atherosclerosis is provided. The method comprises a) identifying a subject that is on a first therapy, wherein the first therapy comprises a non-PCSK9 LDL-C lowering therapy, and b) administering a second therapy to the subject. The second therapy comprises a PCSK9 inhibitor therapy. Both the first and second therapies are administered to the subject in an amount and time sufficient to reverse coronary atherosclerosis in the subject, and the first therapy is not the same as the second therapy.

In some embodiments, the first therapy is selected from at least one of: a statin, including but not limited to atorvastatin (LIPITOR®), cerivastatin, fluvastatin (LESCOL), lovastatin (MEVACOR, ALTOPREV), mevastatin, pitavastatin, pravastatin (PRAVACHOL), rosuvastatin, rosuvastatin calcium (CRESTOR) and simvastatin (ZOCOR); ADVICOR (lovastatin+niacin), CADUET (atorvastatin+amlopidine); a selective cholesterol absorption inhibitor, including but not limited to ezetimibe (ZETIA); a Lipid Lowering Therapy (LLT) including but not limited to fibrates or fibric acid derivatives, including but not limited to gemfibrozil (LOPID), fenofibrate (ANTARA, LOFIBRA, TRICOR, TRIGLIDE) and clofibrate (ATROMID-S); a Resin including but not limited to cholestyramine (QUESTRAN, QUESTRAN LIGHT, PREVALITE, LOCHOLEST, LOCHOLEST LIGHT), cholestipol (CHOLESTID) and cholesevelan HCl (WELCHOL) and/or a combination thereof, including but not limited to VYTORIN (simvastatin+ezetimibe).

In some embodiments, a method of treating coronary atherosclerosis is provided. The method comprises a) identifying a subject that has a LDL-C level of less than 70 mg/dL, and b) administering an anti-PCSK9 neutralizing antibody to the subject, in an amount sufficient and time sufficient to lower the LDL-C level to less than 60 mg/dL.

In some embodiments, a method of decreasing percent atheroma volume (PAV) in a subject is provided. The method comprises a) identifying a subject that has received at least a moderate level of treatment by a statin, and b) administering an anti-PCSK9 neutralizing antibody to the subject in an amount sufficient and time sufficient to lower the LDL-C level to less than 100, e.g., less than 90 mg/dL, thereby decreasing a percent atheroma volume (PAV) in the subject.

In some embodiments, a method of decreasing total atheroma volume (TAV) in a subject is provided. The method comprises a) identifying a subject that has received at least a moderate level of treatment by a statin, and b) administering an anti-PCSK9 neutralizing antibody to the subject in an amount sufficient and time sufficient to lower the LDL-C level to less than 100, e.g., less than 90 mg/dL, thereby decreasing a total atheroma volume in the subject.

In some embodiments, a method of treating coronary atherosclerosis is provided. The method comprises a) administering an optimum statin treatment to a subject, wherein the subject has coronary atherosclerosis, and b) administering an amount of an anti-PCSK9 neutralizing antibody to the subject at the same time.

In some embodiments, a method of treating coronary atherosclerosis is provided. The method comprises a) identifying a statin-intolerant subject, b) administering at least a low dose statin treatment to the statin-intolerant subject, and c) administering an amount of an anti-PCSK9 neutralizing antibody to the subject, thereby treating coronary atherosclerosis.

In some embodiments, a method of providing regression of coronary atherosclerosis is provided, the method comprises providing a subject that is on an optimized level of a statin, and administering to the subject an anti-PCSK9 neutralizing antibody, at a level adequate to regress coronary atherosclerosis, wherein regression is any change in PAV or TAV less than zero.

In some embodiments, a method of decreasing a LDL-C level in a subject beneath 80 mg/dL is provided. The method comprises administering an anti-PCSK9 neutralizing antibody to a subject. The subject has coronary atherosclerotic disease. The subject is on an optimized statin therapy for at least one year, and a LDL-C level in the subject decreases to an average value that is beneath 80 mg/dL for the at least one year.

In some embodiments, a method of reducing a relative risk of a cardiovascular event by at least 10% is provided. The method comprises administering a PCSK9 neutralizing antibody to a subject that is on at least a moderate intensity of a statin, in an amount sufficient to lower a LDL-C level of the subject by about 20 mg/dL.

In some embodiments, a method of reducing an amount of atherosclerotic plaque in a subject is provided. The method comprises administering to a subject having atherosclerotic plaque a monoclonal antibody to human PCSK9. The subject is also receiving optimized statin therapy, and the combination therapy thereby reduces the amount of atherosclerotic plaque in the subject.

In some embodiments, a method of reducing disease progression is provided. The method comprises identifying a subject with a LDL-C level of no more than 60 mg/dL, administering at least a moderate intensity of a statin therapy to the subject, and administering evolocumab at a level sufficient to decrease the LDL-C level of the subject to 30 mg/dL, thereby reducing disease progression.

In some embodiments, a method of combining evolocumab and a statin therapy to produce greater LDL-C lowering and regression of coronary atherosclerosis at a dose that is well tolerated is provided. The method comprises administering at least a moderate intensity of a statin therapy to a subject, administering an adequate amount of evolocumab to the subject such that the subject's LDL-C levels drop to no more than 40 mg/dL, and maintaining the subject's LDL-C levels at no more than 40 mg/dL for at least one year.

In some embodiments, a method of treating coronary atherosclerosis is provided. The method comprises a) identifying a subject that has a LDL-C level of less than 70 mg/dL, and b) administering a PCSK9 inhibitor to the subject, in an amount sufficient and time sufficient to lower the LDL-C level to less than 60 mg/dL.

In some embodiments, a method of decreasing percent atheroma volume (PAV) in a subject is provided. The method comprises a) identifying a subject that has received at least a moderate level of treatment by a non-PCSK9 LDL-C lowering agent, and b) administering a PCSK9 inhibitor to the subject in an amount sufficient and time sufficient to lower the LDL-C level to less than 100 mg/dL, e.g., less than 90 mg/dL, thereby decreasing a percent atheroma volume (PAV) in the subject.

In some embodiments, a method of decreasing total atheroma volume (TAV) in a subject is provided. The method comprises a) identifying a subject that has received at least a moderate level of treatment by a non-PCSK9 LDL-C lowering agent and b) administering a PCSK9 inhibitor to the subject in an amount sufficient and time sufficient to lower the LDL-C level to less than 100 mg/dL, e.g., less than 90 mg/dL, thereby decreasing a total atheroma volume in the subject.

In some embodiments, a method of treating coronary atherosclerosis is provided. The method comprises a) administering an optimum non-PCSK9 LDL-C lowering therapy to a subject, wherein the subject has coronary atherosclerosis, and b) administering an amount of a PCSK9 inhibitor to the subject at the same time.

In some embodiments, a method of treating coronary atherosclerosis is provided. The method comprises a) identifying a statin-intolerant subject, b) administering a low intensity statin treatment or no statin treatment to the statin-intolerant subject, and c) administering an amount of a PCSK9 inhibitor to the subject, thereby treating coronary atherosclerosis.

In some embodiments, a method of providing regression of coronary atherosclerosis is provided. The method comprises providing a subject that is on an optimized level of a non-PCSK9 LDL-C lowering agent and administering to the subject a PCSK9 inhibitor, at a level adequate to regress coronary atherosclerosis. Regression is any change in PAV or TAV less than zero.

In some embodiments, a method of decreasing a LDL-C level in a subject beneath 80 mg/dL is provided. The method comprises administering a PCSK9 inhibitor to a subject. The subject has coronary atherosclerotic disease. The subject is on an optimized non-PCSK9 LDL-C lowering therapy for at least one year. A LDL-C level in the subject decreases to an average value that is beneath 80 mg/dL for the at least one year.

In some embodiments, a method of reducing an amount of atherosclerotic plaque in a subject is provided. The method comprises administering to a subject having atherosclerotic plaque a PCSK9 inhibitor. The subject is receiving optimized non-PCSK9 LDL-C lowering therapy, thereby reducing the amount of atherosclerotic plaque in the subject.

In some embodiments, a method of reducing disease progression is provided. The method comprises identifying a subject with a LDL-C level of no more than 60 mg/dL, administering at least a moderate intensity of a non-PCSK9 LDL-C lowering therapy to the subject, and administering a PCSK9 inhibitor at a level sufficient to decrease the LDL-C level of the subject to 30 mg/dL, thereby reducing disease progression.

In some embodiments, a method of combining a PCSK9 inhibitor therapy and a non-PCSK9 LDL-C lowering therapy to produce greater LDL-C lowering and regression of coronary atherosclerosis at a dose that is well tolerated is provided. The method comprises administering at least a moderate intensity of a non-PCSK9 LDL-C lowering therapy to a subject, administering an adequate amount of a PCSK9 inhibitor to the subject such that the subject's LDL-C levels drop to no more than 40 mg/dL, and maintaining the subject's LDL-C levels at no more than 40 mg/dL for at least one year.

In some embodiments, a method of treating a subject that is unable to tolerate a full therapeutic dose of a non-PCSK9 LDL-C lowering agent is provided. The method comprises identifying said subject and administering a PCSK9 inhibitor to the subject until a LDL cholesterol level of the subject decreases beneath 60 mg/dL.

In some embodiments, a method of treating a subject that is unable to tolerate a full therapeutic dose of a statin is provided. The method comprises identifying said subject and administering a PCSK9 inhibitor to the subject until a LDL cholesterol level of the subject decreases beneath 60 mg/dL.

In some embodiments, a method of treating coronary atherosclerosis is provided. The method comprises a) identifying a subject that has a LDL-C level of less than 70 mg/dL and b) administering a non-PCSK9 LDL-C lowering agent to the subject, in an amount sufficient and time sufficient to lower the LDL-C level to less than 60 mg/dL.

In some embodiments, a method of treating atherosclerotic cardiovascular disease is provided. The method comprises a) identifying a subject that is on a first therapy, wherein the first therapy comprises a non-PCSK9 LDL-C lowering therapy, and b) administering a second therapy to the subject. The second therapy comprises a PCSK9 inhibitor therapy, wherein both the first and second therapies are administered to the subject in an amount and time sufficient to reduce a risk of atherosclerotic cardiovascular disease in the subject. The first therapy is not the same as the second therapy. The risk is a) a composite for cardiovascular death, myocardial infarction, stroke, hospitalization for unstable angina, or coronary revascularization or b) a composite for cardiovascular death, myocardial infarction, or stroke, or c) cardiovascular death, or d) fatal and/or non-fatal MI, or e) fatal and/or non-fatal stroke, or f) transient ischemic attack, or g) hospitalization for unstable angina, or h) elective, urgent, and/or emergent coronary revascularization.

In some embodiments, a method of reducing a risk of a cardiovascular event is provided. The method comprises a) identifying a subject that is on a first therapy, wherein the first therapy comprises a non-PCSK9 LDL-C lowering therapy, and b) administering a second therapy to the subject. The second therapy comprises a PCSK9 inhibitor, wherein both the first and second therapies are administered to the subject in an amount and time sufficient to reduce a risk of a cardiovascular event in the subject. The first therapy is not the same as the second therapy. The risk is a) a composite for cardiovascular death, myocardial infarction, stroke, hospitalization for unstable angina, or coronary revascularization or b) a composite for cardiovascular death, myocardial infarction, or stroke, or c) cardiovascular death, or d) fatal and/or non-fatal MI, or e) fatal and/or non-fatal stroke, or f) transient ischemic attack, or g) hospitalization for unstable angina, or h) elective, urgent, and/or emergent coronary revascularization.

In some embodiments, a method of reducing a risk of urgent coronary revascularization is provided. The method comprises a) identifying a subject that is on a first therapy, wherein the first therapy comprises a non-PCSK9 LDL-C lowering therapy, and b) administering a second therapy to the subject. The second therapy comprises a PCSK9 inhibitor therapy. Both the first and second therapies are administered to the subject in an amount and time sufficient to reduce the risk of atherosclerotic cardiovascular disease in the subject, and wherein the first therapy is not the same as the second therapy.

In some embodiments, a method of reducing a risk of a cardiovascular event is provided. The method comprises a) identifying a subject with cardiovascular disease, and b) administering a PCSK9 inhibitor to the subject in an amount and over time sufficient to reduce a risk of at least one of cardiovascular death, non-fatal myocardial infarction, non-fatal stroke or transient ischemic attack (TIA), coronary revascularization, or hospitalization for unstable angina.

In some embodiments, a method of lowering LDL-C levels in a subject is provided. The method comprising administering: a) a first therapy to a subject, wherein the first therapy comprises a non-PCSK9 LDL-C lowering therapy, and b) administering a second therapy to the subject, wherein the second therapy comprises a PCSK9 inhibitor. Both the first and second therapies are administered to the subject for at least five years, and the first therapy is not the same as the second therapy, and wherein the subject's LDL-C level is maintained beneath 50 mg/dL.

In some embodiments, a method of reducing a risk of a cardiovascular event is provided. The method comprises a) identifying a subject that is on a first therapy, the first therapy comprises a non-PCSK9 LDL-C lowering therapy. The method further comprises b) administering a second therapy to the subject. The second therapy comprises a PCSK9 inhibitor. Both the first and second therapies are administered to the subject in an amount and time sufficient to reduce a risk of a cardiovascular event in the subject. The first therapy is not the same as the second therapy. The risk is at least one of cardiovascular death, myocardial infarction, stroke, hospitalization for unstable angina, or coronary revascularization.

In some embodiments, a method of treating a subject is provided. The method comprises identifying a subject with peripheral artery disease (“PAD”) and reducing a level of PCSK9 activity in the subject.

In some embodiments, a method of reducing a risk of an adverse limb event in a subject is provided, the method comprises reducing a level of PCSK9 activity in a subject, wherein the subject has PAD.

In some embodiments, a method of reducing a risk of a major cardiovascular adverse event (“MACE”) is provided. The method comprises administering a non-statin LDL-C lowering agent to a subject and administering a statin to the subject. The subject has PAD. In some embodiments, a method of reducing a risk of PAD and/or CAD and/or cerebrovascular disease is provided. The method comprises administering a non-statin LDL-C lowering agent to a subject and administering a statin to the subject.

In some embodiments, a method of reducing a risk of a major adverse limb event (“MALE”) is provided. The method comprises administering a non-statin LDL-C lowering agent to a subject and administering a statin to the subject. The subject has PAD.

In some embodiments, a method of reducing a risk of a cardiovascular event is provided. The method comprises providing a first therapy to a subject. The first therapy comprises a non-PCSK9 LDL-C lowering therapy. The method further comprises providing a second therapy to the subject. The second therapy comprises a PCSK9 inhibitor. The first and second therapies are administered to the subject, and wherein the subject has a Lp(a) level of 11.8 mg/dL to 50.

In some embodiments, a method of reducing a risk of a major vascular event in a subject is provided. The method comprises 1) identifying a subject that has at least one of: (a) a recent MI, (b) multiple prior MIs, or (c) multivessel disease. The method further comprises 2) providing a first therapy to a subject, wherein the first therapy comprises a non-PCSK9 LDL-C lowering therapy. The method further comprises 3) providing a second therapy to the subject, wherein the second therapy comprises a PCSK9 inhibitor, thereby reducing a risk that the subject will have a major vascular event.

In some embodiments, a method of treating coronary atherosclerosis is provided that comprises administering, to a subject who has a LDL-C level of greater than 70 mg/dL a PCSK9 inhibitor in an amount sufficient and over a time period sufficient to lower the LDL-C level to less than 40 mg/dL. In some embodiments, a method of reducing a risk of a cardiovascular event is provided that comprises administering, to a subject who has a LDL-C level of greater than 70 mg/dL, a PCSK9 inhibitor in an amount sufficient and over a time period sufficient to lower the LDL-C level to less than 40 mg/dL.

### BRIEF DESCRIPTION OF THE DRAWINGS

FIG. 1 depicts the disposition of the patients during the GLAGOV study.

FIG. 2 depicts the mean (±standard error) percent change in LDL-C in patients treated with placebo (circles) and evolocumab (triangles) during the study.

FIG. 3 depicts the prespecified subgroup analysis of the primary end point, the change in percent atheroma volume (PAV) from baseline to 78-week follow-up. Results are expressed as least square mean±standard error LDL-C, low-density lipoprotein cholesterol; non-HDL-C, non-high-density lipoprotein cholesterol; PCSK9, proprotein convertase subtilisin kexin type 9; TAV, total atheroma volume.

FIG. 4A depicts the change in percent atheroma volume (PAV, left panel) and percentage of patients demonstrating regression of PAV (right panel) in the placebo (white) and evolocumab (black) treatment groups, stratified according to baseline LDL-C.

FIG. 4B depicts the change in total atheroma volume (TAV, left panel) and percentage of patients demonstrating regression of TAV (right panel) in the placebo (white) and evolocumab (black) treatment groups, stratified according to baseline LDL-C.

FIG. 4C depicts the data from an exploratory subgroup of subjects having a baseline LDL-C <70 mg/dL.

FIG. 4D depicts the data from an exploratory subgroup have a baseline LDL-C of <70 mg/dL,

FIG. 5 depicts the local regression (LOESS) curve illustrating the association (with 95% confidence intervals) between achieved LDL-C levels and the change in percent atheroma volume in all patients undergoing serial IVUS evaluation.

FIG. 6 depicts some sequence aspects of some embodiments of PCSK9 inhibitors. The highlighted regions denote the variable regions.

FIG. 7 depicts some sequence aspects of some embodiments of PCSK9 inhibitors. The highlighted regions denote the variable regions.

FIG. 8 depicts some sequence aspects of some embodiments of PCSK9 inhibitors (FIG. 8 is related to FIG. 10).

FIG. 9 depicts some sequence aspects of some embodiments of PCSK9 inhibitors (FIG. 9 is related to FIG. 11).

FIG. 10 depicts some sequence aspects of some embodiments of PCSK9 inhibitors (FIG. 10 is related to FIG. 8).

FIG. 11 depicts some sequence aspects of some embodiments of PCSK9 inhibitors (FIG. 11 is related to FIG. 9).

FIG. 12 depicts some sequence aspects of some embodiments of PCSK9 inhibitors.

FIG. 13 depicts some constant domain sequence aspects of some embodiments of PCSK9 inhibitors.

FIG. 14A depicts an amino acid sequence of the mature form of PCSK9 with the pro-domain underlined.

FIGS. 14B1-14B4 depict the amino acid and nucleic acid sequences of PCSK9 with the pro-domain underlined and the signal sequence in bold.

FIG. 15 is a graph depicting LDL cholesterol levels over time.

FIGS. 16A and 16B are graphs depicting the cumulative incidence of cardiovascular events. Shown are the cumulative event rates for the primary end point (the composite of cardiovascular death, myocardial infarction, stroke, hospitalization for unstable angina, or coronary revascularization; FIG. 16A) and the key secondary efficacy end point (the composite of cardiovascular death, myocardial infarction, or stroke; FIG. 16B).

FIG. 17 is a trial consort diagram for FOURIER.

FIG. 18 is a graph depicting LDL cholesterol values over time. Data are in fixed cohort of 11077 patients who had all measurements through 120 weeks, did not discontinue study drug, and did not change concomitant background lipid lowering therapy. Shown are median values with 95% confidence intervals in the two arms. To convert the values for cholesterol to millimoles per liter, multiply by 0.02586.

FIG. 19 is a series of graphs displaying various lipid parameters. Displayed are mean changes at 48 weeks except for triglycerides and Lp(a), which are median changes. Errors bars denote 95% CI.

FIG. 20 is two graphs showing the landmark analyses for the primary endpoint.

FIG. 21 depicts two graphs showing the landmark analyses for the secondary endpoint.

FIG. 22 depicts the efficacy in various subgroups.

FIG. 23 depicts the hazard ratio (95% CI) per 1 mmol/L reduction in LDL-C.

FIG. 24a is a graph depicting the primary composite endpoint (cardiovascular death, myocardial infarction, stroke, unstable angina, coronary revascularization) by treatment (evolocumab in dark, placebo in lighter) in patients with (solid lines) and without (dashed lines) symptomatic PAD.

FIG. 24b is a graph depicting the key secondary composite endpoint (cardiovascular death, myocardial infarction, stroke) by treatment (evolocumab in dark, placebo in lighter) in patients with and without symptomatic PAD.

FIG. 25a is a graph depicting the major adverse limb events (composite of acute limb ischemia, major amputation or urgent revascularization) by treatment (evolocumab in dark, placebo in lighter) in all randomized patients.

FIG. 25b is a graph depicting the major adverse limb events (composite of acute limb ischemia, major amputation or urgent revascularization) by treatment (evolocumab in dark, placebo in lighter) in patients with symptomatic PAD.

FIG. 26 is a graph depicting the composite of major adverse cardiovascular events (MACE; cardiovascular death, myocardial infarction or stroke) and major adverse limb events (MALE; acute limb ischemia, major amputation or urgent revascularization) by treatment (evolocumab in dark, placebo in lighter) in patients with and without symptomatic PAD.

FIG. 27 is a graph depicting the relationship between achieved LDL-C and major adverse limb events (MALE; acute limb ischemia, major amputation or urgent revascularization).

FIG. 28 are graphs displaying cardiovascular outcomes at 2.5 years in placebo patients by symptomatic PAD at baseline.

FIG. 29 is a graph depicting CV death, MI, or stroke at 2.5 years in a placebo patient by disease state.

FIG. 30 is a graph depicting cardiovascular outcomes at 2.5 years in placebo patients by symptomatic PAD and no MI/stroke at baseline.

FIG. 31 is a graph depicting limb outcomes at 2.5 years in placebo patients by symptomatic PAD and no MI or stroke at baseline.

FIG. 32 is a graph depicting LDL cholesterol by treatment group in patients with symptomatic lower extremity PAD.

FIG. 33A is a graph depicting the primary endpoint in patients with PAD and no MI or stroke.

FIG. 33B is a graph depicting CV death, MI, or stroke in patients with PAD and no MI or stroke.

FIG. 33C is a graph depicting major adverse limb events in patients with PAD and no MI or stroke.

FIG. 34 is a graph depicting MACE or MALE in patients with PAD and no MI or stroke.

FIG. 35 is a graph depicting achieved LDL-C and MACE or MALE in patients with PAD.

FIG. 36 is a graph depicting achieved LDL-C and MACE or MALE in patients with PAD and no MI or stroke.

FIG. 37 depicts a GLAGOV trial schematic.

FIG. 38 depicts a cross-sectional lumen and formula for determining percent atheroma volume.

FIG. 39 depicts graph showing plaque progression and percent atheroma volume as a function of the number of risk factors present.

FIG. 40 depicts a FOURIER trial design.

FIG. 41 depicts graphs depicting the primary results for the FOURIER trial for placebo vs evolucmab.

FIG. 42 is a graph depicting the risk of CVD, MI or stroke based on time from MI.

FIG. 43 is a graph depicting the risk of CVD, MI, or stroke based on the number of prior MIs.

FIG. 44 is a graph depicting the risk of CVD, MI, or stroke based on the presence of multivessel disease.

FIG. 45 are graphs depicting the risk of CVD, MI, or stroke based on time from prior MI.

FIG. 46 are graphs depicting the risk of CVD, MI, or stroke based on time from prior MI and number of prior MIs.

FIG. 47 are graphs depicting the risk of CVD, MI, or stroke based on time from prior MI and presence of multivessel disease.

FIG. 48 is a graph depicting the benefit of evolocumab therapy in subjects with no risk features.

FIG. 49 is a graph depicting the benefit of evolocumab therapy in subjects with 1 or more risk feature.

FIG. 50 is a graph depicting the benefit of evolocumab therapy (for CVD, MI or stroke) in subjects with high-risk MI features.

FIG. 51 is a graph depicting the benefit of evolocumab therapy (for CVD, MI or stroke) in subjects with high-risk MI features.

FIG. 52 is a graph depicting the three year KM rate of CV death, MI or stroke for low, intermediate, or high TIMI risk score.

FIG. 53 is a set of graphs depicting the total primary endpoints prevented (a) and the primary endpoint events using Wei, Lin Weissfeld model.

FIGS. 54A and 54B are set of graphs depicting the types 54A and sizes MB of MI reduced with evolocumab in FOURIER.

FIG. 55 is a graph depicting the adjusted event rate by average postbaseline non-HDL-C up to time-to-event endpoint.

### DETAILED DESCRIPTION OF THE PREFERRED EMBODIMENT

Statins can be used for managing patients with clinically manifest coronary heart disease^{23,24}. However, many patients are not able to achieve optimal LDL-C lowering²⁵ or experience cardiovascular events despite statin therapy.²⁶ Furthermore, some patients report inability to tolerate full therapeutic doses of statins.²⁷ Inadequate LDL-C reduction and high residual risk suggests that additional therapies are required to deliver more effective cardiovascular prevention. Elucidating the role of PCSK9 in regulation of hepatic LDL receptor expression has provided an attractive target for therapeutic modulation. The fact that PCSK9 levels rise in response to statin administration further supports the therapeutic potential of PCSK9 inhibitors to reduce residual cardiovascular risk in statin-treated patients.²⁸

Provided herein are results from a clinical trial (reported in Example 1), in which patients treated with a non-PCSK9 LDL-C lowering agent (e.g., a statin) and a PCSK9 inhibitor (e.g., evolocumab)(or in a some cases, a PCSK9 inhibitor alone), received benefits on LDL-C, atheroma volume and atheroma regression that was additional to the benefit from statin treatment alone.

The presently disclosed trial results (Example 1) provided an opportunity to evaluate the impact of a PCSK9 inhibitor in a number of settings. By studying the effect of a PCSK9 inhibitor on atheroma volume, it provided the first evaluation of PCSK9 inhibition on an efficacy endpoint beyond LDL-C (and/or other lipids, such as ApoB, Lp(a), etc.), providing evidence that LDL-C lowering (and/or other lipids) affects disease activity within the vessel wall. Interestingly, the benefits were observed at a LDL-C level well below that typically encountered in studies of moderate or high-intensity statin monotherapy and represents the first evidence of efficacy in patients who were predominantly treated with either moderate or high-intensity statin therapy.

In light of the presently disclosed study, provided herein are one or more “combined therapies” for the treatment of atherosclerosis (including, e.g., coronary artery disease (CAD)). The “combined therapies” or “combination therapies” combine at least two different therapies so as to achieve a very low LDL-C level such that the subject receiving both therapies will have a reduced risk of atherosclerosis (e.g., CAD and/or PAD and/or cerebrovascular disease). As outlined in more detail below, while, individually, each of the two types of therapy to be combined has been known before, their combination, to provide the very low level of LDL-C lowering benefit, which in turn provides for the treatment of atherosclerosis, has not been demonstrated previously. While there are a variety of possible combinations of therapies for the “combined therapy” approach provided herein, the term denotes a first therapy that can be any non-PCSK9 directed therapy (e.g., a statin) that lowers LDL-C levels, and a second therapy that can be a PCSK9 specific treatment (a PCSK9 inhibitor, for example, a neutralizing antibody to PCSK9 and/or antisense RNA to PCSK9). Not only are these two therapies to be combined, but in some embodiments, the level of the therapies are set such that LDL-C levels can be decreased well below other typical goals attempted for cholesterol lowering therapies (to achieve a very low level of LDL-C), and maintained for a duration adequate for addressing atherosclerosis, including coronary artery disease. Furthermore, as detailed herein, given the value of such low levels of LDL-C in a subject, other, non-combined therapies are also provided herein. Such single therapies do not need to employ a second agent to lower LDL-C levels to the extremely low and highly beneficial levels (such as less than 50, 40, 30, or 20 mg/dL of LDL-C), and can employ a single agent, such as a PCSK9 neutralizing antibody, such as evolocumab. Such a statin-free therapy can be especially useful in situations where the subject is intolerant to statins. In other embodiments, the subject is not intolerant to statins, but a single therapy is used regardless.

Interestingly, the findings presented herein contradict the results and assumptions made in previous studies, such as ASTEROID, from which it was hypothesized that lowering LDL-C below 60.8 mg/dL may not have any regression benefit. In contrast to the findings from ASTEROID, the results presented herein show that regression does not plateau at 60 mg/dL. Instead, the results in Example 1 show that one can obtain surprisingly beneficial regression of atherosclerosis by lowering LDL-C lower than 60 mg/dL. Indeed, the results demonstrate a benefit from achieving LDL-C levels beneath 60 mg/dL, down to levels as low as 25 mg/dL and 20 mg/dL.

In addition, the present disclosure also provides the results and discoveries of the FOURIER study (e.g., Example 17). These finding demonstrate the effectiveness of combined therapies (such as evolocumab on cardiovascular outcomes when combined with a non-PCSK9 therapy (such as a statin)) in subjects with atherosclerotic cardiovascular disease.

The following section provides a brief set of definitions for the present disclosure, followed by a detailed description of various particular embodiments and aspects, followed by a set of examples.

### Definitions and Embodiments

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

“Combined therapies,” or “combination therapies,” as the terms are used herein, are meant to denote a first therapy that can be any non-PCSK9 LDL-C lowering therapy that lowers LDL-C levels (using for example, a statin), and a second therapy that can be a PCSK9 inhibitor therapy (using, for example, a neutralizing antibody to PCSK9 and/or antisense RNA to PCSK9). The combined therapy will employ a non-PCSK9 LDL-C lowering agent and a PCSK9 inhibitor agent. The combined therapies can also have their benefit from lowering other non-LDL cholesterol particles as well. Such embodiments can also be explicitly called out as “non-PCSK9 lipid lowering therapies”.

The term “regression” or “reversal” denotes that one or more of the symptoms and/or aspects of the disorder has been reversed. “Regression” can be defined as any decrease in PAV or TAV from baseline.

The term “very low LDL-C levels” denotes LDL-C levels beneath 40 mg/dL. In some embodiments, very low encompasses 25 mg/dL or lower.

“PCSK9 inhibitor” denotes a molecule or therapy that inhibits PCSK9 activity to thereby lower LDL-C (and/or other lipids, such as non-HDL-C, ApoB, Lp(a), etc.) levels. This can include neutralizing antibodies to PCSK9 and anti-sense molecules to PCSK9, for example. A PCSK9 inhibitor therapy denotes a method that uses a PCSK9 inhibitor agent.

“A non-PCSK9 LDL-C lowering agent” denotes a molecule that lowers LDL-C levels through a pathway other than through PCSK9. A non-PCSK9 LDL-C lowering therapy denotes a method that employs a non-PCSK9 LDL-C lowering agent. Examples of non-PCSK9 LDL-C lowering agents include statins (aka HMG CoA reductase inhibitors), atorvastatin (LIPITOR®), cerivastatin, fluvastatin (LESCOL), lovastatin (MEVACOR, ALTOPREV), mevastatin, pitavastatin, pravastatin (PRAVACHOL), rosuvastatin, rosuvastatin calcium (CRESTOR) and simvastatin (ZOCOR), ADVICOR (lovastatin+niacin), CADUET (atorvastatin+amlopidine); selective cholesterol absorption inhibitors, ezetimibe (ZETIA); a Lipid Lowering Therapy (LLT) fibrates or fibric acid derivatives, including gemfibrozil (LOPID), fenofibrate (ANTARA, LOFIBRA, TRICOR, TRIGLIDE) and clofibrate (ATROMID-S); a Resin (aka bile acid sequestrant or bile acid-binding drugs), cholestyramine (QUESTRAN, QUESTRAN LIGHT, PREVALITE, LOCHOLEST, LOCHOLEST LIGHT), cholestipol (CHOLESTID) and cholesevelan HCl (WELCHOL) and/or a combination thereof, including but not limited to VYTORIN (simvastatin+ezetimibe). The term “non-PCSK9 LDL-C lowering agent” encompasses agents that do more than just reduce LDL-C. In some embodiments, the methods involving “non-PCSK9 LDL-C lowering agents” provided herein can instead be practiced with a “non-PCSK9 lipid lowering agent”, which is an agent that lowers the lipid in a subject, without specifically lowering LDL-C.

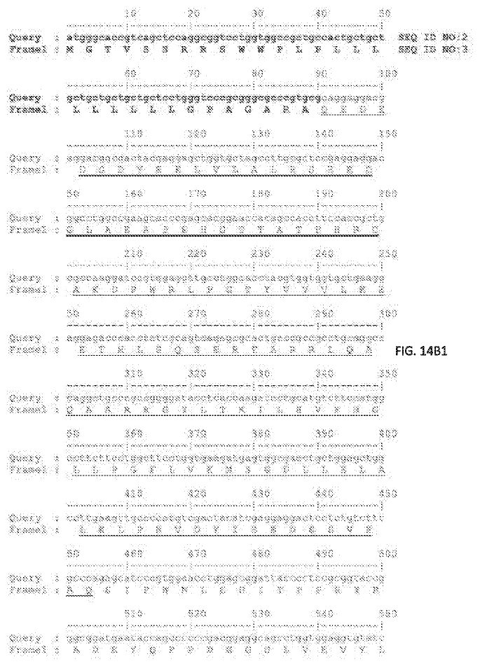

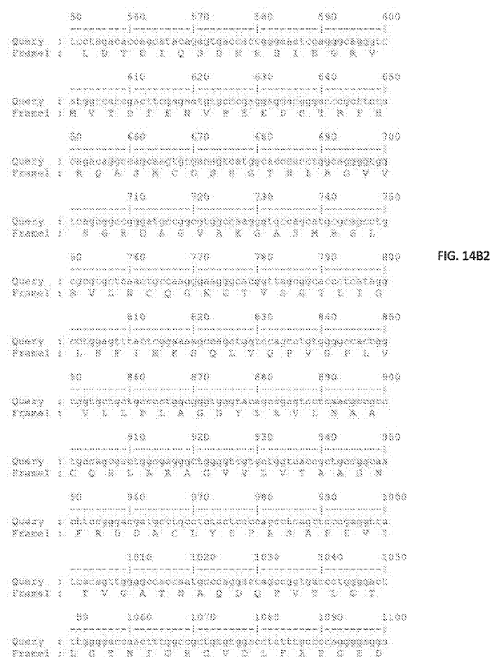

The term “proprotein convertase subtilisin kexin type 9” or “PCSK9” refers to a polypeptide as set forth in SEQ ID NO: 1 and/or 3 in FIGS. 14A, 14B1-B4. “PCSK9” has also been referred to as FH3, NARC1, HCHOLA3, proprotein convertase subtilisin/kexin type 9, and neural apoptosis regulated convertase 1. The PCSK9 gene encodes a proprotein convertase protein that belongs to the proteinase K subfamily of the secretory subtilase family. The term “PCSK9” denotes both the proprotein and the product generated following autocatalysis of the proprotein. When only the autocatalyzed product is being referred to (such as for an antibody that selectively binds to the cleaved PCSK9), the protein can be referred to as the “mature,” “cleaved”, “processed” or “active” PCSK9. When only the inactive form is being referred to, the protein can be referred to as the “inactive”, “pro-form”, or “unprocessed” form of PCSK9.

The term “PCSK9 activity” includes the ability of PCSK9 to reduce the availability of LDLR and/or the ability of PCSK9 to increase the amount of LDL in a subject.

The term “isolated protein” means that a subject protein (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

An antibody is said to “specifically bind” its target antigen when the dissociation constant (K_{d}) is ≤10⁻⁷ M. The antibody specifically binds antigen with “high affinity” when the K_{d} is ≤5×10⁻⁹ M, and with “very high affinity” when the K_{d} is ≤5×10⁻¹⁰ M. In one embodiment, the antibody has a K_{d} of ≤10⁻⁹ M. In one embodiment, the off-rate is <1×10⁻⁵. In other embodiments, the antibodies will bind to human PCSK9 with a K_{d} of between about 10⁻⁹ M and 10⁻¹³ M, and in yet another embodiment the antibodies will bind with a K_{d}≤5×10⁻¹⁰. As will be appreciated by one of skill in the art, in some embodiments, any or all of the antibodies can specifically bind to PCSK9.

An antibody is “selective” when it binds to one target more tightly than it binds to a second target.

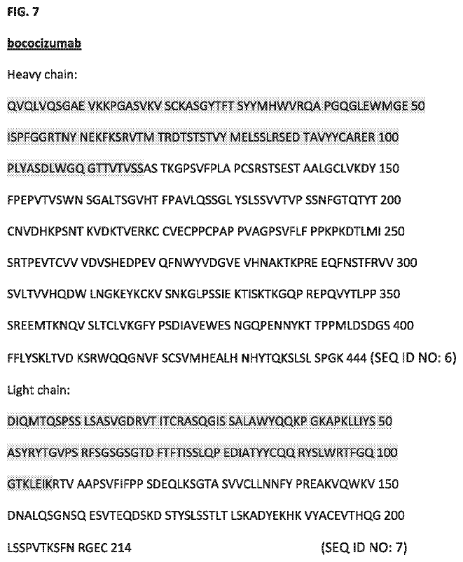

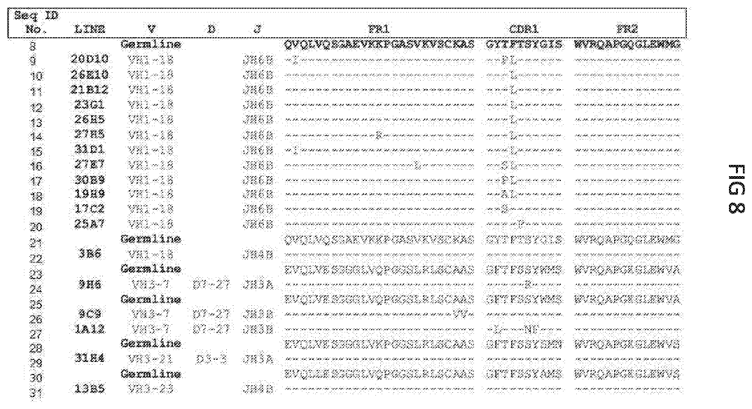

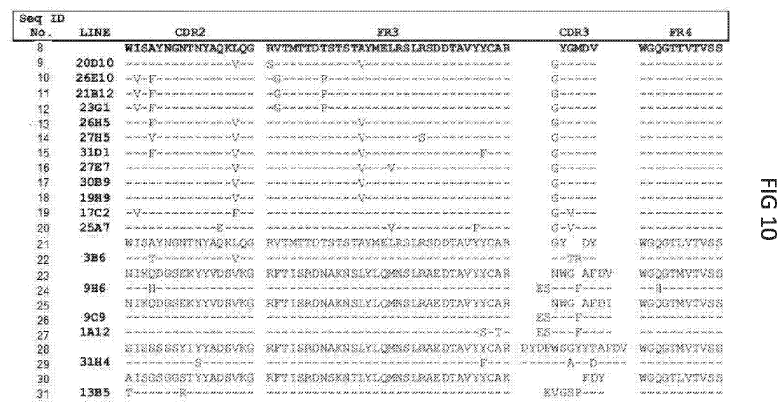

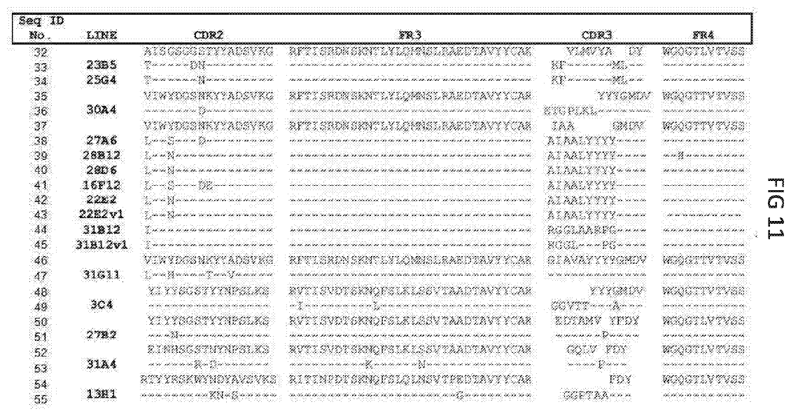

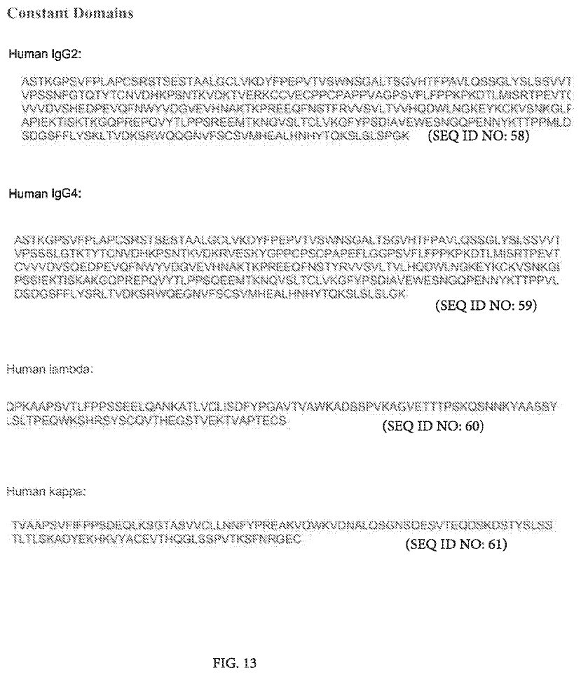

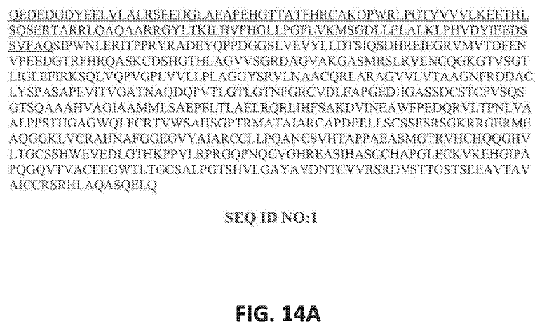

The term “antibody” refers to an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Furthermore, unless explicitly excluded, antibodies include, for example, monoclonal antibodies, polyclonal antibodies, chimeric antibodies, humanized antibodies, human antibodies, bispecific antibodies, and synthetic antibodies. In some sections of the present disclosure, examples of antibodies are described herein in terms of the hybridoma line number as “number/letter/number” (e.g., 21B12). In these cases, the exact name denotes a specific monoclonal antibody derived from a specific hybridoma having a specific light chain variable region and heavy chain variable region. In some embodiments, the antibody can include one or more of the sequences in FIG. 6-13.

Typical antibody structural units comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk, J. Mol. Biol., 196:901-917 (1987); Chothia et al., Nature, 342:878-883 (1989).

In some embodiments, instead of a full length antibody, a “fragment” or “antigen binding fragment” of an antibody is provided. As used herein and unless otherwise specified, an “antibody fragment” refers to the Fab, Fab′, F(ab′)2, and Fv fragments that contain at least one CDR of an immunoglobulin that is sufficient to confer specific antigen binding to the target protein, such as PCSK9. Antibody fragments may be produced by recombinant DNA techniques or by enzymatic or chemical cleavage of intact antibodies.

In some embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition, the AHo definition, and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The AHo definition is a residue numbering scheme based on spatial alignment of known three-dimensional structures of immunoglobulin domains (See, e.g., Honegger and Plueckthun, J. Mol. Biol., 309:657-670, (2001). The contact definition is based on an analysis of the available complex crystal structures. See, e.g., MacCallum et al., J. Mol. Biol., 5:732-45 (1996).

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, V_{H}, and three constant region domains, C_{H}L C_{H}2, and C_{H}3. The V_{H} domain is at the amino-terminus of the polypeptide, and the C_{H} domains are at the carboxyl-terminus, with the C_{H}3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79: 315-321 (1990); Kostelny et al., J. Immunol., 148:1547-1553 (1992).

Some species of mammals also produce antibodies having only a single heavy chain.

Each individual immunoglobulin chain is typically composed of several “immunoglobulin domains,” each consisting of roughly 90 to 110 amino acids and having a characteristic folding pattern. These domains are the basic units of which antibody polypeptides are composed. In humans, the IgA and IgD isotypes contain four heavy chains and four light chains; the IgG and IgE isotypes contain two heavy chains and two light chains; and the IgM isotype contains five heavy chains and five light chains. The heavy chain C region typically comprises one or more domains that can be responsible for effector function. The number of heavy chain constant region domains will depend on the isotype. IgG heavy chains, for example, contain three C region domains known as C_{H}1, C_{H}2 and C_{H}3. The antibodies that are provided can have any of these isotypes and subtypes. In certain embodiments of the present invention, an anti-PCSK9 antibody is of the IgG1 or IgG2 or IgG4 subtype.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target

The term “neutralizing antibody” as used in “anti-PCSK9 neutralizing antibody” refers to an antibody that binds to a target and prevents or reduces the biological activity of that target. This can be done, for example, by directly blocking a binding site on the target or by binding to the target and altering the target's ability to bind through indirect means (such as structural or energetic alterations in the target). In assessing the binding and/or specificity of an antibody or immunologically functional fragment thereof, an antibody or fragment can substantially inhibit binding of a target to its binding partner when an excess of antibody reduces the quantity of binding partner bound to the ligand by at least about 1-20, 20-30%, 30-40%, 40-50%, 50-60%, 60-70%, 70-80%, 80-85%, 85-90%, 90-95%, 95-97%, 97-98%, 98-99% or more (as measured in an in vitro competitive binding assay). In the case of PCSK9 antibodies, such a neutralizing molecule can diminish the ability of PCSK9 to bind the LDLR. In some embodiments, the neutralizing ability is characterized and/or described via a competition assay. In some embodiments, the neutralizing ability is described in terms of an IC₅₀ or EC₅₀ value. In some embodiments, the antibodies neutralize by binding to PCSK9 and preventing PCSK9 from binding to LDLR (or reducing the ability of PCSK9 to bind to LDLR). In some embodiments, the antibodies neutralize by binding to PCSK9, and while still allowing PCSK9 to bind to LDLR, preventing or reducing the PCSK9 mediated degradation of LDLR. Thus, in some embodiments, a neutralizing antibody can still permit PCSK9/LDLR binding, but will prevent (or reduce) subsequent PCSK9 involved degradation of LDLR. In some embodiments, neutralizing results in the lowering LDL-C (and/or other lipids, such as non-HDL-C, ApoB, Lp(a), etc.).

An “antigen binding protein” is a protein comprising an antigen binding fragment that binds to an antigen and, optionally, a scaffold or framework portion that allows the antigen binding fragment to adopt a conformation that promotes binding of the antigen binding protein to the antigen. In some embodiments, the antigen is a PCSK9 protein or a fragment thereof. In some embodiments, the antigen binding fragment comprises at least one CDR from an antibody that binds to the antigen, and in some embodiments comprises the heavy chain CDR3 from an antibody that binds to the antigen. In some embodiments, the antigen binding fragment comprises all three CDRs from the heavy chain of an antibody that binds to the antigen or from the light chain of an antibody that binds to the antigen. In still some embodiments, the antigen binding fragment comprises all six CDRs from an antibody that binds to the antigen (three from the heavy chain and three from the light chain). The antigen binding fragment in certain embodiments is an antibody fragment.

The term “compete” when used in the context of antibodies that compete for the same epitope means competition between antibodies as determined by an assay in which the antibodies being tested prevents or inhibits (e.g., reduces) specific binding of a reference antibody (e.g., a ligand, or a reference antibody) to a common antigen (e.g., PCSK9 or a fragment thereof). Numerous types of competitive binding assays can be used to determine if one antibody competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using I-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antibody and a labeled reference antibody. Competitive inhibition is measured by determining the amount of label bound to the solid surface or cells in the presence of the test antibody. Usually the test antibody is present in excess. Antibodies identified by competition assay include antibodies binding to the same epitope as the reference antibody and antibodies binding to an adjacent epitope sufficiently proximal to the epitope bound by the reference antibody for steric hindrance to occur. Additional details regarding methods for determining competitive binding are provided in the examples herein. Usually, when a competing antibody is present in excess, it will inhibit (e.g., reduce) specific binding of a reference antibody to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

As used herein, “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In other embodiments, the object species is purified to essential homogeneity wherein contaminating species cannot be detected in the composition by conventional detection methods and thus the composition consists of a single detectable macromolecular species.

The term “biological sample”, as used herein, includes, but is not limited to, any quantity of a substance from a living thing or formerly living thing. Such living things include, but are not limited to, humans, mice, monkeys, rats, rabbits, and other animals. Such substances include, but are not limited to, blood, serum, urine, cells, organs, tissues, bone, bone marrow, lymph nodes, and skin.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

The term “therapeutically effective amount” refers to the amount of a therapeutic substance or therapeutic substances (e.g., PCSK9 inhibitor; a non-PCSK9 LDL-C lowering agent (such as a statin or other non-PCSK9 LDL-C lowering therapy); and a PCSK9 inhibitor and a non-PCSK9 LDL-C lowering agent). This will be an amount sufficient to produce a therapeutic response in a mammal. Such therapeutically effective amounts are readily ascertained by one of ordinary skill in the art.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. Treatment encompasses regression.

The term “prevent” does not require the 100% elimination of the possibility of an event. Rather, it denotes that the likelihood of the occurrence of the event has been reduced in the presence of the compound or method.

The phrase “percent atheroma volume (PAV),” can be calculated as follows:

$$PAV = \frac{Σ ( {EEM}_{area} - {Lumen}_{area} )}{Σ {EEM}_{area}} \times 100$$

EEMₐᵣₑₐ is the cross-sectional area of the external elastic membrane and Lumenₐᵣₑₐ is the cross-sectional area of the lumen. A change in PAV can be calculated as the PAV at any particular time minus the PAV at baseline.

Normalized “total atheroma volume” (TAV), can be calculated as follows:

$${TAV}_{Normalized} = \frac{Σ ( {EEM}_{area} - {Lumen}_{area} )}{Number of Images in Pullback} \times Median number of images in cohort$$

The average plaque area in each image was multiplied by the median number of images analyzed in the entire cohort to compensate for differences in segment length between subjects. Change in normalized TAV can be calculated as the TAV at any particular time minus the TAV at baseline.

The term “moderate-intensity” non-PCSK9 LDL-C lowering therapy (such as a statin or other non-PCSK9 LDL-C lowering therapy) denotes lowering LDL-C by approximately 30% to <50%.

The term “high-intensity” non-PCSK9 LDL-C lowering therapy (such as a statin or other non-PCSK9 LDL-C lowering therapy) therapy denotes lowering LDL-C by approximately ≥50%.

The term “optimal” or “optimized” or “maximized” or “maximal” non-PCSK9 LDL-C lowering therapy (such as a statin or other non-PCSK9 LDL-C lowering therapy) denote a dose of the non-PCSK9 LDL-C lowering therapy (such as a statin or other non-PCSK9 LDL-C lowering therapy) that has been administered so as to allow the subject to reach their LDL-C lowering goal. When the subject is on at least some amount of a non-PCSK9 LDL-C lowering therapy (such as a statin or other non-PCSK9 LDL-C lowering therapy), the subject can be described as one receiving a non-PCSK9 LDL-C lowering therapy (such as a statin or other non-PCSK9 LDL-C lowering therapy).

In some embodiments, any of the definitions or classifications employed for any of the levels or disorders identified in Example 17 (including the supplement) can be employed in other FOURIER related embodiments or in non-FOURIER embodiments. The placement of those characterizations of disorders etc. at the end of Example 17 is to clarify that these were the definitions employed for the FOURIER study. While such definitions (from Example 17) need not be applied to all embodiments provided herein in all scenarios, it is contemplated that such definitions can be applied to any of the embodiments provided herein, unless designated otherwise or at odds with other definitions. Unless explicitly stated that the definitions as applied in Example 17 are to apply, the various terms will have their plain and ordinary meaning within any claims. Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

Combined Therapies for the Reduction of Atherosclerosis and Improving Cardiovascular Outcomes in Patients with Cardiovascular Disease

Reducing low-density lipoprotein cholesterol (LDL-C) with inhibitors of 3-hydroxy-3-methylglutaryl coenzyme reductase (statins) is common for management for patients with atherosclerosis. Analysis of data within individual statin trials and through meta-analyses suggests a possible consistent relationship between achieving lower LDL-C levels and reduction in major adverse cardiovascular events.^{1,2} In parallel, trials using intravascular ultrasound (IVUS) have studied the effect of statins on coronary atherosclerosis and demonstrated a linear relationship between achieved LDL-C levels and reduction in atheroma burden.³⁻⁶ However, major clinical outcome trials and IVUS studies have explored a range of LDL-C levels, only extending to a mean of approximately 60 mg/dL.^{3,5}

Proprotein convertase subtilisin kexin type-9 (PCSK9) plays a pivotal role in LDL-C metabolism by preventing LDL receptor recycling to the hepatic surface, thereby limiting removal of LDL particles from the circulation.⁷⁻⁹ Monoclonal antibodies against PCSK9 profoundly lower LDL-C as well as other lipids such as non-HDL-C, ApoB and Lp(a), when administered alone or in combination with statins.^{10,11} Initial studies have demonstrated the feasibility of using the combination of statins and PCSK9 inhibitors to achieve much lower LDL-C levels than previously studied.^{10,11} However, no trials to date have explored whether LDL-C lowering with a PCSK9 inhibitor reduces the rate of progression of coronary atherosclerosis and no data exist assessing whether achieving very low LDL-C levels via combination therapy results in incremental benefits in reducing disease progression compared with statins alone.

Presented herein (in Example 1) are the results of the Global Assessment of Plaque Regression with a PCSK9 Antibody as Measured by Intravascular Ultrasound (GLAGOV) trial, which assessed two principal scientific questions: whether PCSK9 inhibition impacts atherosclerosis and/or reduces progression of atherosclerosis and whether achieving very low LDL-C levels with the combination of statins (representative of non-PCSK9 LDL-C lowering therapies) and a PCSK9 inhibitor (e.g., evolocumab) provide incremental value in further reducing the progression of coronary disease as measured by IVUS.

Given the results of this study (in Example 1), the present application provides for various embodiments involving combined therapies. This is based, in part, upon the observation that reducing low-density lipoprotein cholesterol (LDL-C) with moderate and/or high intensity statin therapy (a non-PCSK9 LDL-C lowering agent) reduces progression of atherosclerosis (e.g., coronary atherosclerosis) in proportion to achieved LDL-C levels and that proprotein convertase subtilisin kexin type-9 (PCSK9) inhibitors further produce incremental LDL-C lowering in statin-treated patients. The results in Example 1 below demonstrate that the addition of a PCSK9 inhibitor, e.g., evolocumab, compared with statin monotherapy (a representative example of a non-PCSK9 LDL-C lowering agent), produced greater LDL-C lowering and significant regression of coronary atherosclerosis at a dose that was well tolerated. Thus, provided herein are combination therapies that involve a PCSK9 inhibitor and a non-PCSK9 LDL-C lowering agent. In some embodiments, the combined therapies can be used for subjects with atherosclerotic cardiovascular disease to improve the subject's cardiovascular outcome.

In some embodiments, a method of treating coronary atherosclerosis is provided. The method can include identifying a subject who is on a first therapy that includes a non-PCSK9 LDL-C lowering agent (e.g., a lipid lowering treatment, such as a statin or other non-PCSK9 LDL-C lowering therapy). The method can further include administering a second therapy to the subject. The second therapy comprises administering a PCSK9 inhibitor to the subject, such as an anti-PCSK9 neutralizing antibody. Both the first and second therapies are administered in an amount and time sufficient to reverse coronary atherosclerosis in the subject (in combination). The PCSK9 inhibitor decreases a level of LDL-C in the subject. The first therapy is different from the second therapy. For example, in some embodiments, the first therapy is not an anti-PCSK9 antibody treatment, but is any other LDL-C lowering agent (such as a statin or other non-PCSK9 LDL-C lowering therapy). In some embodiments, the first therapy is not an antibody treatment. In some embodiments, the combined therapies can be used for subjects with atherosclerotic cardiovascular disease to improve the subject's cardiovascular outcome.

In some embodiments, the first therapy can be any non-antibody, LDL-C lowering therapy. In some embodiments, the first therapy is selected from at least one of: ezetimibe (Zetia) or a statin. In some embodiments, the first therapy is an optimized and/or maximally tolerated statin therapy. In some embodiments, the subject's LDL level decreases to a level beneath 80 mg/dL from the first therapy and then decreases further from the second therapy. In some embodiments, both treatments together result in lowering LDL-C levels at least to 80 mg/dL.

In some embodiments, a method of treating coronary atherosclerosis is provided. The method comprises identifying a subject that has a LDL-C level of less than 70 mg/dL, and administering an anti-PCSK9 neutralizing antibody to the subject, in an amount sufficient and time sufficient to lower the LDL-C level to less than 60 mg/dL. In some embodiments, the subject has been diagnosed with a cardiovascular disease.

In some embodiments, a method of treating coronary atherosclerosis is provided. The method comprises identifying a subject that has a LDL-C level of less than 70 mg/dL, and administering a PCSK9 inhibitor to the subject, in an amount sufficient and time sufficient to lower the LDL-C level to less than 60 mg/dL. In some embodiments, the subject has been diagnosed with a cardiovascular disease.

In some embodiments, a method of treating coronary atherosclerosis is provided. The method comprises identifying a subject that has a LDL-C level of less than 80 mg/dL, and administering a PCSK9 inhibitor (such as an anti-PCSK9 neutralizing antibody) to the subject, in an amount sufficient and time sufficient to lower the LDL-C level to less than 60 mg/dL.

In some embodiments, a method of treating coronary atherosclerosis is provided. The method comprises administering a PCSK9 inhibitor therapy (such as an anti-PCSK9 neutralizing antibody) to the subject who is receiving a non-PCSK9 LDL-C lowering therapy (e.g., an optimized statin therapy), in an amount sufficient and time sufficient to lower the LDL-C level to less than 80 mg/dL. In some embodiments, the result is achieved following at least one year of continuous treatment of both the statin therapy and the antibody therapy. In some embodiments, the subject has further been identified by being diagnosed with coronary atherosclerosis disease or at a high risk of developing with coronary atherosclerosis disease. In some embodiments, the therapies can be used for subjects with atherosclerotic cardiovascular disease to improve the subject's cardiovascular outcome.

In some embodiments, a method of decreasing percent atheroma volume in a subject is provided. The method comprises 1) identifying a subject that has received at least a moderate-intensity treatment by non-PCSK9 LDL-C lowering agent (e.g., a statin), and 2) administering a PCSK9 inhibitor (e.g., an anti-PCSK9 neutralizing antibody) to the subject in an amount sufficient and time sufficient to lower the LDL-C level to less than 100 mg/dL, e.g., less than 90 mg/dL. This can thereby decrease the percent atheroma volume (PAV) in the subject. In some embodiments, the amount and time sufficient is sufficient to lower the LDL-C level to less than 40 mg/dL. In some embodiments, the time period is at least one year and the amount of each of the compounds is as provided herein.

In some embodiments, a method of decreasing total atheroma volume (TAV) in a subject is provided. The method comprises 1) identifying a subject that has received at least a moderate level of treatment by a non-PCSK9 LDL-C lowering agent (e.g., a statin), and 2) administering a PCSK9 inhibitor (e.g., an anti-PCSK9 neutralizing antibody) to the subject in an amount sufficient and time sufficient to lower the LDL-C level to less than 100 mg/dL, e.g., less than 90 mg/dL. This can thereby decrease the total atheroma volume (TAV) in the subject. In some embodiments, the amount and time sufficient is sufficient to lower the LDL-C level to less than 40 mg/dL. In some embodiments, the time period is at least one year and the amount of each of the compounds is as provided herein. In some embodiments, the subject has been diagnosed with a cardiovascular disease.

In some embodiments, both TAV and PAV are reduced in the subject. In some embodiments, the decrease of PAV is at least 0.1 percent, for example, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5% decrease of PAV is achieved. In some embodiments, the decrease of TAV is at least 0.1 percent, for example, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6% decrease of TAV is achieved. In some embodiments, the noted decrease is achieved within about 3 years, 2 years, 18 months, or 1 year. In some embodiments, the PAV is decreased by at least 1% following 18 months of treatment. In some embodiments, the PAV is decreased by at least 2% following 18 months of treatment. In some embodiments, the TAV is decreased by at least 1% following 18 months of treatment. In some embodiments, the TAV is decreased by at least 2% following 18 months of treatment. In some embodiments, the TAV is decreased by at least 3% following 18 months of treatment. In some embodiments, the TAV is decreased by at least 4% following 18 months of treatment. In some embodiments, the TAV is decreased by at least 5% following 18 months of treatment. In some embodiments, the TAV is decreased by at least 6% following 18 months of treatment.

In some embodiments, a method of treating coronary atherosclerosis comprises 1) administering an optimum non-PCSK9 LDL-C lowering therapy (e.g., a statin therapy) to a subject, wherein the subject has coronary atherosclerosis and 2) administering an amount of a PCSK9 inhibitor (e.g., an anti-PCSK9 neutralizing antibody) to the subject at the same time. The steps can occur in order, at the same (or overlapping) time, or in a different order.

In some embodiments, a method of treating coronary atherosclerosis comprises 1) identifying a statin-intolerant subject, 2) administering at least a low intensity statin treatment to the statin-intolerant subject, and 3) administering an amount of an anti-PCSK9 neutralizing antibody to the subject, thereby treating coronary atherosclerosis. The steps can occur in order, at the same (or overlapping) time, or in a different order. In some embodiments, a moderate dose statin therapy is administered. In some embodiments, a high dose statin therapy is administered.

In some embodiments, any of the methods provided herein, including the combination therapies and the therapies where one is lowering LDL-C levels with a single therapy (and/or non-HDL-C levels) to very low levels, involve lowering LDL-C by 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180 mg/dL, or greater decrease in LDL-C (and/or non-HDL-C, which values are adjusted upwards by +30).

In some embodiments, a method of providing regression of coronary atherosclerosis comprises providing a subject that is on an optimized non-PCSK9 LDL-C lowering therapy (e.g., an optimized level of a statin) and administering to the subject a PCSK9 inhibitor (e.g., an anti-PCSK9 neutralizing antibody) at a level adequate to regress coronary atherosclerosis, wherein regression is any change in PAV or TAV less than zero. The steps can occur in order, at the same (or overlapping) time, or in a different order.

In some embodiments, a method of decreasing a LDL-C level in a subject beneath 80 mg/dL is provided. The method comprises administering a PCSK9 inhibitor (e.g., an anti-PCSK9 neutralizing antibody or RNAi to PCSK9) to a subject, wherein the subject has coronary atherosclerotic disease, wherein the subject is on a non-PCSK9 LDL-C lowering therapy (e.g., an optimized statin therapy) for at least one year, and wherein a LDL-C level in the subject decreases to an average value that is beneath 80 mg/dL for the at least one year. The steps can occur in order, at the same (or overlapping) time, or in a different order. In some embodiments, the subject's LDL levels decrease to an average value that is beneath 60 mg/dL for the at least one year, for example, 55, 50, 45, 40, 35, 30, 25, 20 mg/dL or lower for at least one year.

In some embodiments, a method of reducing a relative risk of a cardiovascular event by at least 10% is provided. The method comprises administering a PCSK9 inhibitor (e.g., a PCSK9 neutralizing antibody) to a subject that is on at least a moderate intensity of a non-PCSK9 LDL-C lowering agent (e.g., a statin), in an amount sufficient to lower a LDL-C level of the subject by about 20 mg/dL.

In some embodiments, the cardiovascular event is one selected from the group of non-fatal myocardial infarction, myocardial infarction (MI), stroke/Transient Ischemic Attack (TIA), angina, arterial revascularization, coronary revascularization, fatal and non-fatal stroke, hospitalization for Congestive Heart Failure (CHF), Coronary Heart Disease (CHD) deaths, coronary death. In some embodiments, the combined therapy can reduce and/or slow the progression of atherosclerosis, slow the progression of coronary atherosclerosis, slow the progression of atherosclerosis in patients with CHD, and slow the progression of atherosclerosis in patients with CHD. In some embodiments, the combined therapy can reduce and/or slow atherosclerotic cardiovascular disease (ASCVD), CAD/CHD, cerebrovascular dz, and/or Peripheral Artery Disease (PAD). In some embodiments, any one of the methods provided herein regarding combined therapies can be used to reduce the risk of any one or more these events. In some embodiments, any patient or subject at risk of one of these events is the subject identified as one to receive the combined therapy.

In some embodiments, the subject is one with at least one of the following: an elevated LDL-C level, HoFH, HeFH, and nonfamilial hypercholesterolemia. In some embodiments, the subject is one with a primary hyperlipidemia (heterozygous familial and non-familial) or mixed dyslipidemia or homozygous familial hypercholesterolemia. In some embodiments, a subject that has been identified as being at risk of a cardiovascular event is identified as one to receive the combined therapy. In some embodiments, the subject to receive the combined therapy is one that has at least one or more of: a) elevated total-cholesterol (t-C), b) elevated LDL-C, c) elevated Apo B, d) elevated Lp(a), and/or e) elevated triglycerides (TG), f) elevated non-HDL-C and/or g) low HDL-C and has a primary hyperlipidemia (heterozygous familial and nonfamilial) and/or mixed dyslipidemia. In some embodiments, the subject has one or more of type 1 diabetes, type 2 diabetes, metabolic syndrome, prediabetes, and/or HIV/AIDS.

In some embodiments, the combination therapy provided herein can be used to reduce the risk of or treat at least one or more of the following: CV death, non-fatal myocardial infarction, non-fatal stroke or transient ischemic attack (TIA), coronary revascularization, and hospitalization for unstable angina.

In some embodiments, the combination therapy provided herein can be used in patients with clinically evident atherosclerotic cardiovascular (CV) disease (e.g., prior MI, stroke or symptomatic PAD), to reduce the risk of one or more of: CV death, non-fatal myocardial infarction, non-fatal stroke or transient ischemic attack (TIA), coronary revascularization, and hospitalization for unstable angina. In some embodiments, the combination therapy can be used in patients that are hospitalized for HF (heart failure).

In some embodiments, the combination therapy provided herein can be used in patients with clinically evident atherosclerotic cardiovascular (CV) disease, to reduce the risk of one or more of CV death, non-fatal myocardial infarction, non-fatal stroke or transient ischemic attack (TIA), coronary revascularization, and hospitalization for unstable angina. In some embodiments, the combination therapy can be used in patients that are hospitalized for HF.

In some embodiments, the combination therapy provided herein can be used in patients with clinically evident atherosclerotic cardiovascular (CV) disease (e.g., prior MI, stroke or symptomatic PAD plus 1 major or 2 minor additional CV risk factors), to reduce the risk of CV death, non-fatal myocardial infarction, non-fatal stroke or transient ischemic attack (TIA), coronary revascularization, and hospitalization for unstable angina.

In some embodiments, the combination therapy can be used to treat/prevent/reduce the risk of primary hyperlipidemia and/or mixed dyslipidemia (e.g., heterozygous familial hypercholesterolemia (HeFH), nonfamilial hypercholesterolemia, mixed dyslipidemia, clinical atherosclerotic cardiovascular disease (CVD) or high risk patients without ASCVD (subclinical ASCVD)), coronary atherosclerosis, and/or cardiovascular disease (e.g., CV death, non-fatal myocardial infarction, non-fatal stroke or transient ischemic attack (TIA), coronary revascularization, and hospitalization for unstable angina).

In some embodiments, a method of reducing an amount of atherosclerotic plaque in a subject is provided that comprises administering to a subject having atherosclerotic plaque a PCSK9 inhibitor (e.g., a monoclonal antibody PCSK9, e.g., an anti-PCSK9 neutralizing antibody). The subject is receiving an optimized non-PCSK9 LDL-C lowering therapy (e.g., an optimized statin therapy), thereby reducing the amount of atherosclerotic plaque in the subject. In some embodiments, the method further comprises identifying the subject who is in need of reducing their amount of atherosclerotic plaque. The steps can occur in order, at the same (or overlapping) time, or in a different order.

In some embodiments, a method of reducing disease progression is provided. The method can comprise 1) identifying a subject with a LDL-C level of no more than 80 mg/dL, 2) administering at least a high and/or moderate intensity of a non-PCSK9 LDL-C lowering therapy (e.g., a statin therapy) to the subject; and 3) administering a PCSK9 inhibitor (e.g., evolocumab) at a level sufficient to decrease the LDL-C level of the subject to 30 mg/dL, thereby reducing disease progression. The steps can occur in order, at the same (or overlapping) time, or in a different order. In some embodiments, the subject has had a heart attack. In some embodiments, the subject has a LDL-C level of no more than 60 mg/dL.

In some embodiments, a method of reducing disease progression is provided. The method comprises identifying a subject with a LDL-C level of no more than 80 mg/dL, administering at least a moderate intensity of a statin therapy to the subject, and administering evolocumab at a level sufficient to decrease the LDL-C level of the subject to 30 mg/dL, thereby reducing disease progression. In some embodiments, a high-intensity of a statin therapy is used.

In some embodiments, a method of combining evolocumab and a statin therapy to produce greater LDL-C lowering and regression of coronary atherosclerosis at a dose that is well tolerated is provided. The method comprises administering at least a moderate intensity of a statin therapy to a subject, administering an adequate amount of evolocumab to the subject such that the subject's LDL-C levels drop to no more than 40 mg/dL, and maintaining the subject's LDL-C levels at no more than 40 mg/dL for at least one year. In some embodiments, a high-intensity of a statin therapy is used.

In some embodiments, moderate-intensity non-PCSK9 LDL-C lowering therapy (such as a statin or other non-PCSK9 LDL-C lowering therapy) denotes lowering LDL-C by approximately 30% to <50%. In some embodiments, high-intensity non-PCSK9 LDL-C lowering therapy (such as a statin or other non-PCSK9 LDL-C lowering therapy) therapy denotes lowering LDL-C by approximately ≥50%.

In some embodiments, a method of combining a PCSK9 inhibitor (e.g., evolocumab) and a non-PCSK9 LDL-C lowering therapy (e.g., a statin therapy) to produce greater LDL-C lowering and regression of coronary atherosclerosis at a dose that is well tolerated is provided. The method can comprise 1) administering a high and/or moderate-intensity of a non-PCSK9 LDL-C lowering therapy (e.g., a high and/or moderate-intensity statin therapy) to a subject, 2) administering an adequate amount of a PCSK9 inhibitor (e.g., evolocumab) to the subject such that the subject's LDL-C levels drop to no more than 40 mg/dL, and 3) maintaining the subject's LDL-C levels at no more than 40 mg/dL for at least one year. The steps can occur in order, at the same (or overlapping) time, or in a different order.

In some embodiments, a method of treating a subject that is unable to tolerate a full therapeutic dose of a statin is provided. The method comprises identifying the subject; and administering a PCSK9 inhibitor (e.g., an anti-PCSK9 neutralizing antibody) to the subject until a LDL cholesterol level of the subject decreases beneath 60 mg/dL. In some embodiments, the method comprises identifying the subject; and administering a PCSK9 inhibitor (e.g., an anti-PCSK9 neutralizing antibody) to the subject until a LDL cholesterol level of the subject decreases beneath 80 mg/dL.

In some embodiments, depending upon the context, the first therapy is a non-PCSK9 dependent, LDL-C lowering therapy. That is, it involves the use of a non-PCSK9 LDL-C lowering agent. In particular, while the non-PCSK9 LDL-C lowering agent will lower LDL-C levels, it does not do so through PCSK9. In some embodiments, the first therapy is not an antibody therapy. In some embodiments, the first therapy can be an antibody therapy, wherein the antibody does not bind to PCSK9. The non-PCSK9 LDL-C lowering agent/therapy is not a PCSK9 neutralizing antibody treatment. In some embodiments, the non-PCSK9 LDL-C lowering therapy is a small molecule treatment that can lower LDL-C levels in a subject. In some embodiments, the non-PCSK9 LDL-C lowering therapy is a lipid lowering therapy that excludes PCSK9 driven lipid lowering therapies. In some embodiments, the non-PCSK9 LDL-C lowering therapy is one or more of: niacin; ezetimibe; or a statin (aka HMG CoA reductase inhibitors), atorvastatin (LIPITOR®), cerivastatin, fluvastatin (LESCOL), lovastatin (Mevacor, ALTOPREV), mevastatin, pitavastatin, pravastatin (PRAVACHOL), rosuvastatin, rosuvastatin calcium (CRESTOR) and simvastatin (ZOCOR). Statins are also found in combination medications including: ADVICOR (lovastatin+niacin), CADUET (atorvastatin+amlopidine); selective cholesterol absorption inhibitors, ezetimibe (ZETIA); a Lipid Lowering Therapy (LLT) fibrates or fibric acid derivatives, including gemfibrozil (LOPID), fenofibrate (ANTARA, LOFIBRA, TRICOR, TRIGLIDE) and clofibrate (ATROMID-S); a Resin (aka bile acid sequestrant or bile acid-binding drugs), cholestyramine (QUESTRAN, QUESTRAN LIGHT, PREVALITE, LOCHOLEST, LOCHOLEST LIGHT), cholestipol (CHOLESTID) and cholesevelan Hcl (WELCHOL) and/or a combination thereof, including but not limited to VYTORIN (simvastatin+ezetimibe). In some embodiments, the non-PCSK9 LDL-C lowering therapy comprises a moderate or a high intensity statin therapy. In some embodiments, the non-PCSK9 LDL-C lowering therapy comprises a maximally tolerated dose of the statin. A moderate-intensity therapy denotes lowering LDL-C by approximately 30 to <50%. A high-intensity therapy denotes lowering LDL-C by ≥50%. In some embodiments, the first therapy, the non-PCSK9 dependent therapy lowers lipid levels generally, and non-HDL-C levels specifically. Thus, it is also contemplated that non-PCSK9 dependent lipid lowering therapies can be used as a first therapy, even though the therapy may alter more than just LDL-C levels and/or not emphasize LDL-C levels.

In some embodiments, the non-PCSK9 LDL-C lowering therapy (which can be the statin treatment) is an amount of statin that is at least as effective as a dose of atorvastatin of 20 mg daily or an equivalent to atorvastatin at an equivalent amount. In some embodiments, the amount of the statin is at least as effective as a dose of atorvastatin of at least 40 mg daily or an equivalent to atorvastatin at an equivalent amount. In some embodiments, the statin is at least one of atorvastatin, simvastatin, rosuvastatin, pravastatin, lovastatin, and pitavastatin. In some embodiments, the statin is at least one of atorvastatin at 20, 40, or 80 mg; simvastatin at 40 or 80 mg; rosuvastatin at 5, 10, 20, or 40 mg; pravastatin at 80 mg, lovastatin at 80 mg, or pitavastatin at 4 mg. In some embodiments, the subject is receiving or taking at least atorvastatin 40 or 80 mg; rosuvastatin 10, 20, or 40 mg; or simvastatin 80 mg. In some embodiments, the amount of statin administered is the maximally tolerated amount of statin. In some embodiments, the amount of statin is equivalent to at least atorvastatin 20 mg/day. In some embodiments, the amount of statin is equivalent to at least atorvastatin 40 mg/day.

In some embodiments, the statin is a monotherapy. In some embodiments, the subject is also on an additional lipid lowering therapy (and thus can be on a statin, a PCSK9 antibody, and a third treatment). In some embodiments, the additional lipid lowering therapy is niacin, ezetimibe, or both niacin and ezetimibe. The present treatments are not only options for the first therapy, but, of course, also embodiments for the lipid lowering therapies and/or the statin therapies provided herein. In some embodiments, the additional therapy can be an inhibitor to ASGR1, such as an antibody to ASGR1 or an ASGR1 siRNA. In some embodiments, the additional therapy can be an inhibitor to LDLR, such as an antibody to LDLR or an LDLR siRNA. In some embodiments, the additional therapy can be an inhibitor to Lp(a), such as an antibody to Lp(a) or an Lp(a) siRNA. In some embodiments, the additional therapy can be one or more of: a Lp(a) antagonist (e.g., peptide, mAb, and/or siRNA), an antibody or inhibitor of ANGPTL4 and/or ANGPTL3, an inhibitor of PNPLA3 (e.g., siRNA), an inhibitor of ASGR1, an inhibitor of ASGR2 (siRNA), an inhibitor of ApoC3 (e.g., siRNA), a GLP-1 receptor agonist, and/or a GIPR antagonist.

In some embodiments, the non-PCSK9 LDL-C lowering therapy (which can be a statin treatment) can be administered at any level sufficient to lower cholesterol in the blood. In some embodiments, the non-PCSK9 LDL-C lowering therapy (which can be a statin treatment and/or a LLT) is administered in an amount and time to achieve the maximal level of LDL lowering in the blood. In some embodiments, any one or more of the above statins is administered daily.

In some embodiments, the second therapy, the PCSK9 LDL-C lowering agent, the PCSK9 inhibitor, the non-statin LDL-C lowering agent can be any therapy that lowers LDL-C levels through PCSK9. This can also be described as involving a PCSK9 inhibitor. Such PCSK9 inhibitors can include antibodies evolocumab (CAS Reg. No. 1256937-27-5; WHO No. 9643, IND No. 105188) (REPATHA®), alirocumab (PRALUENT®), bococizumab, REGN728, RG7652, LY3015014, LGT209, 1D05 (U.S. Pat. No. 8,188,234), 1B20 (U.S. Pat. No. 8,188,233). In some embodiments, the antibody is a neutralizing antibody. In some embodiments, the anti-PCSK9 neutralizing antibody is evolocumab. In some embodiments, the inhibitor is an anti-PCSK9 antibody that contains one or more (including all 6) of the CDRs from the antibody constructs shown in any one or more of FIGS. 6-12. In some embodiments, the PCSK9 inhibitor is an anti-PCSK9 antibody that contains one or more of the amino acid heavy and/or light chains of FIGS. 6-12. In some embodiments, antibodies that include any one or more of the CDRs of the antibodies noted herein can be employed. In some embodiments, antibodies that include the heavy and light chain variable regions of the antibodies noted herein can be employed. In some embodiments, the antibody is at least 95, 96, 97, 98, 99% identical in amino acid sequence to an antibody denoted herein. In some embodiments, the anti-PCSK9 antibody is selected from the antibodies in U.S. Pat. No. 8,062,640 (e.g., HCVR/LCVR=SEQ ID NOS:90/92), U.S. Pat. No. 8,501,184 (e.g., REGN728, HCVR/LCVR=SEQ ID NOS:218/226), U.S. Pat. No. 8,080,243 (e.g., bococizumab, HCVR/LCVR=SEQ ID Nos:54/53), U.S. Pat. No. 8,188,234 (e.g., 1D05, HCVR/LCVR=SEQ ID Nos:11/27), U.S. Pat. No. 8,188,233 (e.g., 1B20, HCVR/LCVR=SEQ ID Nos:11/27), LGT209 in U.S. Pat. No. 8,710,192, US2011/0142849, and US2013/0315927, and RG7652 in US2012/0195910, LY3015014 in U.S. Pat. No. 8,530,414 (HCVR/LCVR=SEQ ID Nos:7/8). In some embodiments, the PCSK9 inhibitor includes the specific double stranded sequence of ALN-PCSsc (from U.S. Pat. Nos. 7,605,251, 8,809,292, 9,260,718 and 8,273,869). The entireties of each of which is hereby incorporated by reference including the disclosure of the specifically referenced PCSK9 inhibitors. Such PCSK9 inhibitors can also include RNAi therapies, such as siRNA and ALN-PCSsc. Also contemplated herein are PCSK9 lipid lowering agents that can lower other lipids (apart from LDL-C). Of course, the above “second therapy,” the “PCSK9 LDL-C lowering agent,” the “PCSK9 inhibitor,” and/or the “non-statin LDL-C lowering agent” can lower both LDL-C as well as other lipids. Further contemplated are PCSK9 lipid lowering agents, which can lower lipids generically. All of the embodiments provided in the present paragraph can be employed for one or more of the combination therapies provided herein. Furthermore, for the embodiments provided herein that do not require a combination of therapies (such as those that provide an especially large reduction in LDL-C or non-HDL-c via a single agent), the present therapeutics can be used for those embodiments as well (even though there is no “second therapy” in that context).

The amount of the non-PCSK9 LDL-C lowering therapy administered can be enough to achieve the desired result, when combined with the PCSK9 inhibitor therapy for an adequate period of time.

In some embodiments, at least 50, 60, 70, 75, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450 mg of a PCSK9 inhibitor (such as a neutralizing antibody) is administered to the subject. In some embodiments, evolocumab is administered in an amount of at least 140 mg, for example, at least 150 mg, 300 mg, 400 mg or at least 420 mg. In some embodiments, the amount of the anti-PCSK9 neutralizing antibody is at least 140 mg, for example, at least 150 mg, 300 mg, 400 mg or at least 420 mg.

In some embodiments, the PCSK9 inhibitor (e.g. neutralizing antibody, e.g., evolocumab), is administered at a frequency of at least once a week, at least once a month, at least once every two weeks, once every three months, or at least once a week.

In some embodiments, the non-PCSK9 LDL-C lowering therapy and/or PCSK9 inhibitor therapies can be administered as they would normally be administered for LDL-C lowering. In some embodiments, this is done to a maximally tolerated dosage for the subject. In certain embodiments, the route of administration of the two ingredients in the combined therapy is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices.

In some embodiments, the PCSK9 inhibitor (e.g., neutralizing antibody, e.g., evolocumab), is administered at least monthly to the subject for at least one year. In some embodiments, it is administered for at least 0.5, 12, 18, 24, 30, 36, 42, 48, 54, 60 or more months.

In some embodiments, the LDL-C level of the subject on the combined therapy decreases by at least 40%, for example 40, 45, 50, 55, 60, 65, 70, 75, 80, 85% or more.

In some embodiments, the subject has been treated with a stable non-PCSK9 LDL-C lowering agent (e.g., statin) dose for at least four weeks and has a LDL-C ≥80 mg/dL or between 60 and 80 mg/dL with one major and/or three minor cardiovascular risk factors. The major risk factor can be at least one of: non-coronary atherosclerotic vascular disease, myocardial infarction or hospitalization for unstable angina in the preceding 2 years or type 2 diabetes mellitus. The minor risk factor can be at least one of: current cigarette smoking, hypertension, low levels of high-density lipoprotein cholesterol (HDL-C), family history of premature coronary heart disease, or high sensitivity C-reactive protein (hs-CRP) ≥2 mg/L or age ≥50 years in men and 55 years in women.

In some embodiments, providing regression of coronary atherosclerosis denotes a decrease in PAV and/or TAV. In some embodiments, the decrease in PAV is at least 0.1 percent, for example, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, or 2.5% decrease of PAV is achieved. In some embodiments, the decrease of TAV is at least 0.1 percent, for example, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10% decrease of TAV is achieved.

In some embodiments, the combined therapy provides for a reduction in a risk of atherosclerosis, coronary atherosclerosis, atherosclerotic cardiovascular disease, a coronary artery disease (CAD), cardiovascular event, non-fatal myocardial infarction coronary revascularization, PAD, and/or cerebrovascular disease for the subject. In some embodiments, the combined therapy provides for a reduction in risk of the occurrence of one or more of: death from any cause, CHD deaths, cardiovascular death, angina, myocardial infarction (MI), stroke, fatal and non-fatal stroke arterial revascularization procedures, coronary revascularization procedures, hospitalization for CHF, and/or unstable angina.

In some embodiments, the combined therapies provides for an LDL-C level in the subject to be decreased beneath 80 mg/dL, for example, beneath 70, 60, 50, 40, 30, 20 mg/dL.

In some embodiments, any of the above embodiments (or other embodiments provided herein) regarding atherosclerosis can be applied to improving cardiovascular outcomes in patients with atherosclerotic cardiovascular disease. Such embodiments can employ similar therapy approaches (e.g., a combined therapy), in that the subject can be on two therapies, one of which is, for example a non-PCSK9 inhibitor, such as a statin, while the other is, for example, a PCSK9 inhibitor, such as evolocumab. The non-PCSK9 LDL-C lowering therapy will lower LDL-C levels.

In some embodiments, the cardiovascular method can comprise the inhibition of PCSK9 with evolocumab in a subject who is on a statin therapy. This can result in a lowered LDL cholesterol to 30 mg/dL and a reduced risk of cardiovascular events. In some embodiments, this is achieved with no significant safety downside.

In some embodiments, a method of treating atherosclerotic cardiovascular disease is provided. The method can comprise a) identifying a subject that is on a first therapy, wherein the first therapy comprises a non-PCSK9 LDL-C lowering therapy. The method can further comprise b) administering a second therapy to the subject. The second therapy comprises a PCSK9 inhibitor therapy. Both the first and second therapies are administered to the subject in an amount and time sufficient to reduce a risk of atherosclerotic cardiovascular disease in the subject. The first therapy is not the same as the second therapy, and the risk is a) a composite for cardiovascular death, myocardial infarction, stroke, hospitalization for unstable angina, or coronary revascularization or b) a composite for cardiovascular death, myocardial infarction, or stroke, or c) cardiovascular death, or d) fatal and/or non-fatal MI, or e) fatal and/or non-fatal stroke, or f) transient ischemic attack, or g) hospitalization for unstable angina, or h) elective, urgent, and/or emergent coronary revascularization.

In some embodiments a method of reducing a risk of a cardiovascular event is provided. The method comprises a) identifying a subject that is on a first therapy, wherein the first therapy comprises a non-PCSK9 LDL-C lowering therapy. The method can further comprise b) administering a second therapy to the subject. The second therapy comprises a PCSK9 inhibitor. Both the first and second therapies are administered to the subject in an amount and time sufficient to reduce a risk of a cardiovascular event in the subject. The first therapy is not the same as the second therapy. The risk is a) a composite for cardiovascular death, myocardial infarction, stroke, hospitalization for unstable angina, or coronary revascularization or b) a composite for cardiovascular death, myocardial infarction, or stroke, or c) cardiovascular death, or d) fatal and/or non-fatal MI, or e) fatal and/or non-fatal stroke, or f) transient ischemic attack, or g) hospitalization for unstable angina, or h) elective, urgent, and/or emergent coronary revascularization.

In some embodiments, a method of reducing a risk of urgent coronary revascularization is provided. The method comprises a) identifying a subject that is on a first therapy, wherein the first therapy comprises a non-PCSK9 LDL-C lowering therapy. The method further comprises b) administering a second therapy to the subject. The second therapy comprises a PCSK9 inhibitor therapy. Both the first and second therapies are administered to the subject in an amount and time sufficient to reduce the risk of atherosclerotic cardiovascular disease in the subject. The first therapy is not the same as the second therapy.

In some embodiments, a method of reducing a risk of a cardiovascular event is provided. The method comprises a) identifying a subject with cardiovascular disease, and b) administering a PCSK9 inhibitor to the subject in an amount and overtime sufficient to reduce a risk of at least one of cardiovascular death, non-fatal myocardial infarction, non-fatal stroke or transient ischemic attack (TIA), coronary revascularization, or hospitalization for unstable angina.

In some embodiments, a method of reducing a risk of a cardiovascular event is provided. The method comprises a) identifying a subject that is on a first therapy, wherein the first therapy comprises a non-PCSK9 LDL-C lowering therapy; and b) administering a second therapy to the subject, wherein the second therapy comprises a PCSK9 inhibitor. Both the first and second therapies are administered to the subject in an amount and time sufficient to reduce a risk of a cardiovascular event in the subject. The first therapy is not the same as the second therapy, and the risk is the composite of coronary revascularization, myocardial infarction, cerebral vascular accident.

In some embodiments, a method of reducing a risk of a cardiovascular event is provided. The method comprises a) identifying a subject that is on a first therapy, wherein the first therapy comprises a non-PCSK9 LDL-C lowering therapy, and b) administering a second therapy to the subject, wherein the second therapy comprises a PCSK9 inhibitor. Both the first and second therapies are administered to the subject in an amount and time sufficient to reduce a risk of a cardiovascular event in the subject, and wherein the first therapy is not the same as the second therapy, and wherein the risk is the composite of fatal MI and/or non-fatal MI and fatal and/or non-fatal coronary revascularization.

In some embodiments the risk is any one or more of, combination of, or composite of coronary revascularization, myocardial infarction, cerebral vascular accident. In some embodiments the risk is any one or more of, combination of, or composite of fatal MI and/or non-fatal MI and fatal and/or non-fatal coronary revascularization.

In some embodiments, the combined therapy (or any of the monotherapies provided herein) is continued for more than six months, for example, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or more months, following which, the risk of a cardiovascular event, such as cardiovascular death, myocardial infarction, stroke, hospitalization for unstable angina, or coronary revascularization has decreased at least 5, 10, 15, 20, 25 or greater percent. In some embodiments, the risk is the composite of these disorders (the first occurrence of any one of those, in combination). In some embodiments, the risk is for the combination of these disorders. In some embodiments, the risk is for each of the disorders separately. In some embodiments, the risk is for cardiovascular death, myocardial infarction, or stroke only (but as a composite). In some embodiments, the combined risk of all of these has decreased at least 5, 10, 15, 20, 25% or more, at 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or more months. In some embodiments, the reduced rate is the composite of these disorders (the first occurrence of any one of those, in combination). In some embodiments, the risk is for the combination of these disorders. In some embodiments, the risk is for each of the disorders separately. In some embodiments, the risk is for cardiovascular death, myocardial infarction, or stroke only (but as a composite). In some embodiments, the risk decreases from about 16% during the first year of therapy to about 25% after the first year of therapy.

In some embodiments, the combined therapy (or any of the monotherapies provided herein) is continued for more than six months, for example, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or more months, following which, the risk of a cardiovascular event, such as cardiovascular death, myocardial infarction, or stroke has decreased at least 5, 10, 15, 20, 25 or greater percent. In some embodiments, the risk is the composite of these disorders (the first occurrence of any one of those, in combination). In some embodiments, the risk is for the combination of these disorders. In some embodiments, the risk is for each of the disorders separately. In some embodiments, the combined risk of all of these has decreased at least 5, 10, 15, 20, 25% or more, at 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or more months. In some embodiments, the reduced rate is the composite of these disorders (the first occurrence of any one of those, in combination). In some embodiments, the risk is for the combination of these disorders. In some embodiments, the risk is for each of the disorders separately. In some embodiments, the risk deceases from about 16% during the first year of therapy to about 25% after the first year of therapy.

In some embodiments, any of the methods provided herein related to reducing risk can exclude reducing the risk of cardiovascular death over more than 12 months and less than 36 months when separate from myocardial infarction and stroke. In some embodiments, any of the methods provided herein related to reducing risk can exclude reducing the risk of cardiovascular death over more than 12 months. In some embodiments, any of the methods provided herein related to reducing risk can include reducing the risk of cardiovascular death over more than 36 months.

In some embodiments, the combination therapy (or any of the monotherapies provided herein) allows for a significant reduction in the risk of cardiovascular events, with, for example, a 15% reduction in the risk of the primary composite end point of cardiovascular death, myocardial infarction, stroke, hospitalization for unstable angina, or coronary revascularization (either a) individually or b) as a composite (any one of which, but as a combination) and a 20% reduction in the risk of the clinically stringent key secondary end point of cardiovascular death, myocardial infarction, or stroke (either a) individually or b) as a composite (any one of which, as a combination)). In some embodiments, combination therapy reduces a risk of myocardial infarction by 27%, stroke by 21%, and coronary revascularization by 22%. In some embodiments, the primary end point is a composite (e.g., the first of any one of which, in combination) of time to cardiovascular death, myocardial infarction, stroke, coronary revascularization, or hospitalization for unstable angina, whichever occurs first. Thus, in some embodiments, the method allows one to reduce the risk of (or increase the time to) cardiovascular death, myocardial infarction, stroke, coronary revascularization, or hospitalization for unstable angina, whichever occurs first. In some embodiments, the method allows one to decrease the composite (e.g., the first of any one of which, in combination) of time to cardiovascular death, myocardial infarction, stroke, coronary revascularization, or hospitalization for unstable angina, whichever occurs first.

In some embodiments, the method allows one to reduce the risk of (or increase the time to) cardiovascular death, myocardial infarction, or stroke, whichever occurs first. In some embodiments, the method allows one to decrease the composite (e.g., the first of any one of which, in combination) of time to cardiovascular death, myocardial infarction, or stroke, whichever occurs first.

In some embodiments, the methods provided herein result in lowering LDL cholesterol by a significant amount. In some embodiments, the reduction is at least 50%, for example 59% from a median of 92 to 30 mg/dL (from 2.4 to 0.8 mmol/L). This effect can be sustained over 3 years without evidence of attenuation.

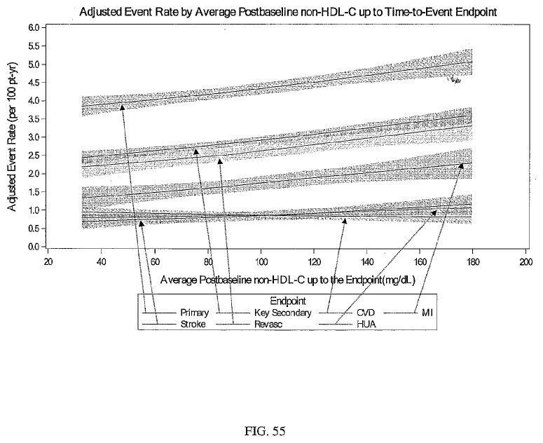

In some embodiments, the subject who is to receive an improved cardiovascular outcome, is (1) on a statin with a potency equivalent to atorvastatin 20 mg daily or greater (see, e.g., table 17.4), and (2) while on that regimen have an LDL-C ≥70 mg/dl or a non-HDL-C ≥100 mg/dl. In some embodiments, the subject to be treated has a non-HDL-c levels that is at least as high as a corresponding level of LDL-C. In some embodiments, this means any LDL-C level provided herein, +30 mg/dLl (as a conversion factor from non-HDL-c to LDL-c). Non-HDL-C denotes its art recognized meaning, and denotes cholesterol minus HDL-C. It includes LDL-C, VLDL-C (determined roughly as tg/5) and Lp(a). As shown in FIG. 55, lowering of non-HDL-C, down to approximately 30 mg/dL) reduces the event rate, and thus risk that the subject will have a wide variety of events. As shown in FIG. 55, reducing non-HDL-C to such very low levels (e.g., less than 50, 40, 30, 20, etc.) lows the event rate of: the primary, secondary, CVD, MI, stroke, pevasc, and hospitalization for unstable angina (“HUA”) of the subject. The primary and secondary endpoints are those as defined in FOURIER. The primary endpoint is: cardiovascular death, MI, stroke, hospitalization for unstable angina, or coronary revascularization. The secondary endpoint was the composite of CV death, MI or stroke. Subjects at risk of any of the indications (or subparts thereof) shown in FIG. 55 can benefit from the methods provided herein. Furthermore, any of the indications that have a benefit described herein from lowering a subject's LCL-C level, can also have their progress tracked by monitoring non-HDL-C levels. That is, it is also contemplated that each LDL-C lowering method can also (or in the alternative focus on) the lowering of non-HDL-C. One of skill in the art will appreciate the overlap between the two approaches, as LDL-C is a component of non-HDL-C.

In some embodiments, the subject has clinically evident atherosclerotic cardiovascular disease. In some embodiments, this is defined as a history of myocardial infarction, history of non-hemorrhagic stroke, or symptomatic peripheral artery disease, and additional characteristics that placed them at higher cardiovascular risk (such as those outlined in the supplemental section of Example 17). In some embodiments, the subject has had a fasting LDL cholesterol ≥70 mg/dL or a non-HDL cholesterol of ≥100 mg/dL on an optimized stable lipid-lowering therapy, preferably a high intensity statin, but must have been at least atorvastatin 20 mg daily or equivalent, with or without ezetimibe. In some embodiments, such subjects, following identification, can receive the combined therapy and obtain improved cardiovascular outcomes.

In some embodiments, the method allows for a reduction in the risk or occurrence of the composite of (e.g., the first of any one of which, in combination) cardiovascular death, myocardial infarction, stroke, hospitalization for unstable angina, or coronary revascularization. In some embodiments, the risk is significantly reduced when P<0.05. In some embodiments, there is a reduction in the risk of recurrence of the composite of (e.g., the first of any one of which, in combination) cardiovascular death, myocardial infarction, stroke, hospitalization for unstable angina, or coronary revascularization.

In some embodiments, the method allows for a reduction in the risk or occurrence of the composite (e.g., the first occurrence of any one of which, in combination) of cardiovascular death, myocardial infarction, or stroke. “Composite denotes the first occurrence (e.g., time to) of an item listed within a group of events. “Composite risk” or other similar term denotes the risk to the time to the first of the events within the list. Thus, a composite risk for cardiovascular death, myocardial infarction, or stroke would describe the risk of first occurrence of any one of those three, considered in combination. In some embodiments, there is a reduction in the risk of occurrence of the composite of cardiovascular death, myocardial infarction, hospitalization for unstable angina, stroke or coronary revascularization. In some embodiments, there is a reduction in the risk of occurrence of the composite of cardiovascular death, myocardial infarction, or stroke. As used herein, the term “composite” will control how the meaning of a list of items is to be interpreted.

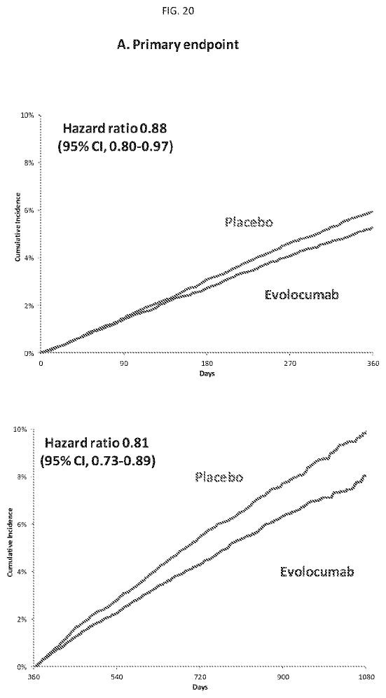

In some embodiments, the combined use of a non-PCSK9 inhibitor and a PCSK9 inhibitor can significantly reduce the rate of: death, myocardial infarction, stroke, coronary revascularization, or hospitalization for unstable angina. In some embodiments, the reduced rate is the composite of these disorders (the first occurrence of any one of those, in combination). In some embodiments, the magnitude of the risk reduction can further increase over time, from 12% (95% CI 3 to 20) in the first year to 19% (95% CI 11 to 27) beyond the first year, for example. Likewise for the secondary endpoints described herein in regard to the FOURIER results, the risk reduction went from 16% (95% CI 4 to 26) in the first year to 25% (95% CI 15 to 34) beyond the first year (see FIG. 20 and Example 17 Supplemental Results). In some embodiments, the combined therapy allows for a hazard ratio in a first year of reduced risk of 0.84 (95% CI, 0.74-0.96) for cardiovascular death, myocardial infarction, or stroke (as a composite). In some embodiments, the combined therapy allows a hazard ratio beyond the first year of reduced risk of 0.75 (95% CI, 0.66-0.85) for cardiovascular death, myocardial infarction, or stroke (as a composite). In some embodiments, the combined therapy allows for a hazard ratio in a first year of reduced risk of 0.88 (95% CI, 0.80-0.97) for cardiovascular death, myocardial infarction, stroke, hospitalization for unstable angina, or coronary revascularization (as a composite). In some embodiments, the combined therapy allows for a hazard ratio beyond the first year of reduced risk of 0.81 (95% CI, 0.73-0.89) for cardiovascular death, myocardial infarction, stroke, hospitalization for unstable angina, or coronary revascularization (as a composite).

In some embodiments, the combined therapy allows for a hazard ratio as shown in Table 17.2b, from following a combined therapy method as outlined herein.

TABLE 17.2b

| | Hazard Ratio (95% CI) | |
|---|---|---|
| Outcome | In first year | Beyond first year |
| Primary end point | 0.88 (0.80-0.97) | 0.81 (0.73-0.89) |
| Key secondary end point | 0.84 (0.74-0.96) | 0.75 (0.66-0.85) |
| Cardiovascular death | 0.96 (0.74-1.25) | 1.12 (0.88-1.42) |
| Myocardial infarction | 0.80 (0.68-0.94) | 0.65 (0.55-0.77) |
| Hospitalization for unstable angina | 0.97 (0.77-1.22) | 0.99 (0.75-1.30) |
| Stroke | 0.83 (0.63-1.08) | 0.76 (0.60-0.97) |
| Coronary revascularization | 0.84 (0.74-0.96) | 0.72 (0.63-0.82) |
| Urgent | 0.84 (0.71-1.00) | 0.63 (0.52-0.75) |
| Elective | 0.86 (0.72-1.03) | 0.81 (0.68-0.97) |
| CTTC composite endpoint | 0.87 (0.79-0.97) | 0.78 (0.71-0.86) |
| Coronary heart death, MI, ischemic | 0.86 (0.76-0.97) | 0.76 (0.68-0.86) |
| stroke, or urgent revascularization | | |
| Coronary heart death, MI, or stroke | 0.84 (0.73-0.95) | 0.73 (0.65-0.83) |
| Fatal or nonfatal MI or stroke | 0.81 (0.70-0.93) | 0.67 (0.59-0.77) |

In some embodiments, the combined therapy allows for a hazard ratio of 0.96 (0.74-1.25) in the first year for cardiovascular death.

In some embodiments, the combined therapy allows for a hazard ratio of 0.80 (0.68-0.94) in the first year for Myocardial infarction. In some embodiments, the combined therapy allows for a hazard ratio of 0.65 (0.55-0.77) beyond the first year for Myocardial infarction.

In some embodiments, the combined therapy allows for a hazard ratio of 0.97 (0.77-1.22) in the first year for Hospitalization for unstable angina. In some embodiments, the combined therapy allows for a hazard ratio of 0.99 (0.75-1.30) beyond the first year for Hospitalization for unstable angina.

In some embodiments, the combined therapy allows for a hazard ratio of 0.83 (0.63-1.08) in the first year for Stroke. In some embodiments, the combined therapy allows for a hazard ratio of 0.76 (0.60-0.97) beyond the first year for Stroke.

In some embodiments, the combined therapy allows for a hazard ratio of 0.84 (0.74-0.96) in the first year for Coronary revascularization. In some embodiments, the combined therapy allows for a hazard ratio of 0.72 (0.63-0.82) beyond the first year for Coronary revascularization.

In some embodiments, the combined therapy allows for a hazard ratio of 0.84 (0.71-1.00) in the first year for urgent coronary revascularization. In some embodiments, the combined therapy allows for a hazard ratio of 0.63 (0.52-0.75) beyond the first year for urgent coronary revascularization.

In some embodiments, the combined therapy allows for a hazard ratio of 0.86 (0.72-1.03) in the first year for elective coronary revascularization. In some embodiments, the combined therapy allows for a hazard ratio of 0.81 (0.68-0.97) beyond the first year for elective coronary revascularization.

In some embodiments, the combined therapy allows for a hazard ratio of 0.87 (0.79-0.97) in the first year for CTTC composite endpoint. In some embodiments, the combined therapy allows for a hazard ratio of 0.78 (0.71-0.86) in the second year for CTTC composite endpoint.

In some embodiments, the combined therapy allows for a hazard ratio of 0.86 (0.76-0.97) in the first year for Coronary heart death, MI, ischemic stroke, or urgent revascularization as a composite. In some embodiments, the combined therapy allows for a hazard ratio of 0.76 (0.68-0.86) in the second year for Coronary heart death, MI, ischemic stroke, or urgent revascularization as a composite.

In some embodiments, the combined therapy allows for a hazard ratio of 0.84 (0.73-0.95) in the first year for Coronary heart death, MI, or stroke (as a composite). In some embodiments, the combined therapy allows for a hazard ratio of 0.73 (0.65-0.83) in the second year for Coronary heart death, MI, or stroke (as a composite).

In some embodiments, the combined therapy allows for a hazard ratio of 0.81 (0.70-0.93) in the first year for Fatal or nonfatal MI or stroke (as a composite). In some embodiments, the combined therapy allows for a hazard ratio of 0.67 (0.59-0.77) in the second year for Fatal or nonfatal MI or stroke (as a composite).

In some embodiments, “reducing the risk” denotes at least one of a) increasing an amount of time to the first of any one of cardiovascular death, myocardial infarction, stroke, hospitalization for unstable angina, or coronary revascularization (as a composite or individually or in combination), or b) increasing an amount of time to the first of any one of cardiovascular death, myocardial infarction, or stroke (as a composite or individually or in combination). In some embodiments, a reduction in the risk can be achieved throughout the treatment period, for example, at month 1, 2, 3, 4, 5, 6, 7, 8, 9, 10, 11, or month 12 or beyond (as a composite or individually or in combination).

In some embodiments, the method can result in a 17% reduction in risk of the key secondary endpoint in patients who start with a median LDL cholesterol from 126, which is then lowered by evolocumab to 43 mg/dL and a 22% reduction in risk in patients who start with a median LDL cholesterol of 73, which is then lowered by evolocumab to 22 mg/dL.

In some embodiments, there is a 21% to 27% reduction in a risk of myocardial infarction, stroke and coronary revascularization (as a composite, individually, or as a combination).

In some embodiments, there is a 17% reduction in risk of cardiovascular death, myocardial infarction, or stroke in a subject, wherein the subject has an initial median LDL cholesterol of 126 mg/dL (as a composite, individually, or as a combination). In some embodiments, the final median LDL cholesterol level of the subject is 43 mg/dL.

In some embodiments, there is a 22% reduction in risk of cardiovascular death, myocardial infarction, or stroke in a subject, wherein the subject has an initial median LDL cholesterol of 73 mg/dL (as a composite, individually, or as a combination). In some embodiments, the final median LDL cholesterol level of the subject is 22 mg/dL.

In some embodiments, the method reduces the composite of myocardial infarction, stroke, or cardiovascular death in patients with established atherosclerotic cardiovascular disease (ASCVD). In some embodiments, the method comprises administering evolocumab to a subject having ASCVD and who is on a standard background therapy (including, for example, statins, resulting in a combined therapy). In some embodiments, the result is that the subject's risk of cardiovascular events including myocardial infarction, ischemic stroke, and cardiovascular death decreases. In some embodiments, the subject's quality-adjusted life-year (QALY) increases. The quality-adjusted life year or quality-adjusted life-year (QALY) is a generic measure of disease burden, including both the quality and the quantity of life lived.

In some embodiments, lifetime cardiovascular event rates can be about 179 per 100 patients with standard background therapy, but can drop down to about 135 with the addition of evolocumab (in a combined therapy). In some embodiments, lifetime cardiovascular event rates can be about 140 to 130 to 120 per 100 patients when standard background therapy is combined with an antibody therapy, such as evolocumab (for a combined therapy). In some embodiments, the treatment is administered to patients with low-density lipoprotein (LDL) cholesterol of ≥80 mg/dL In some embodiments, the 2-year risk for first event (non-fatal myocardial infarction, non-fatal stroke, or cardiovascular death) is less than 13.9%, for subjects on the antibody and standard background therapy (e.g., on a combined therapy), for example, between 13.9 and 7, 13 and 7, 12 and 7, 11 and 7, 10 and 7, 9 and 7, 8 and 7.4%.

In some embodiments, the individual non-fatal myocardial infarction, non-fatal ischemic stroke, and coronary revascularization respective risk reductions can be 21%, 26% and 16% in the first year and 36%, 25% and 28% beyond year 1 on a combined therapy.

In some embodiments, the lifetime QALY can be 7.23 with standard background therapy and can increase to 7.62 with evolocumab (in a combined therapy), with the difference in health effects of 0.39 QALY. In some embodiments, the increase can be at least 0.1, 0.2, 0.3, 0.4, 0.5, 0.6 QALY upon the administration of evolocumab (in a combined therapy). In some embodiments, the QALY itself can be more than 7.23 upon administration with evolocumab, e.g., 7.23, 7.25, 7.3, 7.35, 7.4, 7.45, 7.5, 7.55, 7.6, 7.7, 7.8 or more.

In some embodiments, the method provides a decrease in the rate of subsequent events, health state utilities (the quality of the life-years) and cardiovascular disease events and procedures costs by reducing nonfatal events, even in the absence of direct survival benefit.

In some embodiments, evolocumab, when added to standard background therapy, including high or moderate intensity statin therapy, in patients with established ASCVD provides a 15% relative risk reduction in the composite of cardiovascular death, myocardial infarction, stroke, hospitalization for unstable angina, or coronary revascularization over a median follow-up of 2.2 years. In some embodiments, there can be a 20% risk reduction in the composite of cardiovascular death, myocardial infarction, or stroke. In some embodiments, a greater magnitude of clinical benefits can be observed after the first year of treatment with evolocumab.

In some embodiments, the method provides an incremental reduction in cardiovascular events, corresponding to reductions in hospitalizations, and revascularizations resulting from the addition of evolocumab (in a combined therapy).

In some embodiments, the patient has established ASCVD. In addition, the patient would, with other currently available lipid-modifying therapies including maximally tolerated statins, benefit from additional LDL cholesterol lowering. Such a patient can receive evolocumab, which can facilitate improved clinical outcomes for the subject. In some embodiments, the combined therapy is administered to a patient with ASCVD who is at a particularly high risk for events based on clinical factors, formal risk scores, and/or use of a higher LDL cholesterol.

The table below outlines the baseline characteristics of the atherosclerotic cardiovascular disease U.S. patient population from NHANES. In some embodiments, any one or more of the items below can be used to assist in identifying subjects at higher risk of atherosclerotic cardiovascular disease.

| | LDL-C ≥ 70 | LDL-C ≥ 100 |
|---|---|---|
| | mg/dL | mg/dL |
| Age, years, mean (SD) | 66 (11) | 64 (12) |
| Sex, male, % | 61% | 59% |

Race, %

| | | |
|---|---|---|
| White | 78% | 74% |
| Black or African American | 8% | 11% |
| Asian or other | 14% | 14% |

Cardiovascular risk factors, %

| | | |
|---|---|---|
| Hypertension | 74% | 77% |
| Diabetes mellitus | 26% | 27% |
| Current cigarette use | 26% | 20% |

History of vascular disease, %

| | | |
|---|---|---|
| Established cardiovascular disease | 14% | 29% |
| Myocardial infarction | 52% | 44% |
| Stroke | 34% | 27% |
| Ezetimibe use, % | 7% | 5% |

Lipid parameters at parent study baseline

| | | |
|---|---|---|
| LDL-C, mg/dL | 104 (28) | 130 (27) |
| LDL 70-99 mg/dL, % | 59% | 0% |
| LDL ≥ 100 mg/dL, % | 41% | 100% |
| HDL-C, mg/dL | 50 (12) | 48 (11) |
| Triglycerides, mg/dL | 138 (74) | 164 (85) |

Abbreviations: HDL-C, high-density lipoprotein-cholesterol; LDL-C, low-density lipoprotein-cholesterol; SD, standard deviation.

In some embodiments, the combined therapy allows for an improvement (reduction) in the population event rates per 100 patients (Standard Background Therapy vs. Evolocumab plus Standard Background Therapy), as outlined in the table below.

| | |
|---|---|
| Evolocumab + | |
| SOC | SOC |

10-year Horizon

| | | |
|---|---|---|
| Rate of Non-fatal MI | 18 | 29 |
| Rate of Non-fatal IS | 18 | 26 |
| Rate of CV death | 23 | 25 |
| Rate of revascularization | 27 | 38 |
| Rate of MI, IS or CV death | 58 | 79 |
| Risk of MI, IS or CV death | 44% | 55% |
| (%) | | |

Lifetime Horizon

| | | |
|---|---|---|
| Rate of Non-fatal MI | 41 | 65 |
| Rate of Non-fatal IS | 43 | 58 |
| Rate of CV death | 51 | 56 |
| Rate of revascularization | 58 | 79 |
| Rate of MI, IS or CV death | 135 | 179 |
| Risk of MI, IS or CV death | 74% | 83% |
| (%) | | |

Abbreviations: CV, cardiovascular; IS, ischemic stroke; MI, myocardial infarction; SOC, standard of care.

In some embodiments, a method of reducing a risk of urgent coronary revascularization can comprise a) identifying a subject that is on a first therapy, wherein the first therapy comprises a non-PCSK9 LDL-C lowering therapy, and b) administering a second therapy to the subject, wherein the second therapy comprises a PCSK9 inhibitor therapy. Both the first and second therapies are administered to the subject in an amount and time sufficient to reduce the risk of atherosclerotic cardiovascular disease in the subject, and wherein the first therapy is not the same as the second therapy. In some embodiments, the risk is not cardiovascular death over more than 12 months and less than 36 months separate from myocardial infarction and stroke.

In some embodiments, a method of reducing a risk of a cardiovascular event is provided. The method comprises a) identifying a subject with cardiovascular disease, and b) administering a PCSK9 inhibitor to the subject in an amount and overtime sufficient to reduce a risk of at least one of cardiovascular death, non-fatal myocardial infarction, non-fatal stroke or transient ischemic attack (TIA), coronary revascularization, or hospitalization for unstable angina. In some embodiments, the subject with cardiovascular disease is on a non-PCSK9 LDL-C lowering therapy, wherein the non-PCSK9 LDL-C lowering therapy is not a same therapy as the PCSK9 inhibitor. Both the non-PCSK9 LDL-C lowering therapy and the PCSK9 inhibitor are administered to the subject in an amount and time sufficient to reduce a risk of a cardiovascular event in the subject. In some embodiments, the non-PCSK9 LDL-C lowering therapy comprises a statin. In some embodiments, the risk is not cardiovascular death over more than 12 months and less than 36 months separate from myocardial infarction and stroke.

In some embodiments, a method of lowering LDL-C levels in a subject is provided. The method comprising administering: a) first therapy to a subject, wherein the first therapy comprises a non-PCSK9 LDL-C lowering therapy, and b) a second therapy to the subject, wherein the second therapy comprises a PCSK9 inhibitor. Both the first and second therapies are administered to the subject for at least five years, and the first therapy is not the same as the second therapy. In some embodiments, the subject's LDL-C level is maintained beneath 50 mg/dL.

In some embodiments, a method of reducing a risk of a cardiovascular event is provided. The method comprises a) identifying a subject that is on a first therapy, wherein the first therapy comprises a non-PCSK9 LDL-C lowering therapy, and b) administering a second therapy to the subject. The second therapy comprises a PCSK9 inhibitor. Both the first and second therapies are administered to the subject in an amount and time sufficient to reduce a risk of a cardiovascular event in the subject. The first therapy is not the same as the second therapy. The risk is at least one of myocardial infarction, stroke, hospitalization for unstable angina, or coronary revascularization.

In some embodiments, the subject to receive the combined therapy for an improved cardiovascular outcome has least 1 major risk factor or at least 2 minor risk factors below:

Major Risk Factors:
diabetes (type 1 or type 2)
age ≥65 years at randomization (and ≤85 years at time of informed consent)
MI or non-hemorrhagic stroke within 6 months of screening
additional diagnosis of myocardial infarction or non-hemorrhagic stroke excluding qualifying MI or non-hemorrhagic stroke^{a}
current daily cigarette smoking
history of symptomatic PAD (intermittent claudication with ABI <0.85, or peripheral arterial revascularization procedure, or amputation due to atherosclerotic disease) if eligible by MI or stroke history

Minor Risk Factors:
history of non-MI related coronary revascularization^{a}
residual coronary artery disease with ≥40% stenosis in ≥2 large vessels
Most recent HDL-C <40 mg/dL (1.0 mmol/L) for men and <50 mg/dL (1.3 mmol/L) for women by central laboratory before randomization
Most recent hsCRP >2.0 mg/L by central laboratory before randomization
Most recent LDL-C ≥130 mg/dL (3.4 mmol/L) or non-HDL-C ≥160 mg/dL (4.1 mmol/L) by central laboratory before randomization
metabolic syndrome^{b}

In some embodiments, the subject to receive the combined therapy for an improved cardiovascular outcome has: a most recent fasting LDL-C ≥70 mg/dL (≥1.8 mmol/L) or non-HDL-C ≥100 mg/dL (≥2.6 mmol/L) after ≥2 weeks of stable lipid lowering therapy per discussion in Example 17, and/or a most recent fasting triglycerides ≥400 mg/dL (4.5 mmol/L) by central laboratory before randomization.

### Peripheral Artery Disease

In some embodiments, one or more of the various treatment approaches provided herein can be used in a subject who has, or is at risk of developing peripheral artery disease (“PAD”). The application of a combination therapy to such a subject is outlined in Example 18. By way of context, the presence of peripheral artery disease (PAD) is a marker of a malignant vascular phenotype with event rates exceeding those of other stable populations with atherosclerosis, particularly in the setting of polyvascular disease. (Suarez C, Zeymer U, Limbourg T, et al. Influence of polyvascular disease on cardiovascular event rates. Insights from the REACH Registry. Vasc Med 2010; 15(4): 259-65. Criqui M H, Aboyans V. Epidemiology of peripheral artery disease. Circ Res 2015; 116(9): 1509-26. Bonaca M P, Bhatt D L, Storey R F, et al. Ticagrelor for Prevention of Ischemic Events After Myocardial Infarction in Patients With Peripheral Artery Disease. J Am Coll Cardiol 2016; 67(23): 2719-28.) Thus, patients with symptomatic PAD are at heightened risk of major adverse cardiovascular events (MACE) including myocardial infarction, stroke and cardiovascular death. (Aboyans V, Ricco J B, Bartelink M E L, et al. 2017 ESC Guidelines on the Diagnosis and Treatment of Peripheral Arterial Diseases, in collaboration with the European Society for Vascular Surgery (ESVS): Document covering atherosclerotic disease of extracranial carotid and vertebral, mesenteric, renal, upper and lower extremity arteries Endorsed by: the European Stroke Organization (ESO) The Task Force for the Diagnosis and Treatment of Peripheral Arterial Diseases of the European Society of Cardiology (ESC) and of the European Society for Vascular Surgery (ESVS). Eur Heart J 2017; Gerhard-Herman M D, Gornik H L, Barrett C, et al. 2016 AHA/ACC Guideline on the Management of Patients With Lower Extremity Peripheral Artery Disease: A Report of the American College of Cardiology/American Heart Association Task Force on Clinical Practice Guidelines. Circulation 2016.) In addition, patients with PAD suffer significant morbidity from major adverse limb events (MALE) including acute limb ischemia, urgent peripheral revascularization and major amputation. (Kumbhani D J, Steg P G, Cannon C P, et al. Statin therapy and long-term adverse limb outcomes in patients with peripheral artery disease: insights from the REACH registry. Eur Heart J 2014; 35(41): 2864-72; Jones W S, Baumgartner I, Hiatt W R, et al. Ticagrelor Compared With Clopidogrel in Patients with Prior Lower Extremity Revascularization for Peripheral Artery Disease. Circulation 2016; Bonaca MP, Scirica B M, Creager M A, et al. Vorapaxar in patients with peripheral artery disease: results from TRA2{degrees}P-TIMI 50. Circulation 2013; 127(14): 1522, 9, 1529e1-6.)

Although lipid-lowering therapy has been correlated in reducing MACE in stable patients with coronary heart disease or atherosclerosis risk factors, there have been few well-powered prospective randomized trials of low-density lipoprotein LDL cholesterol (LDL-C) reduction specifically in patients with PAD. (Aung P P, Maxwell H G, Jepson R G, Price J F, Leng G C. Lipid-lowering for peripheral arterial disease of the lower limb. Cochrane Database Syst Rev 2007; (4)(4): CD000123.) Moreover, these trials have not specifically looked at the ability of LDL-C lowering to reduce the risk of MALE, an important cause of morbidity in patients with PAD. (Kumbhani D J, Steg P G, Cannon C P, et al. Statin therapy and long-term adverse limb outcomes in patients with peripheral artery disease: insights from the REACH registry. Eur Heart J 2014; 35(41): 2864-72; Aronow W S, Nayak D, Woodworth S, Ahn C. Effect of simvastatin versus placebo on treadmill exercise time until the onset of intermittent claudication in older patients with peripheral arterial disease at six months and at one year after treatment. Am J Cardiol 2003; 92(6): 711-2; Mohler E R, 3rd, Hiatt W R, Creager M A. Cholesterol reduction with atorvastatin improves walking distance in patients with peripheral arterial disease. Circulation 2003; 108(12): 1481-6; Spring S, Simon R, van der Loo B, et al. High-dose atorvastatin in peripheral arterial disease (PAD): effect on endothelial function, intima-media-thickness and local progression of PAD. An open randomized controlled pilot trial. Thromb Haemost 2008; 99(1): 182-9; Schanzer A, Hevelone N, Owens C D, Beckman J A, Belkin M, Conte M S. Statins are independently associated with reduced mortality in patients undergoing infrainguinal bypass graft surgery for critical limb ischemia. J Vasc Surg 2008; 47(4): 774-81.) Lastly, as PAD has often been used simply as a risk enhancer, little is known about PAD patients without prior MI or stroke. (Bonaca M P, Scirica B M, Creager M A, et al. Vorapaxar in patients with peripheral artery disease: results from TRA2{degrees}P-TIMI 50. Circulation 2013; 127(14): 1522, 9, 1529e1-6. Aung P P, Maxwell H G, Jepson R G, Price J F, Leng G C. Lipid-lowering for peripheral arterial disease of the lower limb. Cochrane Database Syst Rev 2007; (4)(4): CD000123; Hiatt W R, Fowkes F G, Heizer G, et al. Ticagrelor versus Clopidogrel in Symptomatic Peripheral Artery Disease. N Engl J Med 2016; Anand S. et al. <br/>COMPASS PAD—Cardiovascular OutcoMes for People using Anticoagulation StrategieS trial: Results in Patients with Peripheral Artery Disease. European Society of Cardiology Hotline 2017.)

FOURIER was a very large cardiovascular outcomes trial of the PCSK9 inhibitor evolocumab and enrolled patients with atherosclerotic disease, in either the coronary, cerebrovascular or peripheral arterial bed. FOURIER thus allowed one to test the following hypotheses: (1) patients with PAD would be at greater risk of MACE relative to patients with coronary or cerebrovascular disease without PAD; (2) consistent relative risk reductions in MACE with evolocumab would translate to larger absolute risk reductions in patients with PAD relative to those without; and (3) LDL-C reduction with evolocumab would significantly reduce MALE with benefits extending to very low levels of LDL-C. This is examined and its application confirmed in Example 18 below.

As detailed in Example 18 below, patients with symptomatic lower extremity PAD are at heightened risk of major adverse cardiovascular and limb risks. Combination therapies, such as Evolocumab added to statin therapy, significantly and robustly reduced the risk of MACE, even in patients with PAD and no prior MI or stroke. Likewise, combination therapies, such as the addition of evolocumab to a statin, reduced the risk of major adverse limb events, and the relationship between achieved LDL-C and lower risk of limb events extended down to very low achieved levels of LDL. These benefits come with no apparent safety concerns. Thus, LDL-C reduction to very low levels is useful in patients with PAD, regardless of a history of MI or stroke, to reduce the risk of MACE and MALE.

In some embodiments, a method of treating a subject is provided. The method comprises identifying a subject with peripheral artery disease and reducing a level of PCSK9 activity in the subject.

In some embodiments, a method of reducing a risk of an adverse limb event in a subject is provided, the method comprises reducing a level of PCSK9 activity in a subject, wherein the subject has peripheral artery disease.

In some embodiments, a method of reducing a risk of a major cardiovascular adverse event (“MACE”) is provided. The method comprises administering a non-statin LDL-C lowering agent to a subject and administering a statin to the subject. The subject has PAD.

In some embodiments, a method of reducing a risk of a major adverse limb event (“MALE”) is provided. The method comprises administering a non-statin LDL-C lowering agent to a subject and administering a statin to the subject. The subject has peripheral artery disease (“PAD”).

For any of the preceding embodiments regarding PAD, MACE, MALE, or the combination thereof, any of the combination therapies and/or compositions provided in the present application can be employed.

For any of the preceding embodiments regarding PAD, MACE, MALE, or the combination thereof, any of the following aspects are also contemplated (as well as any appropriate aspects provided elsewhere in the present specification).

In some embodiments, the subject is further administered a non-PCSK9 LDL-C lowering therapy. In some embodiments, the non-PCSK9 LDL-C lowering therapy comprises a statin. In some embodiments, any of the non-PCSK9 LDL-C lowering therapies provided herein can be employed. In some embodiments, the amount of the statin can be at least atorvastatin 20 mg daily or equivalent, titrated to achieve LDL-C reduction per regional guidelines. In some embodiments, the amount of the statin can be at least equivalent to atorvastatin 40 mg daily or higher.

In some embodiments, the adverse limb event is selected from the group consisting of at least one of: acute limb ischemia, major amputation and urgent peripheral revascularization.

In some embodiments, the subject has no history of myocardial infarction or stroke. Despite this, the subject still receives a benefit from the therapy. In some embodiments, the subject has a history of myocardial infarction and/or stroke and will still receive a benefit from the therapy. In some embodiments, the subject has not had a prior MI or stroke. In some embodiments, the subject has had a prior MI or stroke.

In some embodiments, the subject is identified to receive therapy if the subject had intermittent claudication and an ankle brachial index of <0.85, if they had a prior peripheral procedure (lower extremity revascularization or amputation), or if they had both.

In some embodiments, the therapy provides a reduction in a risk of a composite of cardiovascular death, myocardial infarction, stroke, hospital admission for unstable angina, or coronary revascularization.

In some embodiments, reducing a level of PCSK9 activity in a subject is achieved via an antibody to PCSK9. In some embodiments, any PCSK9 inhibitor or PCSK9 LDL-C lowering agent or therapy can be used. In some embodiments, any PCSK9 inhibitor or PCSK9 LDL-C lowering agent or therapy provided in the present specification can be employed. In some embodiments, the PCSK9 LDL-C lowering agent comprises an antibody. In some embodiments, the PCSK9 LDL-C lowering agent comprises evolocumab. In some embodiments, the amount of the PCSK9 LDL-C lowering agent administered is as outlined within the present specification. In some embodiments, the amount of the PCSK9 LDL-C lowering agent will be sufficient such that, when combined with the non-PCSK9-LDL-C lowering agent, the subject's LDL-C level is lowered to less than 70, 60, 50, 40, 30, 20, or 10 mg/dL. In some embodiments, the amount of evolocumab administered is between 100 and 840, for example 120 and 700, 140 and 600, 140 and 500, 140 and 420, 210 and 630, 140, or 420 mg. In some embodiments, the amount of evolocumab administered is 140 mg, once every two weeks or 420 mg once a month. In some embodiments, a combination therapy (as provided herein, can be administered to a subject who has a LDL-C level of greater than 70 mg/dL, to reduce the subject's LDL-C level to a very low level, for example, less than 60, such as less than: 55, 50, 45, 40, 35, 30. 25, 20, 15, or 10 mg/dL or lower (including any range between any two of the preceding values. This method can be applied to any one of more of the indications and/or goals provided herein, including, but not limited to, reducing a risk of: a major vascular event, a cardiovascular event, major cardiovascular adverse event, major adverse limb event, adverse limb event, PAD, fatal MI and/or non-fatal MI and fatal and/or non-fatal coronary revascularization, composite of: a) coronary revascularization, b) myocardial infarction, and c) cerebral vascular accident, composite of: a) cardiovascular death, b) myocardial infarction, c) stroke, d) hospitalization for unstable angina, or e) coronary revascularization, urgent coronary revascularization, at least one of: a) cardiovascular death, b) myocardial infarction, c) stroke, d) hospitalization for unstable angina, or e) coronary revascularization, or a cardiovascular event by at least 10%. This method can also be applied to: treating atherosclerotic cardiovascular disease, treating coronary atherosclerosis, providing regression of coronary atherosclerosis, treating a subject that is unable to tolerate a full therapeutic dose of a statin, treating a subject that is unable to tolerate a full therapeutic dose of a non-PCSK9 LDL-C lowering agent, combining a PCSK9 inhibitor therapy and a non-PCSK9 LDL-C lowering therapy to produce greater LDL-C lowering and regression of coronary atherosclerosis at a dose that is well tolerated, reducing disease progression, reducing an amount of atherosclerotic plaque in a subject, combining evolocumab and a statin therapy to produce greater LDL-C lowering and regression of coronary atherosclerosis at a dose that is well tolerated, decreasing a LDL-C level in a subject beneath 80 mg/dL, decreasing total atheroma volume (TAV) in a subject, decreasing percent atheroma volume (PAV) in a subject, for lowering LDL-C level, and for reducing disease progression or any combination thereof. Thus, in some embodiments, any of the combination therapies provided herein can be employed for any of these applications, to a subject with a LDL-C level of at least 70 mg/dL, at a level effective to lower the subject's LDL-C level to a low level of less than, 60, 55, 50, 45, 40, 35, 30, 25, 20, 15, or 10 to achieve one or more of these aspects. With respect to the referenced combination therapy, this can be any described herein, including, a first therapy (e.g., a non-PCSK9 LDL-C lowering agent, a statin, an optimized amount of a statin) with a second therapy (e.g., a PCSK9 LDL-C lowering agent, a PCSK9 inhibitor, a non-statin LDL-C lowering agent, a anti-PCSK9 neutralizing antibody, evolocumab). In some embodiments, this therapy can be administered in an amount of at least 140 mg every two weeks or 420 mg once monthly. In some embodiments, instead of a subject receiving the combination therapy if their LDL-C level is above 70 mg/dl (or other value provided herein), they can receive it from an alternative indicator, such as non-HDL, which can be, (for 70 mg/dL) greater than or equal to 100 (of non-HDL).

In some embodiments, a reduction in risk to a subject is greater in a subject having PAD, than in a subject who does not have PAD.

In some embodiments, the subject has PAD, and following the therapy, the subject has a reduced the risk of MACE, MALE, or MACE and MALE.

In some embodiments, MALE is a composite of acute limb ischemia (ALI), major amputation (above the knee, AKA or below the knee BKA, excluding forefoot or toe), or urgent revascularization (thrombolysis or urgent vascular intervention for ischemia. In some embodiments, MACE is a composite of CV death, MI or stroke.

In some embodiments, the subject's LDL-C level is reduced to at least 50 mg/dL, for example, less than 50, 40, 30, 25, 20, 15, or 10 mg/dL. In some embodiments, the cardiovascular risk is reduced at least 10%, for example, at least 10, 15, 20, 25, 30, 35, 40, 45, or 50% reduction in cardiovascular risk.

In some embodiments, the risk of MALE, following therapy, is reduced at least 10%, for example at least 10, 15, 20, 25, 30, 35, 40, 45, or 50% reduction in risk. In some embodiments, the risk of MACE, following therapy, is reduced at least 10%, for example at least 10, 15, 20, 25, 30, 35, 40, 45, or 50% reduction in risk. In some embodiments, the risk of MALE and MACE is reduced at least 5%, for example, at least 5, 10, 15, 20, 25, or 30%.

In some embodiments, the subject to receive therapy is one identified as having a risk of MACE, MALE, or MACE and MALE. In some embodiments, the subject to receive therapy is one having a risk of, or actually having, PAD.

In some embodiments, subjects with PAD benefit especially from one or more of the methods provided herein, as they are in the highest risk patient group. That is, the subjects who have PAD are considered difficult to treat with other approaches. Thus, the present approach can be especially advantageous over other, less effective, approaches.

In some embodiments, the subject is one with PAD and/or one or more or recent myocardial infarctions (“MIs”).

As depicted in Example 19, in some embodiments, the methods provided herein are more effective in subjects with fewer such risk factors. For example, in some embodiments, the subject to be treated has less than 3 such risk factors, such as 2, 1, or 0 of these risk factors. In some embodiments, the risk factors are at least one of PAV, HbA1c and/or a change in apolipoprotein A-I. In some embodiments, undesirable systolic blood pressure can be a risk factor. In some embodiments, factors associated with a greater propensity to ongoing plaque progression, included the presence of additional atherogenic factors, and thus, in some embodiments, the subject to be treated does not have too many additional atherogenic factors (e.g., less than 3, 2, 1, or has none). In some embodiments, any of the combination therapies provided herein can be employed to assist subjects with recent and/or multiple myocardial infarctions. In some embodiments, the MI is within 4 or more weeks. In some embodiments, the MI is within 1, 2, 3, 4, 5, 6, 7, 8, 9, 10, 11, 12, 13, 14, 15, 16, 17, 18, 19, 20, 21, 22, 23, or 24 months. In some embodiments, the subject has suffered from more than one MI, for example, 2, 3, 4 or more MIs. In some embodiments, the subject has multivessel disease. In some embodiments, the subject has some combination of 1) recent MI (within 2 years), 2) multiple MIs (more than 1), and/or multivessel disease. In some embodiments, a subject with one or more of these, who then receives a therapy as noted herein, can then receive a decreased risk in CVD, MI, and/or stroke. In some embodiments, this additional screening or selection process can be used to identify subject to receive one or more of the combination therapies provided herein, including, for example, any of those within the Summary or the claims. In some embodiments, the risk is decreased by at least 1%, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30%, or more. In some embodiments, a subject having a recent or multiple MIs is administered (or continues to receive) a first therapy, wherein the first therapy comprises a non-PCSK9 LDL-C lowering therapy and a second therapy is also administered to the subject. The second therapy comprises a PCSK9 inhibitor therapy. In some embodiments, both the first and second therapies are administered to the subject in an amount and time sufficient to reverse coronary atherosclerosis in the subject.

As demonstrated in the results in Example 20, in some embodiments, any of the methods provided herein can be applied selectively to subjects with a Lp(a) level of greater than 11.8 mg/dL. In some embodiments, the subject has a Lp(a) level of more than 11.8 mg/dL, and thus, can receive an even greater benefit for plaque regression. In some embodiments, the subject has a Lp(a) level of at least (or between any two of the following) 11.8, 12, 13, 14, 15, 16, 17, 18, 19, 20, 25, 30, 35, 40, 45 or 49 or 50 mg/dL. In some embodiments, the Lp(a) is more than 30 mg/dL. In some embodiments, this additional screening or selection process can be used to identify a subject to receive one or more of the combination therapies provided herein, including, for example, any of those within the Summary or the claims. In some embodiments, the method to be applied, after the subject is identified as having a Lp(a) level above 11.8 mg/dL (but optionally below 30 mg/dL) is to provide (or continue providing) a first therapy, wherein the first therapy comprises a non-PCSK9 LDL-C lowering therapy and to administer a second therapy to the subject. The second therapy comprises a PCSK9 inhibitor therapy. In some embodiments, both the first and second therapies are administered to the subject in an amount and time sufficient to reverse coronary atherosclerosis in the subject. In some embodiments, both the first and second therapies are administered to the subject in an amount and time sufficient to reduce plaque formation.

In some embodiments, any of the following numbered arrangements can be employed.

1. A method of treating coronary atherosclerosis, the method comprising:
a. identifying a subject that is on a first therapy, wherein the first therapy comprises a non-PCSK9 LDL-C lowering therapy; and
b. administering a second therapy to the subject, wherein the second therapy comprises a PCSK9 inhibitor therapy, wherein both the first and second therapies are administered to the subject in an amount and time sufficient to reverse coronary atherosclerosis in the subject, and wherein the first therapy is not the same as the second therapy.

2. The method of arrangement 1, wherein the first therapy is selected from at least one of: a statin, including but not limited to atorvastatin (LIPITOR®), cerivastatin, fluvastatin (LESCOL), lovastatin (MEVACOR, ALTOPREV), mevastatin, pitavastatin, pravastatin (PRAVACHOL), rosuvastatin, rosuvastatin calcium (CRESTOR) and simvastatin (ZOCOR); ADVICOR (lovastatin+niacin), CADUET (atorvastatin+amlopidine); a selective cholesterol absorption inhibitor, including but not limited to ezetimibe (ZETIA); a Lipid Lowering Therapy (LLT) including but not limited to fibrates or fibric acid derivatives, including but not limited to gemfibrozil (LOPID), fenofibrate (ANTARA, LOFIBRA, TRICOR, TRIGLIDE) and clofibrate (ATROMID-S); a Resin including but not limited to cholestyramine (QUESTRAN, QUESTRAN LIGHT, PREVALITE, LOCHOLEST, LOCHOLEST LIGHT), cholestipol (CHOLESTID) and cholesevelan HCl (WELCHOL) and/or a combination thereof, including but not limited to VYTORIN (simvastatin+ezetimibe).

3. The method of any of the numbered arrangements in this section, wherein the first therapy is an optimized statin therapy.

4. The method of any of the numbered arrangements in this section, wherein the subject's LDL level decreases to a level beneath 80 mg/dL.

5. A method of treating coronary atherosclerosis, the method comprising:
a. identifying a subject that has a LDL-C level of less than 70 mg/dL; and
b. administering an anti-PCSK9 neutralizing antibody to the subject, in an amount sufficient and time sufficient to lower the LDL-C level to less than 60 mg/dL.

6. The method of any of the numbered arrangements in this section, wherein the subject has further been identified by being diagnosed with coronary atherosclerosis disease.

7. A method of decreasing percent atheroma volume (PAV) in a subject, the method comprising:
a. identifying a subject that has received at least a moderate level of treatment by a statin; and
b. b) administering an anti-PCSK9 neutralizing antibody to the subject in an amount sufficient and time sufficient to lower the LDL-C level to less than 100 mg/dL, e.g., less than 90 mg/dL, thereby decreasing a percent atheroma volume (PAV) in the subject.

8. The method of any of the numbered arrangements in this section, wherein the amount and time sufficient is sufficient to lower the LDL-C level to less than 40 mg/dL.

9. A method of decreasing total atheroma volume (TAV) in a subject, the method comprising:
a. identifying a subject that has received at least a moderate level of treatment by a statin; and
b. administering an anti-PCSK9 neutralizing antibody to the subject in an amount sufficient and time sufficient to lower the LDL-C level to less than 100 mg/dL, e.g., less than 90 mg/dL, thereby decreasing a total atheroma volume in the subject.

10. The method of any of the numbered arrangements in this section, wherein the amount and time sufficient is sufficient to lower the LDL-C level to less than 40 mg/dL.

11. The method of any of the numbered arrangements in this section, wherein administering the anti-PCSK9 neutralizing antibody to the subject decreases a percent atheroma volume in the subject.

12. The method of any of the numbered arrangements in this section, wherein a decrease of at least 0.1 percent is achieved in the PAV.

13. The method of any of the numbered arrangements in this section, wherein the decrease is achieved within 18 months.

14. The method of any of the numbered arrangements in this section, wherein the PAV is decreased by at least 1% following 18 months of treatment.

15. The method of any of the numbered arrangements in this section, wherein the PAV is decreased by at least 2% following 18 months of treatment.

16. A method of treating coronary atherosclerosis, the method comprising:
a. administering an optimum statin treatment to a subject, wherein the subject has coronary atherosclerosis; and
b. administering an amount of an anti-PCSK9 neutralizing antibody to the subject at the same time.

17. A method of treating coronary atherosclerosis, the method comprising:
a. identifying a statin-intolerant subject;
b. administering at least a low dose statin treatment to the statin-intolerant subject; and
c. administering an amount of an anti-PCSK9 neutralizing antibody to the subject, thereby treating coronary atherosclerosis.

18. A method of providing regression of coronary atherosclerosis, the method comprising:
providing a subject that is on an optimized level of a statin; and
administering to the subject an anti-PCSK9 neutralizing antibody, at a level adequate to regress coronary atherosclerosis, wherein regression is any change in PAV or TAV less than zero.

19. A method of decreasing a LDL-C level in a subject beneath 80 mg/dL, the method comprising: administering an anti-PCSK9 neutralizing antibody to a subject, wherein the subject has coronary atherosclerotic disease, wherein the subject is on an optimized statin therapy for at least one year, and wherein a LDL-C level in the subject decreases to an average value that is beneath 80 mg/dL for the at least one year.

20. The method of any of the numbered arrangements in this section, wherein the subject decreases to an average value that is beneath 60 mg/dL for the at least one year.

21. The method of any of the numbered arrangements in this section, wherein the subject decreases to an average value that is beneath 40 mg/dL for the at least one year.

22. A method of reducing a relative risk of a cardiovascular event by at least 10%, the method comprising administering a PCSK9 neutralizing antibody to a subject that is on at least a moderate intensity of a statin, in an amount sufficient to lower a LDL-C level of the subject by about 20 mg/dL.

23. The method of arrangement 22, wherein the cardiovascular event is one selected from the group of selected from the group of non-fatal myocardial infarction, myocardial infarction (MI), stroke/TIA, angina, arterial revascularization, coronary revascularization, fatal and non-fatal stroke, hospitalization for CHF, CHD deaths, coronary death, cardiovascular.

24. A method of reducing an amount of atherosclerotic plaque in a subject, the method comprising administering to a subject having atherosclerotic plaque a monoclonal antibody to human PCSK9, wherein the subject is receiving optimized statin therapy, thereby reducing the amount of atherosclerotic plaque in the subject.

25. The method of arrangement 24, further comprising, identifying a subject who is in need of reducing the amount of atherosclerotic plaque in the subject.

26. A method of reducing disease progression, the method comprising:
identifying a subject with a LDL-C level of no more than 60 mg/dL;
administering at least a moderate intensity of a statin therapy to the subject; and
administering evolocumab at a level sufficient to decrease the LDL-C level of the subject to 30 mg/dL, thereby reducing disease progression.

27. The method of any of the numbered arrangements in this section, wherein the subject has had a heart attack.

28. A method of combining evolocumab and a statin therapy to produce greater LDL-C lowering and regression of coronary atherosclerosis at a dose that is well tolerated, the method comprising:
administering at least a moderate intensity of a statin therapy to a subject;
administering an adequate amount of evolocumab to the subject such that the subject's LDL-C levels drop to no more than 40 mg/dL; and
maintaining the subject's LDL-C levels at no more than 40 mg/dL for at least one year.

29. A method of treating coronary atherosclerosis, the method comprising:
a. identifying a subject that has a LDL-C level of less than 70 mg/dL; and
b. administering a PCSK9 inhibitor to the subject, in an amount sufficient and time sufficient to lower the LDL-C level to less than 60 mg/dL.

30. A method of decreasing percent atheroma volume (PAV) in a subject, the method comprising:
a. identifying a subject that has received at least a moderate level of treatment by a non-PCSK9 LDL-C lowering agent; and
b. administering a PCSK9 inhibitor to the subject in an amount sufficient and time sufficient to lower the LDL-C level to less than 100 mg/dL, e.g., less than 90 mg/dL, thereby decreasing a percent atheroma volume (PAV) in the subject.

31. A method of decreasing total atheroma volume (TAV) in a subject, the method comprising:
a. identifying a subject that has received at least a moderate level of treatment by a non-PCSK9 LDL-C lowering agent; and
b. administering a PCSK9 inhibitor to the subject in an amount sufficient and time sufficient to lower the LDL-C level to less than 100 mg/dL, e.g., less than 90 mg/dL, thereby decreasing a total atheroma volume in the subject.

32. A method of treating coronary atherosclerosis, the method comprising:
a. administering an optimum non-PCSK9 LDL-C lowering therapy to a subject, wherein the subject has coronary atherosclerosis; and
b. administering an amount of a PCSK9 inhibitor to the subject at the same time.

33. A method of treating coronary atherosclerosis, the method comprising:
a. identifying a statin-intolerant subject;
b. administering at least a low intensity statin treatment to the statin-intolerant subject; and
c. administering an amount of a PCSK9 inhibitor to the subject, thereby treating coronary atherosclerosis.

34. A method of providing regression of coronary atherosclerosis, the method comprising:
providing a subject that is on an optimized level of a non-PCSK9 LDL-C lowering agent; and
administering to the subject a PCSK9 inhibitor, at a level adequate to regress coronary atherosclerosis, wherein regression is any change in PAV or TAV less than zero.

35. A method of decreasing a LDL-C level in a subject beneath 80 mg/dL, the method comprising: administering a PCSK9 inhibitor to a subject, wherein the subject has coronary atherosclerotic disease, wherein the subject is on an optimized non-PCSK9 LDL-C lowering therapy for at least one year, and wherein a LDL-C level in the subject decreases to an average value that is beneath 80 mg/dL for the at least one year.

36. A method of reducing an amount of atherosclerotic plaque in a subject, the method comprising administering to a subject having atherosclerotic plaque a PCSK9 inhibitor, wherein the subject is receiving optimized non-PCSK9 LDL-C lowering therapy, thereby reducing the amount of atherosclerotic plaque in the subject.

37. A method of reducing disease progression, the method comprising:
identifying a subject with a LDL-C level of no more than 60 mg/dL;
administering at least a moderate intensity of a non-PCSK9 LDL-C lowering therapy to the subject; and
administering a PCSK9 inhibitor at a level sufficient to decrease the LDL-C level of the subject to 30 mg/dL, thereby reducing disease progression.

38. A method of combining a PCSK9 inhibitor therapy and a non-PCSK9 LDL-C lowering therapy to produce greater LDL-C lowering and regression of coronary atherosclerosis at a dose that is well tolerated, the method comprising:
administering at least a moderate intensity of a non-PCSK9 LDL-C lowering therapy to a subject;
administering an adequate amount of a PCSK9 inhibitor to the subject such that the subject's LDL-C levels drop to no more than 40 mg/dL; and
maintaining the subject's LDL-C levels at no more than 40 mg/dL for at least one year.

39. A method of treating a subject that is unable to tolerate a full therapeutic dose of a non-PCSK9 LDL-C lowering agent, the method comprising:
identifying said subject; and
administering a PCSK9 inhibitor to the subject until a LDL cholesterol level of the subject decreases beneath 60 mg/dL.

40. The method of any of the numbered arrangements in this section, wherein the PCSK9 inhibitor comprises any of the 6 CDR sequences depicted in FIGS. 6-12.

41. The method of any of the numbered arrangements in this section wherein the first therapy comprises a moderate or a high-intensity statin therapy.

42. The method of any of the numbered arrangements in this section comprising a statin at a level of an effective dose of atorvastatin of at least 20 mg daily or an equivalent to atorvastatin at an equivalent amount.

43. The method of any of the numbered arrangements in this section, wherein the amount of the statin is at least an effective dose of atorvastatin of at least 40 mg daily or an equivalent to atorvastatin at an equivalent amount.

44. The method of any of the numbered arrangements in this section, wherein the statin is at least one of atorvastatin, simvastatin, rosuvastatin, pravastatin, lovastatin, and pitavastatin.

45. The method of any of the numbered arrangements in this section, wherein the statin is at least one of atorvastatin at 20, 40, or 80 mg; simvastatin at 40 or 80 mg; rosuvastatin at 5, 10, 20, or 40 mg; pravastatin at 80 mg, lovastatin at 80 mg, or pitavastatin at 4 mg.

46. The method of any of the numbered arrangements in this section, wherein the subject is on at least atorvastatin 40 or 80 mg; rosuvastatin 10, 20, or 40 mg; or simvastatin 80 mg.

47. The method of any of the numbered arrangements in this section, wherein the statin is a monotherapy for the statin.

48. The method of any of the numbered arrangements in this section, wherein the subject is also on an additional lipid lowering therapy.

49. The method of any of the numbered arrangements in this section, wherein the additional lipid lowering therapy is niacin, ezetimibe, or both niacin and ezetimibe.

50. The method of any of the numbered arrangements in this section, wherein the PCSK9 inhibitor or the anti-PCSK9 antibody is evolocumab, and wherein evolocumab is administered in an amount of at least 140 mg.

51. The method of any of the numbered arrangements in this section, wherein evolocumab is administered in an amount of at least 420 mg.

52. The method of any of the numbered arrangements in this section, wherein the PCSK9 inhibitor or the anti-PCSK9 antibody is evolocumab, and wherein evolocumab is administered at a frequency of at least once a month.

53. The method of any of the numbered arrangements in this section, wherein providing regression of coronary atherosclerosis denotes a decrease in PAV.

54. The method of any of the numbered arrangements in this section, wherein an LDL-C level in the subject is decreased beneath 60 mg/dL.

55. The method of any of the numbered arrangements in this section, wherein an LDL-C level in the subject is decreased beneath 50 mg/dL.

56. The method of any of the numbered arrangements in this section, wherein an LDL-C level in the subject is decreased beneath 40 mg/dL.

57. The method of any of the numbered arrangements in this section, wherein an LDL-C level in the subject is decreased beneath 30 mg/dL.

58. The method of any of the numbered arrangements in this section, wherein an LDL-C level in the subject is decreased beneath 20 mg/dL.

59. The method of any of the numbered arrangements in this section, wherein a risk of a CV death, non-fatal myocardial infarction, non-fatal stroke or transient ischemic attack (TIA), coronary revascularization, and hospitalization for unstable angina for the subject is reduced.

60. The method of any of the numbered arrangements in this section, wherein an amount of the anti-PCSK9 neutralizing antibody is at least 140 mg.

61. The method of any of the numbered arrangements in this section, wherein an amount of the anti-PCSK9 neutralizing antibody is at least 150 mg.

62. The method of any of the numbered arrangements in this section, wherein an amount of the anti-PCSK9 neutralizing antibody is at least 300 mg.

63. The method of any of the numbered arrangements in this section, wherein an amount of the anti-PCSK9 neutralizing antibody is at least 400 mg.

64. The method of any of the numbered arrangements in this section, wherein an amount of the anti-PCSK9 neutralizing antibody is 420 mg.

65. The method of any of the numbered arrangements in this section, further comprising evolocumab.

66. The method of any of the numbered arrangements in this section, wherein evolocumab is administered subcutaneously.

67. The method of any of the numbered arrangements in this section, wherein evolocumab is administered at least monthly to the subject for at least one year.

68. The method of any of the numbered arrangements in this section, wherein a percent atheroma volume (PAV) in the subject decreases by 0.1 to 2.5%.

69. The method of any of the numbered arrangements in this section, wherein the normalized total atheroma volume decreases by 0.1 to 10% 70. The method of any of the numbered arrangements in this section, wherein a LDL-C level of the subject decreases by at least 40%.

71. The method of any of the numbered arrangements in this section, wherein a LDL-C level of the subject decreases by at least 60%.

72. The method of any of the numbered arrangements in this section, wherein the subject has been treated with a stable statin dose for at least four weeks and has a LDL-C ≥80 mg/dL or between 60 and 80 mg/dL with one major or three minor cardiovascular risk factors.

73. The method of any of the numbered arrangements in this section, comprising an anti-PCSK9 neutralizing antibody.

74. The method of any of the numbered arrangements in this section, wherein the anti-PCSK9 neutralizing antibody is evolocumab.

75. The method of any of the numbered arrangements in this section, wherein a major risk factor comprises at least one of: non-coronary atherosclerotic vascular disease, myocardial infarction or hospitalization for unstable angina in the preceding 2 years or type 2 diabetes mellitus.

76. The method of any of the numbered arrangements in this section, wherein a minor risk factor comprises at least one of: current cigarette smoking, hypertension, low levels of high-density lipoprotein cholesterol (HDL-C), family history of premature coronary heart disease, high sensitivity C-reactive protein (hs-CRP) ≥2 mg/L or age ≥50 years in men and 55 years in women.

77. A method of treating a subject that is unable to tolerate a full therapeutic dose of a statin, the method comprising:
identifying said subject; and
administering a PCSK9 inhibitor to the subject until a LDL cholesterol level of the subject decreases beneath 60 mg/dL.

78. A method of treating coronary atherosclerosis, the method comprising:
a. identifying a subject that has a LDL-C level of less than 70 mg/dL; and
b. administering a non-PCSK9 LDL-C lowering agent to the subject, in an amount sufficient and time sufficient to lower the LDL-C level to less than 60 mg/dL.

79. The method of any of the numbered arrangements in this section, wherein a high intensity of a statin is administered to the subject.

80. The method of any of the numbered arrangements in this section, wherein the person has been diagnosed with a cardiovascular disease.

81. The method of any of the numbered arrangements in this section, wherein evolocumab is administered every two weeks.

82. A method of treating atherosclerotic cardiovascular disease, the method comprising:
a. identifying a subject that is on a first therapy, wherein the first therapy comprises a non-PCSK9 LDL-C lowering therapy; and
b. administering a second therapy to the subject, wherein the second therapy comprises a PCSK9 inhibitor therapy, wherein both the first and second therapies are administered to the subject in an amount and time sufficient to reduce a risk of atherosclerotic cardiovascular disease in the subject, and wherein the first therapy is not the same as the second therapy, and wherein the risk is a) a composite for cardiovascular death, myocardial infarction, stroke, hospitalization for unstable angina, or coronary revascularization or b) a composite for cardiovascular death, myocardial infarction, or stroke.

83. The method of arrangement 82, wherein the first and second therapies are continued for at least two years.

84. The method of arrangement 83, wherein a risk of a composite of cardiovascular death, myocardial infarction, stroke, hospitalization for unstable angina, or coronary revascularization is decreased by at least 15%.

85. The method of arrangement 82, wherein a risk of a composite of cardiovascular death, myocardial infarction, or stroke is decreased by at least 20%.

86. A method of reducing a risk of a cardiovascular event, the method comprising:
a. identifying a subject that is on a first therapy, wherein the first therapy comprises a non-PCSK9 LDL-C lowering therapy; and
b. administering a second therapy to the subject, wherein the second therapy comprises a PCSK9 inhibitor, wherein both the first and second therapies are administered to the subject in an amount and time sufficient to reduce a risk of a cardiovascular event in the subject, and wherein the first therapy is not the same as the second therapy, and wherein the risk is a) a composite for cardiovascular death, myocardial infarction, stroke, hospitalization for unstable angina, or coronary revascularization or b) a composite for cardiovascular death, myocardial infarction, or stroke.

87. The method of arrangement 86, wherein the cardiovascular event is selected from at least one of: cardiovascular death, myocardial infarction, stroke, hospitalization for unstable angina, or coronary revascularization, and wherein the first and second therapies are continued for at least two years.

88. The method of arrangement 86, wherein a risk of a composite of cardiovascular death, myocardial infarction, stroke, hospitalization for unstable angina, or coronary revascularization is decreased by at least 15%.

89. The method of arrangement 86, wherein a risk of a composite of cardiovascular death, myocardial infarction, or stroke is decreased by at least 20%.

90. The method of arrangement 86, wherein a hazard ratio in a first year of reducing the risk is 0.84 (95% CI, 0.74-0.96) for cardiovascular death, myocardial infarction, or stroke.

91. The method of arrangement 86, wherein a hazard ratio in a second year of reducing the risk is 0.75 (95% CI, 0.66-0.85) for cardiovascular death, myocardial infarction, or stroke.

92. The method of arrangement 86, wherein a hazard ratio in a first year of reducing the risk is 0.88 (95% CI, 0.80-0.97) for cardiovascular death, myocardial infarction, stroke, hospitalization for unstable angina, or coronary revascularization.

93. The method of arrangement 86, wherein a hazard ratio in a second year of reducing the risk is 0.81 (95% CI, 0.73-0.89) for cardiovascular death, myocardial infarction, stroke, hospitalization for unstable angina, or coronary revascularization.

94. The method of any one of arrangements 82-93, wherein reducing the risk denotes at least one of a) increasing an amount of time to the first of any one of cardiovascular death, myocardial infarction, stroke, hospitalization for unstable angina, or coronary revascularization, or b) increasing an amount of time to the first of any one of cardiovascular death, myocardial infarction, or stroke.

95. The method of arrangement 86, wherein there is a 21% to 27% reduction in the risk of myocardial infarction, stroke and coronary revascularization.

96. The method of arrangement 86, wherein there is a 17% reduction in risk of cardiovascular death, myocardial infarction, or stroke in a subject, wherein the subject has an initial median LDL cholesterol of 126 mg/dL.

97. The method of arrangement 96, wherein a final median LDL cholesterol level of the subject is 43 mg/dL.

98. The method of arrangement 86, wherein there is a 22% reduction in risk of cardiovascular death, myocardial infarction, or stroke in a subject, wherein the subject has an initial median LDL cholesterol of 73 mg/dL.

99. The method of arrangement 98, wherein a final median LDL cholesterol level of the subject is 22 mg/dL.

100. A method of reducing a risk of urgent coronary revascularization, the method comprising:
a. identifying a subject that is on a first therapy, wherein the first therapy comprises a non-PCSK9 LDL-C lowering therapy; and
b. administering a second therapy to the subject, wherein the second therapy comprises a PCSK9 inhibitor therapy, wherein both the first and second therapies are administered to the subject in an amount and time sufficient to reduce the risk of atherosclerotic cardiovascular disease in the subject, and wherein the first therapy is not the same as the second therapy.

101. A method of reducing a risk of a cardiovascular event, the method comprising:
a. identifying a subject with cardiovascular disease;
b. administering a PCSK9 inhibitor to the subject in an amount and overtime sufficient to reduce a risk of at least one of cardiovascular death, non-fatal myocardial infarction, non-fatal stroke or transient ischemic attack (TIA), coronary revascularization, or hospitalization for unstable angina.

102. The method of arrangement 101, wherein the subject with cardiovascular disease is on a non-PCSK9 LDL-C lowering therapy, wherein the non-PCSK9 LDL-C lowering therapy is not a same therapy as the PCSK9 inhibitor, wherein both the non-PCSK9 LDL-C lowering therapy and the PCSK9 inhibitor are administered to the subject in an amount and time sufficient to reduce a risk of a cardiovascular event in the subject.

103. The method of arrangement 102, wherein the non-PCSK9 LDL-C lowering therapy comprises a statin.

104. The method of any one of arrangements 82-103, wherein the risk is for the composite of cardiovascular death, myocardial infarction, or stroke.

105. The method of any one of arrangements 82-103, wherein the risk is for the composite of cardiovascular death, myocardial infarction, stroke, hospitalization for unstable angina, or coronary revascularization.

106. A method of lowering LDL-C levels in a subject, the method comprising administering:
a. a first therapy to a subject, wherein the first therapy comprises a non-PCSK9 LDL-C lowering therapy; and
b. administering a second therapy to the subject, wherein the second therapy comprises a PCSK9 inhibitor, wherein both the first and second therapies are administered to the subject for at least five years, and wherein the first therapy is not the same as the second therapy, and wherein the subject's LDL-C level is maintained beneath 50 mg/dL.

107. A method of reducing a risk of a cardiovascular event, the method comprising:
a. identifying a subject that is on a first therapy, wherein the first therapy comprises a non-PCSK9 LDL-C lowering therapy; and
b. administering a second therapy to the subject, wherein the second therapy comprises a PCSK9 inhibitor, wherein both the first and second therapies are administered to the subject in an amount and time sufficient to reduce a risk of a cardiovascular event in the subject, and wherein the first therapy is not the same as the second therapy, and wherein the risk is at least one of myocardial infarction, stroke, hospitalization for unstable angina, or coronary revascularization.

108. A method of reducing a risk of a cardiovascular event, the method comprising:
a. identifying a subject that is on a first therapy, wherein the first therapy comprises a non-PCSK9 LDL-C lowering therapy; and
b. administering a second therapy to the subject, wherein the second therapy comprises a PCSK9 inhibitor, wherein both the first and second therapies are administered to the subject in an amount and time sufficient to reduce a risk of a cardiovascular event in the subject, and wherein the first therapy is not the same as the second therapy, and wherein the risk is the composite of coronary revascularization, myocardial infarction, cerebral vascular accident.

109. A method of reducing a risk of a cardiovascular event, the method comprising:
a. identifying a subject that is on a first therapy, wherein the first therapy comprises a non-PCSK9 LDL-C lowering therapy; and
b. administering a second therapy to the subject, wherein the second therapy comprises a PCSK9 inhibitor, wherein both the first and second therapies are administered to the subject in an amount and time sufficient to reduce a risk of a cardiovascular event in the subject, and wherein the first therapy is not the same as the second therapy, and wherein the risk is the composite of fatal MI and/or non-fatal MI and fatal and/or non-fatal coronary revascularization.

110. A method of treating a subject, the method comprising:
identifying a subject with peripheral artery disease; and
reducing a level of PCSK9 activity in the subject.

111. A method of reducing a risk of an adverse limb event in a subject, the method comprising: reducing a level of PCSK9 activity in a subject, wherein the subject has peripheral artery disease.

112. The method of arrangement 111, wherein the subject is further administered a non-PCSK9 LDL-C lowering therapy.

113. The method of arrangement 112, wherein the non-PCSK9 LDL-C lowering therapy comprises a statin.

114. The method of arrangement 113, wherein the adverse limb event is selected from the group consisting of at least one of: acute limb ischemia, major amputation and urgent peripheral revascularization.

115. The method of arrangement 113, wherein the subject has no history of myocardial infarction or stroke.

116. The method of arrangement 113, wherein the subject is identified if the subject had intermittent claudication and an ankle brachial index of <0.85, if they had a prior peripheral procedure (lower extremity revascularization or amputation), or if they had both.

117. The method of arrangement 113, wherein there is a reduction in a risk of a composite of cardiovascular death, myocardial infarction, stroke, hospital admission for unstable angina, or coronary revascularization.

118. The method of any one of arrangements 110-117, wherein reducing a level of PCSK9 activity in a subject is achieved via an antibody to PCSK9.

119. The method of arrangement 118, wherein the antibody comprises evolocumab.

120. The method of any one of arrangements 110-119, wherein the reduction in risk to a subject is greater in a subject having PAD, than in a subject that does not have PAD.

121. The method of any one of arrangements 110-119, wherein the subject has PAD and wherein, following the method, the subject has a reduced the risk of MACE.

122. The method of any one of arrangements 110-119, wherein the subject has not had a prior MI or stroke.

123. A method of reducing a risk of a major adverse limb event (“MALE”), said method comprising:
administering a non-statin LDL-C lowering agent to a subject; and
administering a statin to the subject, wherein the subject has peripheral artery disease (“PAD”).

124. The method of arrangement 123, wherein MALE is a composite of acute limb ischemia (ALI), major amputation (above the knee, AKA or below the knee BKA, excluding forefoot or toe), or urgent revascularization (thrombolysis or urgent vascular intervention for ischemia.

125. A method of reducing a risk of a major cardiovascular adverse event (“MACE”), said method comprising:
administering a non-statin LDL-C lowering agent to a subject; and
administering a statin to the subject, wherein the subject has PAD.

126. The method of arrangement 125, wherein MACE is a composite of CV death, MI or stroke.

127. The method of any one of arrangements 110-126, wherein the subject did not have a prior MI or stroke.

128. The method of any one of arrangements, 110-127, wherein the subject's LDL-C level is reduced to at least 50 mg/dL.

129. The method of any one of arrangements 110-128, wherein the subject's LDL-C level is reduced to at least 10 mg/dL.

130. The method of any one of arrangements 110-129, wherein a cardiovascular risk is reduced at least 10%.

131. The method of any one of arrangements 110-129, wherein a cardiovascular risk is reduced at least 40%.

132. The method of any one of arrangements 111-124, wherein the risk of MALE is reduced at least 10%.

133. The method of any one of arrangements 111-124, wherein the risk of MALE is reduced at least 20%.

134. The method of any one of arrangements 110-134, wherein a combined risk of MALE and MACE is reduced at least 10%.

135. The method of any one of arrangements 110-134, wherein a combined risk of MALE and MACE is reduced at least 20%.

136. A method of reducing a risk of a cardiovascular event, the method comprising:
providing a first therapy to a subject, wherein the first therapy comprises a non-PCSK9 LDL-C lowering therapy; and
providing a second therapy to the subject, wherein the second therapy comprises a PCSK9 inhibitor, wherein both the first and second therapies are administered to the subject, and wherein the subject has a Lp(a) level of 11.8 mg/dL to 50.

137. A method of reducing a risk of a major vascular event in a subject, the method comprising:
1) identifying a subject that has at least one of: (a) a recent MI, (b) multiple prior MIs, or (c) multivessel disease;
2) providing a first therapy to a subject, wherein the first therapy comprises a non-PCSK9 LDL-C lowering therapy; and
3) providing a second therapy to the subject, wherein the second therapy comprises a PCSK9 inhibitor,
thereby reducing a risk that the subject will have a major vascular event.

138. The method of arrangement 137, wherein the major vascular even is selected from the group consisting of at least one of: CVD, MI, or stroke.

139. The method of arrangement 137 or 138, wherein a recent MI is one that with within two years.

140. The method of one of arrangements 137-139, wherein the multiple prior MIs is at least 2.

141. The method of any one of arrangements 137-140, wherein the subject has at least two of (a) a recent MI, (b) multiple prior MIs, or (c) multivessel disease.

142. The method of any one of arrangements 137-140, wherein the subject has all three of (a) a recent MI, (b) multiple prior MIs, or (c) multivessel disease.

143. The method of any one of arrangement 1, 16, 18, 19, 32, 34, 35, 36, 82, 86, 100, 106, 107, 108, 109, 123, 125, 136, or 137, wherein the first therapy or the non-PCSK9 LDL-C lowering agent or the statin consists of or comprises an optimized amount of a statin, and wherein the second therapy, the PCSK9 LDL-C lowering agent, the PCSK9 inhibitor, the non-statin LDL-C lowering agent, or the anti-PCSK9 neutralizing antibody consists of or comprises evolocumab, alirocumab, or an antibody that competes with evolocumab or alirocumab.

144. The method of any one of arrangements 5, 7, 9, 17, 18, 19, 22, 29, 30, 31, 33, 37, 38, 39, 77, or 101, wherein the second therapy, the PCSK9 LDL-C lowering agent, the PCSK9 inhibitor, the non-statin LDL-C lowering agent, or the anti-PCSK9 neutralizing antibody consists of or comprises evolocumab, alirocumab, or an antibody that competes with evolocumab or alirocumab.

145. The method of arrangements 143 or 144, wherein the statin is at least one of atorvastatin at 20, 40, or 80 mg; simvastatin at 40 or 80 mg; rosuvastatin at 5, 10, 20, or 40 mg; pravastatin at 80 mg, lovastatin at 80 mg, or pitavastatin at 4 mg, or wherein the first therapy or the non-PCSK9 LDL-C lowering agent is ezetimibe.

146. The method of arrangements 143 or 144, wherein the PCSK9 inhibitor or the anti-PCSK9 antibody is evolocumab, and wherein evolocumab is administered in an amount of at least 140 mg every two weeks.

147. The method of arrangements 143 or 144, wherein evolocumab is administered in an amount of at least 420 mg once monthly.

148. The method of arrangements 143 or 144, wherein an amount of the anti-PCSK9 neutralizing antibody is at least 150 mg.

149. The method of any of arrangements 143 or 144, wherein an amount of the anti-PCSK9 neutralizing antibody is at least 300 mg.

150. The method of any one of arrangement 1, 16, 18, 19, 32, 34, 35, 36, 82, 86, 100, 106, 107, 108, 109, 123, 125, 136, or 137, wherein the first therapy or the non-PCSK9 LDL-C lowering agent or the statin consists of or comprises an optimized amount of a statin, and wherein the second therapy, the PCSK9 LDL-C lowering agent, the PCSK9 inhibitor, the non-statin LDL-C lowering agent, or the anti-PCSK9 neutralizing antibody consists of or comprises evolocumab, and wherein evolocumab is administered in an amount of at least 140 mg every two weeks or 420 mg once monthly.

151. The method of any one of arrangements 5, 7, 9, 17, 18, 19, 22, 29, 30, 31, 33, 37, 38, 39, 77, or 101, wherein the second therapy, the PCSK9 LDL-C lowering agent, the PCSK9 inhibitor, the non-statin LDL-C lowering agent, or the anti-PCSK9 neutralizing antibody consists of or comprises evolocumab, and wherein evolocumab is administered in an amount of at least 140 mg every two weeks or 420 mg once monthly.

152. The method of the arrangement of 150 or 151, wherein the subject has clinical atherosclerotic cardiovascular disease and the method reduces a risk of myocardial infarction, stroke, and/or coronary revascularization.

153. The method of the arrangement of 150 or 151, wherein the subject has primary (heterozygous familial and non-familial) hyperlipidemia.

154. The method of one of the arrangements of 150-153, wherein the evolocumab is administered via an autoinjector or on-body infusor with prefilled cartridge.

155. A method of treating atherosclerotic cardiovascular disease and/or primary (heterozygous familial and non-familial) hyperlipidemia, the method comprising, providing a treatment to a subject, the treatment comprising: a statin; and evolocumab, wherein evolocumab is provided in an amount of at least 140 mg every two weeks or 420 mg once monthly.

156. A method of treating atherosclerotic cardiovascular disease and/or primary (heterozygous familial and non-familial) hyperlipidemia, the method comprising: receiving at least one of: atorvastatin at 20, 40, or 80 mg; simvastatin at 40 or 80 mg; rosuvastatin at 5, 10, 20, or 40 mg; pravastatin at 80 mg, lovastatin at 80 mg, or pitavastatin at 4 mg; and receiving evolocumab in an amount of at least 140 mg every two weeks or 420 mg once monthly.

157. A method of treating atherosclerotic cardiovascular disease and/or primary (heterozygous familial and non-familial) hyperlipidemia, the method comprising: providing or administering at least one of: atorvastatin at 20, 40, or 80 mg; simvastatin at 40 or 80 mg; rosuvastatin at 5, 10, 20, or 40 mg; pravastatin at 80 mg, lovastatin at 80 mg, or pitavastatin at 4 mg; and providing or administering evolocumab in an amount of at least 140 mg every two weeks or 420 mg once monthly.

158. A method of treating coronary atherosclerosis, the method comprising: identifying a subject that has a LDL-C level of greater than 70 mg/dL; and administering an anti-PCSK9 neutralizing antibody to the subject, in an amount sufficient and time sufficient to lower the LDL-C level to less than 40 mg/dL, less than 30 or less than 20 mg/dL.

159. A method of any one of the above arrangements, wherein the indication and/or goal in any one of the above arrangements is applied instead to at least one of: A) reducing a risk of at least one of: a major vascular event, a cardiovascular event, major cardiovascular adverse event, major adverse limb event, adverse limb event, PAD, fatal MI and/or non-fatal MI and fatal and/or non-fatal coronary revascularization, composite of: a) coronary revascularization, b) myocardial infarction, and c) cerebral vascular accident, composite of: a) cardiovascular death, b) myocardial infarction, c) stroke, d) hospitalization for unstable angina, or e) coronary revascularization, urgent coronary revascularization, at least one of: a) cardiovascular death, b) myocardial infarction, c) stroke, d) hospitalization for unstable angina, or e) coronary revascularization, or a cardiovascular event by at least 10%, or B) at least one of: treating atherosclerotic cardiovascular disease, treating coronary atherosclerosis, providing regression of coronary atherosclerosis, treating a subject that is unable to tolerate a full therapeutic dose of a statin, treating a subject that is unable to tolerate a full therapeutic dose of a non-PCSK9 LDL-C lowering agent, combining a PCSK9 inhibitor therapy and a non-PCSK9 LDL-C lowering therapy to produce greater LDL-C lowering and regression of coronary atherosclerosis at a dose that is well tolerated, reducing disease progression, reducing an amount of atherosclerotic plaque in a subject, combining evolocumab and a statin therapy to produce greater LDL-C lowering and regression of coronary atherosclerosis at a dose that is well tolerated, decreasing a LDL-C level in a subject beneath 80 mg/dL, decreasing total atheroma volume (TAV) in a subject, decreasing percent atheroma volume (PAV) in a subject, for lowering LDL-C level, and for reducing disease progression or any combination thereof.

160. Any of the methods of the arrangements provided above that includes a combination therapy, wherein a non-PCSK9 lipid lowering therapy or the non-PCSK9 LDL-C lowering agent or the statin is used as the first therapy.

161. A method of treating coronary atherosclerosis comprising a) identifying a statin-intolerant subject, b) administering a low dose or no dose statin treatment to the statin-intolerant subject, and c) administering an amount of an anti-PCSK9 neutralizing antibody to the subject to lower the LDL-C level of the statin intolerant subject to less than 60 mg/dL, thereby treating coronary atherosclerosis, such as 55, 50, 45, 40, 35, 30, 25, 20, or less mg/dL.

162. Any of the methods of the arrangements provided above, wherein the subject's non-HDL-C level is reduced to less than 100, 90, 80, 70, 60, 50, or 40.

163. The method of the arrangement in 162, wherein a risk of a primary, secondary, CVD, MI, stroke, pevasc, and/or hospitalization for unstable angina (“HUA”) of the subject is reduced.

164. The method of any one of arrangements 1, 16, 18, 19, 32, 34, 35, 36, 82, 86, 100, 106, 107, 108, 109, 123, 125, 136, 137, 7, 9, 17, 18, 19, 22, 29, 30, 31, 33, 37, 38, 39, 77, or 101, wherein the second therapy, the PCSK9 LDL-C lowering agent, the PCSK9 inhibitor, the non-statin LDL-C lowering agent, or the anti-PCSK9 neutralizing antibody comprises at least one of the six CDRs of evolocumab.

165. The method of arrangement 164, wherein the second therapy, the PCSK9 LDL-C lowering agent, the PCSK9 inhibitor, the non-statin LDL-C lowering agent, or the anti-PCSK9 neutralizing antibody comprises all 6 CDRs of evolocumab.

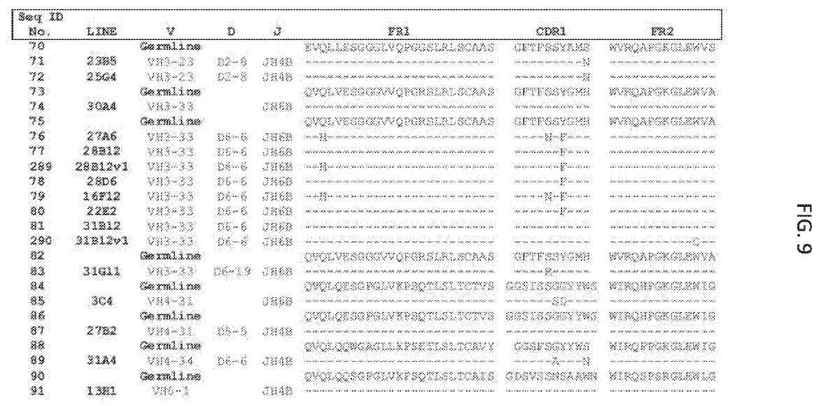

166. The method of arrangement 165, wherein the 6 CDRs are the 6 CDRs in FIGS. 8-11 of the construct designated as 21B12.

167. The method of arrangement 164, wherein the second therapy, the PCSK9 LDL-C lowering agent, the PCSK9 inhibitor, the non-statin LDL-C lowering agent, or the anti-PCSK9 neutralizing antibody comprises the heavy and light chain amino acid sequence of evolocumab.

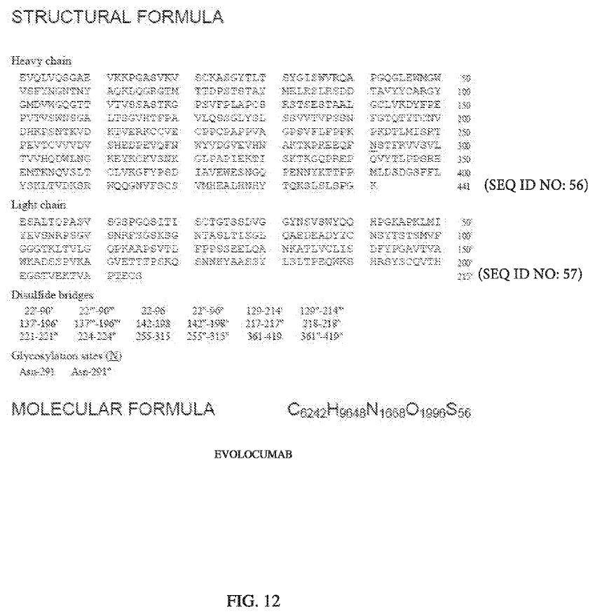

168. The method of arrangement 167, wherein the second therapy, the PCSK9 LDL-C lowering agent, the PCSK9 inhibitor, the non-statin LDL-C lowering agent, or the anti-PCSK9 neutralizing antibody comprises an evolocumab heavy chain and light chain, as shown in FIG. 12.

In some embodiments, a method of treating coronary atherosclerosis is provided. The method comprises a) identifying a statin-intolerant subject, b) administering a low dose or no dose statin treatment to the statin-intolerant subject, and c) administering an amount of at least one of: a PCSK9 LDL-C lowering agent, a PCSK9 inhibitor, a non-statin LDL-C lowering agent, an anti-PCSK9 neutralizing antibody, evolocumab, alirocumab, and/or an antibody that competes with evolocumab or alirocumab to the subject to lower the LDL-C level of the statin intolerant subject to less than 60 mg/dL, thereby treating coronary atherosclerosis. In some embodiments, the subject is treated long enough and with enough anti-PCSK9 neutralizing antibody to lower their LDL-C to 55, 50, 45, 40, 35, 30, 25, 20, or less mg/dL. In some embodiments, the antibody is evolocumab. When only a single therapy is employed, the therapy is not considered to be a “combination therapy” as the term is used herein. However, any of the embodiments provided herein for combination therapies are also contemplated for the present very low LDL-C therapy, as long as they allow for appropriate modification. In particular, the use of at least one of: a PCSK9 LDL-C lowering agent, a PCSK9 inhibitor, a non-statin LDL-C lowering agent, an anti-PCSK9 neutralizing antibody, evolocumab, alirocumab, and/or an antibody that competes with evolocumab or alirocumab will result in an exceptionally low LDL-C level in the subject, which will provide for the noted benefit (for that particular embodiment).

In some embodiments, a composition for achieving any of the above methods is provided. In some embodiments, the composition can be a combination of the first and second therapies. In some embodiments, the therapy can be provided as separate components, and each component can be administered separately or at the same time to the subject. In some embodiments, the secondary therapy is administered to the abdomen, thigh, or upper arm.

In some embodiments, one or more of the methods provided herein can be used to reduce the risk of myocardial infarction, stroke, and coronary revascularization in adults with clinical atherosclerotic cardiovascular disease.

In some embodiments, one or more of the methods provided herein can be used as an adjunct to diet, alone or in combination with other lipid-lowering therapies (e.g., statins, ezetimibe), for treatment of adults with primary (heterozygous familial and non-familial) hyperlipidemia to reduce low-density lipoprotein cholesterol (LDL-C).

In some embodiments, one or more of the methods provided herein can be used as an adjunct to diet and other LDL-lowering therapies (e.g., statins, ezetimibe, LDL apheresis) in patients with homozygous familial hypercholesterolemia (HoFH) who require additional lowering of LDL-C. In some embodiments, a non-PCSK9 lipid lowering therapy includes procedures, like apheresis. Thus, in some embodiments, any of the combination therapies provided herein can include a non-PCSK9 lipid lowering lowering treatment and/or a statin therapy and/or a PCSK9 therapy. In some embodiments, any of the combination therapies provided herein can include a non-PCSK9 lipid lowering lowering treatment and/or a PCSK9 therapy. In some embodiments, any of the combination therapies provided herein can include a non-PCSK9 lipid lowering treatment and/or a statin therapy.

In some embodiments, a 420 mg dose of REPATHA can be administered: over 9 minutes by using the single-use on-body infusor with prefilled cartridge, or by giving 3 injections consecutively within 30 minutes using the single-use prefilled autoinjector or single-use prefilled syringe.

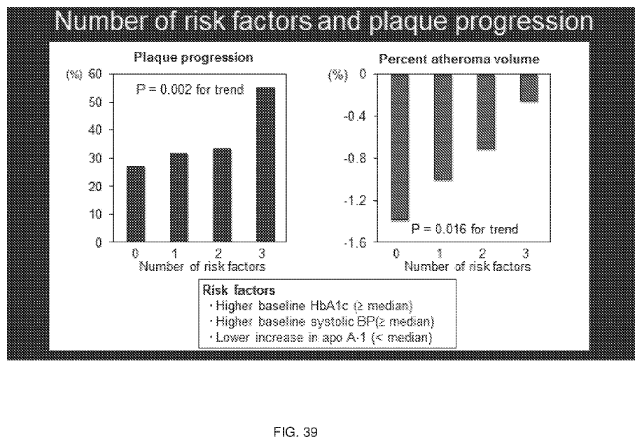

In some embodiments, for subjects receiving a combination therapy for plaque reduction, the subject has no or relatively few risk factors (as outlined in FIG. 39, for example and Example 19). In some embodiments, the subject lacks PAV, HbA1c and change in apolipoprotein A-I (p=0.01) that indicate risk or a risky systolic blood pressure.

In some embodiments, the subject to be treated by any of the methods provided herein has a Lp(a) level between 11.8 and 49 mg/dL. In some embodiments, the combination therapies provided herein can be applied to a subject with a normal Lp(a) level and the subject can still receive a benefit with respect to reduced atherosclerosis risk, from the intensive lipid lowering results provided by the combination therapy. Thus, the subject can receive an additional benefit by having their LDL-C level lowered to less than 70, less than 60, less than 50, less than 40, or, for example less than 30 mg/dL.

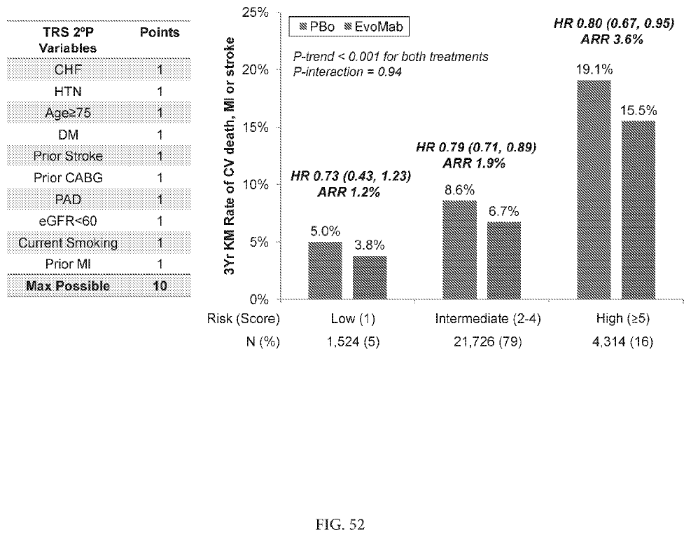

In some embodiments the subject receives a greater absolute reduction in major CV events. Support for this conclusion can be found, for example, in Example 22. In some embodiments, a high risk subject receives a combination therapy, as provided herein (e.g., a statin and evolocumab) so as to reduce the subject's LDL-C level to a level lower than 70, less than 60, less than 50, less than 40, or, for example less than 30 mg/dL. The risk to an intermediate risk subject (intermediate risk of atherosclerotic CV disease; TRS 2° P Score=24; 79% of population) can have at least a 1.9% absolute risk reduction (ARR) in CV death, MI or stroke at 3 yrs with EvoMab compared to Pbo alone. The risk to a high-risk subject (high risk of atherosclerotic CV disease, Score ≥5; 16%) can have a 3.6% ARR in CV death, MI or stroke (see, e.g., FIG. 52 and Example 22).

In some embodiments, any of the methods provided herein can be employed to reduce a total number of major vascular events in a subject, not just a risk of a first event. Support for this can be found in present Example 23, for example. In some embodiments, subjects on one of the combination therapies provided herein can have their LDL-C level lowered to less than 70, less than 60, less than 50, less than 40, or, for example less than 30 mg/dL, which can in turn reduce a risk of not just a first major cardiovascular event, but should one occur, it will reduce the risk of any subsequent cardiovascular event. This can be over 2, 4, 6, 8, 10, 12 months or 1, 1.2, 1.4, 1.6, 1.8, 2, 2, 2.2, 2.4, 2.6, 2.8, 3 or years or more. In some embodiments, the risk of a subsequent MI, stroke, or coronary revascularization is decreased both in likelihood of occurrence and in the time to such an event in the subject.

In some embodiments, any of the methods provided herein can be employed to reduce a risk of MI across the various subtypes of MI related to plaque rupture, smaller and larger MIs and both STEMI and NSTEMI, and/or types 1-4. Support for this can be found in present Example 24, for example. In some embodiments, subjects on one of the combination therapies provided herein can have their LDL-C level lowered to less than 70, less than 60, less than 50, less than 40, or, for example less than 30 mg/dL, which will allow for a reduced risk of MI across various subtypes of MI related to plaque rupture, smaller and larger MIs and/or both STEMI, NSTEMI, type 1, type 2, type 3, and/or type 4. In some embodiments, it is especially useful for STEMI, NSTEMI, type 1, and/or type 4 subtypes of MI. In some embodiments, MIs of various troponin thresholds can also be reduced in risk. In some embodiments, any of the combination methods provided herein are especially useful for subjects with elevated troponin. As outlined in the example below, in some embodiments, one can employ the combination therapy to reduce MIs in subjects with large with Tn≥10×ULN. Thus, the methods can be especially advantageous in subjects with elevated troponin, and this can be used as a screen for subjects that will have an additional benefit from the method (e.g., 10 fold greater level of troponin, for example).

In some embodiments, a method of treating a subject is provided. The method comprises providing a first therapy to a subject, wherein the first therapy comprises a non-PCSK9 LDL-C lowering therapy, and administering a second therapy to the subject, wherein the second therapy comprises a PCSK9 inhibitor. The subject has a history of stroke and/or diabetes. The method can be combined with any of the other combination embodiments provided herein.

For the embodiments provided herein regarding “stroke,” the disclosure of “stroke” discloses all embodiments related to stroke, including “fatal stroke”, “non-fatal stroke”, and both “fatal stroke” and “non-fatal stroke”. Similarly, the disclosure of “fatal stroke” also denotes the contemplation of the use of the method in non-fatal strokes or for the broad use for both as well.

For the embodiments provided herein regarding “MI,” the disclosure of “MI” discloses all embodiments related to MI, including “fatal MI”, “non-fatal MI”, and both “fatal MI” and “non-fatal MI”. Similarly, the disclosure of “fatal MI” also denotes the contemplation of the use of the method in non-fatal MI or for the broad use for both as well.

For the embodiments provided herein regarding “coronary revascularization,” the disclosure of “coronary revascularization” discloses all embodiments related thereto, including: “urgent coronary revascularization”, “non-urgent coronary revascularization”, and both “urgent coronary revascularization” and “non-urgent coronary revascularization”. Similarly, the disclosure of “urgent coronary revascularization” also denotes the contemplation of the use of the method in coronary revascularization or for the broad use for both as well.

### EXAMPLES

### Example 1

### Introduction

The present example outlines and presents the results of the Global Assessment of Plaque Regression with a PCSK9 Antibody as Measured by Intravascular Ultrasound (GLAGOV) trial. This trial assessed several principal scientific questions, including: whether PCSK9 inhibition reduces progression of atherosclerosis and whether achieving very low LDL-C levels with the combination of statins and a PCSK9 inhibitor provide incremental value in further reducing the progression of coronary disease as measured by IVUS. The results also demonstrated that the result of the combination therapy (achieving very low LDL-C levels), not only reduces progression, but can actually reverse the disorder.

### Methods

### Study Design

The GLAGOV trial was a randomized, multicenter, double-blind, institutional review boards at each site approved the protocol, and patients provided written informed consent. The protocol and statistical analysis plan are available at JAMAnetwork.com and the design of the trial has been described previously.¹²

Patients aged ≥18 years were eligible if they demonstrated at least one epicardial coronary stenosis ≥20% on clinically-indicated coronary angiography and had a target vessel suitable for imaging with ≤50% visual obstruction. Patients were required to have been treated with a stable statin dose for at least four weeks and to have a LDL-C ≥80 mg/dL or between 60 and 80 mg/dL with one major or three minor cardiovascular risk factors. Major risk factors included non-coronary atherosclerotic vascular disease, myocardial infarction or hospitalization for unstable angina in the preceding 2 years or type 2 diabetes mellitus. Minor risk factors included current cigarette smoking, hypertension, low levels of high-density lipoprotein cholesterol (HDL-C), family history of premature coronary heart disease, high sensitivity C-reactive protein (hs-CRP) ≥2 mg/L or age ≥50 years in men and 55 years in women. By design, patients with an entry LDL-C between 60-80 mg/dL were limited to 25% of the total patient cohort. A four-week lipid stabilization period was included for patients not currently taking lipid-modifying therapy at screening. Inclusion of patients intolerant to statins was limited to 10% of the total cohort. Patients were excluded if they had uncontrolled diabetes or hypertension or heart failure, renal dysfunction or liver disease. Patients were asked to identify race according to fixed categories determined by the study protocol, in order to evaluate potential differences in concomitant treatment and disease progression.

Patients underwent randomization in a 1:1 allocation ratio with a block size of 4 using an interactive voice response system to treatment with evolocumab 420 mg or placebo administered monthly via subcutaneous injection for 76 weeks. During the treatment period, patients underwent clinic visits at weeks 4, 12, 24, 36, 52, 64, 76 and repeat IVUS imaging at week 78. A clinical events committee, blinded to treatment assignment, adjudicated cardiovascular events. An independent, unblinded data monitoring committee, led by an academic cardiologist, reviewed clinical trial safety during the study.

### Acquisition and Analysis of Ultrasound Images

Following coronary angiography, baseline intravascular ultrasonography was performed. Previous reports have described the methods of image acquisition and analysis.^{3,5,6,13-18} Imaging was performed in a single artery and screened by a core laboratory. Patients meeting prespecified requirements for image quality were eligible for randomization. At week 78, patients underwent a second ultrasonographic examination within the same artery. Using digitized images, personnel, unaware of the treatment status, performed measurements of the lumen and external elastic membrane in images within a matched artery segment. Measurement personnel were blinded to the sequence of imaging studies (baseline vs. follow up). The accuracy and reproducibility of this method have been reported previously.^{3,5,6,13-18}

The primary efficacy measure, percent atheroma volume (PAV), was calculated as follows:

$$PAV = \frac{Σ ( {EEM}_{area} - {Lumen}_{area} )}{Σ {EEM}_{area}} \times 100$$

where EEMₐᵣₑₐ the cross-sectional area of the external elastic membrane and Lumenₐᵣₑₐ is the cross-sectional area of the lumen. The change in PAV was calculated as the PAV at 78 weeks minus the PAV at baseline. A secondary measure of efficacy, normalized total atheroma volume (TAV), was calculated as follows:

$${TAV}_{Normalized} = \frac{Σ ( {EEM}_{area} - {Lumen}_{area} )}{Number of Images in Pullback} \times Median number of images in cohort$$

where the average plaque area in each image was multiplied by the median number of images analyzed in the entire cohort to compensate for differences in segment length between subjects. Change in normalized TAV was calculated as the TAV at 78 weeks minus the TAV at baseline. Regression was defined as any decrease in PAV or TAV from baseline.

### Efficacy Endpoints

The primary efficacy endpoint was the nominal change in PAV from baseline to week 78 as described above. Secondary efficacy endpoints included, in sequential order of testing, nominal change in TAV from baseline to week 78 as described above, any reduction of PAV from baseline and any reduction of TAV from baseline. Exploratory endpoints included the incidence of adjudicated events (all-cause mortality, cardiovascular death, myocardial infarction, hospitalization for unstable angina, coronary revascularization, stroke, transient ischemic attack [TIA], and hospitalization for heart failure) and change in lipid parameters. Additional exploratory post hoc analyses included comparison of the change in PAV and percentage of patients undergoing regression of PAV in those with a LDL-C less than or greater than 70 mg/dL at baseline. Locally weighted polynomial regression (LOESS) curve fitting was performed to examine the association between achieved LDL-C levels and disease progression.

### Statistical Analysis

All statistical analyses were performed using SAS version 9.4 (SAS Inc, Cary N.C.). For continuous variables with an approximately normal distribution, means and standard deviations are reported. For variables not normally distributed, medians and interquartile ranges are reported. IVUS efficacy parameters are reported as least square means (95% confidence intervals [CI]) and treatment groups compared using analysis of covariance (ANCOVA) on rank-transformed data with adjustment for baseline value and geographic region. On-treatment lipoprotein levels are reported as time-weighted means (95% confidence intervals [CI) and compared using ANCOVA with adjustment for treatment group and geographic region. Time-weighted averages for each laboratory parameter were created by the summation of the product between each measurement and time interval between each visit divided by the total time.

A step down statistical approach was applied to investigate the primary and secondary endpoints. First the primary endpoint was tested at the 0.05 significance level, then the secondary endpoints tested at the significance level of 0.05 in the sequential order as listed in Section 4.1.2 in the statistical analysis plan. A sensitivity analysis using multiple imputation was performed to impute missing primary endpoint data. The imputation model included variables for treatment group, background statin therapy intensity, region, baseline LDL, baseline PAV, age and sex as covariates. Subgroup analyses on the primary endpoint were conducted using subgroups specified in section 7.4 of the statistical analysis plan. Subgroup by treatment interactions were tested. An additional exploratory analysis was conducted in patients with baseline LDL-C less than or greater than 70 mg/dL.

For the change in the primary efficacy parameter, PAV, a sample size of 356 subjects in each treatment group was required to provide 90% power at a two-sided alpha of 0.05 to detect a nominal treatment difference of 0.71% assuming a 2.9% standard deviation. A difference of 0.5% has been previously reported to distinguish patients who experience cardiovascular events, from those who do not.¹⁹ Assuming a withdrawal rate of 25%, 950 randomized patients were required. All reported p-values are 2-sided. A p-value <0.05 was considered statistically significant.

### Results

### Subject Characteristics

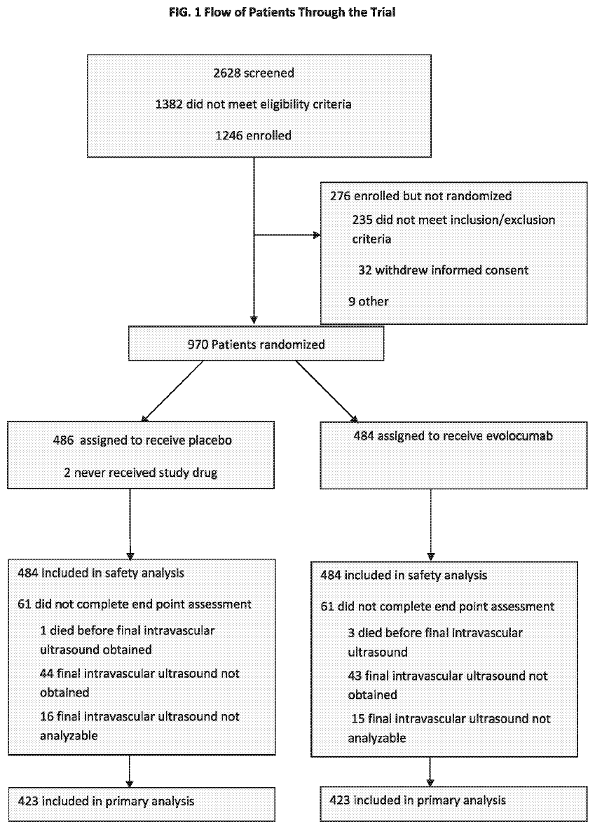

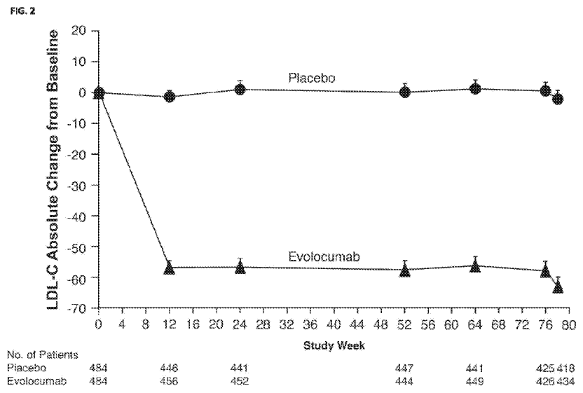

The disposition of patients enrolled in the study is illustrated in FIG. 1. From May 3, 2013 to Jan. 12, 2015, at 197 centers, 970 patients were randomized and 968 received study drug, 484 to the evolocumab treatment group and 484 to the placebo group. 846 patients (87.2%) had evaluable IVUS imaging at both baseline and follow-up. Of these patients, 423 were in the placebo group and 423 in the evolocumab group. Mean exposure to study drug was 17.6 months. Table 1 reports the baseline characteristics of randomized patients.

TABLE 1
Baseline Characteristics of Subjects in the Randomized
Population who Received Study Drug (N = 968)

| | Placebo | Evolocumab |
|---|---|---|
| Parameter | (N = 484) | (N = 484) |
| Age | 59.8 ± 8.8 | 59.8 ± 9.6 |
| Males n (%) | 350 (72.3%) | 349 (72.1%) |
| White n (%) | 452 (93.4%) | 456 (94.2%) |
| BMI | 29.5 ± 5.0 | 29.4 ± 5.0 |
| Hypertension n (%) | 405 (83.7%) | 398 (82.2%) |
| Previous PCI n (%) | 188 (38.8%) | 189 (39.0%) |
| Previous MI n (%) | 171 (35.3%) | 169 (34.9%) |
| Smoking n (%) | 113 (23.3%) | 124 (25.6%) |
| Diabetes n (%) | 104 (21.5%) | 98 (20.2%) |
| Baseline statin use n (%)† | 476 (98.3%) | 478 (98.8%) |
| High intensity n (%) | 290 (59.9%) | 280 (57.9%) |
| Moderate intensity n (%) | 185 (38.2%) | 196 (40.5%) |
| Low intensity n (%) | 1 (0.2%) | 2 (0.4%) |
| Baseline ezetimibe use n (%)† | 9 (2.1%) | 9 (2.1%) |
| Baseline medications n (%)† | | |
| Anti-platelet therapy therapy | 465 (96.1%) | 454 (93.8%) |
| Beta-blocker | 370 (76.4%) | 362 (74.8%) |
| ACE inhibitor | 264 (54.5%) | 260 (53.7%) |
| ARB | 92 (19.0%) | 87 (18.0%) |

Age and BMI expressed as mean ± standard deviation.
†Baseline statin and ezetimibe use is defined as subject treated with statin or ezetimibe therapy at the end of the lipid stabilization period at randomization.

Table 1 (above) outlines the clinical characteristics and concomitant medications of patients treated with placebo or evolocumab with evaluable imaging at baseline and follow-up. Results expressed as mean±standard deviation for continuous variables and frequency (percentage) for categorical variables. ACE, angiotensin converting enzyme; ARB, angiotensin receptor blocker; BMI, body mass index; MI, myocardial infarction; PCI, percutaneous coronary intervention.

At the time of randomization, 58.9% were receiving high intensity statin and 39.4% moderate statin therapy with 1.4% of patients not treated with a statin. At baseline, patients had a mean LDL-C 92.5±27.2 mg/dL and median hsCRP of 1.6 (interquartile range 0.8, 3.4) mg/L. No significant differences in these parameters were observed between patients who had evaluable follow-up IVUS imaging and those who did not (see Table 1.1).

TABLE 1.1
Baseline Characteristics of Subjects in the Randomized Population
who Received Study Drug with and without Evaluable
Follow-up IVUS Imaging (N = 968)

| | No IVUS | IVUS |
|---|---|---|
| Parameter | (N = 122) | (N = 846) |
| Age | 61.0 ± 9.7 | 59.6 ± 9.1 |
| Males n (%) | 85 (69.7) | 614 (72.6) |
| White n (%) | 112 (91.8) | 796 (94.1) |
| BMI | 29.2 ± 4.7 | 29.4 ± 5.0 |
| Diabetes n (%) | 27 (22.1) | 175 (20.7) |
| Hypertension n (%) | 108 (88.5) | 695 (82.2) |
| Smoking n (%) | 32 (26.2) | 205 (24.2) |
| Previous MI n (%) | 53 (43.4) | 287 (33.9) |
| Previous PCI n (%) | 45 (36.9) | 332 (39.2) |
| Baseline statin use n (%)† | 117 (95.9) | 837 (98.9) |
| High intensity n (%) | 62 (50.8) | 50.8 (60.0) |
| Moderate intensity n (%) | 55 (45.1) | 326 (38.5) |
| Low intensity n (%) | 0 (0) | 3 (0.4) |
| Baseline ezetimibe use n (%)† | 5 (4.1) | 13 (1.5) |
| Baseline medications n (%)† | | |
| Anti-platelet therapy | 115 (94.3) | 804 (95.0) |
| Beta-blocker | 88 (72.1) | 644 (76.1) |
| ACE inhibitor | 66 (54.1) | 458 (54.1) |
| ARB | 18 (14.8) | 161 (19.0) |

Age and BMI expressed as mean ± standard deviation.
†Baseline medication use is defined as subject treated with statin or ezetimibe therapy at the end of the lipid stabilization period at randomization.

### Biochemical Measurements

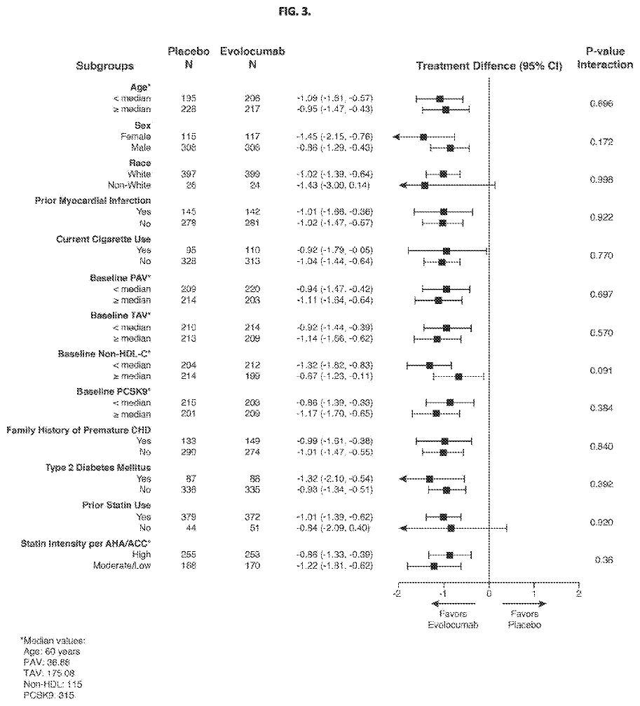

Table 2 below summarizes the baseline and on-treatment laboratory values for the 846 patients who underwent follow-up IVUS imaging. During 78 weeks of treatment, time-weighted mean LDL-C levels were 93.0 mg/dL (a 3.9% change from baseline, resulting in a 90 mg/dL of LDL-C) in the placebo group and 36.6 mg/dL (a −59.8% change from baseline, resulting in 29 mg/dL of LDL-C) in the evolocumab group (P<0.001), representing an increase in LDL-C by 0.5 mg/dL in the placebo group compared with a decrease by 56.1 mg/dL in the evolocumab group, between groups difference −56.5 mg/dL (95% CI −59.7, −53.4, P<0.001). (FIG. 2) Evolocumab-treated patients demonstrated greater reductions in apoB (−38.8 vs. +2.7 mg/dL, between groups difference −40.6 mg/dL [95% CI −42.9, −38.3], P<0.001), triglycerides (−9.6 vs. +5.6 mg/dL, between groups difference −19.1 mg/dL [95% CI −27.5, −10.6], P<0.001) and Lp(a) (−3.8 vs. −0.2 mg/dL, between groups difference −6.7 mg/dL [95% CI −7.9, −5.5], P<0.001) and greater increases in HDL-C levels (+4.0 vs. +1.2 mg/dL, between groups difference 2.5 mg/dL [95% CI 1.7, 3.4], P<0.001). Median hsCRP levels during treatment were 1.4 mg/L (IQR 0.7, 3.0) in the placebo group and 1.4 mg/L (IQR 0.7, 3.0) in the evolocumab group, P=0.48.

TABLE 2

| | Baseline | | | On-Treatment | | | Absolute Change | | |
|---|---|---|---|---|---|---|---|---|---|
| | Placebo | Evolocumab | P | Placebo | Evolocumab | P | Placebo | Evolocumab | P |
| Parameter | (N = 484) | (N = 484) | Value^{@} | (N = 484) | (N = 484) | Value^{@} | (N = 484) | (N = 484) | Value^{@} |
| Total | 166.2 | 166.1 (34.1) | 0.96 | 169.1 | 108.6 (29.8) | <0.001 | 1.8 (−2.0, | −59.0 (−62.8, | <0.001 |
| cholesterol | (34.2) | | | (31.5) | | | 5.6) | −55.2) | |
| (mg/dL) | | | | | | | | | |
| LDL | 92.4 | 92.6 (27.5) | 0.95 | 93.0 | 36.6 (23.5) | <0.001 | 0.2 (−2.9, | −56.3 (−59.4, | <0.001 |
| cholesterol | (26.9) | | | (26.8) | | | 3.4) | −53.1) | |
| (mg/dL)* | | | | | | | | | |
| HDL | 45.4 | 46.7 (12.6) | 0.13 | 47.1 | 51.0 (13.1) | <0.001 | 0.7 (−0.1, | 3.3 (2.4, 4.1) | <0.001 |
| cholesterol | (12.9) | | | (12.3) | | | 1.6) | | |
| (mg/dL) | | | | | | | | | |
| Triglycerides | 124.5 | 117.0 (88.0, | 0.10 | 130.5 | 105.1 (82.5, | <0.001 | 8.1 (−0.4, | −19.1 (−27.5. | <0.001 |
| (mg/dL) ^{†} | (90.0, | 155.0) | | (100.3, | 141.6) | | 16.6) | −2.5) | |
| | 173.0) | | | 560.4) | | | | | |
| Non HDL | 120.8 | 119.4 (32.0) | 0.51 | 122.0 | 57.7 (28.4) | <0.001 | 1.1 (−2.7, | −62.3 (−66.0, | <0.001 |
| cholesterol | (32.2) | | | (30.3) | | | 4.8) | −58.5) | |
| (mg/dL) | | | | | | | | | |
| Total/HDL | 3.9 (1.1) | 3.7 (1.1) | 0.10 | 3.8 (1.1) | 2.3 (0.8) | <0.001 | −0.1(−−0.2, | −1.5(−−1.6, | <0.001 |
| cholesterol | | | | | | | 0.2, 0.04) | −1.4) | |
| ApoB (mg/dL) | 81.9 | 81.1 (20.2) | 0.55 | 83.5 | 42.4 (17.8) | <0.001 | 0.3 (−2.0, | −40.3 (−42.6, | <0.001 |
| | (19.8) | | | (18.6) | | | 2.6) | −38.0) | |
| ApoA-I | 139.5 | 140.5 (25.3) | 0.55 | 145.4 | 151.6 (23.4) | <0.001 | 3.5 (1.5, | 8.5 (6.5, 10.5) | <0.001 |
| (mg/dL) | (26.0) | | | (22.2) | | | 5.5) | | |
| ApoB/A-I | 0.60 | 0.59 (0.18) | 0.38 | 0.59 | 0.29 (0.14) | <0.001 | −0.02 | −0.3 (−0.33, | <0.001 |
| | (0.17) | | | (0.16) | | | (−0.04, | −0.29) | |
| | | | | | | | −0.001) | | |
| hsCRP(mg/L) ^{†¶} | 1.6 (0.8, | 1.6 (0.8, 3.4) | 0.96 | 1.4 (0.7, | 1.4 (0.7, 3.0) | 0.26 | −0.3 | −0.4 (−1.3, | 0.35 |
| | 3.4) | | | 3.0) | | | (−1.3, 0.6) | 0.6) | |
| Lp(a) (mg/dL) ^{#} | 10.9 | 12.1 (4.6, | 0.03 | 8.9 (3.9, | 7.1 (2.5, 46.7) | 0.07 | −1.0 | −7.8 (−9.0, | <0.001 |
| | (3.9, | 57.1) | | 48.1) | | | (−2.2, 0.2) | −6.6) | |
| | 50.7) | | | | | | | | |
| PCSK9 | 322.5 | 325.4 (95.3) | 0.65 | 307.8 | 146.9 (66.8) | <0.001 | −7.2 | −172.8 | <0.001 |
| (ng/mL) | (99.6) | | | (66.6) | | | (−19.4, 5.0) | (−184.9, −160.7) | |
| Glucose | 107.3 | 104.0 (24.1) | 0.06 | 109.4 | 110.1 (25.6) | 0.72 | 3.9 (1.3, | 7.8 (5.3, 10.4) | 0.02 |
| (mg/dL) ^{†¶} | (30.3) | | | (28.2) | | | 6.5) | | |
| HbA1c (%) ^{¶} | 5.9 (0.9) | 5.8 (0.7) | 0.44 | 6.0 (0.9) | 6.0 (0.8) | 0.85 | 0.2 (0.1, | 0.2 (0.15, | 0.09 |
| | | | | | | | 0.2) | 0.25) | |
| Systolic BP | 129.6 | 131.4 (14.9) | 0.07 | 131.9 | 131.5 (11.5) | 0.55 | 0.9 (−0.7, | −1.3 (−2.9 0.4) | 0.007 |
| (mmHg) | (15.5) | | | (12.7) | | | 2.5) | | |
| Diastolic BP | 76.7 | 78.0 (9.6) | 0.03 | 78.5 | 78.6 (7.1) | 0.94 | 2.2 (1.0, | 0.9 (−0.2, | 0.01 |
| (mmHg) | (10.0) | | | (7.8) | | | 3.3) | 1.99) | |

^{@}P value for between treatment group comparison. Baseline laboratory variables are presented using means and standard deviation except where indicated.
^{†} Median and interquartile range are presented for non-normally distributed parameters and tested using Wilcoxon rank-sum test.
‡ On-treatment laboratory parameters are the time-weighted averages (±standard error) of all post-baseline values and estimates are derived from an ANOVA model with factors for treatment group and region.
^{¶} Final measurements are used for on-treatment values Absolute changes are presented as least square means (95% confidence intervals).
*When the calculated LDL-C is < 40 mg/dL or triglycerides are > 400 mg/dL, ultracentrifugation LDL-C was determined from the same blood sample.
^{#} Lp(a) converted from nmol/L to mg/dL by dividing by 2.8. Table 2 shows baseline and time-weighted average on-treatment values and percentage changes of laboratory measures and blood pressure of patients treated with placebo or evolocumab with evaluable imaging at baseline and follow-up. Results expressed as mean ± standard deviation at baseline and least-square mean ± standard error for on-treatment values.
Apo, apolipoprotein; BP, blood pressure; HbA1c, glycosylated hemoglobin; HDL, high-density lipoprotein; hsCRP, high sensitivity C-reactive protein; LDL, low-density lipoprotein; Lp(a), lipoprotein (a); PCSK9, proprotein convertase subtilisin kexin type 9.

### Primary and Secondary IVUS Endpoints

Changes in IVUS measures of plaque burden are summarized in Table 3 below. Table 3 provides the primary and secondary end points as evaluated on intravascular ultrasonography at baseline and 78-week follow-up with changes from baseline. Results expressed as mean±SD and median (95% confidence interval) for continuous variables and percentage for categorical variables at baseline and follow-up. Change in parameters expressed as least square mean±standard error.

TABLE 3
Primary and Secondary Study End Points as Evaluated on Intravascular
Ultrasonography.

| | | | | |
|---|---|---|---|---|
| | | | Between | |
| | | Evolocumab | Group | |
| Parameter | Placebo (N = 423) | (N = 423) | Differences | P Value |

Baseline

| | | | | |
|---|---|---|---|---|
| Percent Atheroma Volume (%) | | | | |
| Mean ± SD | 37.2 ± 8.5 | 36.4 ± 8.7 | −0.76 (−1.9, 0.4) | 0.18 |
| Median (95% Cl) | 37.1 (36.0, 38.0) | 36.4 (35.5, 37.5) | | |
| Total Atheroma Volume (mm³) | | | | |
| Mean ± SD | 191.4 ± 85.7 | 187.0 ± 81.8 | −4.3 (−15.6, 7.0) | 0.63 |
| Median (95% C1) | 175.8 (164.0, 187.4) | 174.6 (164.1, 183.1) | | |

Follow-up at 78 weeks

| | | | | |
|---|---|---|---|---|
| Percent Atheroma Volume (%) | | | | |
| Mean ± SD | 37.3 ± 8.2 | 35.6 ± 8.2 | −1.7 (−2.8, −0.6) | 0.002 |
| Median (95% Cl) | 36.8 (35.7, 37.8) | 35.7 (34.8, 36.5) | | |
| Total Atheroma Volume (mm³) | | | | |
| Mean ± SD | 190.6 + 84.4 | 181.5 + 77.6 | −8.9 (−19.9, 2.0) | 0.23 |
| Median (95% Cl) | 174.4 (164.3, 186.6) | 169.6 (160.9, 180.7) | | |
| | | | | Between |
| | | | | groups P |
| Percent Atheroma Volume (%) | | | | Value † |

Change from baseline

| | | | | |
|---|---|---|---|---|
| LS mean (95% Cl) | 0.05 (−0.32, 0.42) | −0.95 (−1.33, −0.58) | −1.01 (−1.78, 0.64) | <0.001 |
| P value for change from | P = 0.78 | P < 0.001 | | |
| baseline | | | | |
| Total Atheroma Volume (mm³) | | | | |
| LS mean (95% Cl) | −0.91 (−3.29, 1.47) | −5.80 (−8.19, −3.41) | −4.9 (−7.3, 2.5) | <0.001 |
| P value for change from | P = 0.45 | P < 0.001 | | |
| baseline | | | | |
| Percentage of patients with | 47.3 (42.5, 52.0) | 64.2 (59.7, 68.9) | 17.0 (10.4, 23.6) | <0.001 |
| regression of percent | | | | |
| atheroma volume (%) (95% Cl) | | | | |
| Percentage of patients with | 48.9 (44.2, 53.7) | 61.3 (56.8, 66.1) | 12.5 (5.9, 19.2) | <0.001 |
| regression of total atheroma | | | | |
| volume (%) (95% Cl) | | | | |

† The p-value for comparison between treatments for change from baseline were generated from an analysis of covariance.

The primary efficacy measure, PAV, did not change in the placebo group (+0.05%, P=0.78 compared with baseline) and decreased by 0.95% in the evolocumab group (P<0.001 compared with baseline; between groups difference −1.01% (95% CI −1.78, 0.64) P<0.001). The secondary efficacy measure, TAV, did not change in the placebo group (−0.9 mm3, P=0.45 compared with baseline) and decreased by 5.8 mm3 in the evolocumab group (P<0.001 compared with baseline; between groups difference −4.9 mm3 [95% CI −7.3, 2.5] P<0.001). More evolocumab-treated patients exhibited PAV regression with 64.2% vs. 47.3%, P<0.001) and TAV regression with 61.3% vs. 48.9%, P<0.001). For all prespecified subgroups, there was no statistical evidence of interaction (FIG. 3). Specifically, there was no difference in treatment effect observed in patients stratified according to baseline LDL-C. Imputation modeling for patients that did not have evaluable IVUS imaging at follow up demonstrated similar findings with a decrease in PAV with placebo (−0.02%) and evolocumab (−1.05%), between groups difference −1.03% (95% CI −1.51, −0.55), P<0.001.

### Exploratory Post Hoc Analyses

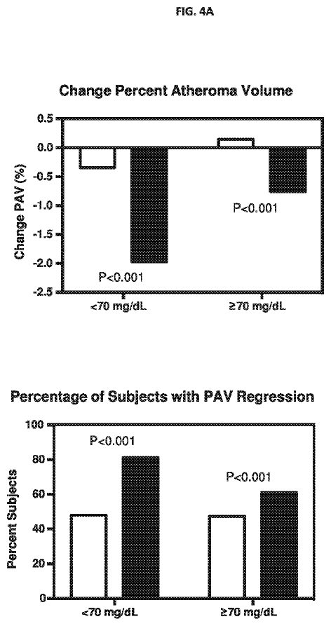

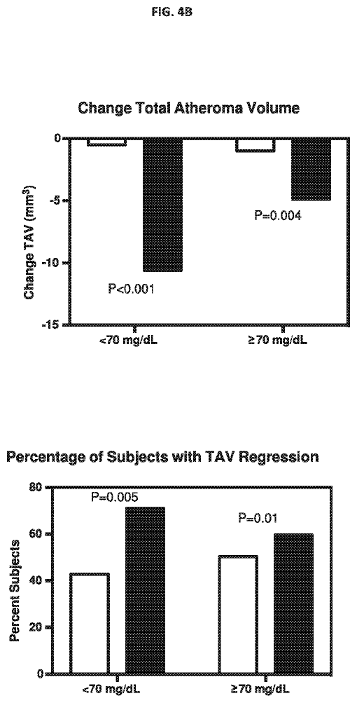

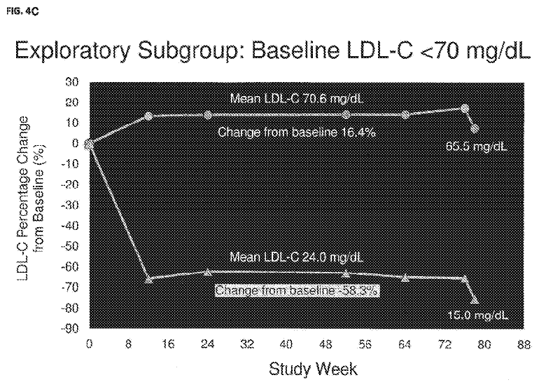

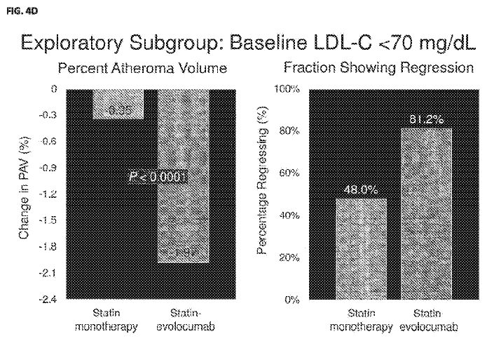

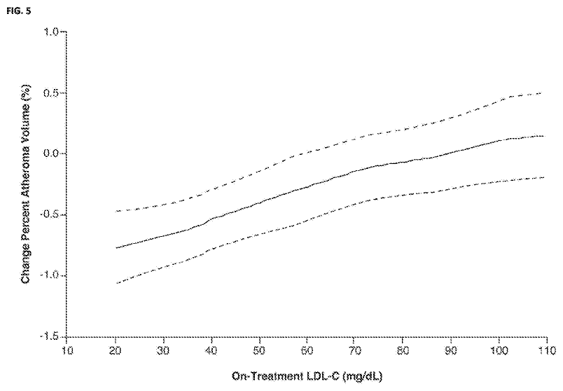

In 144 patients with a baseline LDL-C <70 mg/dL, evolocumab treatment, compared with placebo, was associated with favorable effects on the change in PAV (−1.97% vs. −0.35%, between groups difference −1.62% [95% CI −2.50, −0.74], P<0.001). In this subgroup, the percentage of patients with regression of PAV for evolocumab compared with placebo was 81.2% vs. 48.0%, between groups difference 33.2% [95% CI 18.6, 47.7] P<0.001). (FIG. 4A, black bars represent statin combined with evolocumab, white bars are statin monotherapy). The change in PAV for statin monotherapy was 0.05%, the change in PAV for statin+evolocumab was −0.95% (across all groups treated). A similar association was observed for the TAV secondary endpoint. (FIG. 4B black bars represent statin combined with evolocumab, white bars are statin monotherapy). The change in TAV for statin monotherapy was −0.9%, and the change in TAV for statin+evolocumab was −5.8% (across all groups treated). The right hand panel of FIG. 4A depicts the percent of subjects with PAV regression (the sum of the <70 and ≥70 is monotherapy: 47.3% regressors, 52.7% progressors; and statin+evolocumab: 64.3% regressors and 35.7% progressors). The right hand panel of FIG. 4B depicts the percent of subjects with TAV regression.

FIG. 4C depicts the data from an exploratory subgroup of subjects having a baseline LDL-C <70 mg/dL. The mean LDL-C was 70.6 mg/dL for the monotherapy (a 16.4% change from baseline to end at 65.5 mg/dL) and 24.0 mg/dL for the combination therapy (a −58.3% change to end at 15.0 mg/dL). FIG. 4D depicts the data from an exploratory subgroup having a baseline LDL-C of <70 mg/dL, showing the change in PAV at −0.35% for the statin monotherapy and −1.97% for the combination therapy, with 48.0% showing regression on the monotherapy and 81.2% showing regression on the combination therapy.

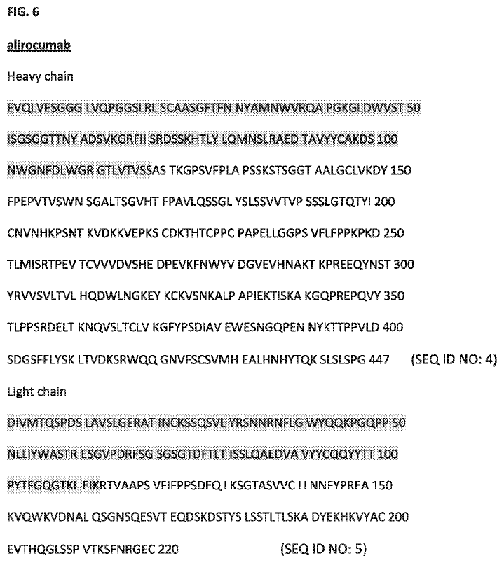

A LOESS plot showed a linear relationship between achieved LDL-C and PAV progression for LDL-C levels ranging from 110 mg/dL to as low as 20 mg/dL. (FIG. 5, plot shows 95% confidence limits).

### Exploratory Clinical Events and Laboratory Adverse Events

Table 4 describes centrally adjudicated clinical events, clinical adverse events, laboratory abnormalities and reasons for study discontinuation. Table 4 summarizes the clinical and laboratory adverse events and reasons for discontinuation in the safety population. Results expressed as frequency (percentage). ULN, upper limit of normal.

TABLE 4
Clinical and Biochemical Adverse Events and Reasons
for Discontinuation in the Safety Population

| | Placebo | Evolocumab |
|---|---|---|
| Parameter | (N = 484) | (N = 484) |
| Cardiovascular events - n (%) ‡ | | |
| Death | 4 (0.8%) | 3 (0.6%) |
| Nonfatal myocardial infarction | 14 (2.9%) | 10 (2.1%) |
| Nonfatal stroke | 3 (0.6%) | 2 (0.4%) |
| Hospitalization for unstable angina | 4 (0.8%) | 3 (0.6%) |
| Coronary revascularization | 66 (13.6%) | 50 (10.3%) |
| First major adverse cardiovascular event | 74 (15.3%) | 59 (12.2%) |
| Clinically important adverse events - n (%) | | |
| Injection site reaction | 0 (0) | 2 (0.4%) |
| Myalgia | 28 (5.8%) | 34 (7.0%) |
| Neurocognitive events* | 6 (1.2%) | 7 (1.4%) |
| New diagnosis diabetes mellitus* | 18 (3.7%) | 17 (3.6%) |
| Abnormality in laboratory value - n (%) † | | |
| Aspartate or alanine aminotransferase >3xULN | 2 (0.5%) | 2 (0.5%) |
| Total bilirubin >2xULN | 2 (0.5%) | 1 (0.3%) |
| Creatine phosphokinase >5xULN | 3 (0.7%) | 3 (0.7%) |
| Creatinine >ULN | 5 (1.0%) | 3 (0.6%) |
| Anti-Evolocumab binding antibody | N/A | 1 (0.2%) |
| Anti-Evolocumab neutralizing antibody | N/A | 0 (0) |
| Discontinuation from treatment - n (%) | | |
| Number of patients | 35 (7.2%) | 38 (7.9%) |
| Reason for discontinuation | | |
| Preference of patient | 21 (4.3%) | 15 (3.1%) |
| Adverse event | 7 (1.4%) | 15 (3.1%) |
| Lost to follow-up | 2 (0.4%) | 3 (0.6%) |
| Physician decision | 2 (0.4%) | 3 (0.6%) |
| Other | 2 (0.4%) | 3 (0.6%) |

† The denominator for both placebo and evolocumab with normal value at baseline is 958. There were a total of 10 subjects with missing safety laboratory data.
‡ Total number of cardiovascular events included 2 events occurring during the period between the last scheduled visit and the end of safety assessment period.
*Neurocognitive events and new diagnosis diabetes mellitus as reported by investigators as adverse events.
N/A: not applicable.

Although the study was not powered to assess effects on cardiovascular events, exploratory analysis revealed numerically fewer adverse cardiovascular outcomes (12.2% vs. 15.3%), non-fatal myocardial infarction (2.1% vs. 2.9%) and coronary revascularization (10.3% vs. 13.6%) in the evolocumab versus placebo groups. Administration of evolocumab was well tolerated with no significant excess in rate of injection site reactions (0.4% vs. 0%), myalgia (7.0% vs. 5.8%) and neurocognitive events (1.4% vs. 1.2%). The rates of laboratory abnormalities were low in both groups. Only 1 patient (0.2%) developed anti-evolocumab antibodies and none had neutralizing antibodies detected. Glycosylated hemoglobin levels did not change in either treatment group.

### Discussion of Example 1

The above trial demonstrated that addition of the PCSK9 inhibitor evolocumab in patients treated with moderate or intensive statin therapy (a combination therapy) had a favorable effect on progression of coronary atherosclerosis as measured by IVUS. Both the primary and secondary IVUS efficacy measures showed atherosclerosis regression during 18 months of therapy in patients treated with the combination of evolocumab and statins and absence of regression in patients treated with a statin alone. Compared with baseline, for the primary IVUS endpoint, PAV, patients in the placebo treatment group demonstrated no decrease in atheroma burden (+0.05%, P=0.78) whereas patients in the evolocumab group showed a significant reduction in PAV (−0.95%, P<0.001), between-groups difference of −1.01%, P<0.001. Similar results were observed for the principal secondary endpoint, TAV (between groups difference −4.9 mm3, P<0.001). These findings provide evidence that PCSK9 inhibition produces incremental benefits on coronary disease progression in statin-treated patients.

The percentage of patients demonstrating regression of coronary atherosclerosis, defined as any change in PAV or TAV less than zero was evaluated. Using this definition, for the primary endpoint, PAV, approximately 47% of patients in the placebo group experienced regression, compared with 67% of the treatment group receiving the combination of a statin and PCSK9 inhibitor (between groups difference 17.0%, P<0.001). Similar results were observed for TAV with more patients achieving regression with combination therapy, (between groups difference 12.5%, P<0.001). This is the first clinical trial to show incremental effects on regression in patients who had been treated with moderate or intensive statin therapy prior to entry into the study. It is also the first demonstration of a reduction in atherosclerotic disease progression by IVUS for a non-statin LDL lowering therapy.

After demonstrating major clinical benefits in multiple large outcomes trials19-22, statins are considered essential in global guidelines for managing patients with clinically manifest coronary heart disease23,24. However, many patients do not achieve optimal LDL-C reduction25 or experience cardiovascular events despite statin therapy.27 Furthermore, some patients report inability to tolerate full therapeutic doses of statins.27 Inadequate LDL-C reduction and presence of high residual risk suggests that additional therapies could be useful. PCSK9 regulation of hepatic LDL receptor expression has provided a potentially useful target for therapeutic modulation to address residual cardiovascular risk in statin-treated patients, particularly with the observation that PCSK9 levels rise in response to statin administration.28 In the current trial, almost every patient was treated with a statin prior to study entry and addition of the PCSK9 inhibitor, evolocumab, provided incremental reduction in LDL-C levels and atheroma volume.

Favorable effects were observed in the Trial summarized in Example 1 on disease progression without an increase in the incidence of myalgias, elevations in hepatic transaminases or new onset diabetes. However, the number of treated patients was relatively small. Subcutaneous injections were well tolerated, with injection site reactions reported in only two evolocumab-treated patients, a low rate of detection of anti-drug antibodies and no neutralizing antibodies. These safety findings are consistent with prior observations showing no apparent excess in adverse events in statin-treated patients achieving very low LDL-C levels.

Subgroup analyses showed no heterogeneity in the favorable effects of PCSK9 inhibition on disease progression. Regression with evolocumab was observed regardless of baseline LDL-C levels. An LDL-C of 70 mg/dL represents the most stringent target level recommended by any global guideline for cholesterol treatment.24,25 In patients with a baseline LDL-C <70 mg/dL, post hoc analysis in the current trial demonstrated regression in PAV in >80% of patients with combination therapy. This observation is supportive of current treatment guidelines recommending intensive lipid lowering in patients at high cardiovascular risk.23,24 These findings are reassuring from a safety perspective.

The definitive evidence supporting PCSK9 inhibitors as a clinically effective therapeutic strategy relies on the ability of these drugs to reduce cardiovascular adverse events. Prior reports have demonstrated an association between both the burden and rate of progression of coronary atherosclerosis and cardiovascular outcomes.30,31 While the current findings of the effect of evolocumab on disease progression are promising, completion of ongoing large cardiovascular outcome trials of PCSK9 inhibitors can provide further conformation of the efficacy and safety of these drugs.

The majority (approximately two-thirds) of patients achieved atheroma regression, despite achieving very low LDL-C levels with evolocumab. However, the Trial in Example 1 evaluated patients following 18 months of treatment, a relatively short duration of therapy in comparison with other recent studies of high intensity statin treatment which treated patients for 24 months. It remains possible that a greater percentage of patients would demonstrate regression at these low LDL levels with more prolonged treatment.

The above trial examined the effects of PCSK9 inhibition on disease progression in patients presenting for a clinically indicated coronary angiogram. It is assumed that similar effects will be observed in asymptomatic patients with manifest atherosclerosis. While patient retention (87%) was better than previous IVUS studies, as in any study, the results may have been influenced by patients who did not complete the trial.

During the two decades following the seminal observations that statins reduce adverse cardiovascular outcomes, there has been an ongoing search to identify additional therapies that produce incremental clinical benefit. The PCSK9 inhibitor, evolocumab, reduced LDL-C to very low levels resulting in marked regression of coronary atherosclerosis. While the large outcomes trials of PCSK9 inhibitors are in progress, the current findings indicate that combining a PCSK9 inhibitor with statins provides substantial incremental reduction in disease progression over a broad range of baseline LDL levels.

To summarize the results in Example 1, among the 968 treated patients, (mean age, 59.8 [9.2]; 269 [27.8%] women; LDL-C 92.5 mg/dL [27.2]), 846 had evaluable imaging at follow-up. Compared with placebo, the evolocumab group achieved lower mean, time-weighted LDL-C levels, 93.0 vs. 36.6 mg/dL, difference −56.5 mg/dL (95% confidence interval [CI]-59.7, −53.4), P<0.001. The primary efficacy parameter, PAV increased 0.05%, with placebo and decreased 0.95%, with evolocumab, difference −1.01% (95% CI −1.78, 0.64), P<0.001. The secondary efficacy parameter, normalized TAV, decreased 0.9 mm3 with placebo and 5.8 mm3 with evolocumab, difference −4.9 mm3 (95% CI −7.3, 2.5), P<0.001). Evolocumab induced plaque regression in a greater percentage of patients than placebo, for PAV, 64.3% vs. 47.3%, P<0.001 and for TAV 61.5% vs. 48.9%, P<0.001.

Among patients with angiographic coronary disease treated with statins, addition of evolocumab, compared with placebo, resulted in greater decrease in PAV after 78 weeks Evolocumab was well tolerated with a low incidence of laboratory safety abnormalities and cardiovascular events. The combined therapy not only prevented disease progression, but actually reversed it, in terms of PAV and TAV.

The above results confirm that lower LDL-C levels were observed in the evolocumab combined therapy group (36.6 vs. 93.0 mg/dL), which was associated with a reduction in percent atheroma volume for evolocumab (−0.95%), but not placebo (+0.05%) and a greater percentage of patients demonstrating plaque regression (64.3% vs. 47.3%). Thus, addition of the PCSK9 inhibitor, evolocumab, to statin therapy produced greater LDL-C lowering and atheroma regression. Furthermore, the data indicates that any treatment that achieves LDL-C levels as low as 20 mg/dL will show a benefit for the subject. In addition, the above benefits also support an approach where benefits are achieved by lowering LDL-C levels below the lowest levels currently recommended by global guidelines (<70 mg/dL). No safety issues were identified at the mean LDL-C levels of 36.6 mg/dL achieved in the trial, including: no excess in new onset diabetes, no myalgias, and no neurocognitive adverse effects.
1. LaRosa J C, Grundy S M, Waters D D, et al. Intensive lipid lowering with atorvastatin in patients with stable coronary disease. N Engl J Med. 2005; 352(14):1425-1435.
2. Cholesterol Treatment Trialists C, Baigent C, Blackwell L, et al. Efficacy and safety of more intensive lowering of LDL cholesterol: a meta-analysis of data from 170,000 participants in 26 randomised trials. Lancet. 2010; 376(9753):1670-1681.
3. Nicholls S J, Ballantyne C M, Barter P J, et al. Effect of two intensive statin regimens on progression of coronary disease. N Engl J Med. 2011; 365(22):2078-2087.
4. Nicholls S J, Tuzcu E M, Sipahi I, et al. Statins, high-density lipoprotein cholesterol, and regression of coronary atherosclerosis. JAMA. 2007; 297(5):499-508.
5. Nissen S E, Nicholls S J, Sipahi I, et al. Effect of very high-intensity statin therapy on regression of coronary atherosclerosis: the ASTEROID trial. JAMA. 2006; 295(13):1556-1565.
6. Nissen S E, Tuzcu E M, Schoenhagen P, et al. Effect of intensive compared with moderate lipid-lowering therapy on progression of coronary atherosclerosis: a randomized controlled trial. JAMA. 2004; 291(9):1071-1080.
7. Abifadel M, Varret M, Rabes J P, et al. Mutations in PCSK9 cause autosomal dominant hypercholesterolemia. Nature genetics. 2003; 34(2):154-156.
8. Maxwell K N, Breslow J L. Adenoviral-mediated expression of Pcsk9 in mice results in a low-density lipoprotein receptor knockout phenotype. Proc Natl Acad Sci USA. 2004; 101(18):7100-7105.
9. Seidah N G, Benjannet S, Wickham L, et al. The secretory proprotein convertase neural apoptosis-regulated convertase 1 (NARC-1): liver regeneration and neuronal differentiation. Proc Natl Acad Sci USA. 2003; 100(3):928-933.
10. Robinson J G, Nedergaard B S, Rogers W J, et al. Effect of evolocumab or ezetimibe added to moderate- or high-intensity statin therapy on LDL-C lowering in patients with hypercholesterolemia: the LAPLACE-2 randomized clinical trial. JAMA. 2014; 311(18):1870-1882.
11. Blom D J, Hala T, Bolognese M, et al. A 52-week placebo-controlled trial of evolocumab in hyperlipidemia. N Engl J Med. 2014; 370(19):1809-1819.
12. Puri R, Nissen S E, Somaratne R, et al. Impact of PCSK9 inhibition on coronary atheroma progression: Rationale and design of Global Assessment of Plaque Regression with a PCSK9 Antibody as Measured by Intravascular Ultrasound (GLAGOV). Am Heart J. 2016; 176:83-92.
13. Nissen S E, Nicholls S J, Wolski K, et al. Comparison of pioglitazone vs glimepiride on progression of coronary atherosclerosis in patients with type 2 diabetes: the PERISCOPE randomized controlled trial. JAMA. 2008; 299(13):1561-1573.
14. Nissen S E, Nicholls S J, Wolski K, et al. Effect of rimonabant on progression of atherosclerosis in patients with abdominal obesity and coronary artery disease: the STRADIVARIUS randomized controlled trial. JAMA. 2008; 299(13):1547-1560.
15. Nissen S E, Tardif J C, Nicholls S J, et al. Effect of torcetrapib on the progression of coronary atherosclerosis. N Engl J Med. 2007; 356(13):1304-1316.
16. Nissen S E, Tsunoda T, Tuzcu E M, et al. Effect of recombinant ApoA-I Milano on coronary atherosclerosis in patients with acute coronary syndromes: a randomized controlled trial. JAMA. 2003; 290(17):2292-2300.
17. Nissen S E, Tuzcu E M, Brewer H B, et al. Effect of ACAT inhibition on the progression of coronary atherosclerosis. N Engl J Med. 2006; 354(12):1253-1263.
18. Nissen S E, Tuzcu E M, Libby P, et al. Effect of antihypertensive agents on cardiovascular events in patients with coronary disease and normal blood pressure: the CAMELOT study: a randomized controlled trial. JAMA. 2004; 292(18):2217-2225.
19. Randomised trial of cholesterol lowering in 4444 patients with coronary heart disease: the Scandinavian Simvastatin Survival Study (4S). Lancet. 1994; 344(8934):1383-1389.
20. Prevention of cardiovascular events and death with pravastatin in patients with coronary heart disease and a broad range of initial cholesterol levels. The Long-Term Intervention with Pravastatin in Ischaemic Disease (LIPID) Study Group. N Engl J Med. 1998; 339(19):1349-1357.
21. Sacks F M, Pfeffer M A, Moye L A, et al. The effect of pravastatin on coronary events after myocardial infarction in patients with average cholesterol levels. Cholesterol and Recurrent Events Trial investigators. N Engl J Med. 1996; 335(14):1001-1009.
22. Shepherd J, Cobbe S M, Ford I, et al. Prevention of coronary heart disease with pravastatin in men with hypercholesterolemia. West of Scotland Coronary Prevention Study Group. N Engl J Med. 1995; 333(20):1301-1307.
23. Stone N J, Robinson J G, Lichtenstein A H, et al. 2013 ACC/AHA guideline on the treatment of blood cholesterol to reduce atherosclerotic cardiovascular risk in adults: a report of the American College of Cardiology/American Heart Association Task Force on Practice Guidelines. J Am Coll Cardiol. 2014; 63(25 Pt B):2889-2934.
24. Catapano A L, Graham I, De Backer G, et al. 2016 ESC/EAS Guidelines for the Management of Dyslipidaemias: The Task Force for the Management of Dyslipidaemias of the European Society of Cardiology (ESC) and European Atherosclerosis Society (EAS) Developed with the special contribution of the European Assocciation for Cardiovascular Prevention & Rehabilitation (EACPR). Eur Heart J. 2016.
25. Jones P H, Nair R, Thakker K M. Prevalence of dyslipidemia and lipid goal attainment in statin-treated subjects from 3 data sources: a retrospective analysis. Journal of the American Heart Association. 2012; 1(6):e001800.
26. Libby P. The forgotten majority: unfinished business in cardiovascular risk reduction. J Am Coll Cardiol. 2005; 46(7):1225-1228.
27. Nissen S E, Stroes E, Dent-Acosta R E, et al. Efficacy and Tolerability of Evolocumab vs Ezetimibe in Patients With Muscle-Related Statin Intolerance: The GAUSS-3 Randomized Clinical Trial. JAMA. 2016; 315(15):1580-1590.
28. Mayne J, Dewpura T, Raymond A, et al. Plasma PCSK9 levels are significantly modified by statins and fibrates in humans. Lipids in health and disease. 2008; 7:22.
29. Wiviott S D, Cannon C P, Morrow D A, Ray K K, Pfeffer M A, Braunwald E. Can low-density lipoprotein be too low The safety and efficacy of achieving very low low-density lipoprotein with intensive statin therapy: a PROVE IT-TIMI 22 substudy. J Am Coll Cardiol. 2005; 46(8):1411-1416.
30. Nicholls S J, Hsu A, Wolski K, et al. Intravascular ultrasound-derived measures of coronary atherosclerotic plaque burden and clinical outcome. J Am Coll Cardiol. 2010; 55(242399-2407.
31. Puri R, Nissen S E, Shao M, et al. Coronary atheroma volume and cardiovascular events during maximally intensive statin therapy. Eur Heart J. 2013; 34(41):3182-3190.

### Example 2

### Uses of PCSK9 Antibodies and Statins for the Reduction of Atherosclerosis

A human subject at risk of developing atherosclerosis is identified. The subject is administered a therapeutically effective amount of evolocumab, with a statin at an optimized level of statin administration. The combined therapy is maintained for at least one year. Throughout the year, the subject's LDL-C levels drop beneath 90 mg/dL, thereby reducing their risk of atherosclerosis in comparison to patients not receiving the treatment.

### Example 3

A patient with clinically evident atherosclerotic cardiovascular (CV) disease is identified. The patient is administered a therapeutically effective amount of evolocumab, with 40 mg/day of atorvastatin (or an equivalent thereto). The combined therapy is maintained for at least one year. Throughout the year, the subject's LDL-C levels drop beneath 90 mg/dL, thereby reducing their risk of CV death, non-fatal myocardial infarction, non-fatal stroke or transient ischemic attack (TIA), and coronary revascularization.

### Example 4

A patient with clinically evident atherosclerotic cardiovascular (CV) disease is identified. The patient is administered 420 mg/month of evolocumab, with 80 mg/day of atorvastatin (or an equivalent thereto). The combined therapy is maintained for at least one year. The combined therapy thereby reduces their risk of CV death, non-fatal myocardial infarction, non-fatal stroke or transient ischemic attack (TIA), coronary revascularization, and hospitalization for unstable angina.

### Example 5

A patient having atherosclerotic plaque is identified. The patient is administered evolocumab, with an amount of a statin that is equivalent to 40, or in the alternative, 80 mg/day of atorvastatin. The combined therapy is maintained for at least one year. The combined therapy thereby reduces the patient's PAV.

### Example 6

A patient having atherosclerotic plaque is identified. The patient is administered evolocumab, with an amount of a statin that is equivalent to 40, or in the alternative, 80 mg/day of atorvastatin. The combined therapy is maintained for at least one year. The combined therapy thereby reduces the patient's TAV.

### Example 7

A patient having coronary atherosclerosis is identified. The patient is receiving a non-PCK9 LDL-C lowering therapy (e.g., a statin). The patient is administered a PCSK9 inhibitor therapy. The amount and time of the PCSK9 inhibitor therapy (e.g., an anti-PCSK9 neutralizing antibody), combined with the continued application of the non-PCK9 LDL-C lowering therapy, is sufficient to reverse coronary atherosclerosis in the subject.

### Example 8

A patient having coronary artery disease is identified. The patient is administered an amount of an anti-PCSK9 neutralizing antibody and a maximally tolerated dose of a statin. The combined therapy is maintained for at least one year. The combined therapy thereby reduces the patient's TAV and PAV.

### Example 9

A patient having atherosclerosis is identified. The patient is administered an amount of an anti-PCSK9 neutralizing antibody and a maximally tolerated dose of a statin. The combined therapy is maintained for at least one year such that the patient's LDL-C level is maintained beneath 90 mg/dL. The combined therapy thereby reduces the patient's TAV and PAV.

### Example 10

A patient having plaques and/or atherosclerosis is identified. The patient is administered an amount of a PCSK9 inhibitor and a maximally tolerated dose of a statin. The combined therapy is maintained for at least one year such that the patient's LDL-C level is beneath 60 mg/dL. The combined therapy thereby results in plaque regression and regression in atherosclerosis.

### Example 11

A patient having atherosclerosis is identified. The patient is administered an amount of a PCSK9 inhibitor and an optimized dose of a statin. The combined therapy is maintained for at least one year such that the patient's LDL-C level is beneath 60 mg/dL. The combined therapy thereby results in regression in atherosclerosis.

### Example 12

A subject at risk of developing atherosclerosis is identified. The subject is administered an amount of a PCSK9 inhibitor and an optimized dose of a statin. The combined therapy is maintained for at least one year such that the subject's LDL-C level is beneath 60 mg/dL. The combined therapy thereby results in decreasing the risk that the subject will develop atherosclerosis.

### Example 13

A patient having atherosclerosis is identified. The patient is administered an amount of a PCSK9 inhibitor in an amount and time such that the patient's LDL-C level is maintained beneath 60 mg/dL for at least one year. The therapy thereby results in regression in atherosclerosis.

### Example 14

A patient having atherosclerotic plaque is identified. The patient is administered an amount of a PCSK9 inhibitor in an amount and time such that the patient's LDL-C level is maintained between 20 mg/dL and 40 mg/dL for at least one year. The therapy thereby results in regression in the atherosclerotic plaque.

### Example 15

A patient having atherosclerotic plaque is identified. The patient is administered an amount of a statin in an amount and time such that the patient's LDL-C level is maintained between 20 mg/dL and 40 mg/dL for at least one year. The therapy thereby results in regression in the atherosclerotic plaque.

### Example 16

A patient having atherosclerotic cardiovascular disease is identified. The patient is administered an amount of a statin in an amount and time such that the patient's LDL-C level is maintained between 20 mg/dL and 50 mg/dL for at least two years. The therapy thereby results in a 15% reduction in the risk of the composite of cardiovascular death, myocardial infarction, stroke, hospitalization for unstable angina, or coronary revascularization and a 20% reduction in the risk of the cardiovascular death, myocardial infarction, or stroke.

### Example 17

A randomized, double-blind, placebo-controlled trial was conducted involving 27,564 patients with atherosclerotic cardiovascular disease and LDL cholesterol ≥70 mg/dL or non-HDL ≥100 on statin therapy. Patients were randomized to receive evolocumab (either 140 mg every 2 weeks or 420 mg monthly) or matching placebo injections subcutaneously. The primary efficacy endpoint was the composite of cardiovascular death, myocardial infarction, stroke, hospitalization for unstable angina, or coronary revascularization, whichever occurs first. The key secondary efficacy endpoint was the composite of cardiovascular death, myocardial infarction, or stroke, whichever occurs first. Median followup was 2.2 years.

Summary of Results:

Evolocumab lowered LDL cholesterol by 59%, from a median of 92 to 30 mg/dL (P<0.001). Evolocumab significantly reduced the risk of the primary endpoint [1344 (9.8%) vs. 1563 (11.3%) patients; HR 0.85, 95% CI 0.79-0.92, P<0.001] and the key secondary endpoint [816 (5.9%) vs. 1013 (7.4%) patients; HR 0.80, 95% CI 0.73-0.88, P<0.001]. Results were consistent across key subgroups, including those in the lowest quartile of baseline LDL cholesterol (median 74 mg/dL). The incidence of adverse events including muscle-related, diabetes and neurocognitive were similar in the two arms.

Summary of Conclusions:

Inhibition of PCSK9 with evolocumab on a background of statin therapy lowered LDL cholesterol to 30 mg/dL and reduced the risk of cardiovascular events with no major safety concerns. These findings demonstrate that patients with atherosclerotic cardiovascular disease benefit from LDL cholesterol lowering below current targets.

The present example outlines the results of a study entitled “Further cardiovascular OUtcomes Research with PCSK9 Inhibition in subjects with Elevated Risk” (FOURIER). FOURIER was a dedicated cardiovascular outcomes trial that tested the clinical efficacy and safety of evolocumab when added to high or moderate intensity statin therapy in patients with clinically evident atherosclerotic vascular disease.

### Detailed Discussion of the Methods of Example 17

### Study Design

The present example (the “FOURIER trial”) was a randomized, double-blind, placebo-controlled multinational clinical trial that randomized patients at 1,242 sites in 49 countries.

### Study Population

Eligible patients were between 40 and 85 years of age with clinically evident atherosclerotic cardiovascular disease, defined as a history of myocardial infarction, non-hemorrhagic stroke, or symptomatic peripheral artery disease, and additional characteristics that placed them at higher cardiovascular risk (full eligibility criteria in the Supplementary Appendix). Patients must have had a fasting LDL cholesterol ≥70 mg/dL or a non-HDL cholesterol of ≥100 mg/dL on an optimized stable lipid-lowering therapy, preferably a high intensity statin, but must have been at least atorvastatin 20 mg daily or equivalent, with or without ezetimibe.

### Randomization and Study Treatment

Eligible patients were randomized 1:1 to receive either evolocumab (either 140 mg every 2 weeks or 420 mg every month according to patient preference) or matching placebo injections subcutaneously. Randomized allocation of study treatment was performed via a central computerized system with stratification by final screening LDL cholesterol (<85 vs ≥85 mg/dL) and region, and was double-blind.

### End Points

The primary efficacy end point was major cardiovascular events defined as the composite of cardiovascular death, myocardial infarction, stroke, hospitalization for unstable angina, or coronary revascularization. The key secondary efficacy endpoint was the composite of cardiovascular death, myocardial infarction, or stroke. Other efficacy endpoints are listed in the Supplemental section of Example 17. Safety was assessed through collection of adverse events and central laboratory testing (see Supplemental section of Example 17). Descriptions of the endpoints are in the Supplementary section of Example 17.

### Statistical Considerations

The primary efficacy analysis was based on the time from randomized treatment assignment to the first occurrence of any element of the primary composite endpoint. If the primary endpoint was significantly reduced (P<0.05), then, in a hierarchical fashion, the key secondary endpoint and then cardiovascular death were to be tested at a significance level of 0.05. See the Supplementary section in Example 17 for further details. All efficacy analyses were conducted on an intention-to-treat basis. Safety evaluations included all randomized patients who received at least one dose of study treatment and for whom post-dose data are available. Trial sample size was based on the key secondary endpoint, and it was estimated that 1630 such end points were required to provide 90% power to detect a 15% relative risk reduction with evolocumab. (Sabatine M S, Giugliano R P, Keech A, et al. Rationale and design of the Further cardiovascular OUtcomes Research with PCSK9 Inhibition in subjects with Elevated Risk trial. Am Heart J 2016; 173:94-101.) Hazard ratios and 95% confidence intervals were generated using a Cox proportional hazards model with stratification factors as covariates, and P values for time-to-event analyses are from log-rank tests.

### Results of Example 17

### Patients

A total of 27,564 patients were randomized between February 2013 and June 2015. The baseline characteristics of the patients in the two arms were well matched and are shown in Table 17.1.

TABLE 17.1
Baseline Characteristics of the Patients

| | Evolocumab | |
|---|---|---|
| Characteristics | (N = 13.784) | Placebo (N = 13.780) |
| Age - yr | 62.5 ± 9.1 | 62.5 ± 8.9 |
| Male sex - no. (%) | 10,397 (75.4) | 10,398 (75.5) |
| White race - no. (%)† | 11,748 (85.2) | 11,710 (85.0) |
| Weight - kg | 85.0 ± 17.3 | 85.5 ± 017.4 |
| Region | | |
| North America | 2,287 (16.6) | 2,284 (16.6) |
| Europe | 8,666 | 8,669 (62.9) |
| Latin America | 913 (6.6) | 910 (6.6) |
| Asia Pacific and South Africa | 1,918 (13.9) | 1,917 (13.9) |
| Type of atherosclerosis‡ | | |
| Myocardial infarction - no. (%) | 11,145 (80.9) | 11,206 (81.3) |
| Median time from most recent previous | 3.4 (1.0-7.4) | 3.3 (0.9-7.7) |
| myocardial infarction (IQR) - yr | | |
| Nonhemorrhagic stroke | 2686 (19.5) | 2651 (19.2) |
| Median time from most recent previous | 3.2 (1.1-7.1) | 3.3 (1.1-7.3) |
| stroke (IQR) - yr | | |
| Peripheral artery disease - no. (%) | 1,858 (13.5) | 1,784 (12.9) |
| Cardiovascular risk factors | | |
| Hypertension - no./total no. (%) | 11,045/13,784 (80.1) | 11,039/13,779 (80.1) |
| Diabetes mellitus - no. (%) | 5,054 (36.7) | 5,027 (36.5) |
| Current cigarette use - no./total no. (%) | 3854/13,783 (28.0) | 3923/13,779 (28.5) |
| Statin use - no. (%)†† | | |
| High intensity | 9,585 (69.5) | 9,518 (69.1) |
| Moderate intensity | 4,161 (30.2) | 4,231 (30.7) |
| Low intensity, unknown intensity, or no data | 38 (0.3) | 31 (0.2) |
| Ezetimibe - no. (%) | 726 (5.3) | 714 (5.2) |
| Other cardiovascular medications - | | |
| no./total no. (%) | | |
| Aspirin, P2Y₁₂ inhibitor, or both | 12,766/13,772 (92.7) | 12,666/13,767 (92.0) |
| Beta-blocker | 10,441/13,772 (75.8) | 10,374/13,767 (75.4) |
| ACE inhibitor or ARB, aldosterone | 10,803/13,772 (78.4) | 10.730/13,767 (77.9) |
| antagonist, or both | | |
| Median lipid measures (IQR) | | |
| LDL cholesterol - mg/dl | 92 (80-109 | 92 (80-109) |
| Total cholesterol - mg/dl | 168 (151-188) | 168 (151-189) |
| HDL cholesterol - mg/dl | 44 (37-53) | 44 (37-53) |
| Triglycerides - mg/dl | 134 (101-183) | 133 (99-181) |
| Lipoprotein(a) - nmol/liter | 37 (13-166) | 37 (13-164) |

*There were no nominally signficant differences between the two groups in baseline characteristics with the exception of weight (P = 0.01) and the use of aspirin, a P2Y₁₂ inhibitor, or both (P = 0.03). To convert the values for cholesterol to millimoles per liter, multiply by 0.02586. To convert the values for triglycerides to millimoles per liter, multiply by 0.01129. ACE denotes angiotensin-converting enzyme, ARB antiotensin-receptor blocker, HDL high-density lipoprotein, IQR interquartile range, and LDL low-density lipoprotein.
†Race was reported by the patients.
‡Patients could have more than one type of atheroscloersis.
††Statin intensity was categorized in accordance with the guidelines of the American College of Cardiology and American Hearth Association.¹²

The average age of the patients was 63 years, 25% were women; 81% had a history of myocardial infarction, 19% prior non-hemorrhagic stroke, and 13% symptomatic peripheral artery disease. At baseline a total of 69.3% patients were on high intensity statin therapy (defined as per ACC/AHA guidelines (Stone N J, Robinson J G, Lichtenstein A H, et al. 2013 ACC/AHA guideline on the treatment of blood cholesterol to reduce atherosclerotic cardiovascular risk in adults: a report of the American College of Cardiology/American Heart Association Task Force on Practice Guidelines. Circulation 2014; 129:S1-45.), see Supplementary section in Example 17) and 30.4% on moderate intensity statin therapy; 5.2% were also taking ezetimibe. Over the duration of the trial, only 9.8% of patients altered background lipid-lowering therapy (see Supplemental section in Example 17, Results for details). Use of secondary preventive therapies was high with 93% of patients taking antiplatelet therapy, 76% taking beta-blockers, and 78% taking an ACE (angiotension-converting enzyme) inhibitor or ARB (angiotensin receptor blocker) and/or an aldosterone antagonist at trial entry.

A total of 27,525 patients (99.9%) received at least one dose of study drug. Premature permanent discontinuation of study drug occurred in 12.5% of patients (5.7% per annum), withdrawal of consent in 0.7% (0.3% per annum), and loss to follow up in <0.1% (0.03% per annum), with similar rates in the two study groups (FIG. 17). The median duration of follow-up was 26 months (IQR 22-30) resulting in 59,865 patient years of follow-up. Ascertainment of the primary end point was complete for 99% of potential patient-years of follow-up.

### Lipid Data

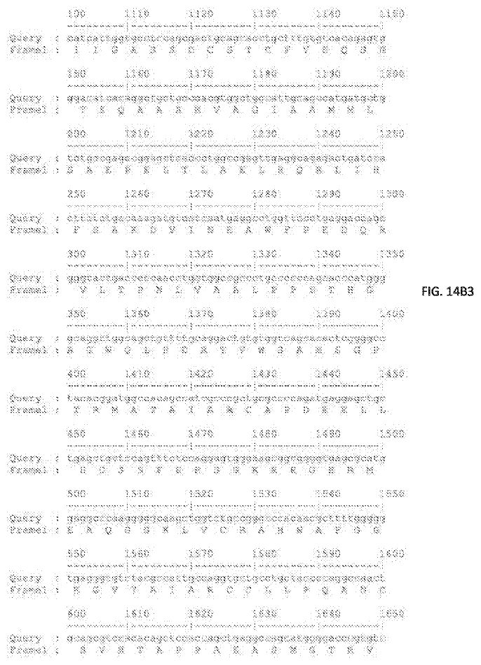

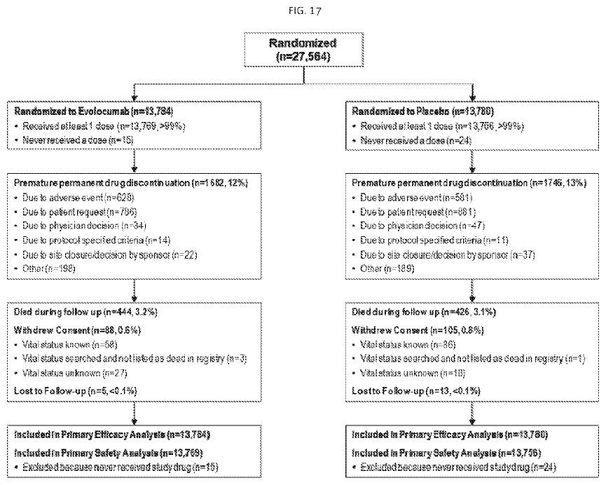

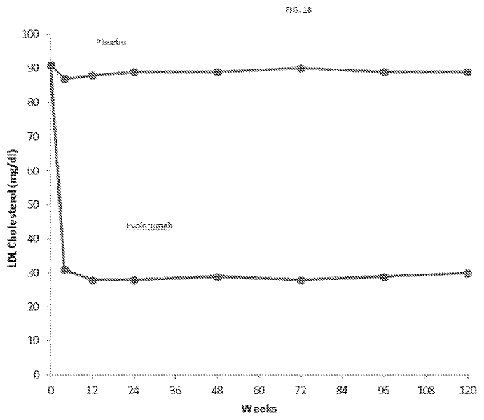

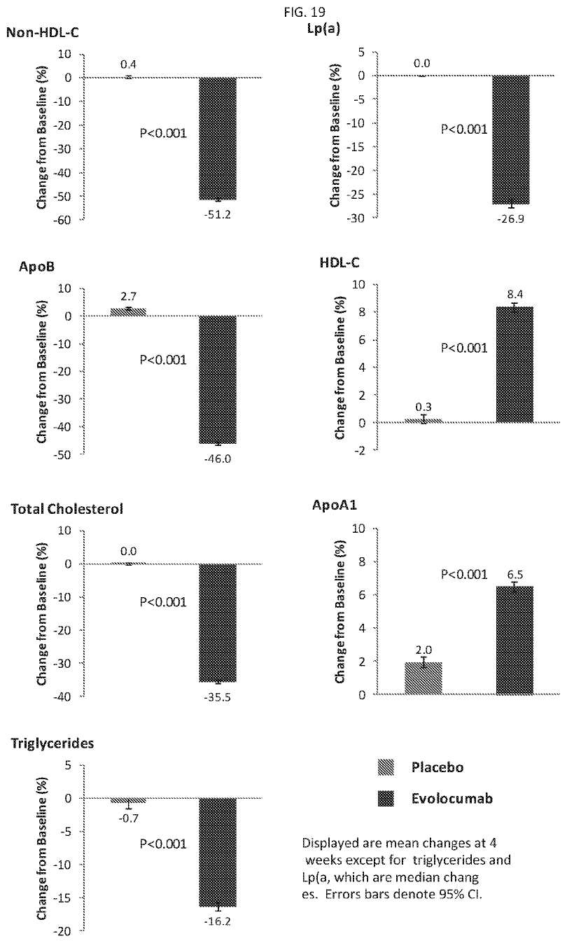

The median baseline LDL cholesterol was 92 mg/dL (IQR 80 to 109 mg/dL). Evolocumab as compared with placebo lowered LDL cholesterol by a mean of 59% (95% CI 58 to 60; P<0.001) at 48 weeks, for a mean absolute reduction of 56 mg/dL (95% CI 55 to 57) to a median of 30 mg/dL (IQR 19 to 46 mg/dL). The reduction in LDL cholesterol was maintained over time (FIG. 15 and FIG. 18). At 48 weeks the LDL cholesterol was reduced to ≤70 mg/dL in 87%, ≤40 mg/dL in 67%, and ≤25 mg/dL in 42% of the evolocumab group, as compared with 18%, 0.5%, and <0.1%, respectively in the placebo group (P<0.001 for all treatment comparisons). Evolocumab similarly lowered related atherogenic lipid measures, with placebo-controlled reductions at 48 weeks of 52% in non-HDL cholesterol and 49% in apolipoprotein B (P<0.001 for both). See Supplemental Results in Example 17 and FIG. 19 for further details.

### Efficacy End Points

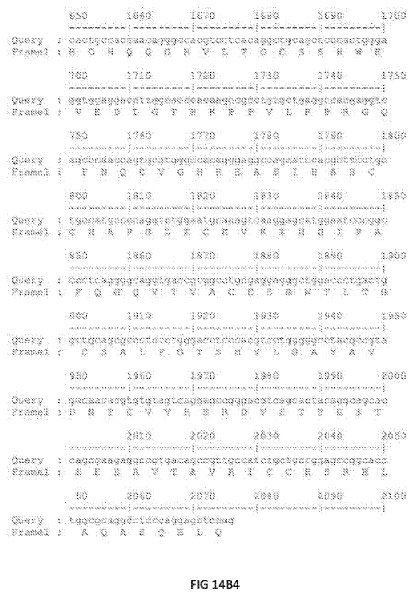

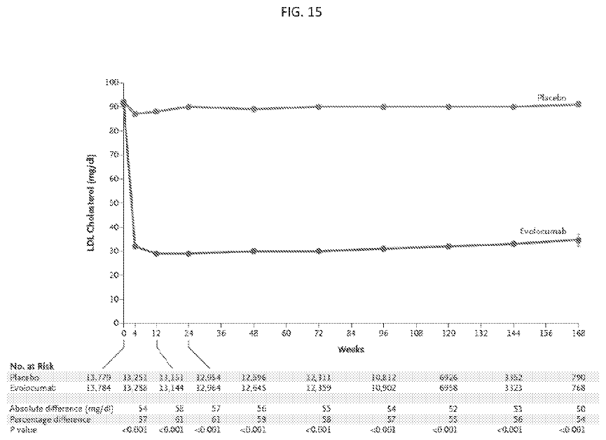

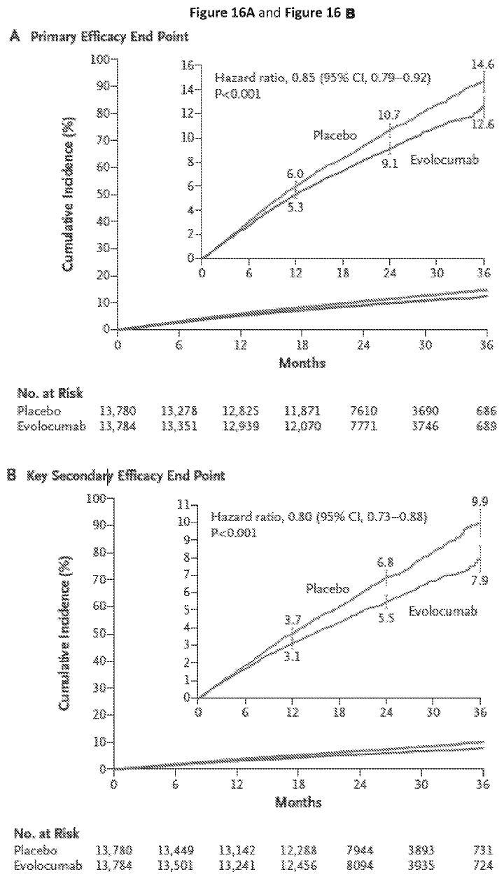

Evolocumab significantly reduced the risk of the primary composite end point of cardiovascular death, myocardial infarction, stroke, hospitalization for unstable angina, or coronary revascularization. The primary endpoint occurred in 1344 patients (9.8%) in the evolocumab arm and 1563 patients (11.3%) in the placebo arm (HR 0.85, 95% CI 0.79-0.92, P<0.001) (Table 17.2a and FIG. 16A). For FIGS. 16A and 16B, the Kaplan-Meier rates for the primary endpoint at 1, 2, and 3 years were 5.3% (95% CI 4.9-5.7) vs. 6.0% (95% CI 5.6-6.4), 9.1% (95% CI 8.6-9.6) vs. 10.7% (95% CI 10.1-11.2), and 12.6% (95% CI 11.7-13.5) vs. 14.6% (95% CI 13.8-15.5), respectively for the evolocumab and placebo arms. The Kaplan-Meier rates for the key secondary endpoint at 1, 2, and 3 years were 3.1% (95% CI 2.8-3.4) vs. 3.7% (95% CI 3.4-4.0), 5.5% (95% CI 5.1-5.9) vs. 6.8% (95% CI 6.4-7.3), and 7.9% (95% CI 7.2-8.7) vs. 9.9% (95% CI 9.2-10.7), respectively for the evolocumab and placebo arms. P values were calculated using log-rank tests.

TABLE 17.2a

| | Evolocumab | Placebo | Hazard Ratio | |
|---|---|---|---|---|
| Outcome | (N = 13,784) | (N = 13,780) | (95% Cl) | P Value* |
| | no. of patients (%) | | | |
| Primary end point: cardiovascular death, | 1344 (9.8) | 1563 (11.3) | 0.85 (0.79-0.92) | <0.001 |
| myocardial infarction, stroke, hospitalization | | | | |
| for unstable angina, or coronary | | | | |
| revascularization | | | | |
| Key secondary end point: cardiovascular death, | 816 (5.9) | 1013 (7.4) | 0.80 (0.73-0.88) | <0.001 |
| myocardial infarction, or stroke | | | | |
| Other end points | | | | |
| Cardiovascular death | 251 (1.8) | 240 (1.7) | 105 (0.88-1.25) | 0.62 |
| Due to acute myocardial infarction | 25 (0.18) | 30 (0.22) | 0.84 (0.49-1.42) | |
| Due to stroke | 31 (0.22) | 3 (0.24) | 0.94 (0.58-1.54) | |
| Other cardiovascular death | 195 (1.4) | 177 (1.3) | 1.10 (0.90-1.35) | |
| Death from any cause | 444 (3.2) | 426 (3.1) | 1.04 (0.91-1.19) | 0.54 |
| Myocardial infarction | 468 (3.4) | 639 (4.6) | 0.73 (0.65-0.82) | <0.001 |
| Hospitalization for unstable angina | 236 (1.7) | 239 (1.7) | 0.99 (0.82-1.18) | 0.89 |
| Stroke | 207 (1.5) | 262 (1.9) | 0.79 (0.66-0.95) | 0.01 |
| lschemic | 171 (1.2) | 226 (1.6) | 0.75 (0.62-0.92) | |
| Hemorrhagic | 29 (0.21) | 25 (0.18) | 1.16 (0.68-1.98) | |
| Unknown | 13 (0.09) | 14 (0.10) | 0.93 (0.44-1.97) | |
| Coronary revascularization | 759 (5.5) | 965 (7.0) | 0.78 (0.71-0.86) | <0.001 |
| Urgent | 403 (2.9) | 547 (4.0) | 0.73 (0.64-0.83) | |
| Elective | 420 (3.0) | 504 (3.7) | 0.83 (0.73-0.95) | |
| Cardiovascular death or hospitalization for | 402 (2.9) | 408 (3.0) | 0.98 (0.86-1.13) | 0.82 |
| worsening heart failure | | | | |
| Ischemic stroke or transient ischemic attack | 229 (1.7) | 295 (2.1) | 0.77 (0.65-0.92) | 0.003 |
| CTTC composite end pointt | 1271 (9.2) | 1512 (11.0) | 0.83 (0.77-0.90) | <0.001 |

*Given the hierarchical nature of the statistical testing, the P values for the primary and key secondary end points should be considered significant, whereas all other P values should be considered exploratory.
†The Cholesterol Treatment Trialists Collaboration (CTTC) composite end point consists of coronary heart death, nonfatal MI, strok,e or coronary revascularization.

CTTC stands for Cholesterol Treatment Trialists Collaboration and the composite endpoint of coronary heart death, nonfatal MI, stroke, or coronary revascularization. Given the hierarchical nature of the statistical testing, the P values for the primary and key secondary endpoint should be considered statistically significant whereas all other P values should be considered exploratory.

Likewise, evolocumab significantly reduced the rate of the key secondary composite end point of cardiovascular death, myocardial infarction, or stroke. The key secondary endpoint occurred in 816 patients (5.9%) in the evolocumab arm and 1013 (7.4%) in the placebo arm (HR 0.80, 95% CI 0.73-0.88, P<0.001) (Table 17.2, FIG. 16B). The magnitude of the risk reduction in the primary endpoint tended to increase over time, from 12% (95% CI 3 to 20) in the first year to 19% (95% CI 11 to 27) beyond the first year. Likewise for the key secondary endpoint the risk reduction went from 16% (95% CI 4 to 26) in the first year to 25% (95% CI 15 to 34) beyond the first year (see FIG. 20, Table 17.2b and Example 17 Supplemental Results).

TABLE 17.2b

| | Hazard Ratio (95% CI) | |
|---|---|---|
| Outcome | In first year | Beyond first year |
| Primary end point | 0.88 (0.80-0.97) | 0.81 (0.73-0.89) |
| Key secondary end point | 0.84 (0.74-0.96) | 0.75 (0.66-0.85) |
| Cardiovascular death | 0.96 (0.74-1.25) | 1.12 (0.88-1.42) |
| Myocardial infarction | 0.80 (0.68-0.94) | 0.65 (0.55-0.77) |
| Hospitalization for unstable angina | 0.97 (0.77-1.22) | 0.99 (0.75-1.30) |
| Stroke | 0.83 (0.63-1.08) | 0.76 (0.60-0.97) |
| Coronary revascularization | 0.84 (0.74-0.96) | 0.72 (0.63-0.82) |
| Urgent | 0.84 (0.71-1.00) | 0.63 (0.52-0.75) |
| Elective | 0.86 (0.72-1.03) | 0.81 (0.68-0.97) |
| CTTC composite endpoint | 0.87 (0.79-0.97) | 0.78 (0.71-0.86) |
| Coronary heart death, MI, ischemic | 0.86 (0.76-0.97) | 0.76 (0.68-0.86) |
| stroke, or urgent revascularization | | |
| Coronary heart death, MI, or stroke | 0.84 (0.73-0.95) | 0.73 (0.65-0.83) |
| Fatal or nonfatal MI or stroke | 0.81 (0.70-0.93) | 0.67 (0.59-0.77) |

Primary end point consists of cardiovascular death, myocardial infarction, stroke, hospitalization for unstable angina, or coronary revascularization. The key secondary end point consists of cardiovascular death, myocardial infarction, or stroke.

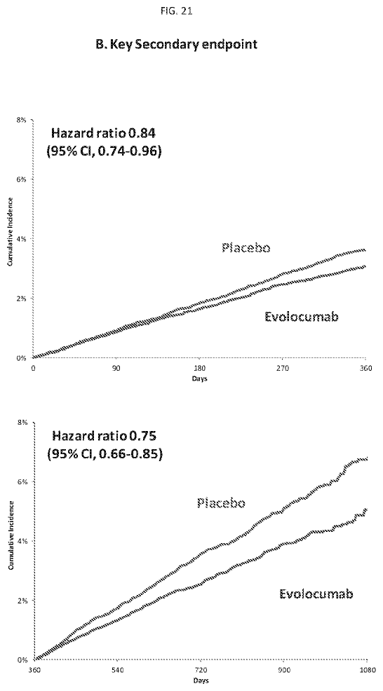

There were 21 to 27% reductions in the risk of MI, stroke and coronary revascularization, but no observed effect on hospitalization for unstable angina, hospitalization for worsening heart failure, or death from any cause (Table 17.2). The benefits of evolocumab on the risk of the primary and key secondary composite end points were largely consistent across major subgroups including age, sex, and type of atherosclerotic vascular disease (FIG. 22). It was also consistent across quartiles of baseline LDL cholesterol, ranging from patients in the top quartile starting with a median LDL cholesterol of 126 mg/dL (IQR 116 to 143) down to those in the lowest quartile starting with a median LDL cholesterol of 74 mg/dL (IQR 69 to 77). The benefit of evolocumab was also consistent across statin intensity, regardless of ezetimibe use and in both the 140 mg every 2 weeks and 420 mg monthly dosing regimens (FIG. 22).

### Safety

No statistically significant between-group differences were seen in the overall rate of adverse events, serious adverse events, or adverse events thought to be related to study drug and leading to study drug discontinuation (Table 17.3).

TABLE 17.3

| | Evolocumab | Placebo |
|---|---|---|
| Outcome | (N = 13,769) | (N = 13,756) |
| Adverse events - no. of patients (%) | | |
| Any | 10,664 (77.4) | 10,664 (77.4) |
| Serious | 3410 (24.8) | 3404 (24.7) |
| Thought to be related to the study | 226 (1.6) | 201 (1.5) |
| agent and leading to | | |
| discontinuation of study regimen | | |
| Injection-site reaction* | 296 (2.1) | 219 (1.6) |
| Allergic reaction | 420 (3.1) | 393 (2.9) |
| Muscle-related event | 682 (5.0) | 656 (4.8) |
| Rhabdomyolysis | 8 (0.1) | 11 (0.1) |
| Cataract | 228 (1.7) | 242 (1.8) |
| Adjudicated case of new-onset | 677 (8.1) | 644 (7.7) |
| diabetes† | | |
| Neurocognitive event | 217 (1.6) | 202 (1.5) |
| Laboratory results - | | |
| no. of patients/total no. (%) | | |
| Aminotransferase level >3 times | 240/13,543 (1.8) | 242/13,523 (1.8) |
| the upper limit of the normal range | | |
| Creatinine kinase level >5 times | 95/13,543 (0.7) | 99/13,523 (0.7) |
| the upper limit of the normal range | | |

Likewise rates of muscle-related, cataract, neurocognitive adverse events and hemorrhagic stroke were not significantly different between the two arms. Injection site reactions were rare, but more frequent with evolocumab (2.1% vs 1.6%). The vast majority of reactions (−90% in each arm) were classified as mild and only 0.1% of patients in each arm stopped study drug because of an injection site reaction. The rates of adjudicated new onset diabetes were not significantly different between the two arms (HR 1.05, 95% CI 0.94-1.17). Rates of allergic reactions were also not significantly different (3.1% vs. 2.9%). In the evolocumab arm, new binding antibodies were detected in 43 patients (0.3%) and neutralizing antibodies in none.

### Discussion of Results of Example 17

When added to statin therapy, the PCSK9 inhibitor evolocumab lowered LDL cholesterol by 59% from a median of 92 to 30 mg/dL (from 2.4 to 0.8 mmol/L). This effect was sustained over 3 years without evidence of attenuation. The present results confirm, for the first time in a dedicated cardiovascular outcomes study, that the addition of a PCSK9 inhibitor to statin therapy significantly reduces the risk of cardiovascular events, with a 15% reduction in the risk of the primary composite end point of cardiovascular death, myocardial infarction, stroke, hospitalization for unstable angina, or coronary revascularization and a 20% reduction in the risk of the harder key secondary end point of cardiovascular death, myocardial infarction, or stroke. Furthermore, there were no major safety concerns with evolocumab.

The data from the present example (FOURIER) provide insight into the benefit of decreasing LDL cholesterol to heretofore unprecedented low levels (as median values). Previously, significant reductions in major cardiovascular events were seen in the PROVE-IT TIMI 22 and TNT trials, in which the more intensive statin arm lowered LDL cholesterol from approximately 100 to 70 mg/dL. Cannon C P, Braunwald E, McCabe C H, et al. Intensive versus moderate lipid lowering with statins after acute coronary syndromes. N Engl J Med 2004; 350:1495-504; and LaRosa J C, Grundy S M, Waters D D, et al. Intensive lipid lowering with atorvastatin in patients with stable coronary disease. N Engl J Med 2005; 352:1425-35.) More recently, the addition of ezetimibe to statin therapy in the IMPROVE-IT trial lowered LDL cholesterol from 70 to 54 mg/dL and significantly reduced major vascular events. (Cannon C P, Blazing M A, Giugliano R P, et al. Ezetimibe Added to Statin Therapy after Acute Coronary Syndromes. N Engl J Med 2015; 372:2387-97.) In the present example (FOURIER) there were consistent reductions in cardiovascular events across the range of baseline LDL cholesterol. Specifically, there was a 17% reduction in risk of the key secondary endpoint in patients in the top quartile of baseline LDL cholesterol, in whom evolocumab lowered the median LDL cholesterol from 126 to 43 mg/dL (similar to the level achieved with ezetimibe in patients in the lowest quartile of admission LDL cholesterol levels in IMPROVE-IT (Giugliano R P, Cannon C, Blazing M, et al. Baseline LDL-C and clinical outcomes with addition of ezetimibe to statin in 18,144 patients post ACS. J Am Coll Cardiol 2015; 65:A4.) and a 22% reduction in risk in patients in the lowest quartile of LDL cholesterol, in whom evolocumab lowered the median LDL cholesterol from 73 to 22 mg/dL. These observations align well with the effects of evolocumab on coronary atherosclerotic plaque volume from the GLAGOV trial, (Nicholls S J, Puri R, Anderson T, et al. Effect of Evolocumab on Progression of Coronary Disease in Statin-Treated Patients: The GLAGOV Randomized Clinical Trial. JAMA 2016; 316:2373-84) and show that continued cardiovascular benefit can be accrued even when reducing LDL cholesterol down to the 20-25 mg/dL range, levels well below current targets. (Lloyd-Jones D M, Morris P B, Ballantyne C M, et al. 2016 ACC Expert Consensus Decision Pathway on the Role of Non-Statin Therapies for LDL-Cholesterol Lowering in the Management of Atherosclerotic Cardiovascular Disease Risk: A Report of the American College of Cardiology Task Force on Clinical Expert Consensus Documents. J Am Coll Cardiol 2016; 68:92-125; Landmesser U, John Chapman M, Farnier M, et al. European Society of Cardiology/European Atherosclerosis Society Task Force consensus statement on proprotein convertase subtilisin/kexin type 9 inhibitors: practical guidance for use in patients at very high cardiovascular risk. Eur Heart J 2016; Sabatine M S. Proprotein convertase subtilisin/kexin type 9 (PCSK9) inhibitors: comparing and contrasting guidance across the Atlantic. Eur Heart J 2017.)

In FOURIER, the magnitude of the risk reduction in the key secondary endpoint appeared to grow over time, from 16% over the first year to 25% beyond 12 months, suggesting that the translation of LDL cholesterol reduction into cardiovascular clinical benefit requires time. Overall, the number needed to treat to prevent a cardiovascular death, myocardial or stroke was 74 over 2 years or 50 over 3 years.

Consistent with prior trials of more intensive LDL cholesterol lowering therapy compared with moderate intensity statin therapy, (Cannon C P, Blazing M A, Giugliano R P, et al. Ezetimibe Added to Statin Therapy after Acute Coronary Syndromes. N Engl J Med 2015; 372:2387-97; Cholesterol Treatment Trialists C, Baigent C, Blackwell L, et al. Efficacy and safety of more intensive lowering of LDL cholesterol: a meta-analysis of data from 170,000 participants in 26 randomised trials. Lancet 2010; 376:1670-81) there was no observed effect of additional LDL cholesterol lowering on cardiovascular mortality. Use of evidence-based cardiovascular pharmacotherapies that lower cardiovascular mortality was very high in FOURIER, in which the rates of cardiovascular mortality were one third of the rates in the 4S trial (Scandinavian Simvastatin Survival Study Group. Randomised trial of cholesterol lowering in 4444 patients with coronary heart disease: the Scandinavian Simvastatin Survival Study (4S). Lancet 1994; 344:1383-89.) The relatively short duration of the trial may have precluded emergence of a cardiovascular mortality benefit. Similar to the findings in SEARCH and IMPROVE-IT, there was no effect on hospitalization for unstable angina. The advent of increasingly more sensitive cardiac troponin assays likely makes cardiac ischemia as the true cause of a hospitalization for chest pain symptoms without biochemical evidence of myocyte injury increasingly questionable. (Braunwald E, Morrow DA. Unstable angina: is it time for a requiem? Circulation 2013; 127:2452-7.) Lastly, urgent coronary revascularization appeared to be more modifiable than elective revascularization.

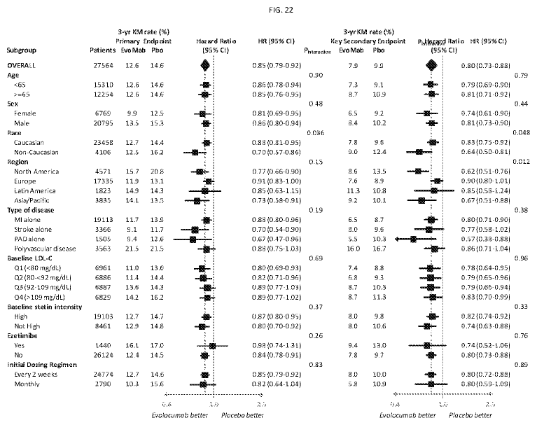

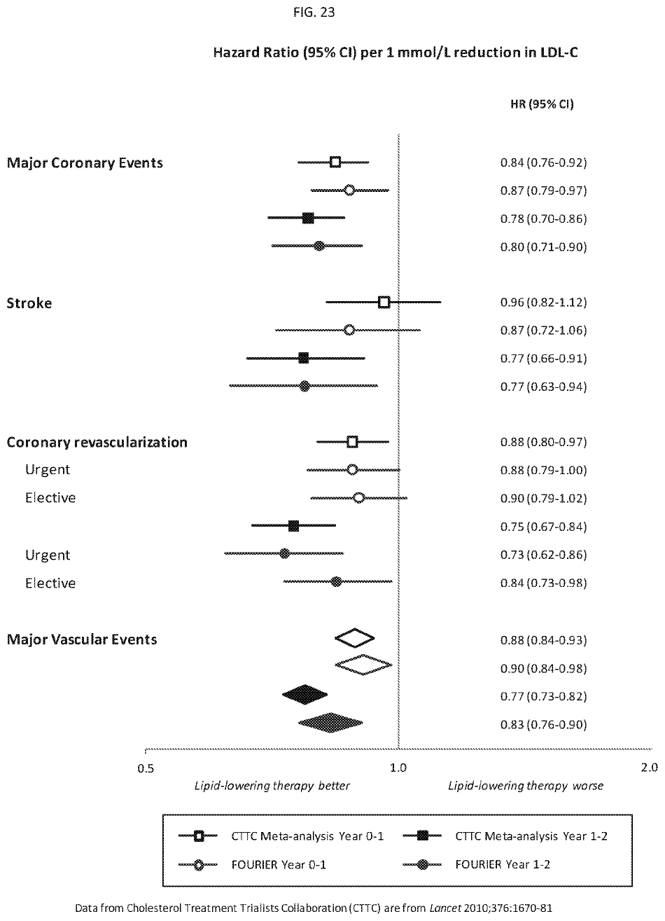

Given these caveats, the magnitude of benefit of evolocumab for reducing the risk of major coronary events, stroke, and urgent coronary revascularization is largely consistent with the benefit seen with statins on a per mmol/L basis of LDL cholesterol lowering (FIG. 23). These observations are in accord with data from meta-analyses of clinical trial data of different lipid-lowering interventions showing consistent clinical benefits per unit reduction of LDL cholesterol. (Silverman M G, Ference B A, Im K, et al. Association Between Lowering LDL-C and Cardiovascular Risk Reduction Among Different Therapeutic Interventions: A Systematic Review and Meta-analysis. JAMA 2016; 316:1289-97.) Likewise these observations are supported from data from a recent Mendelian randomization study in which variants in PCSK9 and in HMGCR were associated with nearly identical lower risk of cardiovascular events per unit lower LDL cholesterol. (Ference B A, Robinson J G, Brook R D, et al. Variation in PCSK9 and HMGCR and Risk of Cardiovascular Disease and Diabetes. N Engl J Med 2016; 375:2144-53.)

Achievement of these very low LDL cholesterol levels with evolocumab did not lead to any notable differences between the two study groups in the rates of adverse events or study drug discontinuation. The rate of evolocumab discontinuation due to adverse events ascribed to study drug was similar to placebo (0.76%/year vs. 0.67%/year) and compares favorably to the rates seen for atorvastatin 80 mg/d (1.5%/year) and ezetimibe (1.1%/year) (LaRosa J C, Grundy S M, Waters D D, et al. Intensive lipid lowering with atorvastatin in patients with stable coronary disease. N Engl J Med 2005; 352:1425-35 and Cannon C P, Blazing M A, Giugliano R P, et al. Ezetimibe Added to Statin Therapy after Acute Coronary Syndromes. N Engl J Med 2015; 372:2387-97.) There was not a statistically significant increase in new-onset diabetes with evolocumab, although the 95% confidence intervals do not exclude the point estimates observed with statins. (Sattar N, Preiss D, Murray H M, et al. Statins and risk of incident diabetes: a collaborative meta-analysis of randomised statin trials. Lancet 2010; 375:735-42; Preiss D, Seshasai S R, Welsh P, et al. Risk of incident diabetes with intensive-dose compared with moderate-dose statin therapy: a meta-analysis. JAMA 2011; 305:2556-64.) Potential concerns over an increased risk of neurocognitive adverse events were not borne out in this study. In contrast to recent data for bococizumab (a humanized but not fully human monoclonal antibody against PCSK9), (Pfizer Discontinues Global Development of Bococizumab, Its Investigational PCSK9 Inhibitor 2017. (Accessed Feb. 2, 2017, 2017, at website world wide web.pfizer.com/news/press-release/press-release-detail/pfizer_discontinues_global_development_of_bococizumab_its_investigational_pcsk9_inhibitor)) for evolocumab binding antibodies were rare, no neutralizing antibodies were detected, and the overall LDL cholesterol-lowering effect continued without attenuation. Furthermore, similarly reassuring findings with evolocumab were observed over 4 years in OSLER-1. (Koren M J, Sabatine M S, Giugliano R P, et al. Long-Term LDL-C Lowering Efficacy, Persistence, and Safety of Evolocumab in Chronic Treatment of Hypercholesterolemia: Results up to 4 years from the Open-Label OSLER-1 Extension Study. JAMA Cardiology 2017:in press).

One consideration of this study was a relatively short duration of follow-up compared with other lipid-lowering trials, which have averaged approximately 5 years. (Silverman M G, Ference B A, Im K, et al. Association Between Lowering LDL-C and Cardiovascular Risk Reduction Among Different Therapeutic Interventions: A Systematic Review and Meta-analysis. JAMA 2016; 316:1289-97) Although the median follow-up in FOURIER was originally planned to be approximately 4 years, an event rate that was approximately 50% higher than had been postulated led to a shorter required duration of follow-up to accrue the prespecified number of events. Based on apparent increasing efficacy over time, this shorter duration may have attenuated the overall proportional event reduction in FOURIER. The relatively short duration of the trial may have also limited the ability to detect delayed adverse events. The majority but not all patients received high intensity statin therapy and ezetimibe use was infrequent. However, the benefit of evolocumab was consistent regardless of the intensity of statin therapy or ezetimibe use.

In conclusion, inhibition of PCSK9 with evolocumab on a background of statin therapy further lowered LDL cholesterol down to a median 30 mg/dL and reduced the risk of cardiovascular events without any offsetting adverse events over the timeframe studied. These findings demonstrate that patients with atherosclerotic cardiovascular disease benefit from LDL cholesterol lowering below current targets.

Additional information regarding the present material can be found in, for example, the following references: Giugliano R P, Sabatine M S. Are PCSK9 Inhibitors the Next Breakthrough in the Cardiovascular Field? J Am Coll Cardiol 2015; 65:2638-51; Blom D J, Hala T, Bolognese M, et al. A 52-week placebo-controlled trial of evolocumab in hyperlipidemia. N Engl J Med 2014; 370:1809-19; Robinson J G, Nedergaard B S, Rogers W J, et al. Effect of evolocumab or ezetimibe added to moderate- or high-intensity statin therapy on LDL-C lowering in patients with hypercholesterolemia: the LAPLACE-2 randomized clinical trial. JAMA 2014; 311:1870-82; Koren M J, Lundqvist P, Bolognese M, et al. Anti-PCSK9 monotherapy for hypercholesterolemia: the MENDEL-2 randomized, controlled phase III clinical trial of evolocumab. J Am Coll Cardiol 2014; 63:2531-40; Stroes E, Colquhoun D, Sullivan D, et al. Anti-PCSK9 antibody effectively lowers cholesterol in patients with statin intolerance: the GAUSS-2 randomized, placebo-controlled phase 3 clinical trial of evolocumab. J Am Coll Cardiol 2014; 63:2541-8; Raal F J, Honarpour N, Blom D J, et al. Inhibition of PCSK9 with evolocumab in homozygous familial hypercholesterolaemia (TESLA Part B): a randomised, double-blind, placebo-controlled trial. Lancet 2015; 385:341-50; Cohen J C, Boerwinkle E, Mosley T H, Jr., Hobbs H H. Sequence variations in PCSK9, low LDL, and protection against coronary heart disease. N Engl J Med 2006; 354:1264-72; Kathiresan S. A PCSK9 missense variant associated with a reduced risk of early-onset myocardial infarction. N Engl J Med 2008; 358:2299-300; Sabatine M S, Giugliano R P, Wiviott S D, et al. Efficacy and safety of evolocumab in reducing lipids and cardiovascular events. N Engl J Med 2015; 372:1500-9; Robinson J G, Farnier M, Krempf M, et al. Efficacy and safety of alirocumab in reducing lipids and cardiovascular events. N Engl J Med 2015; 372:1489-99; Sabatine M S, Giugliano R P, Keech A, et al. Rationale and design of the Further cardiovascular OUtcomes Research with PCSK9 Inhibition in subjects with Elevated Risk trial. Am Heart J 2016; 173:94-101; Stone N J, Robinson J G, Lichtenstein A H, et al. 2013 ACC/AHA guideline on the treatment of blood cholesterol to reduce atherosclerotic cardiovascular risk in adults: a report of the American College of Cardiology/American Heart Association Task Force on Practice Guidelines. Circulation 2014; 129:S1-45; Cannon C P, Braunwald E, McCabe C H, et al. Intensive versus moderate lipid lowering with statins after acute coronary syndromes. N Engl J Med 2004; 350:1495-504; LaRosa J C, Grundy S M, Waters D D, et al. Intensive lipid lowering with atorvastatin in patients with stable coronary disease. N Engl J Med 2005; 352:1425-35; Cannon C P, Blazing M A, Giugliano R P, et al. Ezetimibe Added to Statin Therapy after Acute Coronary Syndromes. N Engl J Med 2015; 372:2387-97; Giugliano R P, Cannon C, Blazing M, et al. Baseline LDL-C and clinical outcomes with addition of ezetimibe to statin in 18,144 patients post ACS. J Am Coll Cardiol 2015; 65:A4; Nicholls S J, Puri R, Anderson T, et al. Effect of Evolocumab on Progression of Coronary Disease in Statin-Treated Patients: The GLAGOV Randomized Clinical Trial. JAMA 2016; 316:2373-84; Lloyd-Jones D M, Morris P B, Ballantyne C M, et al. 2016 ACC Expert Consensus Decision Pathway on the Role of Non-Statin Therapies for LDL-Cholesterol Lowering in the Management of Atherosclerotic Cardiovascular Disease Risk: A Report of the American College of Cardiology Task Force on Clinical Expert Consensus Documents. J Am Coll Cardiol 2016; 68:92-125; Landmesser U, John Chapman M, Farnier M, et al. European Society of Cardiology/European Atherosclerosis Society Task Force consensus statement on proprotein convertase subtilisin/kexin type 9 inhibitors: practical guidance for use in patients at very high cardiovascular risk. Eur Heart J 2016; Sabatine M S. Proprotein convertase subtilisin/kexin type 9 (PCSK9) inhibitors: comparing and contrasting guidance across the Atlantic. Eur Heart J 2017; Collins R, Reith C, Emberson J, et al. Interpretation of the evidence for the efficacy and safety of statin therapy. Lancet 2016; 388:2532-61; Lipid Research Clinics Program. The Lipid Research Clinics Coronary Primary Prevention Trial results. JAMA 1984; 251:351-64; Frick M H, Elo O, Haapa K, et al. Helsinki Heart Study: primary-prevention trial with gemfibrozil in middle-aged men with dyslipidemia. N Engl J Med 1987; 317:1237-45; Buchwald H, Varco R L, Matts J P, et al. Effect of partial ileal bypass surgery on mortality and morbidity from coronary heart disease in patients with hypercholesterolemia. Report of the Program on the Surgical Control of the Hyperlipidemias (POSCH). N Engl J Med 1990; 323:946-55; Cholesterol Treatment Trialists C, Baigent C, Blackwell L, et al. Efficacy and safety of more intensive lowering of LDL cholesterol: a meta-analysis of data from 170,000 participants in 26 randomised trials. Lancet 2010; 376:1670-81; Scandinavian Simvastatin Survival Study Group. Randomised trial of cholesterol lowering in 4444 patients with coronary heart disease: the Scandinavian Simvastatin Survival Study (4S). Lancet 1994; 344:1383-89; Braunwald E, Morrow D A. Unstable angina: is it time for a requiem? Circulation 2013; 127:2452-7; Silverman M G, Ference B A, Im K, et al. Association Between Lowering LDL-C and Cardiovascular Risk Reduction Among Different Therapeutic Interventions: A Systematic Review and Meta-analysis. JAMA 2016; 316:1289-97; Ference B A, Robinson J G, Brook R D, et al. Variation in PCSK9 and HMGCR and Risk of Cardiovascular Disease and Diabetes. N Engl J Med 2016; 375:2144-53; Sattar N, Preiss D, Murray H M, et al. Statins and risk of incident diabetes: a collaborative meta-analysis of randomised statin trials. Lancet 2010; 375:735-42; Preiss D, Seshasai S R, Welsh P, et al. Risk of incident diabetes with intensive-dose compared with moderate-dose statin therapy: a meta-analysis. JAMA 2011; 305:2556-64; Pfizer Discontinues Global Development of Bococizumab, Its Investigational PCSK9 Inhibitor 2017. (Accessed Feb. 2, 2017, 2017, at http://world wide web.pfizer.com/news/press-release/press-release-detail/pfizer_discontinues_global_development_of_bococizumab_its_investigational_pcsk9_inhibitor.); Koren M J, Sabatine M S, Giugliano R P, et al. Long-Term LDL-C Lowering Efficacy, Persistence, and Safety of Evolocumab in Chronic Treatment of Hypercholesterolemia: Results up to 4 years from the Open-Label OSLER-1 Extension Study. JAMA Cardiology 2017:in press.

### Example 17 Supplemental Information

### Supplemental Methods

### Statin Intensity

Classification is based on the 2013 ACC/AHA Guideline on the Treatment of Blood Cholesterol. (Stone N J, Robinson J G, Lichtenstein A H, et al. 2013 ACC/AHA guideline on the treatment of blood cholesterol to reduce atherosclerotic cardiovascular risk in adults: a report of the American College of Cardiology/American Heart Association Task Force on Practice Guidelines. Circulation. Jun. 24, 2014; 129(25 Suppl 2): S1-45.) Table 17.4 present exemplary ranges.

TABLE 17.4

| | Intensity | | |
|---|---|---|---|
| Statin | High | Moderate | Low |
| Atorvastatin | ≥40 mg | 10 to <40 mg | <10 mg |
| Rosuvastatin | ≥20 mg | 5 to <20 mg | <5 mg |
| Simvastatin | 80 mg | 20 to <80 mg | <20 mg |
| Pravastatin | | ≥40 mg | <40 mg |
| Lovastatin | | ≥40 mg | <40 mg |
| Fluvastatin | | 80 mg | <80 mg |
| Pitavastatin | | ≥2 mg | <2 mg |

### Total Daily Doses

### Endpoints

Additional secondary efficacy end points included: the individual components of the key secondary endpoint; death by any cause; the composite of cardiovascular death or hospitalization for heart failure; coronary revascularization; and ischemic stroke or transient ischemic attack. In addition, the Cholesterol Treatment Trialists Collaboration composite endpoint of major coronary events (coronary heart death or nonfatal myocardial infarction), stroke, or coronary revascularization was examined. (Cholesterol Treatment Trialists C, Baigent C, Blackwell L, et al. Efficacy and safety of more intensive lowering of LDL cholesterol: a meta-analysis of data from 170,000 participants in 26 randomised trials. Lancet. Nov. 13, 2010; 376(9753): 1670-1681.)

Adverse events of interest including muscle-related, cataracts, injection site, allergic and neurocognitive. These adverse events were categorized by the TIMI Safety Desk according to lower level MedDRA terms. New-onset diabetes was centrally adjudicated. Central laboratory testing included LDL cholesterol and other lipid parameters (to which investigators and subjects were blinded), liver function tests, creatine kinase, fasting glucose, HbA1c, and anti-evolocumab antibodies. LDL cholesterol was calculated using the Friedewald equation, except if <40 mg/dL or if the triglycerides were >400 mg/dL, in which case LDL cholesterol was measured by preparative ultracentrifugation.

### Statistical Considerations

Between group differences in lipid parameters were calculated using a repeated measures linear mixed effects model using all measurements from baseline up to the end of the study and are reported as least squared means. The model included terms for treatment group, stratification factors, scheduled visit and the interaction of treatment with scheduled visit. In cases where there was insufficient data for the model to run (after 120 weeks), the mean percent change was calculated using the difference between the descriptive mean changes in the evolocumab and placebo arms. Changes in triglycerides and Lp(a) were expressed as medians and P values from Wilcoxon ranksum tests.

In terms of the hierarchical efficacy end point analyses, if cardiovascular death was significantly reduced, then all-cause mortality was to be analyzed at a significance level of 0.04 and additional secondary endpoints at an overall significant level of 0.01 by applying the Hochberg method. (Benjamini Y, Hochberg Y. Controlling the false discovery rate: a practical and powerful approach to multiple testing. J R Statist Soc B. 1995; 57:289-300.) The statistical analysis plan is available with the full text of this article at NEJM.org.

Patients who discontinued study drug continued to be followed in the same fashion as adherent patients for outcome events. For patients who withdrew consent or were lost to follow-up, no imputation was done for events.

Schoenfeld residuals were examined to ensure that proportional hazards assumptions were not violated when using Cox modeling.

Landmark analsyes were performed in which patients who were alive and in follow-up at the start of the period of interest formed the group at risk. For comparison to Cholesterol Treatment Trialists Collaborators (CTTC) data, the between group difference in LDL cholesterol at 48 weeks was calculated as per the approach of the CTTC. (Baigent C, Keech A, Kearney P M, et al. Efficacy and safety of cholesterol-lowering treatment: prospective meta-analysis of data from 90,056 participants in 14 randomised trials of statins. Lancet. Oct. 8, 2005; 366(9493):1267-1278.). The number needed to treat to prevent one element of the CTTC composite endpoint over 5 years was calculated by taking the annualized incident rate for the CTTC composite endpoint in the placebo arm (5.34%), multiplying that rate by 5, and applying the relative risk reduction (22%) in the CTTC endpoint after the first year (analogous to the CTTC approach to quantifying longterm benefit), (Collins R, Reith C, Emberson J, et al. Interpretation of the evidence for the efficacy and safety of statin therapy. Lancet. Nov. 19, 2016; 388(10059):2532-2561) which yields an absolute risk reduction of 5.9%, or a number needed to treat of 17.

### Inclusion and Exclusion Criteria

Inclusion Criteria

| | |
|---|---|
| 4.1.1 | Signed informed consent |
| 4.1.2 | Male or female ≥40 to ≤85 years of age at signing of informed |
| | consent |
| 4.1.3 | History of clinically evident cardiovascular disease as evidenced |
| | by ANY of the following: |
| | diagnosis of myocardial infarction |
| | diagnosis of non-hemorrhagic stroke (TIA does not qualify as |
| | stroke for inclusion) |
| | symptomatic peripheral arterial disease (PAD), as evidenced by |
| | intermittent claudication with ankle-brachial index (ABI) <0.85, or |
| | peripheral arterial revascularization procedure, or amputation due to |
| | atherosclerotic disease |
| | Note: |
| | the proportion of subjects with history of MI or non-hemorrhagic |
| | stroke >5 years prior to screening was to be determined by the |
| | sponsor |
| 4.1.4 | At least 1 major risk factor or at least 2 minor risk factors below: |
| | Major Risk Factors (1 Required): |
| | diabetes (type 1 or type 2) |
| | age ≥65 years at randomization (and ≤85 years at time of informed |
| | consent) |
| | MI or non-hemorrhagic stroke within 6 months of screening |
| | additional diagnosis of myocardial infarction or non-hemorrhagic |
| | stroke excluding qualifying MI or non-hemorrhagic stroke^{a} |
| | current daily cigarette smoking |
| | history of symptomatic PAD (intermittent claudication with ABI |
| | <0.85, or peripheral arterial revascularization procedure, or |
| | amputation due to atherosclerotic disease) if eligible by MI or stroke |
| | history |
| | Minor Risk Factors (2 Required): |
| | history of non-MI related coronary revascularization^{a} |
| | residual coronary artery disease with ≥40% stenosis in ≥2 large |
| | vessels |
| | Most recent HDL-C <40 mg/dL (1.0 mmol/L) for men |
| | and <50 mg/dL (1.3 mmol/L) for women by central laboratory |
| | before randomization |
| | Most recent hsCRP >2.0 mg/L by central laboratory before |
| | randomization |
| | Most recent LDL-C ≥130 mg/dL (3.4 mmol/L) or non-HDL-C |
| | ≥160 mg/dL (4.1 mmol/L) by central laboratory before |
| | randomization metabolic syndrome^{b} |
| 4.1.5 | Most recent fasting LDL-C ≥70 mg/dL (≥1.8 mmol/L) or non- |
| | HDL-C ≥100 mg/dL (≥2.6 mmol/L) by central laboratory during |
| | screening after 2 weeks of stable lipid lowering therapy per |
| | discussion below |
| 4.1.6 | Most recent fasting triglycerides ≤400 mg/dL (4.5 mmol/L) by |
| | central laboratory before randomization |

^{a}Note:
there is no time limit on additional qualifying medical history.
^{b}Definition: metabolic syndrome for this protocol is defined as ≥3 of the following (Alberti et al, 2009):
waist circumference >102 cm (>40 in.) for men and >88 cm (>35 in.) for women (Asian men, including Japanese >90 cm; Asian women, except Japanese >80 cm; Japanese women >90 cm)
triglycerides ≥150 mg/dL (1.7 mmol/L) by central laboratory at final screening
HDL-C <40 mg/dL (1.0 mmol/L) for men and <50 mg/dL (1.3 mmol/L) for women by central laboratory at final screening (Note: if the HDL-C level is one of criterion used to make the diagnosis of metabolic syndrome, it was not used as a separate risk factor)
systolic blood pressure (SBP) ≥130 mmHg or diastolic BP (DBP) ≥85 mmHg or hypertension treated with medication
fasting glucose ≥100 mg/dL (≥5.6 mmol/L) by central laboratory at final screening

Exclusion Criteria

| | |
|---|---|
| 4.2.1 | Subject must not be randomized within 4 weeks of their most recent MI or |
| | stroke |
| 4.2.2 | NYHA class III or IV, or last known left ventricular ejection fraction <30% |
| 4.2.3 | Known hemorrhagic stroke at any time |
| 4.2.4 | Uncontrolled or recurrent ventricular tachycardia |
| 4.2.5 | Planned or expected cardiac surgery or revascularization within 3 months after |
| | randomization |
| 4.2.6 | Uncontrolled hypertension defined as sitting systolic blood pressure (SBP) >180 |
| | mmHg or diastolic BP (DBP) >110 mmHg |
| 4.2.7 | Use of cholesteryl ester transfer protein (CETP) inhibition treatment, |
| | mipomersen, or lomitapide within 12 months prior to randomization. |
| | Fenofibrate therapy must be stable for at least 6 weeks prior to final screening |
| | at a dose that is appropriate for the duration of the study in the judgment of the |
| | investigator. Other fibrate therapy (and derivatives) are prohibited |
| 4.2.8 | Prior use of PCSK9 inhibition treatment other than evolocumab or use of |
| | evolocumab <12 weeks prior to final lipid screening |
| 4.2.9 | Untreated or inadequately treated hyperthyroidism or hypothyroidism as |
| | defined by thyroid stimulating hormone (TSH) < lower limit of normal (LLN) |
| | or >1.5 times the upper limit of normal (ULN), respectively, and free |
| | thyroxine (T4) levels that are outside normal range at final screening |
| 4.2.10 | Severe renal dysfunction, defined as an estimated glomerular filtration rate |
| | (eGFR) <20 mL/min/1.73 m² at final screening |
| 4.2.11 | Active liver disease or hepatic dysfunction, defined as aspartate |
| | aminotransferase (AST) or alanine aminotransferase (ALT) >3 times the |
| | ULN as determined by central laboratory analysis at final screening |
| 4.2.12 | Recipient of any major organ transplant (e.g., lung, liver, heart, bone marrow, |
| | renal) |
| 4.2.13 | Personal or family history of hereditary muscular disorders |
| 4.2.14 | LDL or plasma apheresis within 12 months prior to randomization |
| 4.2.15 | Severe, concomitant non-cardiovascular disease that is expected to reduce life |
| | expectancy to less than 3 years |
| 4.2.16 | CK >5 times the ULN at final screening |
| 4.2.17 | Known major active infection or major hematologic, renal, metabolic, |
| | gastrointestinal or endocrine dysfunction in the judgment of the investigator |
| 4.2.18 | Malignancy (except non-melanoma skin cancers, cervical in-situ carcinoma, |
| | breast ductal carcinoma in situ, or stage 1 prostate carcinoma) within the last |
| | 10 years |
| 4.2.19 | Subject has received drugs via a systemic route that have known major |
| | interactions with background statin therapy (see Appendix F) within 1 month |
| | prior to randomization or is likely to require such treatment during the study |
| | period |
| 4.2.20 | Currently enrolled in another investigational device or drug study, or less than |
| | 30 days since ending another investigational device or drug study(s), or |
| | receiving other investigational agent(s) |
| 4.2.21 | Female subject who has either (1) not used acceptable method(s) of birth |
| | control for at least 1 month prior to screening or (2) is not willing to use such |
| | a method during treatment with IP and for an additional 15 weeks after the |
| | end of treatment with IP, unless the subject is sterilized or postmenopausal; |
| | menopause is defined as 12 months of spontaneous and continuous |
| | amenorrhea in a female ≥55 years old or 12 months of spontaneous |
| | and continuous amenorrhea with a follicle-stimulating hormone (FSH) |
| | level >40 IU/L (or according to the definition of ″postmenopausal |
| | range″ for the laboratory involved) in a female <55 years old unless |
| | the subject has undergone bilateral oophorectomy |
| | acceptable methods of preventing pregnancy include not having |
| | intercourse, birth control pills, injections, implants, or patches, |
| | intrauterine devices (IUDs), tubal ligation/occlusion, sexual activity |
| | with a male partner who has had a vasectomy, condom or occlusive |
| | cap (diaphragm or cervical/vault caps) used with spermicide |
| 4.2.22 | Subject is pregnant or breast feeding, or planning to become pregnant or to |
| | breastfeed during treatment with IP and/ or within 15 weeks after the end of |
| | treatment with IP |
| 4.2.23 | Known sensitivity to any of the active substances or their excipients to be |
| | administered during dosing |
| 4.2.24 | Subject likely to not be available to complete all protocol-required study visits |
| | or procedures, to the best of the subject's and investigator's knowledge |
| 4.2.25 | History or evidence of any other clinically significant disorder, condition or |
| | disease other than those outlined above that, in the opinion of the Investigator |
| | or Amgen physician, if consulted, may compromise the ability of the subject |
| | to give written informed consent, would pose a risk to subject safety, or |
| | interfere with the study evaluation, procedures or completion. |

### Endpoint Definitions

### A. I. Death

A. Definition of Cardiovascular Death

Cardiovascular death includes death resulting from an acute myocardial infarction (MI), sudden cardiac death, death due to heart failure (HF), death due to stroke, death due to cardiovascular (CV) procedures, death due to CV hemorrhage, and death due to other CV causes.

Death due to Acute Myocardial Infarction refers to a death by any cardiovascular mechanism (e.g., arrhythmia, sudden death, heart failure, stroke, pulmonary embolus, peripheral arterial disease) ≤30 days 1 (the 30-day cut-off is arbitrary) after a MI related to the immediate consequences of the MI, such as progressive heart failure or recalcitrant arrhythmia. There may be assessable mechanisms of cardiovascular death during this time period, but for simplicity, if the cardiovascular death occurs ≤30 days of the myocardial infarction, it was considered a death due to myocardial infarction.

Acute MI should be verified to the extent possible by the diagnostic criteria outlined for acute MI (see Definition of Myocardial Infarction) or by autopsy findings showing recent MI or recent coronary thrombosis.

Death resulting from a procedure to treat a MI (percutaneous coronary intervention (PCI), coronary artery bypass graft surgery [CABG]), or to treat a complication resulting from MI, should also be considered death due to acute MI.

Death resulting from an elective coronary procedure to treat myocardial ischemia (i.e., chronic stable angina) or death due to a MI that occurs as a direct consequence of a CV investigation/procedure/operation should be considered as a death due to a CV procedure

Sudden Cardiac Death refers to a death that occurs unexpectedly, not following an acute MI, and includes the following deaths:
a. Death witnessed and occurring without new or worsening symptoms
b. Death witnessed within 60 minutes of the onset of new or worsening cardiac symptoms, unless the symptoms suggest acute MI
c. Death witnessed and attributed to an identified arrhythmia (e.g., captured on an electrocardiographic [ECG] recording, witnessed on a monitor, or unwitnessed but found on implantable cardioverter-defibrillator review)
d. Death after unsuccessful resuscitation from cardiac arrest
e. Death after successful resuscitation from cardiac arrest and without identification of a specific cardiac or non-cardiac etiology
f. Unwitnessed death in a subject seen alive and clinically stable ≤24 hours prior to being found dead without any evidence supporting a specific non-cardiovascular cause of death (information regarding the patient's clinical status preceding death should be provided, if available)

### General Considerations

Unless additional information suggests an alternate specific cause of death (e.g., Death due to Other Cardiovascular Causes), if a patient is seen alive ≤24 hours of being found dead, sudden cardiac death should be recorded. For patients who were not observed alive within 24 hours of death, undetermined cause of death should be recorded (e.g., a subject found dead in bed, but who had not been seen by family for several days).
1. Death due to Heart Failure refers to a death in association with clinically worsening

symptoms and/or signs of heart failure regardless of HF etiology (see Definition of Heart Failure Event). Deaths due to heart failure can have various etiologies, including single or recurrent myocardial infarctions, ischemic or non-ischemic cardiomyopathy, hypertension, or valvular disease.

Death due to Stroke refers to death after a stroke that is either a direct consequence of the stroke or a complication of the stroke. Acute stroke should be verified to the extent possible by the diagnostic criteria outlined for stroke (see Definition of Transient Ischemic Attack and Stroke).

Death due to Cardiovascular Procedures refers to death caused by the immediate complications of a cardiac procedure.

Death due to Cardiovascular Hemorrhage refers to death related to hemorrhage such as a non-stroke intracranial hemorrhage (see Definition of Transient Ischemic Attack and Stroke), non-procedural or non-traumatic vascular rupture (e.g., aortic aneurysm), or hemorrhage causing cardiac tamponade.

Death due to Other Cardiovascular Causes refers to a CV death not included in the above categories but with a specific, known cause (e.g., pulmonary embolism or peripheral arterial disease).

### B. Definition of Non-Cardiovascular Death

Non-cardiovascular death is defined as any death with a specific cause that is not thought to be cardiovascular in nature, as listed in Definition of Cardiovascular Death. Detailed recommendations on the classification of non-CV causes of death are beyond the scope of this document. The level of detail required and the optimum classification will depend on the nature of the study population and the anticipated number and type of non-CV deaths. Any specific anticipated safety concern should be included as a separate cause of death. The following is a suggested list of non-CV causes of death:
Pulmonary
Renal
Gastrointestinal
Hepatobiliary
Pancreatic
Infection (includes sepsis)
Inflammatory (e.g., Systemic Inflammatory Response Syndrome [SIRS]/Immune (including autoimmune)
Hemorrhage that is neither cardiovascular bleeding or a stroke (See Definition of Cardiovascular Death and Definition of Transient Ischemic Attack and Stroke)
Non-CV procedure or surgery
Trauma
Suicide
Non-prescription drug reaction or overdose
Prescription drug reaction or overdose
Neurological (non-cardiovascular)
Malignancy
Other non-CV, in which case specify:

C. Definition of Undetermined Cause of Death

Undetermined Cause of Death refers to a death not attributable to one of the above categories of CV death or to a non-CV cause. Inability to classify the cause of death may be due to lack of information (e.g., the only available information is “patient died”) or when there is insufficient supporting information or detail to assign the cause of death. In general, most deaths should be classifiable as CV or non-CV.

### C. II. Cardiac Ischemic Events Acute Coronary Syndromes

A. Definition of Myocardial Infarction

1. General Considerations

The term myocardial infarction (MI) should be used when there is evidence of myocardial necrosis in a clinical setting consistent with myocardial ischemia.

In general, the diagnosis of MI requires the combination of:
Evidence of myocardial necrosis (either changes in cardiac biomarkers or post-mortem pathological findings); and
Supporting information derived from the clinical presentation, electrocardiographic changes, or the results of myocardial or coronary artery imaging

The totality of the clinical, electrocardiographic, and cardiac biomarker information should be considered to determine whether or not a MI has occurred. Specifically, timing and trends in cardiac biomarkers and electrocardiographic information require careful analysis. The adjudication of MI should also take into account the clinical setting in which the event occurs. MI may be adjudicated for an event that has characteristics of a MI but which does not meet the strict definition because biomarker or electrocardiographic results are not available.

2. Criteria for Myocardial Infarction

a. Clinical Presentation

The clinical presentation should be consistent with diagnosis of myocardial ischemia and infarction. Other findings that can support the diagnosis of MI should be taken into account because a number of conditions are associated with elevations in cardiac biomarkers (e.g., trauma, surgery, pacing, ablation, heart failure, hypertrophic cardiomyopathy, pulmonary embolism, severe pulmonary hypertension, stroke or subarachnoid hemorrhage, infiltrative and inflammatory disorders of cardiac muscle, drug toxicity, burns, critical illness, extreme exertion, and chronic kidney disease). Supporting information can also be considered from myocardial imaging and coronary imaging. The totality of the data may help differentiate acute MI from the background disease process.

b. Biomarker Elevations

For cardiac biomarkers, laboratories reported an upper reference limit (URL). If the 99th percentile of the upper reference limit (URL) from the respective laboratory performing the assay is not available, then the URL for myocardial necrosis from the laboratory was used. If the 99th percentile of the URL or the URL for myocardial necrosis was not available, the MI decision limit for the particular laboratory was used as the URL. Laboratories also reported both the 99th percentile of the upper reference limit and the MI decision limit. Reference limits from the laboratory performing the assay are preferred over the manufacturer's listed reference limits in an assay's instructions for use. In general, troponins are preferred. CK-MB should be used if troponins are not available, and total CK may be used in the absence of CK-MB and troponin.

In many studies, particularly those in which patients present acutely to hospitals which are not participating sites, it is not practical to stipulate the use of a single biomarker or assay, and the locally available results are to be used as the basis for adjudication. However, if possible, using the same cardiac biomarker assay and preferably, a core laboratory, for all measurements reduces inter-assay variability.

Since the prognostic significance of different types of myocardial infarctions (e.g., periprocedural myocardial infarction versus spontaneous myocardial infarction) may be different, people were to consider evaluating outcomes for these subsets of patients separately.

c. Electrocardiogram (ECG) Changes

Electrocardiographic changes can be used to support or confirm a MI. Supporting evidence may be ischemic changes and confirmatory information may be new Q waves.

ECG Manifestations of Acute Myocardial Ischemia (in Absence of Left Ventricular Hypertrophy (LVH) and Left Bundle Branch Block (LBBB)):

ST Elevation

New ST elevation at the J point in two contiguous leads with the cut-points: ≥0.1 mV in all leads other than leads V2-V3 where the following cut-points apply: ≥0.2 mV in men ≥40 years (≥0.25 mV in men <40 years) or ≥0.15 mV in women.

ST Depression and T-Wave Changes

New horizontal or down-sloping ST depression ≥0.05 mV in two contiguous leads and/or new T inversion ≥0.1 mV in two contiguous leads with prominent R wave or R/S ratio >1.

The above ECG criteria illustrate patterns consistent with myocardial ischemia. In patients with abnormal biomarkers, it is recognized that lesser ECG abnormalities may represent an ischemic response and may be accepted under the category of abnormal ECG findings.

Criteria for Pathological Q-Wave
Any Q-wave in leads V2-V3 ≥0.02 seconds or QS complex in leads V2 and V3
Q-wave ≥0.03 seconds and ≥0.1 mV deep or QS complex in leads I, II, aVL, aVF, or V4-V6 in any two leads of a contiguous lead grouping (I, aVL; V1-V6; II, III, and aVF)^{a} ^{a}The same criteria are used for supplemental leads V7-V9, and for the Cabrera frontal plane lead grouping.

ECG Changes Associated with Prior Myocardial Infarction
Pathological Q-waves, as defined above
R-wave ≥0.04 seconds in V1-V2 and R/S ≥1 with a concordant positive T-wave in the absence of a conduction defect

Criteria for Prior Myocardial Infarction

Any one of the following criteria meets the diagnosis for prior MI:
Pathological Q waves with or without symptoms in the absence of non-ischemic causes
Imaging evidence of a region of loss of viable myocardium that is thinned and fails to contract, in the absence of a non-ischemic causes
Pathological findings of a prior myocardial infarction

d. ST-Segment Elevation MI Versus Non-ST-Segment Elevation MI

All events meeting criteria for MI* were also classified as either ST-segment elevation MI (STEMI), non-ST-segment elevation MI (NSTEMI), or unknown.
STEMI—To be classified as a STEMI the event must have met all of the above criteria for myocardial infarction and one of the four criteria below.
   New ST segment elevation at the J point in contiguous leads, defined as: ≥0.2 mV in men (>0.25 mV in men <40 years) or ≥0.15 mV in women in leads V2-V3 and/or ≥0.1 mV in other leads. Subjects must have had an interpretable ECG (i.e., without evidence of left ventricular hypertrophy or pre-existing left bundle branch block), or
   New left bundle branch block
NSTEMI—To be classified as a NSTEMI the event must have met all of the above criteria for myocardial infarction and not met criteria for classification as STEMI. In order to be classified as NSTEMI there must have been adequate interpretable ECG documentation associated with the event.
Unknown—Events which met criteria as specified above for MI but did not meet criteria for STEMI or NSTEMI. All cases where ECG documentation of the acute event is missing, inadequate, or uninterpretable were classified as Unknown.

e. Criteria for Universal Classification of Myocardial Infarction

Type 1: Spontaneous Myocardial Infarction

Spontaneous myocardial infarction related to atherosclerotic plaque rupture, ulceration, fissuring, erosion, or dissection with resulting intraluminal thrombus in one or more of the coronary arteries leading to decreased myocardial blood flow or distal platelet emboli with ensuing myocyte necrosis. The patient may have had underlying severe CAD but on occasion non-obstructive or no CAD.

Type 2: Myocardial Infarction Secondary to an Ischemic Imbalance

In instances of myocardial injury with necrosis where a condition other than CAD contributes to an imbalance between myocardial oxygen supply and/or demand, e.g. coronary endothelial dysfunction, coronary artery spasm, coronary embolism, tachy-/brady-arrhythmias, anaemia, respiratory failure, hypotension, and hypertension with or without LVH.

Type 3: Myocardial Infarction Resulting in Death when Biomarker Values are Unavailable

Cardiac death with symptoms suggestive of myocardial ischaemia and presumed new ischemic ECG changes or new LBBB, but death occurring before blood samples could be obtained, before cardiac biomarkers could rise, or in rare cases cardiac biomarkers were not collected.

Type 4a: Myocardial Infarction Related to Percutaneous Coronary Intervention (PCI)

Myocardial infarction associated with PCI is arbitrarily defined by elevation of cTn values >5×99th percentile URL in patients with normal baseline values (≤99^{th} percentile URL) or a rise of cTn values ≥20% if the baseline values are elevated and are stable or falling. In addition, either (i) symptoms suggestive of myocardial ischemia, or (ii) new ischemic ECG changes or new LBBB, or (iii) angiographic loss of patency of a major coronary artery or a side branch or persistent slow or no-flow or embolization, or (iv) imaging demonstration of new loss of viable myocardium or new regional wall motion abnormality are required.

Type 4b: Myocardial Infarction Related to Stent Thrombosis

Myocardial infarction associated with stent thrombosis is detected by coronary angiography or autopsy in the setting of myocardial ischemia and with a rise and/or fall of cardiac biomarkers values with at least one value above the 99th percentile URL.

Type 4c: Myocardial Infarction Related to Restenosis

Restenosis is defined as ≥50% stenosis at coronary angiography or a complex lesion associated with a rise and/or fall of cTn values >99th percentile URL and no other significant obstructive CAD of greater severity following: (i) initially successful stent deployment or (ii) dilatation of a coronary artery stenosis with balloon angioplasty (<50%).

Type 5: Myocardial Infarction Related to Coronary Artery Bypass Grafting (CABG)

Myocardial infarction associated with CABG is arbitrarily defined by elevation of cardiac biomarker values >10×99th percentile URL in patients with normal baseline cTn values (≤99th percentile URL). In addition, either (i) new pathological Q waves or new LBBB, or (ii) angiographic documented new graft or new native coronary artery occlusion, or (iii) imaging evidence of new loss of viable myocardium or new regional wall motion abnormality.
Note: As noted in criterion 2b, although language states troponin, CKMB can be used with similar cut points.

### D. IIB. Coronary Revascularization

1. Percutaneous Coronary Intervention (PCI):

Placement of an angioplasty guide wire, balloon, or other device (e.g., stent, atherectomy catheter, brachytherapy delivery device, or thrombectomy catheter) into a native coronary artery or coronary artery bypass graft for the purpose of mechanical coronary revascularization. In the assessment of the severity of coronary lesions with the use of intravascular ultrasound, CFR, or FFR, insertion of a guide wire was NOT considered PCI.

a. Elective:

The procedure can be performed on an outpatient basis or during a subsequent hospitalization without significant risk of myocardial infarction (MI) or death. For stable in-patients, the procedure is being performed during this hospitalization for convenience and ease of scheduling and NOT because the patient's clinical situation demands the procedure prior to discharge.

b. Urgent:

The procedure should be performed on an inpatient basis and prior to discharge because of significant concerns that there is risk of myocardial ischemia, MI, and/or death. Patients who are outpatients or in the emergency department at the time that the cardiac catheterization is requested would warrant hospital admission based on their clinical presentation.

c. Emergency:

The procedure should be performed as soon as possible because of substantial concerns that ongoing myocardial ischemia and/or MI could lead to death. “As soon as possible” refers to a patient who is of sufficient acuity that one would cancel a scheduled case to perform this procedure immediately in the next available room during business hours, or one would activate the on-call team were this to occur during off-hours.

d. Salvage:

The procedure is a last resort. The patient is in cardiogenic shock when the PCI begins (i.e., the time at which the first guide wire or intracoronary device is introduced into a coronary artery or bypass graft for the purpose of mechanical revascularization) OR within the last ten minutes prior to the start of the case or during the diagnostic portion of the case, the patient has also received chest compressions or has been on unanticipated circulatory support (e.g., intra-aortic balloon pump, extracorporeal mechanical oxygenation, or cardiopulmonary support).

C. Definition of Hospitalization for Unstable Angina

Unstable angina requiring hospitalization is defined as
1. Ischemic discomfort (angina, or symptoms thought to be equivalent) ≥10 minutes in duration occurring:
   at rest, or
   in an accelerating pattern with frequent episodes associated with progressively decreased exercise capacity.
AND
2. Prompting an unscheduled hospitalization within 24 hours of the most recent symptoms. Hospitalization is an admission to an inpatient unit or a visit to an emergency department that results in at least a 24* hour stay (or a change in calendar date if the hospital admission or discharge times are not available).
AND
3. At least one of the following:
   a) New or worsening ST or T wave changes on resting ECG (in the absence of confounders, such as LBBB or LVH)
      Transient ST elevation (duration <20 minutes)
      New ST elevation at the J point in two contiguous leads with the cut-points: ≥0.1 mV in all leads other than leads V2-V3 where the following cut-points apply: ≥0.2 mV in men ≥40 years (>0.25 mV in men <40 years) or ≥0.15 mV in women.
      ST depression and T-wave changes
      New horizontal or down-sloping ST depression ≥0.05 mV in two contiguous leads and/or new T inversion ≥0.3 mV in two contiguous leads with prominent R wave or R/S ratio >1.
   b) Definite evidence of inducible myocardial ischemia as demonstrated by:
      an early positive exercise stress test, defined as ST elevation or ≥2 mm ST depression prior to 5 mets
   OR
      stress echocardiography (reversible wall motion abnormality) OR
      myocardial scintigraphy (reversible perfusion defect), OR
      MRI (myocardial perfusion deficit under pharmacologic stress),
   and believed to be responsible for the myocardial ischemic symptoms/signs.
   c) Angiographic evidence of new or worse ≥70% lesion and/or thrombus in an epicardial coronary artery that is believed to be responsible for the myocardial ischemic symptoms/signs.
   d) Need for coronary revascularization procedure (PCI or CABG) for the presumed culprit lesion(s). This criterion would be fulfilled if revascularization was undertaken during the unscheduled hospitalization, or subsequent to transfer to another institution without interceding home discharge.
AND
4. Negative cardiac biomarkers and no evidence of acute MI

General Considerations

(1) Escalation of pharmacotherapy for ischemia, such as intravenous nitrates or increasing dosages of β-blockers, should be considered supportive but not diagnostic of unstable angina. However, a typical presentation and admission to the hospital with escalation of pharmacotherapy, without any of the additional findings listed under category 3, would be insufficient to support classification as hospitalization for unstable angina.

(2) If subjects are admitted with suspected unstable angina, and subsequent testing reveals a non-cardiac or non-ischemic etiology, this event should not be recorded as hospitalization for unstable angina. Potential ischemic events meeting the criteria for myocardial infarction should not be adjudicated as unstable angina.

(3) Planned hospitalization or rehospitalization for performance of an elective revascularization in patients who do not fulfill the criteria for unstable angina should not be considered a hospitalization for unstable angina. For example,
Hospitalization of a patient with stable exertional angina for coronary angiography and PCI that is prompted by a positive outpatient stress test should not be considered hospitalization for unstable angina.
Rehospitalization of a patient meeting the criteria for unstable angina that was stabilized, discharged, and subsequently readmitted for revascularization, does not constitute a second hospitalization for unstable angina.

(4) A patient who undergoes an elective catheterization where incidental coronary artery disease is found and who subsequently undergoes coronary revascularization will not be considered as meeting the hospitalization for unstable angina end point.

### E. III. Heart Failure

A Heart Failure Event includes hospitalization for heart failure and may include urgent outpatient visits. HF hospitalizations should remain delineated from urgent visits. If urgent visits are included in the HF event endpoint, the number of urgent visits needs to be explicitly presented separately from the hospitalizations. A Heart Failure Hospitalization is defined as an event that meets ALL of the following criteria:
1. The patient is admitted to the hospital with a primary diagnosis of HF
2. The patient's length-of-stay in hospital extends for at least 24 hours (or a change in calendar date if the hospital admission and discharge times are unavailable)
3. The patient exhibits documented new or worsening symptoms due to HF on presentation, including at least ONE of the following:
   a) Dyspnea (dyspnea with exertion, dyspnea at rest, orthopnea, paroxysmal nocturnal dyspnea)
   b) Decreased exercise tolerance
   c) Fatigue
   d) Other symptoms of worsened end-organ perfusion or volume overload
4. The patient has objective evidence of new or worsening HF, consisting of at least TWO physical examination findings a) OR one physical examination finding and at least ONE laboratory criterion b), including:
   a) Physical examination findings considered to be due to heart failure, including new or worsened:
      1) Peripheral edema
      2) Increasing abdominal distention or ascites (in the absence of primary hepatic disease)
      3) Pulmonary rales/crackles/crepitations
      4) Increased jugular venous pressure and/or hepatojugular reflux
      5) S₃ gallop
      6) Clinically significant or rapid weight gain thought to be related to fluid retention
   b) Laboratory evidence of new or worsening HF, if obtained within 24 hours of presentation, including:
      1) Increased B-type natriuretic peptide (BNP)/N-terminal pro-BNP (NT-proBNP) concentrations consistent with decompensation of heart failure (such as BNP >500 pg/mL or NT-proBNP >2,000 pg/mL). In patients with chronically elevated natriuretic peptides, a significant increase should be noted above baseline.
      2) Radiological evidence of pulmonary congestion
      3) Non-invasive or invasive diagnostic evidence of clinically significant elevated left- or right-sided ventricular filling pressure or low cardiac output. For example, echocardiographic criteria could include: E/e′>15 or D-dominant pulmonary venous inflow pattern, plethoric inferior vena cava with minimal collapse on inspiration
         OR
      4) Invasive diagnostic evidence with right heart catheterization showing a pulmonary capillary wedge pressure (pulmonary artery occlusion pressure) ≥18 mmHg, central venous pressure ≥12 mmHg, or a cardiac index <2.2 L/min/m2
5. The patient receives initiation or intensification of treatment specifically for HF, including at least ONE of the following:
   a) Augmentation in oral diuretic therapy
   b) Intravenous diuretic, inotrope, or vasodilator therapy
   c) Mechanical or surgical intervention, including
      1) Mechanical circulatory support (e.g., intra-aortic balloon pump, ventricular assist device)
      2) Mechanical fluid removal (e.g., ultrafiltration, hemofiltration, dialysis)

An Urgent Heart Failure Visit is defined as an event that meets all of the following:
1) The patient has an urgent, unscheduled office/practice or emergency department visit for a primary diagnosis of HF, but not meeting the criteria for a HF hospitalization
2) All signs and symptoms for HF hospitalization (i.e., 3) symptoms, 4) physical examination findings, and 5) laboratory evidence of new or worsening HF, as indicated above) must be met
3) The patient receives initiation or intensification of treatment specifically for HF, as detailed in the above section with the exception of oral diuretic therapy, which will not be sufficient

### F. IV. Cerebrovascular Events

A. Definition of Transient Ischemic Attack and Stroke

The distinction between a Transient Ischemic Attack and an Ischemic Stroke is the presence of infarction. Persistence of symptoms is an acceptable indicator of acute infarction.

Transient Ischemic Attack

Transient ischemic attack (TIA) is defined as a transient episode of focal neurological

dysfunction caused by brain, spinal cord, or retinal ischemia, without acute infarction.

Stroke is defined as an acute episode of focal or global neurological dysfunction caused by brain, spinal cord, or retinal vascular injury as a result of hemorrhage or infarction.

Classification:

1. Ischemic Stroke

Ischemic stroke is defined as an acute episode of focal cerebral, spinal, or retinal dysfunction caused by infarction of central nervous system tissue.

Hemorrhage may be a consequence of ischemic stroke. In this situation, the stroke is an ischemic stroke with hemorrhagic transformation and not a hemorrhagic stroke.

2. Hemorrhagic Stroke

Hemorrhagic stroke is defined as an acute episode of focal or global cerebral or spinal dysfunction caused by intraparenchymal, intraventricular, or subarachnoid hemorrhage

3. Undetermined Stroke

Undetermined stroke is defined as an acute episode of focal or global neurological dysfunction caused by presumed brain, spinal cord, or retinal vascular injury as a result of hemorrhage or infarction but with insufficient information to allow categorization as 1 or 2.

Disability should be measured by a reliable and valid scale in all cases, typically at each visit and 90 days after the event. For example, the modified Rankin Scale may be used to address this requirement, as outlined in table 17.5:

TABLE 17.5

| Scale | Disability |
|---|---|
| 0 | No symptoms at all |
| 1 | No significant disability despite symptoms; able to carry out all |
| | usual duties and activities |
| 2 | Slight disability; unable to carry out all previous activities, but able |
| | to look after own affairs without assistance |
| 3 | Moderate disability; requiring some help, but able to walk without |
| | assistance |
| 4 | Moderately severe disability; unable to walk without assistance and |
| | unable to attend to own bodily needs without assistance |
| 5 | Severe disability; bedridden, incontinent and requiring constant |
| | nursing care and attention |
| 6 | Dead |
| 7 | Unable to Determine |

General Considerations

Evidence of vascular central nervous system injury without recognized neurological dysfunction including microhemorrhage, silent infarction, and silent hemorrhage, if appropriate, will not be adjudicated as cerebrovascular events for this trial.

Subdural hematomas are intracranial hemorrhagic events and not strokes

Epidural hemorrhages are intracranial bleeds and not strokes

### REFERENCES

Hicks K A, Hung H M J, Mahaffey K W, et al. Standardized definitions for cardiovascular and stroke end point events in clinical trials. Nov. 9, 2012.

### G. V. New Onset Diabetes

### Diabetes Definition

Diabetes mellitus, a group of metabolic disorders, is characterized by hyperglycemia and abnormal protein, fat, and carbohydrate metabolism due to defects in insulin secretions, inadequate and deficient insulin action on target organs, or both. For the purpose of clinical adjudication, diabetes will be defined according to the criteria below, based on the American Diabetes Association¹ and National Diabetes Information Clearinghouse² definitions.

### Diabetes

Type 2 diabetes (adult-onset diabetes) is the most common form of diabetes. Although people can develop type 2 diabetes at any age, even during childhood type 2 diabetes can develop most often in middle-aged and older people. It is anticipated that most subjects converting to diabetes during the course of the study will develop type 2.

Acute complications include diabetic ketoacidosis and hyperosmolar hyperglycemic nonketotic coma (HHNC). Chronic complications included accelerated vascular disease and can be microvascular or macrovasular. Microvascular complications include neuropathy, nephropathy and retinopathy. Macrovascular complications include myocardial infarction, stroke, coronary heart disease, and peripheral vascular disease.

Diabetes mellitus is diagnosed on the basis of elevated plasma glucose levels. The criteria for diagnosis of diabetes within the trial are as any of the following:
1. Symptoms (e.g. polyuria, polydipsia, polyphagia, unexplained weight loss) of diabetes and casual/random (any time of day without regard to time since last meal) plasma glucose levels of ≥200 mg/dL (11.1 mmol/L).
   OR
2. Fasting (no caloric intake for at least 8 hours) plasma glucose (FPG) level ≥126 mg/dL (7.0 mmol/L), on 2 occasions separated by at least 24 hours.
   OR
3. Two-hour plasma glucose level ≥200 mg/dL (11.1 mmol/L) during an oral glucose tolerance test (OGTT performed as per WHO criteria with glucose load of 75 g anhydrous glucose dissolved in water).
   OR
4. A1c level ≥6.5% using a NGSP³ certified method and standardized to the Diabetes Control and Complications Trial (DCCT) assay.
   OR
5. Use of oral or injected diabetes medication and an established diagnosis of diabetes mellitus per the medical record. Note that the use of diabetes medication for pre-diabetes with the intent of preventing diabetes does not meet the definition.

### Additional Guidance:

Clinical judgment and the totality of information was used to make the diagnosis. In general it was expected that more than one of the diagnostic criteria above would be present unless unequivocal symptoms/signs are present. For example, a single fasting glucose of 180 mg/dl prompting initiation of diabetes therapy would meet the criteria.
1. If two different tests are used e.g., OGTT and A1c and both indicate diabetes, consider the diagnosis confirmed.
2. If the two different tests are discordant, it may be reasonable to request that additional information be obtained, if available.

Secondary Diabetes Mellitus

Hyperglycemia caused as a result of certain conditions, such as pancreatic surgery, chronic pancreatitis, chronic liver disease, or various forms of endocrinopathy, such as Cushing's syndrome, acromegaly, pheochromocytoma, or aldosteronism, or by medication use, such as chronic glucocorticoid therapy or hyperglycemia associated with a number of relatively uncommon genetic conditions. Those events where elevated blood glucose levels are definitely caused by such conditions should not be considered as new onset diabetes and should be adjudicated as not an event for the purpose of this trial.

Supplemental Results

A total of 1,209 (8.8%) patients allocated to evolocumab and 1,120 (8.1%) patients allocated to placebo either switched to a less intensive statin regimen or discontinued a statin during FOURIER. Conversely, 95 (0.7%) patients allocated to evolocumab and 141 (1.0%) patients allocated to placebo switched to a more intensive statin regimen. Ezetimibe was started in 67 (0.5%) and 145 (1.1%) patients in the evolocumab and placebo arms, respectively, during the trial, and 2 patients in the evolocumab arm stopped it.

The placebo-controlled mean LDL cholesterol reduction at 12 weeks was 61.1% (95% CI 60.5-61.7) for patients who chose twice weekly dosing and 56.9% (95% CI 55.3-58.6) for those who chose monthly dosing.

The between-group difference in LDL cholesterol at 48 weeks with imputation for missing values as per the Cholesterol Treatment Trialists Collaboration approach was 53.4 mg/dL (1.38 mmol/L).

The level of C-reactive protein was 1.7 mg/L (IQR 0.9-3.6) at baseline and by 48 weeks was 1.4 mg/L (IQR 0.7-3.1) in both arms.

The above definitions in the supplemental section of Example 17 describe the definitions of the terms as used in the FOURIER study. While there are embodiments in which such definitions can be applied in other scenarios and uses, it is to be understood that, unless explicitly designated otherwise, the denoted terms have their plain and ordinary meaning to one of skill in the art. In some embodiments, the definitions supplied in the supplemental section of Example 17 can be used for the same term in any of the other embodiments provided herein.

### Example 18

In this analysis of FOURIER, the cardiovascular efficacy and safety of evolocumab was investigated in patients with peripheral artery disease (PAD) as well as the effect of LDL cholesterol lowering with evolocumab on major adverse limb events.

### Outline of Methods for Example 18:

FOURIER was a randomized trial of evolocumab versus placebo in 27,564 patients with atherosclerotic disease on statin therapy followed for a median of 2.2 years. Patients were identified as having PAD at baseline if they had intermittent claudication and an ankle brachial index of <0.85 or if they had a prior peripheral vascular procedure. The primary endpoint was a composite of cardiovascular death, myocardial infarction, stroke, hospital admission for unstable angina, or coronary revascularization. The key secondary endpoint was a composite of cardiovascular death, myocardial infarction, or stroke. An additional outcome of interest was major adverse limb events (MALE) defined as acute limb ischemia (ALI), major amputation or urgent peripheral revascularisation for ischemia. FOURIER is registered with ClinicalTrials “dot” gov, number NCT01764633.

### Outline of Findings:

3,642 patients (13.2%) had PAD (1505 with no prior MI or stroke). Evolocumab significantly reduced cardiovascular outcomes consistently in patients with and without PAD (PEP PAD HR 0.79, 95% CI 0.66-0.94; p=0.0098; no PAD HR 0.86, 95% CI0.80-0.93; p=0.0003, p-interaction=0.40). For the key secondary endpoint, the HRs were 0.73 (0.59-0.91; p=0.0040) for those with PAD and 0.81 (0.73-0.90; p<0.0001) for those without PAD (pinteraction=0.41). Due to their higher risk, patients with PAD had larger absolute risk reductions for the PEP (3.5% PAD, 1.6% no PAD) and the key secondary endpoint (3.5% PAD, 1.4% no PAD). Evolocumab reduced the risk of MALE HR 0.58 (95% CI 0.38-0.88, p=0.0093). There was a monotonic relationship between lower achieved LDL-C and lower risk of limb events (P=0.0049) that extended down to 0.25 mmol/L. Patients with PAD were at high risk of cardiovascular events and PCSK9 inhibition with evolocumab significantly reduced that risk with large absolute risk reductions. Moreover, lowering of LDL-C with evolocumab reduced the risk of major adverse limb events. These data show LDL-C lowering in patients with PAD can lead reduce clinical complications of atherosclerotic disease across multiple vascular beds.

The findings of the present example show that PCSK9 inhibition with evolocumab added to background statin therapy lowered LDL cholesterol and significantly reduced cardiovascular risk with similar efficacy in patients with and without PAD, but greater absolute risk reduction in patients with PAD. LDL-C reduction with evolocumab also reduced major adverse limb events including acute limb ischemia, major amputation or urgent peripheral revascularization. The is the first study to show a reduction in major adverse limb events with PCSK9 inhibition.

Taken together, the data with statins and now with the PCSK9 inhibitor evolocumab added to a statin show that intensive LDL-C lowering in patients with PAD provides substantial reductions in the clinical complications of atherosclerotic disease across multiple vascular beds.

### Abbreviations Used in Example 18

ALI—acute limb ischemia
MACE—major adverse cardiovascular events
MALE—major adverse limb events
MI—myocardial infarction
PAD—peripheral artery disease
AKA—above the knee amputation
BKA—below the knee amputation

### Methods: Study Population

The FOURIER trial design is described in Sabatine M S, Giugliano R P, Keech A, et al. Rationale and design of the Further cardiovascular Outcomes Research with PCSK9 Inhibition in subjects with Elevated Risk trial. Am Heart J 2016; 173: 94-101. Patients with clinically evident atherosclerotic cardiovascular disease including prior myocardial infarction, prior ischemic stroke, or symptomatic peripheral artery disease were randomized in a 1:1 ratio to evolocumab or placebo. Patients were eligible to qualify with symptomatic peripheral artery disease if they had either: intermittent claudication and an ankle brachial index (ABI)<0.85, a history of a peripheral artery revascularization procedure, or a history of amputation due to atherosclerotic disease. In addition to the prespecified subgroup based on symptomatic lower extremity PAD, as part of a post-hoc exploratory analysis a more restricted population, defined as patients with symptomatic lower extremity PAD but with no history of MI or stroke, was also examined.

### Endpoints

The primary efficacy endpoint in FOURIER was major cardiovascular events, defined as the composite of cardiovascular death, MI, stroke, hospitalization for unstable angina, or coronary revascularization. The key secondary endpoint was the composite of CV death, MI or stroke. Other secondary endpoints included the components of the primary endpoint. Cardiovascular events were adjudicated by a blinded clinical event committee (CEC). Limb outcomes were prospectively ascertained through investigator reporting on dedicated electronic case report form pages and through adverse event forms. Limb outcomes were adjudicated by two blinded vascular medicine specialists. Similar to other recent trials evaluating medical therapies in patients with PAD, MALE was defined as the composite of acute limb ischemia (ALI), major amputation (above the knee, AKA or below the knee BKA, excluding forefoot or toe), or urgent revascularization (thrombolysis or urgent vascular intervention for ischemia).^{3,8,14,15,17} Acute limb ischemia (ALI) required both a clinical presentation consistent with acute ischemia including findings on physical examination and/or imaging.¹⁷ Acute limb ischemia and urgent revascularization for ischemia were identified by trained vascular medicine specialists blinded to treatment assignment.³ In addition, all peripheral artery revascularization and amputation procedures were recorded by the site in the electronic case report form. Analogous to other trials, a combined endpoint of MACE and MALE was examined.^{14,15,18} Prespecified safety endpoints as defined in the primary analysis were included for the PAD subgroup.¹⁹

### Statistical Considerations

As part of a prespecified analysis, patients were stratified into those with or without symptomatic lower extremity PAD at baseline as described above. Baseline characteristics of the subgroups were compared using Wilcoxon rank sum tests for continuous data and χ2 tests for categorical data. All efficacy analyses of evolocumab versus placebo were done on an intention-to-treat basis (i.e., all patients who were randomly assigned were analysed, irrespective of study drug compliance). Safety analyses included all randomly assigned patients who received at least one dose of study treatment and for whom post-dose data were available. P values for time-to-event analyses are from log-rank tests; Kaplan-Meier event rates were calculated up to 2.5 years. Hazard ratios (HRs) and 95% CIs for the effect of evolocumab versus placebo were generated by use of a Cox proportional hazards model, without adjustment (because of the randomised design) but stratifying by region and screening LDL-C values. The effect modification by PAD on the efficacy of evolocumab was tested by incorporating interaction terms into Cox models. For the analysis of risk of cardiovascular outcomes comparing patients with and without PAD in the placebo group, a multivariable-adjusted HR was obtained from a Cox model that included the following baseline covariates: age, sex, race, BMI, hypertension, diabetes, smoking status, renal dysfunction, CHF, prior MI, CABG or PCI and prior stroke or TIA. Proportional hazards assumptions were not violoated. A repeated measures linear mixed effects model was used to obtain the least square means percentage and absolute reduction in LDL-C between the two treatment groups. For analyses evaluating the relationship of achieved LDL-C at one month and outcomes, the relationship between composite efficacy endpoints and achieved LDL cholesterol was plotted using a smoothing function applied to the averages of estimated event rates at each LDL level based on the unadjusted Cox models, as has been done previously applying the same exclusion criteria. 20 P values below 0.05 were regarded as significant. SAS (version 9.4) was used for the statistical analyses.

### Results

### Populations

Of the 27,564 patients randomized, 3,642 (13.2%) had a history of symptomatic lower extremity PAD at baseline. A total of 2,067 patients (56.8%) had a history of prior peripheral revascularization, 126 (3.5%) had a history of amputation for vascular cause, and 2,518 (69.3%) had an ABI <0.85 and symptoms of claudication (with some patients having more than one of these factors). Patients with PAD were older, more frequently female, and had a greater prevalence of risk factors including hypertension, current smoking, renal insufficiency and diabetes (Table 18.1). At baseline 89% of patients were taking antiplatelet therapy, 69% high-intensity statin therapy, 30% moderate-intensity statin therapy, and 6.6% were taking ezetimibe. Of the PAD subgroup, 1,812 patients (49.8%) had a history of MI and 545 (15.0%) had a history of stroke; there were 1,505 (41% of those with PAD and 5% of the total population) who had PAD and no prior MI or stroke.

TABLE 18.1
BASELINE CHARACTERISTICS

| | No PAD | PAD |
|---|---|---|
| | N = 23,922 | N = 3,642 |
| Age, median (IQR) | 63 (56, 69) | 64 (58, 69) |
| Female sex, n (%) | 5743 (24.0) | 1026 (28.2) |
| Body Mass Index, median (IQR) | 29 (26, 32) | 29 (26, 32) |
| Caucasian, n (%) | 20156 (84.3) | 3302 (90.7) |
| History Hypertension, n (%) | 18993 (79.4) | 3091 (84.9) |
| Current Smoker, n (%) | 6451 (27.0) | 1326 (36.4) |
| Renal Insufficiency, n (%) | 1323 (5.5) | 340 (9.3) |
| History of Atrial Fibrillation, n (%) | 2022 (8.5) | 320 (8.8) |
| History of Diabetes, n (%) | 8501 (35.5) | 1580 (43.4) |
| History of Stroke/TIA, n (%) | 5101 (21.3) | 685 (18.8) |
| History of Myocardial Infarction, n (%) | 20539 (85.9) | 1812 (49.8) |
| History of CHF, n (%) | 5625 (23.5) | 769 (21.1) |
| Prior CABG, n (%) | 4387 (18.4) | 839 (23.0) |
| History of PCI, n (%) | 14029 (58.7) | 1444 (39.7) |
| Peripheral Artery Disease History | | |
| Symptomatic Peripheral Artery Disease | 0 | 1505 (41.3) |
| and no prior MI or Stroke | | |
| Current intermittent claudication & | 0 | 2518 (69.3) |
| ABI <0.85, n (%) | | |
| Prior Peripheral Revascularization, | 0 | 2067 (56.8) |
| n (%) | | |
| Time from Peripheral Revascularization, | 0 | 3.7 (1.3, 7.8) |
| years, median (IQR) | | |
| Limb amputation for vascular cause, | 0 | 126 (3.5) |
| n (%) | | |
| Medications at Baseline | | |
| High Intensity Statin use at baseline, | 16579 (69.3) | 2524 (69.3) |
| n (%) | | |
| Moderate Intensity Statin use at | 7282 (30.4) | 1110 (30.5) |
| baseline, n (%) | | |
| Low Intensity Statin use at baseline, | 51 (0.2) | 5 (0.1) |
| n (%) | | |
| Ezetimibe use at baseline, n (%) | 1200 (5.0) | 240 (6.6) |
| Antiplatelet therapy, n (%) | 22216 (92.9) | 3246 (89.3) |
| Anticoagulant therapy, n (%) | 1805 (7.6) | 391 (10.8) |
| ACE-I or ARB use at baseline, n (%) | 18526 (77.5) | 2747 (75.3) |

All p-value <0.05 except history of atrial fibrillation (p = 0.50) and statin use/intensity
(p = 0.57)
Statin dose at baseline missing in 10 (0.0%) without PAD and 3 (0.1%) with PAD

### Peripheral Artery Disease and Risk in Patients Randomized to Placebo

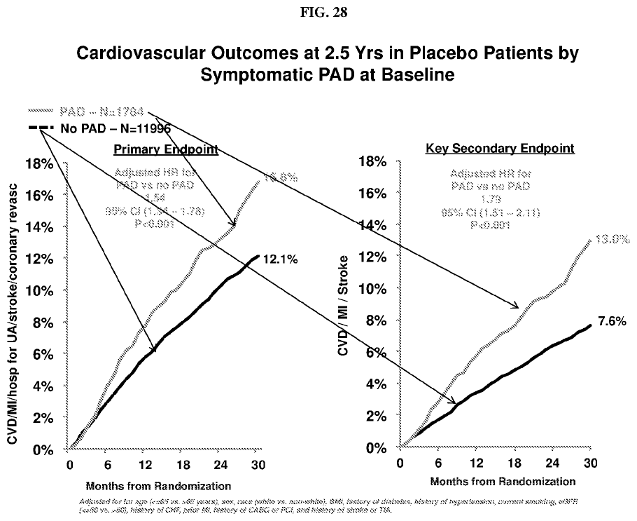

Among patients in the placebo arm, patients with PAD as compared with patients without PAD had higher rates of both the primary endpoint (Kaplan-Meier rate at 2.5 years: 16.8% vs 12.1%, P<0.001) and the key secondary endpoint (13.0% vs 7.6%, P<0.001) (Table 18.2, FIG. 28). After adjusting for baseline differences, patients with PAD remained at significantly higher risk of the primary endpoint (Adj. HR 1.57, 95% CI 1.36-1.80, p<0.001) and the key secondary endpoint (Adj. HR 1.81, 95% CI 1.53-2.14, p<0.001, Table 18.2, FIG. 28).

TABLE 18.2
RATES AND ADJUSTED HAZARD OF ISCHEMIC EVENTS
IN PLACEBO PATIENTS WITH PAD VS NO PAD

| | Symptomatic | No Symptomatic | | |
|---|---|---|---|---|
| | PAD N | PAD N | | |
| | (2.5 yr KM rate) | (2.5 yr KM | Adjusted HR | |
| | n = 1.784 | rate) n = 11,996 | (95% Cl) | P-value |
| Primary composite | 257 (16.80%) | 1,306 (12.13%) | 1.57 (1.36-1.80) | <0.001 |
| Key Secondary | 195 (13.01%) | 818 (7.63%) | 1.81 (1.54-2.14) | <0.001 |
| Cardiovascular death | 55 (3.78%) | 185 (1.73%) | 2.04 (1.48-2.82) | <0.001 |
| Myocardial Infarction | 115 (7.88%) | 524 (4.87%) | 1.85 (1.50-2.30) | <0.001 |
| Stroke | 50 (3.13%) | 212 (2.01%) | 1.52 (1.09-2.11) | 0.013 |
| Coronary | 142 (9.55%) | 823 (7.67%) | 1.45 (1.19-1.75) | <0.001 |
| revascularization | | | | |
| All cause mortality | 97 (6.66%) | 329 (3.01%) | 1.94 (1.52-2.47 | <0.001 |
| MALE | 40 (2.40%) | 19 (0.16%) | 11.67 (6.25-21.79) | <0.001 |
| ALI or major amputation | 25 (1.47%) | 15 (0.12%) | 7.88 (3.67-16.92) | <0.001 |
| ALI | 18 (1.06%) | 15 (0.12%) | 5.92 (2.59-13.53) | <0.001 |
| Major amputation | 7 (0.41%) | 0 (0.00%) | — | — |
| Urgent revascularization | 20 (1.25%) | 6 (0.06%) | 22.35 (8.26-60.47) | <0.001 |
| Any peripheral | 200 (12.40%) | 93 (0.90%) | 14.75 (11.28-19.29) | <0.001 |
| revascularization | | | | |
| DVD, MI, Stroke, or | 228 (15.03%) | 834 (7.77%) | 2.05 (1.75-2.40) | <0.001 |
| MALE | | | | |

PEP - primary endpoint composite of CV death, myocardial infarction, stroke, hospitalization for unstable angina or coronary revascularization
Key Secondary - composite of CV death, myocardial infarction or stroke
MALE - composite of acute limb ischemia (ALI), major amputation (AKA or BKA), or urgen peripheral revascularization for ischemia
MI = myocardial infarction, AKA = above knee amputation, BKA = below knee amputation, ALI = acute limb ischemia
Adjusted for age (65 vs. >=65), sex, race (white vs. non-white), BMI, history of diabetes, history of hypertension, smoking status (never, current, former), eGFR (<=60 vs. >60), history of congestive heart failure, prior mi, history of CABG or PCI, and history of non-hemorrhagic stroke or TIA
Note:
For any peripheral revascularization, smoking status was collapsed to current smoker vs. non-current smoker and age was changed to 75 vs. >=75

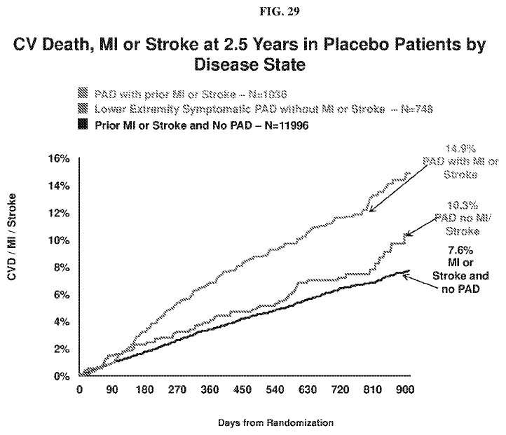

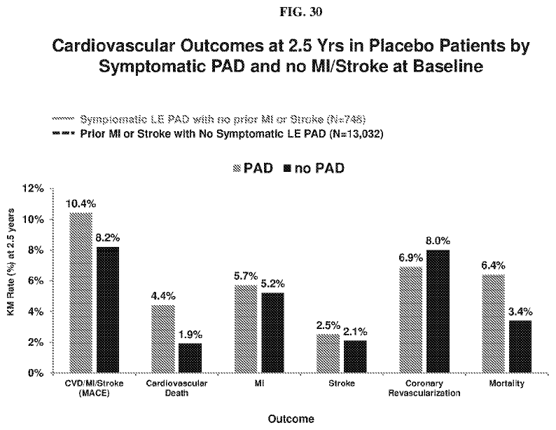

When stratifying the population with PAD by history of concomitant prior MI or stroke (polyvascular disease), those with polyvascular disease had higher rates of CV death, MI or stroke compared to those without (14.9% vs. 10.3%, p=0.0028, FIG. 29). Patients with PAD and no prior MI or stroke, however, still had higher rates of CV death, MI or stroke than patients with prior MI or stroke and no symptomatic PAD (10.3% vs 7.6%, Adjusted HR 2.07, 95% CI 1.42-3.01, p=0.0001, FIG. 29). When evaluating individual components CV death appeared especially higher (4.4% vs. 1.9%, p<0.001) although rates of MI and stroke were also numerically higher (FIG. 30).

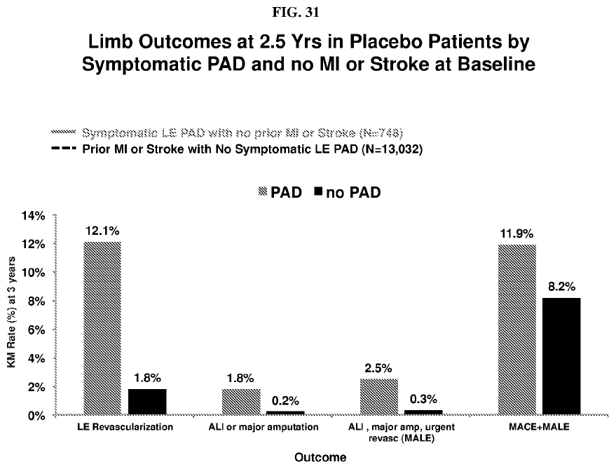

Patients with symptomatic PAD had higher rates of limb outcomes relative to those without PAD including MALE (2.4% vs 0.2%, adjusted HR 11.67, 95% CI 6.25-21.79, p<0.001) and the composite of ALI and major amputation (1.5% vs. 0.1%, adjusted HR 7.88, 95% CI 3.67-16.92, p<0.001, Table 18.2). Findings were consistent in the subgroup with PAD and no MI or Stroke vs patients with no PAD (FIG. 31).

LDL-Cholesterol Lowering with Evolocumab

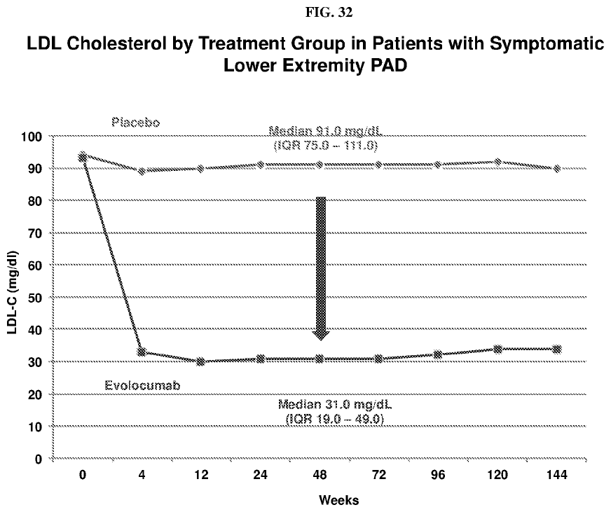

The median LDL-C level at baseline among the symptomatic PAD group was 94 mg/dL (IQR 81-112). At 48 weeks, the least-squares mean percentage reduction in LDL-C with evolocumab, relative to placebo, was 59% (95% CI 57 to 61, p<0.001) and 57 mg/dL (mean absolute reduction, 95% CI 55 to 60) to a median of 31.0 mg/dL (IQR 19.0-49.0, FIG. 32). The reduction in LDL cholesterol levels was maintained over time (FIG. 32).

Cardiovascular Efficacy with Evolocumab

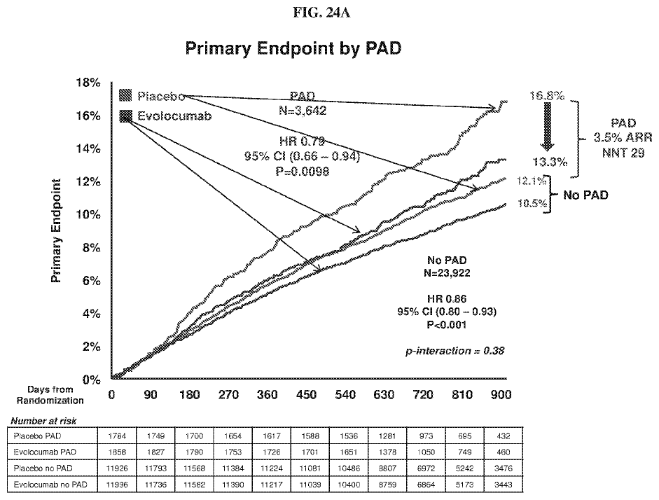

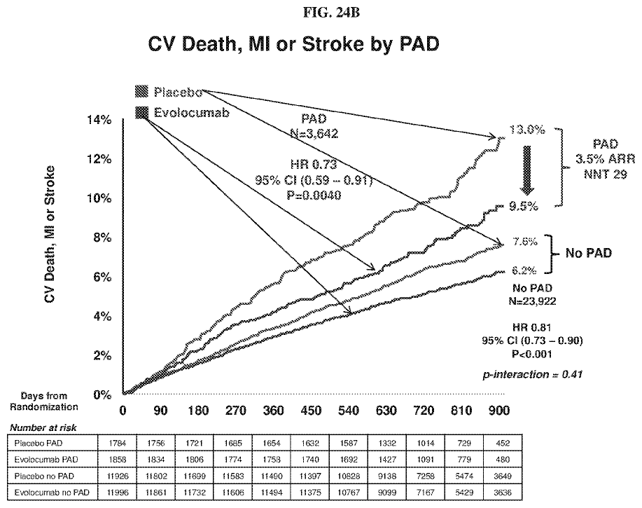

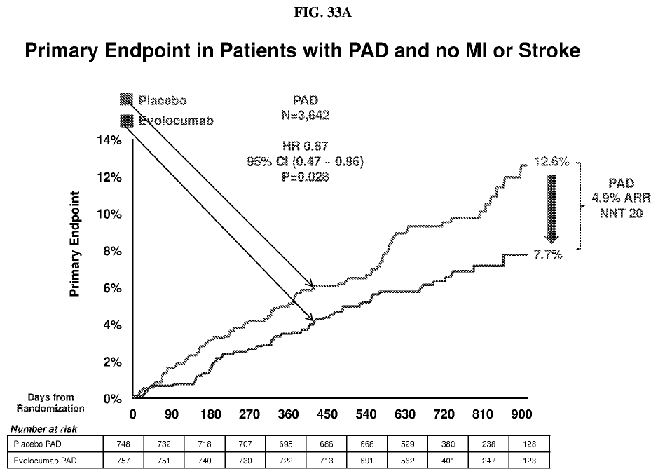

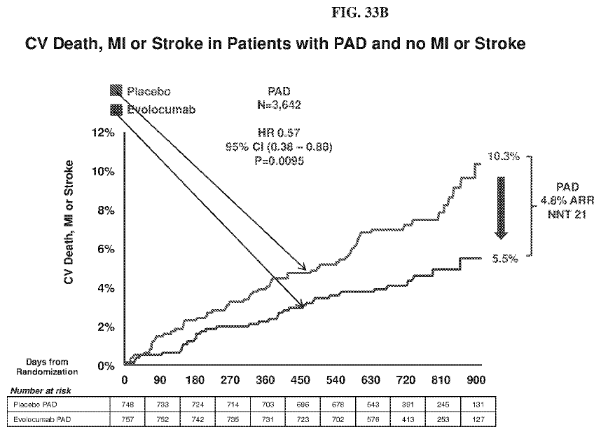

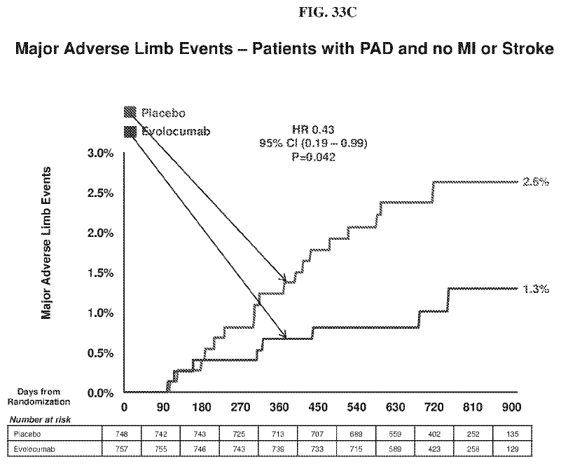

In patients with prior PAD, evolocumab significantly reduced the primary endpoint by 21% (2.5-year KM rate 13.3% vs. 16.8%, HR 0.79, 95% CI 0.66-0.94, p=0.0089, Table 18.3, FIG. 24A) and the composite of CV death, MI or stroke by 27% (9.5% vs. 13.0%, HR 0.73, 95% CI 0.59-0.91, p=0.0040, Table 18.3, FIG. 24B). The relative risk reductions for both endpoints were consistent in patients with and without PAD (p-interaction 0.40 and 0.41 respectively), however, due to higher absolute risk in patients with PAD, the absolute risk reductions for both endpoints were greater in those with PAD vs. those without [absolute risk reduction (ARR) for primary endpoint 3.5% (95% CI 0.8%-6.2%) in PAD; 1.6% (95% CI 0.7%-2.5%) without PAD; ARR for CV death, MI or stroke 3.5% (95% CI 1.0%-6.0%) in PAD; 1.4% (95% CI 0.7%-2.1%) without PAD]. Relative and absolute risk reductions were consistent in the population of patients with PAD and no prior MI or stroke including a 4.9% ARR (95% CI 1.0%-8.8%) in the primary endpoint and a 4.8% ARR (95% CI 1.2%-8.4%) in the composite of CV death, MI or stroke translating in NNT2.5y of 21 for each (Table 18.3, FIG. 33A and FIG. 33B.

TABLE 18.3
EFFICACY EVOLOCUMAB IN PATIENTS WITH PERIPHERAL
ARTERY DISEASE
Table 2. Efficacy of Evolocumab in Patient with Peripheral Artery Disease

| | | | | | Symptomatic PAD | | | |
|---|---|---|---|---|---|---|---|---|
| Efficacy | Symptomatic PAD | | | | without prior MI or Stroke | | | |
| Outcomes | | | Hazard | | | | Hazard | |
| Outcome, n, 2.5 | Placebo | Evolocumab | Ratio | | Placebo | Evolocumab | Ratio | |
| yr KM rage (%) | N = 1,784 | N = 1,858 | (95% CI) | p-value | N = 748 | N = 757 | (95% CI) | p-value |
| Primary | 257, 16.8% | 217, 13.3% | 0.79 | 0.0098 | 74, 12.6% | 51, 7.7% | 0.67 | 0.0283 |
| Endpoint | | | (0.66-0.94) | | | | (0.47-0.96) | |
| CV Death, MI, | 195, 13.0% | 152, 9.5% | 0.73 | 0.0040 | 58, 10.3% | 34, 5.5% | 0.57 | 0.0095 |
| Stroke (MACE) | | | (0.59-0.91) | | | | (0.38-0.88) | |
| CVD | 55, 3.8% | 58, 4.0% | 1.02 | | 18, 4.4% | 14, 2.9% | 0.78 | |
| | | | (0.71-1.48) | | | | (0.39-1.57) | |
| MI | 115, 7.9% | 84, 5.2% | 0.69 | | 32, 5.7% | 21, 2.9% | 0.66 | |
| | | | (0.52-0.91) | | | | (0.38-1.14) | |
| Stroke | 50, 3.1% | 31, 1.8% | 0.59 | | 16, 2.5% | 5, 0.7% | 0.30 | |
| | | | (0.38-0.92) | | | | (0.11-0.82) | |
| Ischemic Stroke | 47, 2.9% | 28, 1.7% | 0.57 | | 15, 2.4% | 4, 0.5% | 0.25 | |
| | | | (0.35-0.90) | | | | (0.08-0.77) | |
| Coronary | 142, 9.6% | 119, 7.0% | 0.79 | | 42, 6.9% | 30, 4.0% | 0.70 | |
| revascularization | | | (0.62-1.01) | | | | (0.44-1.13) | |
| All death | 97, 6.7% | 93, 6.2% | 0.92 | 0.58 | 31, 6.4% | 27, 4.9% | 0.86 | 0.58 |
| | | | (0.69-1.23) | | | | (0.51-1.45) | |
| MALE | 40, 2.4% | 27, 1.5% | 0.63 | 0.063 | 18, 2.60% | 8, 1.3% | 0.43 | 0.042 |
| | | | (0.39-1.03) | | | | (0.19-0.99) | |
| ALI or major | 25, 1.5% | 16, 0.9% | 0.60 | | 12, 1.8% | 4, 0.6% | 0.33 | |
| amputation | | | (0.32-1.13) | | | | (0.10-1.01) | |
| ALI | 18, 1.1% | 14, 0.8% | 0.73 | | 8, 1.2% | 4, 0.6% | 0.48 | |
| | | | (0.37-1.48) | | | | (0.15-1.61) | |
| Major | 7, 0.4% | 3, 0.2% | 0.41 | | 4, 0.58% | 1, 0.1% | 0.26 | |
| amputation | | | (0.11-1.57) | | | | (0.03-2.32) | |
| Urgent | 20, 1.2% | 16, 0.9% | 0.75 | | 8, 1.2% | 6, 0.9% | 0.72 | |
| revascularization | | | (0.39-1.45) | | | | (0.25-2.08) | |
| Any peripheral | 200, 12.4% | 215, 13.2% | 1.01 | 0.88 | 81, 12.1% | 95, 14.9% | 1.17 | 0.30 |
| revascularization | | | (0.84-1.23) | | | | (0.87-1.57) | |
| CV Death, MI, | 228, 15.0% | 177, 10.9% | 0.73 | 0.0014 | 75, 12.8% | 40, 6.5% | 0.52 | 0.0006 |
| Stroke, ALI, | | | (0.60-0.88) | | | | (0.35-0.76) | |
| major amp. or | | | | | | | | |
| urgent revasc. | | | | | | | | |

MALE-composite of acute limb ischemia (ALI), major amputation (AKA or BKA), or urgent peripheral revascularization for ischemia
MI = myocardia infarction, AKA = above knee amputation, BKA = below knee amputation, ALI = acute limb ischemia

Major Adverse Limb Event Reduction with Evolocumab

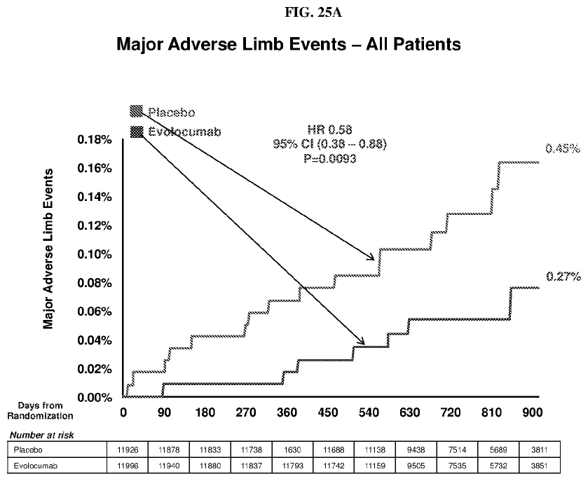

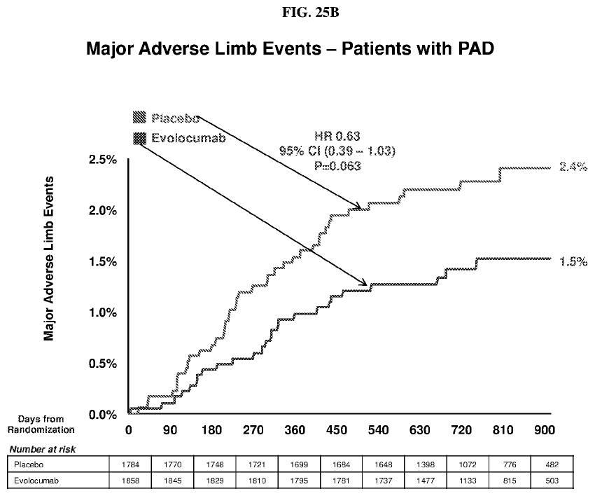

Overall evolocumab reduced the risk of MALE by 42% (0.45% vs 0.26%. HR 0.58, 95% CI 0.38-0.88, p=0.0093, Table 18.4, FIG. 25A) and the pattern of efficacy was consistent across all components of MALE (Table 18.4). In the 3642 patients with PAD, the pattern of efficacy for MALE was consistent (HR 0.63, 95% 0.39-1.03) but rates were higher, translating into greater absolute risk reductions (Table 18.3, FIG. 25B) with similar findings in patients with PAD and no prior MI or stroke (FIG. 33C).

Overall evolocumab reduced the risk of MALE by 42% (0.45% vs 0.26%. HR 0.58, 95% CI 0.38-0.88, p=0.0093, Table 18.4, FIG. 25A) and the pattern of efficacy was consistent across all components of MALE (Table 18.4). In the 3642 patients with PAD, the pattern of efficacy for MALE was consistent (HR 0.63, 95% 0.39-1.03), but rates were higher, translating into greater absolute risk reductions (Table 18.3, FIG. 25B) with similar findings in patients with PAD and no prior MI or stroke (Table 18.3, FIG. 33C).

TABLE 18.4
MAJOR ADVERSE LIMB OUTCOMES WITH EVOLOCUMAB
Efficacy Outcomes

| | | | | |
|---|---|---|---|---|
| | Placebo | Evolocumab | | |
| | N = 13,780 | N = 13,784 | Hazard | |
| | n, 2.5 yr | n, 2.5 yr | Ratio | |
| Outcome | KM rate (%) | KM rate (%) | (95% CI) | p-value |

Limb Outcomes

| | | | | |
|---|---|---|---|---|
| MALE | 59, 0.45% | 34, 0.27% | 0.58 | 0.0093 |
| | | | (0.38-0.88) | |
| ALI or major | 40, 0.29% | 21, 0.17% | 0.52 | |
| amputation | | | (0.31-0.89) | |
| ALI | 33, 0.24% | 18, 0.15% | 0.55 | |
| | | | (0.31-0.97) | |
| Major | 7, 0.05% | 4, 0.03% | 0.57 | |
| amputation | | | (0.17-1.95) | |
| Urgent | 26, 0.21% | 18, 0.13% | 0.69 | |
| revascularization | | | (0.38-1.26) | |
| Any peripheral | 293, 2.37% | 317, 2.59% | 1.08 | 0.33 |
| revascularization | | | (0.92-1.27) | |

Composite of MACE + MALE

| | | | | |
|---|---|---|---|---|
| CV Death, MI, | 1062, 8.70% | 847, 6.91% | 0.79 | <0.001 |
| Stroke, MALE | | | (0.72-0.87) | |

MALE—composite of acute limb ischemia (ALI), major amputation (AKA or BKA), or urgent peripheral revascularization for ischemia
MI = myocardial infarction,
AKA = above knee amputation,
BKA = below knee amputation,
ALI = acute limb ischemia

Composite Outcomes in Patients with PAD

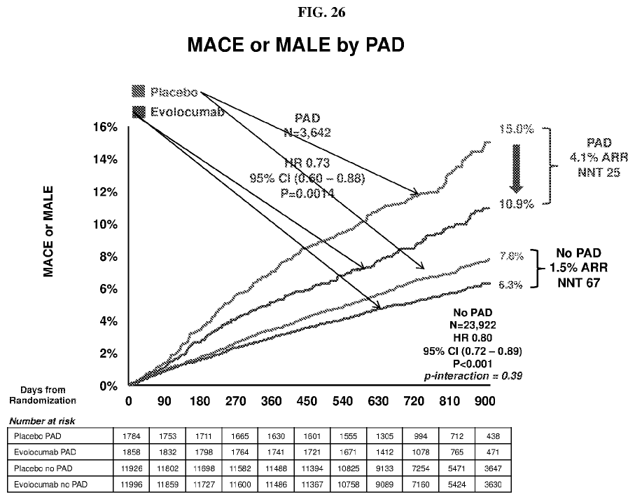

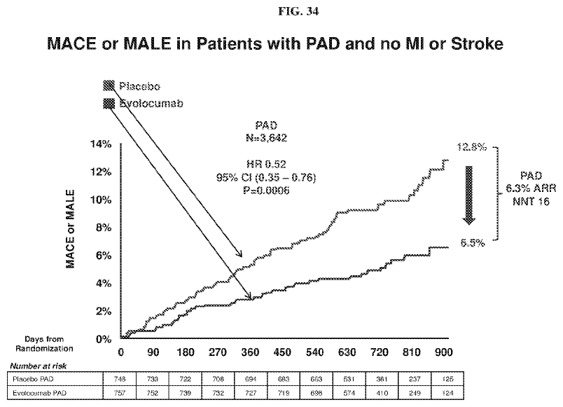

Overall evolocumab reduced the composite of MACE (CV death, MI or stroke) or MALE (ALI, major amputation or urgent revascularization) by 21% (8.70% vs 6.91%, HR 0.79, 95% CI 0.72-0.87, p<0.001). The relative risk reduction was similar in those with and without PAD (p-interaction 0.39) but due to their higher absolute risk (placebo rate 15.0% in those with PAD vs 10.9% without PAD) there was a numerically greater absolute risk reduction at 2.5 years in those with PAD (ARR 4.1%, 95% CI 2.5-6.7, FIG. 26) relative to those without PAD (ARR 1.5%, 95% CI 0.7-2.2, FIG. 26). Similarly, in those with PAD and no prior MI or stroke, there was a significant reduction in the composite of MACE or MALE (6.5% vs. 12.8%, HR 0.52, 95% CI 0.35-0.76, p=0.0006; ARR 6.3%, NNT 16, FIG. 34).

Safety of Evolocumab in Patients with PAD

There were no differences in incidence adverse or serious adverse events with evolocumab relative to placebo in patients with PAD (Table 18.5). There was no excess of adverse events leading to treatment discontinuation (1.3% evolocumab vs 1.5% placebo, p=0.57).

TABLE 18.5
SAFETY OF EVOLOCUMAB IN PATIENTS WITH PERIPHERAL
ARTERY DISEASE

| | Placebo | | Evolocumab | |
|---|---|---|---|---|
| Adverse events, n (%) | n = 1,780 | N | n = 1,856 | N |
| Any | 1,408 (79.1%) | 1780 | 1,481 (79.8%) | 1856 |
| Serious | 624 (35.1%) | 1780 | 601 (32.4%) | 1856 |
| Thought to be related to | 27 (1.5%) | 1780 | 24 (1.3%) | 1856 |
| study agent and leading | | | | |
| to the discontinuation of | | | | |
| study regimen | | | | |
| Injection-Site reaction | 32 (1.8%) | 1780 | 26 (1.4%) | 1856 |
| Allergic reaction | 47 (2.6%) | 1780 | 54 (2.9%) | 1856 |
| Muscle-related event | 79 (4.4%) | 1780 | 94 (5.1%) | 1856 |
| Rhabdomyolysis | 1 (0.1%) | 1780 | 2 (0.1%) | 1856 |
| Cataract | 43 (2.4%) | 1780 | 24 (1.3%) | 1856 |
| Adjudicated case of new- | 67 (6.7%) | 996 | 80 (8.3%) | 963 |
| onset diabetes | | | | |
| Neurocognitive event | 31 (1.7%) | 1780 | 28 (1.5%) | 1856 |
| Laboratory results, n (%) | | | | |
| Aminotransferase level > | 31 (1.8%) | 1747 | 27 (1.5%) | 1812 |
| 3 times the upper limit of | | | | |
| the normal range | | | | |
| Creatine Kinase level > 5 | 15 (0.9%) | 1747 | 5 (0.3%) | 1812 |
| times the upper limite of | | | | |
| the normal range | | | | |

Note:
P-value was calculated by chi-square test
All p-values > 0.05 except nominal p = 0.0119 for cataracts and 0.0201 for CK > 5

### Association of Achieved LDL-Cholesterol and Risk of MACE and MALE

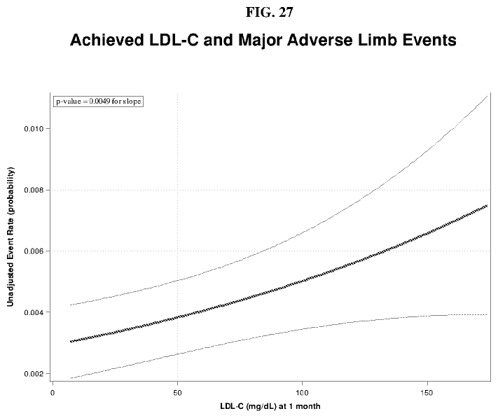

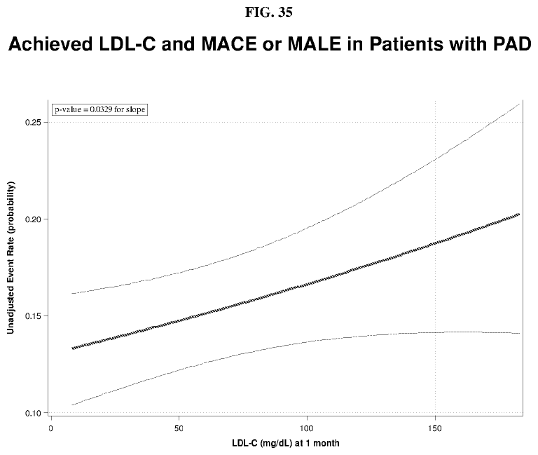

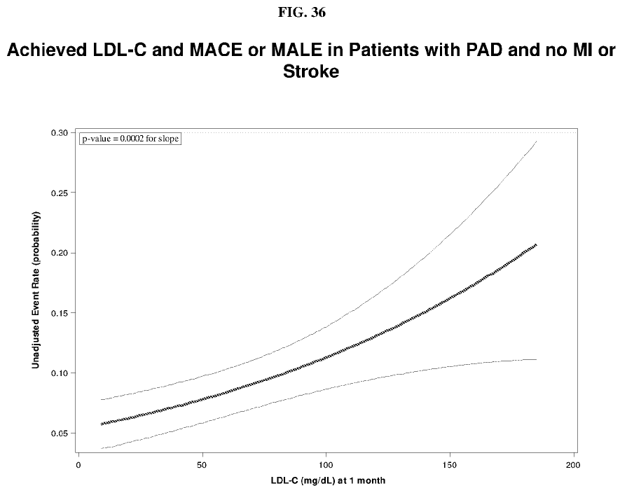

Overall lower achieved LDL-C was associated with a significantly lower risk of MALE with a roughly linear relationship down to LDL-C of 10 mg/dL (p=0.0049 for slope FIG. 27). There was no apparent inflection or plateau in the relationship between LDL-C and outcome. This pattern was consistent for the broader composite outcome of MACE or MALE overall and for patients with PAD (FIG. 35) and patients with PAD and no prior MI or stroke (FIG. 36).

### Discussion of Results

This study demonstrates that patients with symptomatic lower extremity PAD are at higher risk of both MACE and MALE relative to patients with prior MI or stroke and no PAD. Evolocumab significantly reduced the risk of MACE in patients with symptomatic PAD, including those without prior MI or stroke, and the higher risk in PAD patients translated into greater absolute risk reductions. Furthermore, LDL-C lowering with evolocumab reduced the risk of MALE including ALI and major amputation. Thus when considering both MACE and MALE, the absolute risk reduction with LDL-C lowering in patients with PAD was quite robust, with an NNT over 2.5 years of only 25. Lastly, akin to what has been observed for MACE, there was a monotonic lower risk of MALE with lower levels of achieved LDL-C, down to 10 mg/dL.

The higher ischemic risk in patients with symptomatic PAD as compared to those without has been recognized.^{14,21,22} This observation, however, is complex as there is heterogeneity in risk within the broad population of patients with PAD. Those patients with multiple symptomatic territories (e.g. PAD and prior MI or prior stroke), called polyvascular disease, are at clearly heightened risk and appear to derive robust reductions in MACE risk from more intensive antithrombotic therapy.^{3,23} For patients with symptomatic PAD and no prior MI or stroke, the benefits of intensive antithrombotic therapy for MACE reduction are less compelling with studies showing neutral results or modest efficacy. 14,24 This distinction has practical implications both for clinicians and guidelines where distinguishing the risks and benefits in patients with PAD and no history of MI or stroke from those with prior MI or stroke may guide recommendations and treatment decisions and assist in personalizing treatment selection.^{4,5}

In the current Example, two symptomatic PAD populations have been shown, a broad population including those with polyvascular disease as well as a restricted population that has never experienced an acute atherothrombotic event (MI or stroke). In contrast to intensive antithrombotic therapies, however, the benefits of intensive lipid lowering with evolocumab were consistent in both populations. These findings therefore highlight a distinct population where lipid lowering provides robust benefits and supports the hypothesis that the biology of MACE risk in this population is responsive to LDL-C lowering.

There are limited prior randomized, controlled data on the effect of LDL-C lowering on clinical outcomes in PAD. The Heart Protection Study randomized 20,536 patients with vascular disease with a total cholesterol of at least 3.5 mmol/L to simvastatin 40 mg daily or placebo and included 6,748 patients with PAD.²⁵ Over 5 years of follow up, simvastatin reduced major vascular events relative to placebo with consistent relative risk reductions in those with and without PAD.²⁶ An exploratory outcome of non-coronary vascular intervention (including carotid intervention) was also lower with simvastatin.²⁶ There was no difference in the risk of amputation with simvastatin vs. placebo. Beyond these observations, there are no well-powered randomised studies showing that achieving lower LDL-C or that the use of a non-statin agent to a statin is beneficial in PAD. This lack of data has led some to conclude that until further evidence on the relative effectiveness of different lipid-lowering agents is available, use of a statin in patients with PAD should be limited to those with a total cholesterol level ≥3.5 mmol/L, a threshold far higher than in most other patients with ASCVD.⁹

The current Example now adds data from a well-powered randomized trial that achieving lower LDL-C with a non-statin agent added to high or moderate intensity statin therapy is beneficial in patients with symptomatic lower extremity PAD, including those without prior MI or stroke.⁹

In addition to robust benefits for MACE, the current Example is the first randomized trial to demonstrate a benefit for intensive LDL-C lowering for MALE risk. The Heart Protection Study noted a reduction in the outcome of non-coronary revascularization procedures; however, this was not specific to etiology and included procedures beyond the lower extremities such as carotid revascularization.²⁶ Major adverse limb events were not reported and there was no difference in amputations.²⁶ Prior small studies have described potential symptomatic benefits with statin therapy but have not been powered for MALE.^{10,11,27} Analyses from large registries have observed an association between lower amputation rates and statin therapy; however, potential for residual confounding has remained and intensity of statin therapy or achieved LDL-C was not reported.^{6,29,30} The current Example demonstrates that non-statin LDL-C lowering added to statins reduces MALE and that the benefits extend to very low achieved LDL-C.

The reduction in MALE with evolocumab was consistent for all the components, which have now been established as modifiable limb endpoints in three randomized trials of more intensive antithrombotic therapy and endpoints that have been adopted as elements of primary or key secondary endpoints in trials including patients with PAD.^{3,8,14,15,31} There was no apparent benefit for reducing peripheral revascularizations including elective procedures for claudication as has been described for other therapies including cilostazol and vorapaxar.⁸ Possible explanations for the lack of benefit for this broad endpoint include that lipid lowering does not improve symptoms or alternatively, it does but over a longer period of exposure and therefore was not seen in the relatively short duration of follow up (median 2.2 years) in the current study. Supporting the latter is the observation that benefits for peripheral revascularization and symptoms with vorapaxar were not apparent until almost 2 years of exposure and were not significant until 3 years.

In evaluating the overall benefits of preventive therapies in patients with PAD, recent and ongoing trials have utilized a composite endpoint including both cardiovascular and limb outcomes.^{15,31} This composite provides a global picture of benefit in patients with PAD against which harms and cost can be weighed. In the current Example, in patients with PAD and, robust reductions in both MACE and MALE resulted in an absolute risk reduction at 2.5 years of 4.1% and an NNT of 21. Extending this observation to 5 years, as is typically done for lipid lowering therapy, translates to a NNT approximately 11. In contrast to anti-thrombotic therapies, this benefit comes with no safety tradeoff in terms of bleeding or other adverse events. These considerations may be important to clinicians in personalizing intensive therapies to their patients.

### Analysis

Subgroup analyses were generally utilized to evaluate for consistency of findings with the overall trial and therefore may be underpowered for efficacy and safety outcomes. In the current analysis the PAD subgroup was adequately powered to demonstrate statistically significant benefits for the primary endpoint and key secondary. The power to detect differences in safety events may have been more limited but the pattern of safety was consistent with the overall trial and are not be anticipated to be modified by the presence of PAD. Limb outcomes were collected on broad eCRF pages for peripheral outcomes and not focused specifically on ALI. This may have resulted in under ascertainment of ALI outcomes but would not bias treatment effects. Finally, relationships between achieved LDL-C and outcome were not randomized and while adjusted for confounders the potential for residual confounding remains and should be recognized.

### Conclusions

Patients with symptomatic lower extremity PAD are at heightened risk of major adverse cardiovascular and limb risks. Evolocumab added to statin therapy significantly and robustly reduces the risk of MACE, even in patients with PAD and no prior MI or stroke. Likewise, the addition of evolocumab to a statin reduced the risk of major adverse limb events (MALE), and the relationship between achieved LDL-C and lower risk of limb events extended down to very low achieved levels of LDL (e.g., 10 mg/dL). These benefits come with no apparent safety concerns. Thus, LDL-C reduction to very low levels is useful in patients with PAD, regardless of a history of MI or stroke, to reduce the risk of MACE and MALE.

### Reference for Example 18

1. Suarez C, Zeymer U, Limbourg T, Baumgartner I, Cacoub P, Poldermans D, Rother J, Bhatt D L, Steg P G, REACH Registry Investigators. Influence of polyvascular disease on cardiovascular event rates. Insights from the REACH Registry. Vasc Med. 2010; 15: 259-265.
2. Criqui M H, Aboyans V. Epidemiology of peripheral artery disease. Circ Res. 2015; 116: 1509-1526.
3. Bonaca M P, Bhatt D L, Storey R F, Steg P G, Cohen M, Kuder J, Goodrich E, Nicolau J C, Parkhomenko A, Lopez-Sendon J, Dellborg M, Dalby A, Spinar J, Aylward P, Corbalan R, Abola M T, Jensen E C, Held P, Braunwald E, Sabatine M S. Ticagrelor for Prevention of Ischemic Events After Myocardial Infarction in Patients With Peripheral Artery Disease. J Am Coll Cardiol. 2016; 67: 2719-2728.
4. Aboyans V, Ricco J B, Bartelink M E L, Bjorck M, Brodmann M, Cohnert T, Collet J P, Czerny M, De Carlo M, Debus S, Espinola-Klein C, Kahan T, Kownator S, Mazzolai L, Naylor A R, Roffi M, Rother J, Sprynger M, Tendera M, Tepe G, Venermo M, Vlachopoulos C, Desormais I. 2017 ESC Guidelines on the Diagnosis and Treatment of Peripheral Arterial Diseases, in collaboration with the European Society for Vascular Surgery (ESVS): Document covering atherosclerotic disease of extracranial carotid and vertebral, mesenteric, renal, upper and lower extremity arteriesEndorsed by: the European Stroke Organization (ESO) The Task Force for the Diagnosis and Treatment of Peripheral Arterial Diseases of the European Society of Cardiology (ESC) and of the European Society for Vascular Surgery (ESVS). Eur Heart J. 2017.
5. Gerhard-Herman M D, Gornik H L, Barrett C, Barshes N R, Corriere M A, Drachman D E, Fleisher L A, Fowkes F G, Hamburg N M, Kinlay S, Lookstein R, Misra S, Mureebe L, Olin J W, Patel R A, Regensteiner J G, Schanzer A, Shishehbor M H, Stewart K J, Treat-Jacobson D, Walsh M E. 2016 AHA/ACC Guideline on the Management of Patients With Lower Extremity Peripheral Artery Disease: A Report of the American College of Cardiology/American Heart Association Task Force on Clinical Practice Guidelines. Circulation. 2016.
6. Kumbhani D J, Steg P G, Cannon C P, Eagle K A, Smith S C, Jr, Goto S, Ohman E M, Elbez Y, Sritara P, Baumgartner I, Banerjee S, Creager M A, Bhatt D L, REACH Registry Investigators. Statin therapy and long-term adverse limb outcomes in patients with peripheral artery disease: insights from the REACH registry. Eur Heart J. 2014; 35: 2864-2872.
7. Jones W S, Baumgartner I, Hiatt W R, Heizer G, Conte M S, White C J, Berger J S, Held P, Katona B G, Mahaffey K W, Norgren L, Blomster J, Millegard M, Reist C, Patel M R, Fowkes G R, International Steering Committee and Investigators of the EUCLID Trial (Examining Use of tiCagreLor In paD). Ticagrelor Compared With Clopidogrel in Patients with Prior Lower Extremity Revascularization for Peripheral Artery Disease. Circulation. 2016.
8. Bonaca M P, Scirica B M, Creager M A, Olin J, Bounameaux H, Dellborg M, Lamp J M, Murphy S A, Braunwald E, Morrow D A. Vorapaxar in patients with peripheral artery disease: results from TRA2{degrees}P-TIMI 50. Circulation. 2013; 127: 1522-9, 1529e1-6.
9. Aung P P, Maxwell H G, Jepson R G, Price J F, Leng G C. Lipid-lowering for peripheral arterial disease of the lower limb. Cochrane Database Syst Rev. 2007; (4): CD000123.
10. Aronow W S, Nayak D, Woodworth S, Ahn C. Effect of simvastatin versus placebo on treadmill exercise time until the onset of intermittent claudication in older patients with peripheral arterial disease at six months and at one year after treatment. Am J Cardiol. 2003; 92: 711-712.
11. Mohler E R, 3rd, Hiatt W R, Creager M A. Cholesterol reduction with atorvastatin improves walking distance in patients with peripheral arterial disease. Circulation. 2003; 108: 1481-1486.
12. Spring S, Simon R, van der Loo B, Kovacevic T, Brockes C, Rousson V, Amann-Vesti B, Koppensteiner R. High-dose atorvastatin in peripheral arterial disease (PAD): effect on endothelial function, intima-media-thickness and local progression of PAD. An open randomized controlled pilot trial. Thromb Haemost. 2008; 99: 182-189.
13. Schanzer A, Hevelone N, Owens C D, Beckman J A, Belkin M, Conte M S. Statins are independently associated with reduced mortality in patients undergoing infrainguinal bypass graft surgery for critical limb ischemia. J Vasc Surg. 2008; 47: 774-781.
14. Hiatt W R, Fowkes F G, Heizer G, Berger J S, Baumgartner I, Held P, Katona B G, Mahaffey K W, Norgren L, Jones W S, Blomster J, Millegard M, Reist C, Patel M R, EUCLID Trial Steering Committee and Investigators. Ticagrelor versus Clopidogrel in Symptomatic Peripheral Artery Disease. N Engl J Med. 2016.
15. Anand S. et al. <br/>COMPASS PAD—Cardiovascular OutcoMes for People using Anticoagulation StrategieS trial: Results in Patients with Peripheral Artery Disease. European Society of Cardiology Hotline. 2017.
16. Sabatine M S, Giugliano R P, Keech A, Honarpour N, Wang H, Liu T, Wasserman S M, Scott R, Sever P S, Pedersen T R. Rationale and design of the Further cardiovascular OUtcomes Research with PCSK9 Inhibition in subjects with Elevated Risk trial. Am Heart J. 2016; 173: 94-101.
17. Bonaca M P, Gutierrez J A, Creager M A, Scirica B M, Olin J, Murphy S A, Braunwald E, Morrow D A. Acute Limb Ischemia and Outcomes With Vorapaxar in Patients With Peripheral Artery Disease: Results From the Trial to Assess the Effects of Vorapaxar in Preventing Heart Attack and Stroke in Patients With Atherosclerosis-Thrombolysis in Myocardial Infarction 50 (TRA2 degrees P-TIMI 50). Circulation. 2016; 133: 997-1005.
18. Eikelboom J W, Connolly S J, Bosch J, Dagenais G R, Hart R G, Shestakovska O, Diaz R, Alings M, Lonn E M, Anand S S, Widimsky P, Hori M, Avezum A, Piegas L S, Branch K R H, Probstfield J, Bhatt D L, Zhu J, Liang Y, Maggioni A P, Lopez-Jaramillo P, O'Donnell M, Kakkar A K, Fox K A A, Parkhomenko A N, Ertl G, Stork S, Keltai M, Ryden L, Pogosova N, Dans A L, Lanas F, Commerford P J, Torp-Pedersen C, Guzik T J, Verhamme P B, Vinereanu D, Kim J H, Tonkin A M, Lewis B S, Felix C, Yusoff K, Steg P G, Metsarinne K P, Cook Bruns N, Misselwitz F, Chen E, Leong D, Yusuf S, COMPASS Investigators. Rivaroxaban with or without Aspirin in Stable Cardiovascular Disease. N Engl J Med. 2017; 377: 1319-1330.
19. Sabatine M S, Giugliano R P, Pedersen T R. Evolocumab in Patients with Cardiovascular Disease. N Engl J Med. 2017; 377: 787-788.
20. Giugliano R P, Pedersen T R, Park J G, De Ferrari G M, Gaciong Z A, Ceska R, Toth K, Gouni-Berthold I, Lopez-Miranda J, Schiele F, Mach F, Ott B R, Kanevsky E, Pineda A L, Somaratne R, Wasserman S M, Keech A C, Sever P S, Sabatine M S, FOURIER Investigators. Clinical efficacy and safety of achieving very low LDL-cholesterol concentrations with the PCSK9 inhibitor evolocumab: a prespecified secondary analysis of the FOURIER trial. Lancet. 2017.
21. Cacoub P P, Bhatt D L, Steg P G, Topol E J, Creager M A, CHARISMA Investigators. Patients with peripheral arterial disease in the CHARISMA trial. Eur Heart J. 2009; 30: 192-201.
22. Bhatt D L, Peterson E D, Harrington R A, Ou F S, Cannon C P, Gibson C M, Kleiman N S, Brindis R G, Peacock W F, Brener S J, Menon V, Smith S C, Jr, Pollack C V, Jr, Gibler W B, Ohman E M, Roe M T, CRUSADE Investigators. Prior polyvascular disease: risk factor for adverse ischaemic outcomes in acute coronary syndromes. Eur Heart J. 2009; 30: 1195-1202.
23. Franzone A, Piccolo R, Gargiulo G, Ariotti S, Marino M, Santucci A, Baldo A, Magnani G, Moschovitis A, Windecker S, Valgimigli M. Prolonged vs Short Duration of Dual Antiplatelet Therapy After Percutaneous Coronary Intervention in Patients With or Without Peripheral Arterial Disease: A Subgroup Analysis of the PRODIGY Randomized Clinical Trial. JAMA Cardiol. 2016.
24. Warfarin Antiplatelet Vascular Evaluation Trial Investigators, Anand S, Yusuf S, Xie C, Pogue J, Eikelboom J, Budaj A, Sussex B, Liu L, Guzman R, Cina C, Crowell R, Keltai M, Gosselin G. Oral anticoagulant and antiplatelet therapy and peripheral arterial disease. N Engl. J Med. 2007; 357: 217-227.
25. Collins R, Armitage J, Parish S, Sleight P, Peto R, Heart Protection Study Collaborative Group. Effects of cholesterol-lowering with simvastatin on stroke and other major vascular events in 20536 people with cerebrovascular disease or other high-risk conditions. Lancet. 2004; 363: 757-767.
26. Heart Protection Study Collaborative Group. Randomized trial of the effects of cholesterol-lowering with simvastatin on peripheral vascular and other major vascular outcomes in 20,536 people with peripheral arterial disease and other high-risk conditions. J Vasc Surg. 2007; 45: 645-654; discussion 653-4.
27. Giri J, McDermott M M, Greenland P, Guralnik J M, Criqui M R, Liu K, Ferrucci L, Green D, Schneider J R, Tian L. Statin use and functional decline in patients with and without peripheral arterial disease. J Am Coll Cardiol. 2006; 47: 998-1004.
28. Rajamani K, Colman P G, Li L P, Best J D, Voysey M, D'Emden M C, Laakso M, Baker J R, Keech A C, FIELD study investigators. Effect of fenofibrate on amputation events in people with type 2 diabetes mellitus (FIELD study): a prespecified analysis of a randomised controlled trial. Lancet. 2009; 373: 1780-1788.
29. Dosluoglu H H, Davari-Farid S, Pourafkari L, Harris L M, Nader N D. Statin use is associated with improved overall survival without affecting patency and limb salvage rates following open or endovascular revascularization. Vasc Med. 2014; 19: 86-93.
30. Feringa H H, Karagiannis S E, van Waning V H, Boersma E, Schouten O, Bax J J, Poldermans D. The effect of intensified lipid-lowering therapy on long-term prognosis in patients with peripheral arterial disease. J Vasc Surg. 2007; 45: 936-943.
31. Bayer. <br/>Efficacy and Safety of Rivaroxaban in Reducing the Risk of Major Thrombotic Vascular Events in Subjects With Symptomatic Peripheral Artery Disease Undergoing Peripheral Revascularization Procedures of the Lower Extremities (VOYAGER PAD)-NCT02504216<br >. Available at: world wide web “dot” clinicaltrials “dot” gov.

### Example 19

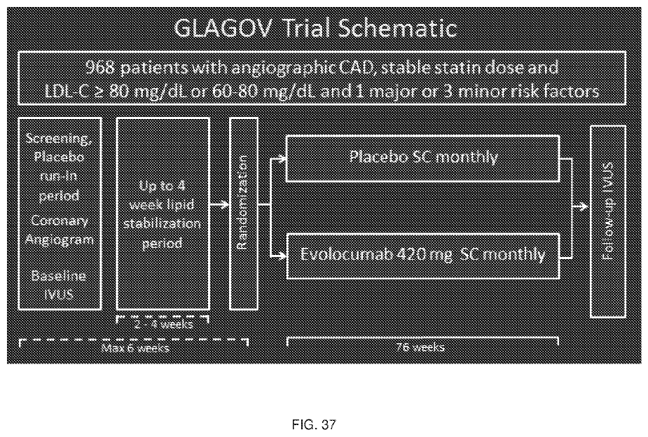

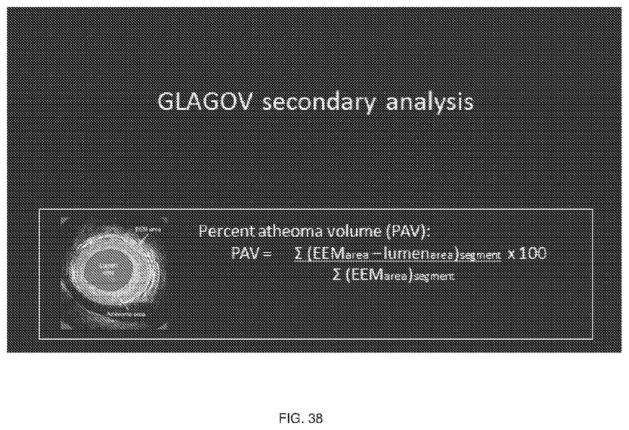

The present example examines predictors of residual plaque progression despite achieving low levels of LDL-C with the PCSK9 inhibitor, evolocumab. Intravascular ultrasound (IVUS) trials have shown that statins slow progression or induce regression of coronary disease in proportion to the magnitude of LDL-C reduction. In addition to statins, non-statin LDL-C lowering agents, such as proprotein convertase subtilisin/kexin type 9 (PCSK9) antibodies, have emerged as a new class of drugs that effectively lower LDL-C levels. For example, in the GLAGOV trial, evolocumab reduced LDL-C levels from 93 to 37 mg/dL and induced greater plaque regression than placebo in statin-treated patients (−0.95% vs. +0.05%, P<0.0001). FIG. 37 depicts the GLAGOV trial schematic for the context of this study. While evolocumab induced regression in a greater percentage of patients (64% vs. 36%, P<0.0001); more than one third of subjects with evolocumab still exhibited some plaque progression despite very low LDL-C levels. The present example examines the factors associated with ongoing disease progression in the setting of evolocumab treatment. The parameters of the subjects and the study are outlined in Tables 19.1-19.5. FIG. 38 depicts a cross-sectional lumen and formula for determining percent atheroma volume. Subjects with a PAV>0 were “progressors” while subjects with a PAV<0 were regressors.

TABLE 19.1
Patient Demographics

| | Progressors | Regressors | |
|---|---|---|---|
| | (n = 151) | (n = 272) | P value |
| Age, yrs | 59.5 | 59.4 | 0.94 |
| Female, % | 29.8 | 26.5 | 0.46 |
| BMI, kg/m² | 28.7 | 28.4 | 0.19 |
| Hypertension, % | 79.5 | 82.0 | 0.53 |
| Diabetes mellitus, % | 21.9 | 20.2 | 0.69 |
| Previous MI, % | 34.4 | 33.1 | 0.78 |
| Current smoker, % | 24.5 | 26.8 | 0.60 |
| Baseline statin use, % | 98.0 | 99.3 | 0.35 |
| High-Intensity, % | 59.6 | 59.9 | |
| Moderate-intensity, % | 37.7 | 39.0 | 0.60 |
| Low-intensity, % | 0.7 | 0.4 | |
| β-blocker, % | 78.8 | 72.8 | 0.17 |
| ACE inhibitor, % | 52.3 | 54.0 | 0.73 |

Plaque progressions were observed in 151 (35.7%) of evolocumab-treated patients. No differences in clinical demographics were observed between progressors and regressors (Table 19.1).

TABLE 19.2
Risk factor control (1)

| | Progressors | Regressors | |
|---|---|---|---|
| | (n = 151) | (n = 272) | P value |
| LDL cholesterol, mg/dL | | | |
| Baseline | 94.4 | 91.2 | 0.24 |
| On-treatment | 37.8 | 34.3 | 0.14 |
| Change | −58.3 | −57.9 | 0.89 |
| HDL cholesterol, mg/dL | | | |
| Baseline | 46.6 | 46.6 | 0.99 |
| On-treatment | 49.6 | 51.4 | 0.16 |
| Change | 2.0 | 3.8 | 0.008 |
| Triglycerides, mg/dL | | | |
| Baseline | 121.0 | 117.5 | 0.66 |
| On-treatment | 107.5 | 104.3 | 0.13 |
| Change | −11.4 | −9.8 | 0.87 |
| Non-HDL cholesterol, mg/dL | | | |
| Baseline | 122.4 | 117.8 | 0.17 |
| On-treatment | 59.4 | 55.3 | 0.15 |
| Change | −65.0 | −62.9 | 0.50 |

TABLE 19.3
Risk factor control (2)

| | Progressors | Regressors | |
|---|---|---|---|
| | (n = 151) | (n = 272) | P value |
| Apolipoprotein B, mg/dL | | | |
| Baseline | 83.7 | 79.9 | 0.07 |
| On-treatment | 43.7 | 40.6 | 0.08 |
| Change | −42.7 | −41.2 | 0.44 |
| Apolipoprotein A-1, mg/dL | | | |
| Baseline | 142.8 | 140.1 | 0.28 |
| On-treatment | 150.0 | 152.6 | 0.26 |
| Change | 5.5 | 10.7 | <0.001 |
| Apolipoprotein B/A-1 ratio | | | |
| Baseline | 0.60 | 0.58 | 0.28 |
| On-treatment | 0.30 | 0.27 | 0.03 |
| Change | −0.32 | −0.33 | 0.80 |
| Lipoprotein (a), mg/dL | | | |
| Baseline | 8.9 | 14.6 | 0.09 |
| On-treatment | 5.1 | 8.2 | 0.22 |
| Change | −3.1 | −4.3 | 0.08 |

TABLE 19.4a
Risk factor control (3)

| | Progressors | Regressors | |
|---|---|---|---|
| | (n = 151) | (n = 272) | P value |
| HbA1c, % | | | |
| Baseline | 6.0 | 5.8 | 0.03 |
| On-treatment | 6.1 | 6.0 | 0.20 |
| Change | 0.2 | 0.2 | 0.09 |
| Glucose, mg/dL | | | |
| Baseline | 106.2 | 103.1 | 0.22 |
| On-treatment | 110.8 | 110.5 | 0.90 |
| Change | 6.6 | 9.3 | 0.22 |
| hs-CRP, mg/L | | | |
| Baseline | 1.7 | 1.5 | 0.60 |
| On-treatment | 1.8 | 1.4 | 0.09 |
| Change | 0.3 | −0.5 | 0.32 |
| Systolic blood pressure, mmHg | | | |
| Baseline | 132.8 | 130.5 | 0.12 |
| On-treatment | 132.0 | 130.9 | 0.34 |
| Change | −2.7 | −1.4 | 0.32 |

TABLE 19.4(b)

| | Progressors (n = 151) | Regressors (n = 272) | P value |
|---|---|---|---|
| LDL cholesterol, mean (95% CI), mg/dL | | | |
| Baseline | 94.4 (90.0, 98.9) | 91.2 (87.9, 94.4) | 0.24 |
| On-treatment | 37.8 (33.7, 41.8) | 34.3 (31.8, 36.9) | 0.14 |
| Change | −58.3 (−63.8, −52.7) | −57.9 (−62.6, −53.1) | 0.89 |
| HDL cholesterol, mean (95% CI), mg/dL | | | |
| Baseline | 46.6 (44.6, 48.7) | 46.6 (45.1, 48.2) | 0.99 |
| On-treatment | 49.6 (47.6, 51.6) | 51.4 (49.9, 52.9) | 0.16 |
| Change | 2.0 (0.6, 3.4) | 3.8 (2.6, 5.0) | 0.008 |
| Triglycerides, median (IOR), mg/dL | | | |
| Baseline | 121.0 (91.0, 171.0) | 117.5 (92.0, 152.0) | 0.66 |
| On-treatment | 107.5 (90.8, 146.0) | 104.3 (82.3, 139.1) | 0.13 |
| Change | −11.4 (−37.0, 14.0) | −9.8 (−37.8, 10.8) | 0.87 |
| Non-HDL cholesterol, mean (95% CI), mg/dL | | | |
| Baseline | 122.4 (117.1, 127.7) | 117.8 (113.9, 121.7) | 0.17 |
| On-treatment | 59.4 (54.6, 64.1) | 55.3 52.1 58.5) | 0.15 |
| Change | −65.0 (−71.5, −58.4) | −62.9 (−68.4, −57.3) | 0.50 |
| Apolipoprotein B, mean (95% CI), mg/dL | | | |
| Baseline | 83.7 (80.3, 87.0) | 79.9 (77.4, 82.3) | 0.07 |
| On-treatment | 43.7 (40.7, 46.8) | 40.6 (38.7, 42.6) | 0.08 |
| Change | −42.7 (−46.8, −38.5) | −41.2 (−44.7, −37.7) | 0.44 |
| Apolipoprotein A-1, mean (95% CI), mg/dL | | | |
| Baseline | 142.8 (139.0, 146.6) | 140.1 (137.1, 143.1) | 0.28 |
| On-treatment | 150.0 (146.3, 153.8) | 152.6 (149.9, 155.3) | 0.26 |
| Change | 5.5 (2.3, 8.8) | 10.7 (8.0, 13.4) | <0.001 |
| Apolipoprotein B/A-1 ratio, mean (95% CI), | | | |
| Baseline | 0.60 (0.57, 0.63) | 0.58 (0.56, 0.60) | 0.28 |
| On-treatment | 0.30 (0.28, 0.33) | 0.27 (0.26, 0.29) | 0.03 |
| Change | −0.32 (−0.35, −0.29) | −0.33 (−0.35, −0.30) | 0.80 |
| Lipoprotein (a), median (IOR), mg/dL | | | |
| Baseline | 8.9 (4.3, 48.4) | 14.6 (4.6, 62.1) | 0.09 |
| On-treatment | 5.1 (2.4, 40.8) | 8.2 (2.4, 50.7) | 0.22 |
| Change | −3.1 (−9.6, −0.6) | −4.3 (−12.3, −0.9) | 0.08 |
| Glucose, mean (95% CI), mg/dL † | | | |
| Baseline | 106.2 (101.4, 111.0) | 103.1 (100.5, 105.6) | 0.22 |
| On-treatment | 110.8 (106.3, 115.4) | 110.5 (107.5, 113.5) | 0.90 |
| Change | 6.6 (2.0, 11.2) | 9.3 (5.4, 13.2) | 0.22 |
| Hemoglobin A1c, mean (95% CI), % † | | | |
| Baseline | 6.0 (5.8, 6.1) | 5.8 (5.7, 5.9) | 0.03 |
| On-treatment | 6.1 (5.9, 6.2) | 6.0 (5.9, 6.0) | 0.20 |
| Change | 0.2 (0.1, 0.3) | 0.2 (0.2, 0.3) | 0.09 |
| hs-CRP, median (IOR), mg/L † | | | |
| Baseline | 1.7 (0.8, 3.3) | 1.5 (0.8. 3.2) | 0.60 |
| On-treatment | 1.8 (0.9, 3.2) | 1.4 (0.7, 2.8) | 0.09 |
| Change | 0.3 (−1.3, 1.9) | −0.5 (−1.8, 0.9) | 0.32 |
| Systolic blood pressure, mean (95% CI), mmHg | | | |
| Baseline | 132.8 (130.4, 135.2) | 130.5 (128.7, 132.2) | 0.12 |
| On-treatment | 132.0 (130.1, 133.9) | 130.9 (129.6, 132.2) | 0.34 |
| Change | −2.7 (−5.3, −0.1) | −1.4 (−3.6, 0.9) | 0.28 |

The values are shown in Tables 19.4-19.4(b). Changes in levels of LDL-C (−58.3 ± 2.82 mg/dL vs. −57.9 ± 2.41 mg/dL, P = 0.89), apolipoprotein B (−42.7 ± 2.1 mg/dL vs. −41.2 ± 1.8 mg/dL, P = 0.44) and hsCRP (0.29 vs. −0.46 mg/L, P = 0.32) did not differ between the groups. Disease progressors demonstrated higher levels of baseline HbA1c (6.0 ± 0.8% vs. 5.8 ± 0.6%, P = 0.03), on-treatment levels of the apolipoprotein B/A-I ratio (0.30 ± 0.15 vs. 0.27 ± 0.12, P = 0.03) and smaller increases in levels of HDL-C (2.0 ± 0.72 mg/dL vs. 3.8 ± 0.61 mg/dL, P = 0.008) and apolipoprotein A-I (5.5 ± 1.63 mg/dL vs. 10.7 ± 1.39 mg/dL, P < 0.001) compared with patients undergoing plaque regression with evolocumab.
*Results are expressed as mean (95% CI) at baseline and least squares mean (95% CI) for on-treatment values.
† Time-weighted averages are used for on-treatment values. Absolute changes are presented as least squares means (95% CIs).

TABLE 19.5
IVUS parameters

| | Progressors | Regressors | |
|---|---|---|---|
| | (n = 151) | (n = 272) | P value |
| Baseline | | | |
| Percent atheroma volume, % | 33.1 | 38.3 | <0.001 |
| Total atheroma volume, mm³ | 167.2 | 197.9 | <0.001 |
| Follow-up at 78 wks | | | |
| Percent atheroma volume, % | 35.2 | 35.9 | 0.40 |
| Total atheroma volume, mm³ | 173.7 | 185.9 | 0.05 |
| Change from baseline | | | |
| Percent atheroma volume, % | 1.91 | −2.33 | <0.001 |
| Total atheroma volume, mm³ | 4.54 | −12.11 | <0.001 |

As shown in Table 19.5, progressors had lower percent atheroma volume at baseline (33.1% vs. 38.3%, P<0.001) than regressors.

TABLE 19.6
Determinants of plaque progression

| | OR | 95% CI | P value |
|---|---|---|---|
| Baseline percent atheroma volume (%) | 0.93 | 0.90-0.95 | <0.001 |
| Baseline HbA1c (%) | 1.48 | 1.10-2.00 | 0.01 |
| Change in apolipoprotein A-1 (%) | 0.98 | 0.97-0.99 | 0.01 |
| Baseline systolic BP (mmHg) | 1.01 | 0.99-1.03 | 0.06 |

Despite achieving extremely low LDL-C levels, 36% of patients with evolocumab still exhibited plaque progression. There were no significant differences in LDL-C levels between progressors and regressors.

FIG. 39 depicts the results of the analysis outlined in the tables above. The graphs in FIG. 39 show plaque progression and percent atheroma volume as a function of the number of risk factors present. An increase in the number of risk factors results in an increase in the risk of plaque progression, with the greatest increase in risk occurring in subjects with 3 or more risk factors.

Table 19.6 above, summarizes the various risk factors. Factors independently associated with ongoing progression were PAV (p<0.001), HbA1c (p=0.01) and change in apolipoprotein A-I (p=0.01), while systolic blood pressure was marginally significant (p=0.06). A greater number of additional atherogenic risk factors was associated with greater propensity to ongoing plaque progression and attenuated atheroma regression.

Factors associated with a greater propensity to ongoing plaque progression, despite evolocumab treatment, included the presence of additional atherogenic factors. These finding highlight the value of multifactorial risk modification even in the setting of very low LDL-C levels in order to prevent atherosclerotic progression in patients with coronary artery disease.

### Example 20

### Regression of Coronary Atherosclerosis with the PCSK9 Inhibitor, Evolocumab, in Patients with Greater Lp(a) Levels

Lp(a) levels can predict cardiovascular risk. Proprotein convertase subtilisin kexin type 9 (PCSK9) inhibitors can reduce Lp(a) by 21-30%. The present study provides additional insight into the impact of PCSK9 inhibition on plaque at different Lp(a) levels. The GLAGOV study compared the effects of the PCSK9 inhibitor, evolocumab, and placebo for 78 weeks on progression of coronary atherosclerosis in statin-treated patients with coronary artery disease. The impact of evolocumab on plaque progression was observed in patients stratified according to baseline Lp(a) levels.

Evolocumab reduced percent atheroma volume (PAV) by 0.8% (P<0.001 compared with baseline) and 1.2% (P<0.001 compared with baseline) and total atheroma volume (TAV) by 5.3 mm³ (P<0.001 compared with baseline) and 7.7 mm³ (P<0.001 compared with baseline) in patients with Lp(a) levels below and above the median baseline Lp(a) level (11.8 mg/dL) respectively.

Patients with higher Lp(a) levels were more likely to demonstrate PAV regression (70.6% vs 58.7%, P=0.01). Additional analysis demonstrated increasing plaque regression with evolocumab in patients with increasing baseline Lp(a) levels >11.8 mg/dL (P=0.04), while a similar degree of regression with evolocumab was observed regardless of Lp(a) levels <11.8 mg/dL (P=0.35). This greater benefit at higher Lp(a) levels >11.8 mg/dL just failed to meet statistical significance following adjustment for baseline plaque burden (P=0.09).

Evolocumab treated patients with a baseline Lp(a) >11.8 mg/dL were less likely to have diabetes (16.1% vs 25.5%, P=0.02), hypertension (75.4% vs 86.5%, P=0.001), have lower baseline CRP levels (1.3 vs 1.77 mg/L, P=0.02) and higher on-treatment LDL-C levels (33.9 vs 32.6 mg/dL, P=0.02). After adjustment for clinical and biochemical risk factors, increasing Lp(a) levels >11.8 mg/dL a trend towards greater plaque regression with evolocumab treatment (P=0.07), although this just failed to meet statistical significance.

While evolocumab produced plaque regression in statin-treated patients at all Lp(a) levels, greater baseline values, even within the normal range, identified patients likely to derive a greater degree of regression. This suggests that Lp(a), even within the normal range, may identify patients with a more modifiable form of atherosclerosis for treatment with intensive lipid lowering.

### Example 21

The present example demonstrates that the long-term use of an antibody to PCSK9 (e.g., evolocumab) can be used to reduce risk of recurrent cardiovascular events in patients with a history of multiple events and across heart attack types. Additional analysis found that patients closer to their most recent heart attack experienced substantial risk reductions with the antibody (e.g., evolocumab). In addition, it is shown that reducing LDL-C with a PCSK9 antibody (such as evolocumab) significantly and safely reduces risk of cardiovascular events in patients with peripheral artery disease. Patients with a history of MI within 2 years of enrollment had absolute risk reductions (ARR; 2.9 percent).

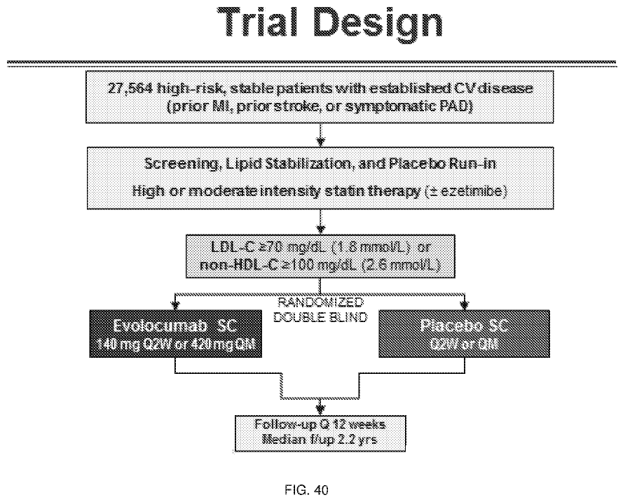

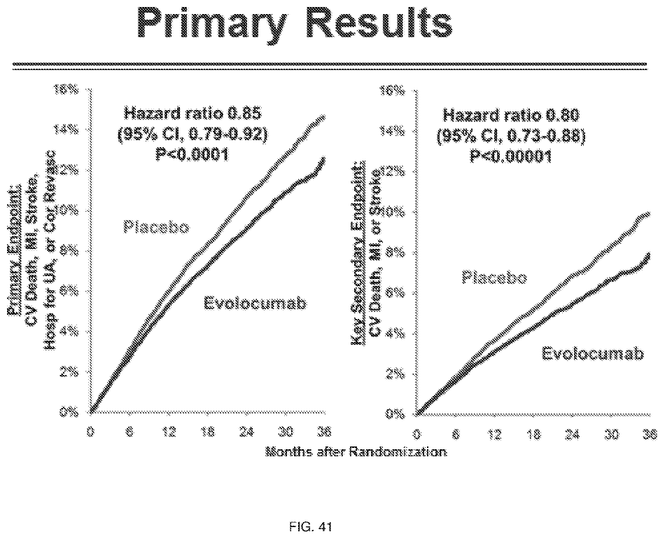

The efficacy of evolocumab (in combination with statin therapy) was evaluated in different myocardial infarction (MI) subgroups. Patients with a history of MI (N=22,351) were characterized according to the time since their most recent MI event, number of previous MIs and presence of multivessel coronary artery disease (CAD). Treatment with evolocumab resulted in an absolute risk reduction of 2.9 percent in patients within two years of their most recent MI (N=8,402), 2.6 percent in those with multiple prior MIs (N=5,282) and 3.4 percent in patients with a history of multivessel CAD (N=5618) respectively. The design of the study is depicted in FIG. 40 and FIG. 41 depicts the primary results.

The analysis was restricted to 22,351 Pts with prior MI. These were divided into subgroups based on three factors: 1) time from qualifying prior MI (min. 4 weeks per protocol) 2) number of prior MIs, and 3) presence of residual multivessel disease (≥40% stenosis in ≥2 vessels. The outcome of interest was: CV death, MI, or stroke. The analyses considered risk of CV events in placebo arm in different subgroups and the efficacy of Repatha in different subgroups.

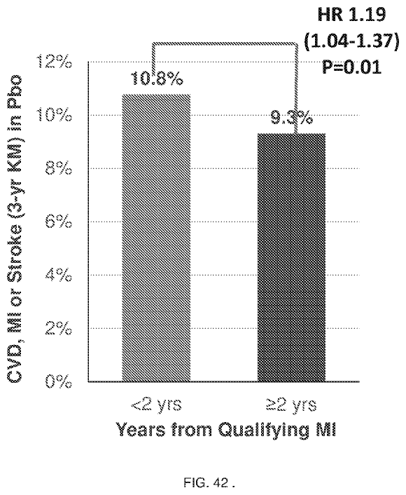

The baseline characteristics of the subjects are shown in table 21.0 and FIG. 42

TABLE 21.0

| | Prior MI | |
|---|---|---|
| | <2 y ago | Prior MI |
| | N = 840 | ≥2 y ago |
| Characteristic | 2 (38%) | N = 13,918 |
| Age, mean (SD) | 60 (9) | 63 (9) |
| Male sex (%) | 77 | 79 |
| Hypertension (%) | 75 | 81 |
| Diabetes mellitus (%) | 31 | 38 |
| Current smoker (%) | 28 | 28 |
| High-intensity statin (%) | 76 | 69 |
| LDL-C, mg/dL (IQR) | 90 | 93 |
| | (79-106) | (80-110) |

Achieved LDL-C at 48 wk, mg/dL (IQR)

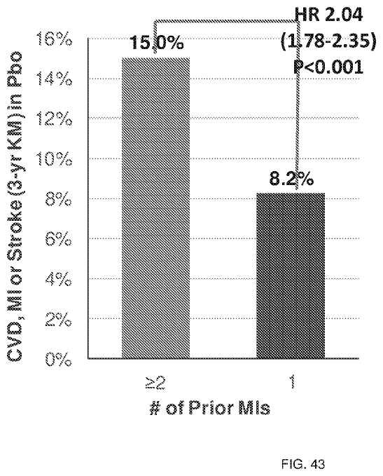

The characteristics of the subjects are shown in table 21.1 for the relationship to the number of prior MIs, depicted in FIG. 43.

TABLE 21.1

| | ≥2 Prior | |
|---|---|---|
| | MIs | |
| | N = 5285 | 1 Prior MI |
| Characteristic | (24%) | N = 17,047 |
| Age, mean (SD) | 62 (9) | 62 (9) |
| Male sex (%) | 82 | 77 |
| Hypertension (%) | 81 | 78 |
| Diabetes mellitus (%) | 36 | 35 |
| Current smoker (%) | 26 | 28 |
| High-intensity statin (%) | 75 | 70 |
| LDL-C, mg/dL (IQR) | 92 | 92 |
| | (81-105) | (80-108) |

Achieved LDL-C at 48 wk, mg/dL (IQR)

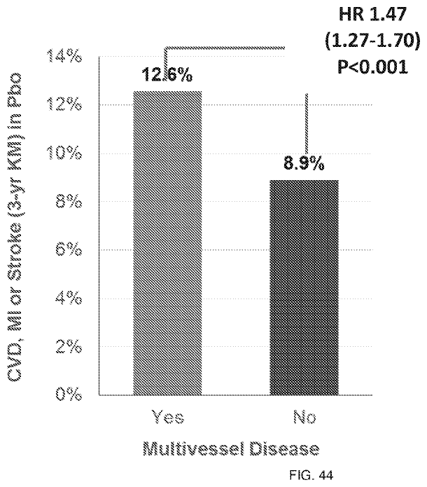

The characteristics of the subjects are shown in table 21.2 for the relationship to multivessel CAD, depicted in FIG. 44

TABLE 21.2

| | ≥2 Prior | |
|---|---|---|
| | MIs | |
| | N = 5285 | 1 Prior MI |
| Characteristic | (24%) | N = 17,047 |
| Age, mean (SD) | 62 (9) | 62 (9) |
| Male sex (%) | 82 | 77 |
| Hypertension (%) | 81 | 78 |
| Diabetes mellitus (%) | 36 | 35 |
| Current smoker (%) | 26 | 28 |
| High-intensity statin (%) | 75 | 70 |
| LDL-C, mg/dL (IQR) | 92 | 92 |
| | (81-105) | (80-108) |

Achieved LDL-C at 48 wk, mg/dL(IQR)

For every 1,000 patients treated for three years, evolocumab prevented 22 first primary endpoint events and 52 total primary endpoint events. An evaluation of all the primary endpoint events during the course of the study revealed that the addition of evolocumab to statin therapy improved clinical outcomes with significant reductions in total primary endpoint events driven by decreases in MI, stroke, and coronary revascularization. Evolocumab reduced total primary endpoint events by 18 percent (incidence-rate ratio 0.82, 95 percent CI 0.75-0.90, p<0.001).

Lowering LDL-C with an antibody to PCSK9 (e.g, evolocumab) was shown to reduce the risk of MI (27 percent,) and a new analysis revealed a robust benefit across multiple subtypes of MI. Evolocumab was also effective in reducing the risk for MI regardless of size (significant reductions observed regardless of fold elevations in troponin levels) and severity (STEMI or non-STEMI). Treatment with evolocumab was associated with a 36 percent reduction in the risk for STEMI, which accounted for one-fifth of MIs in the study population.

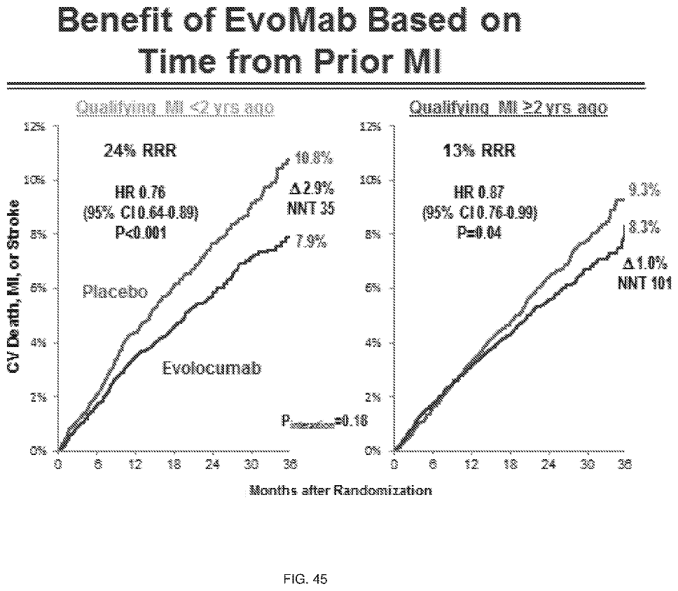

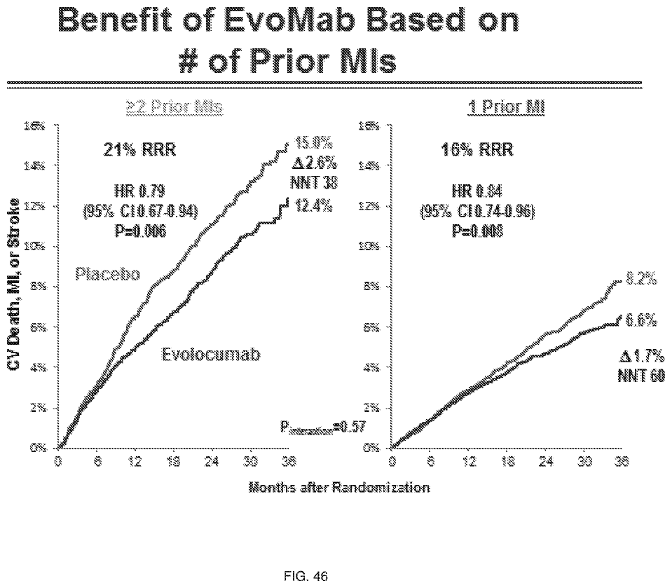

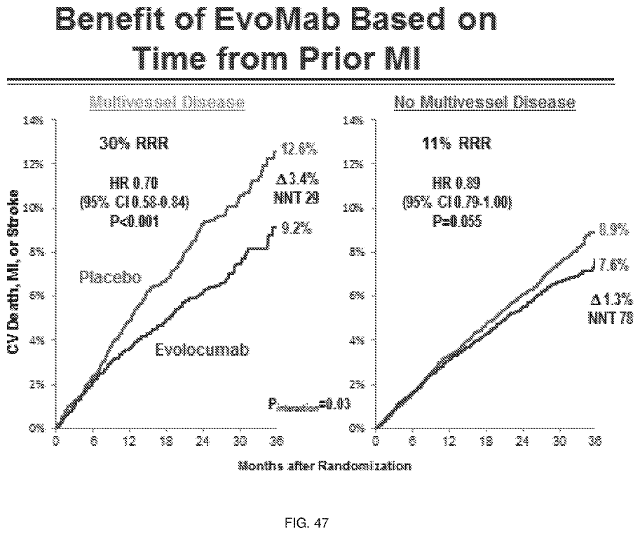

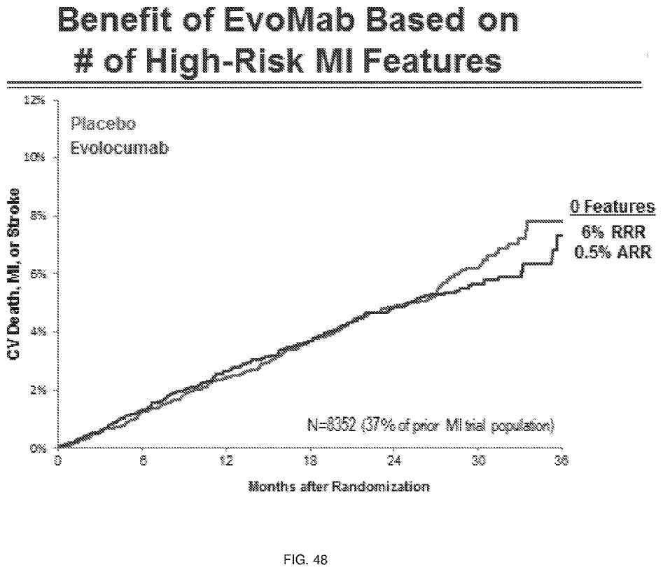

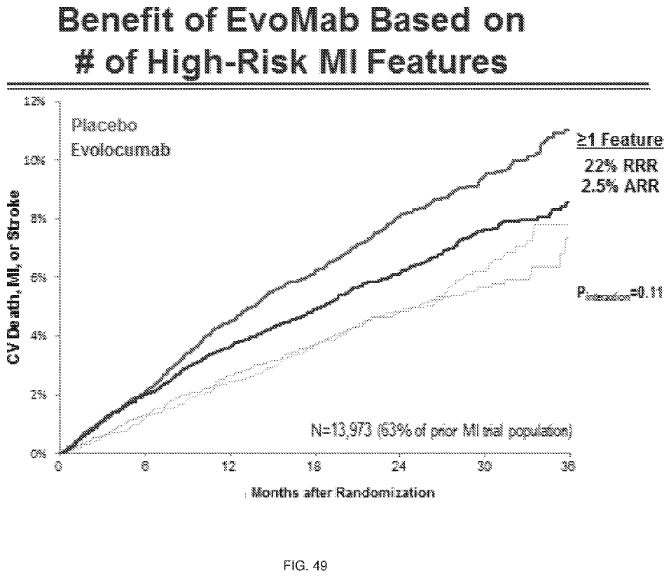

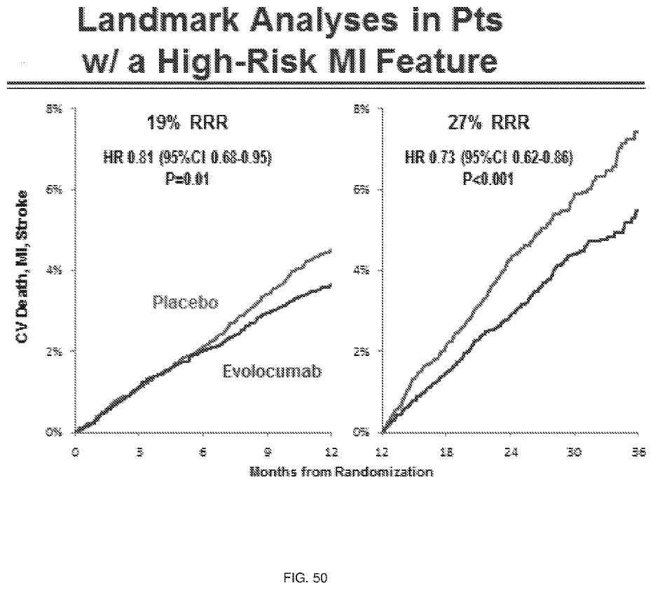

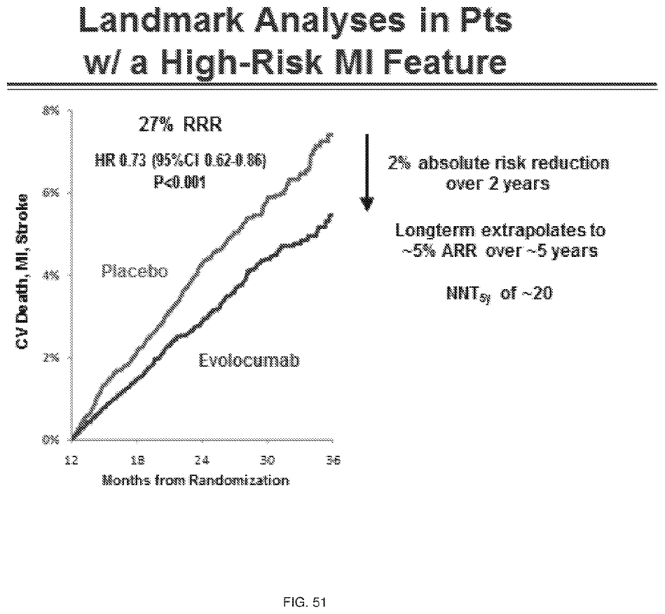

FIGS. 42-51 depict the results of this study. As shown in FIGS. 42-44, those with recent MI (less than 2 years, FIGS. 42, 45, and 47), 2 or more MIs (FIGS. 43 and 46), or multivessel disease (FIGS. 44 and 47) had an increased benefit from the combination therapy provided herein. Indeed, as shown in FIGS. 48-51, the presence of various high-risk MI features (one or more) allowed for identification of those subjects that would benefit from a combination therapy (in this example, statin and evolocumab).

Patients (1) closer to their most recent MI, (2) with multiple prior MIs, or (3) with multivessel disease are at an increased risk for major vascular events. These patients experience substantial relative and absolute risk reductions with intensive LDL-C lowering with evolocumab. These readily ascertainable clinical features offer an approach to tailoring therapy to particular subjects with an increased benefit to those subjects.

Participants in the present example (evolocumab cardiovascular outcomes study) were prospectively stratified according to their Thrombolysis in Myocardial Infarction (TIMI) Risk Score for Secondary Prevention to identify those with the greatest potential for clinical benefit following treatment with Repatha. Consistent with previous results, higher risk was associated with greater absolute risk reductions.

### Example 22

### Atherothrombotic Risk Stratification and Magnitude of Benefit of Evolocumab in FOURIER

Introduction:

Evolocumab (EvoMab) significantly reduced the relative risk of cardiovascular (CV) death, MI or stroke by 20% (absolute risk reduction 2% at 3 years) in patients with atherosclerotic CV disease. However, such patients vary in their risk for CV events.

Hypothesis:

Risk stratification with the TIMI Risk Score for Secondary Prevention (TRS 2° P) will identify patients who have the greatest potential for benefit from EvoMab. Methods: The TRS 2° P was applied prospectively to 27,564 pts with atherosclerotic CV disease and an LDLC ≥70 mg/dL randomized to EvoMab or placebo (Pbo) in FOURIER. The baseline risk as well as the relative and absolute risk reductions in CV death, MI or stroke with EvoMab were calculated by TRS 2° P strata.

Results:

The 10 point integer-based scheme showed a strong graded relationship with the rate of CV death, MI or stroke and the individual components (ptrend<0.0001 for all). Intermediate risk patients (TRS 2° P Score=24; 79% of population) had a 1.9% absolute risk reduction (ARR) in CV death, MI or stroke at 3 yrs with EvoMab compared to Pbo alone and high-risk patients (Score ≥5; 16%) had a 3.6% ARR, translating to a number needed to treat for 3 years of 53 and 28, respectively (FIG. 52).

Conclusion:

The TRS 2° P identifies high-risk patients with atherosclerotic CV disease who demonstrate a pattern of greater absolute risk reduction in major CV events with EvoMab.

### Example 23

### Reduction in Total Cardiovascular Events with the PCSK9 Inhibitor Evolocumab in Patients with Cardiovascular Disease in the FOURIER Trial

Introduction:

Intensive LDL-C lowering with evolocumab (EvoMab) significantly reduced the risk of major vascular events in patients with stable atherosclerotic disease treated on background statin therapy in the FOURIER trial. Although traditional survival analyses focus on time to first event, from a patient perspective all events matter.

Hypothesis:

EvoMab would significantly reduce total major vascular events including those after the first event.

Methods:

All PEP events (composite of CV death, MI, stroke, unstable angina, or coronary revascularization) were evaluated during a median 2.2 yr follow-up in FOURIER. Negative binomial regression & other sensitivity models were used.

Results:

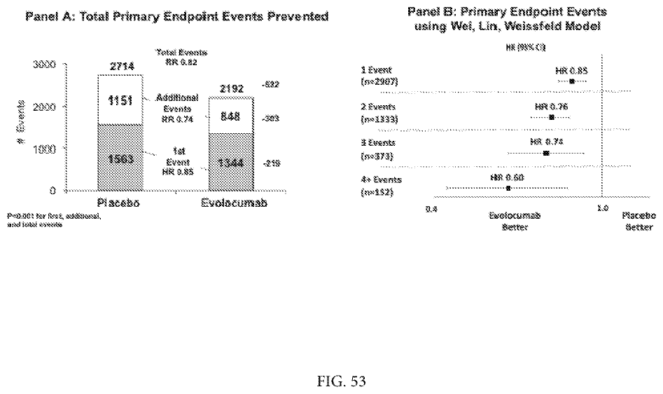

There were 2907 first PEP events and 4,906 total events PEP events (41% subsequent events) in 27,564 pts, with 1.7±1.0 (range 1.11) events on average in those with an event. EvoMab reduced total PEP events by 18% (incidence-rate ratio [RR] 0.82, 95% Cl 0.75-0.90, p<0.001), including both first events (HR 0.85 [0.79-0.92], p<0.001) and subsequent events (RR 0.74 [0.65-0.85], p<0.001; FIG. 53, panel A). A time to event model showed similar reductions (FIG. 53, panel B). For every 1000 pts treated for 3 yrs, EvoMab prevented 22 first PEP events and 52 total PEP events. Reductions in total events were driven by fewer total MIs (RR 0.74, p<0.001), strokes (RR 0.77, p=0.007), and coronary revascularization (RR 0.78, p<0.001).

Conclusions:

The addition of evolocumab to statin therapy improved clinical outcomes with significant reductions in total PEP events, driven by decreases in MI, stroke, and coronary revascularization, which revealed more than double the number of events prevented as compared with an analysis of just first events. These data indicate the long-term use of evolocumab to prevent recurrent CV events.

### Example 24

### Characterization of Types and Sizes of Myocardial Infarction Reduced with Evolocumab in FOURIER

Introduction:

The FOURIER trial described herein showed that the PCSK9 inhibitor evolocumab reduced major vascular events compared to placebo in patients with stable atherosclerotic CV disease, including reducing myocardial infarction (MI) by 27%. The present example reviews the types and sizes of MI in FOURIER.

Hypothesis:

Evolocumab reduces spontaneous MI, regardless of size and type (NSTEMI or STEMI).

Methods:

27,564 patients were randomized to evolocumab or placebo and followed for a median of 26 months. Clinical endpoints were evaluated by the TIMI clinical events committee which was not aware of treatment assignment. MI was defined based on the Third Universal MI Definition, and further classified according to MI type (Universal MI subclass, STEMI vs NSTEMI) and by MI size (peak biomarker). Rates presented are 3-year KM estimates.

Results:

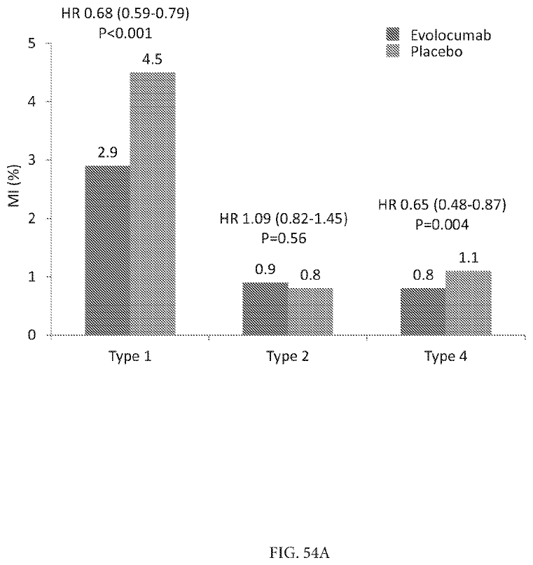

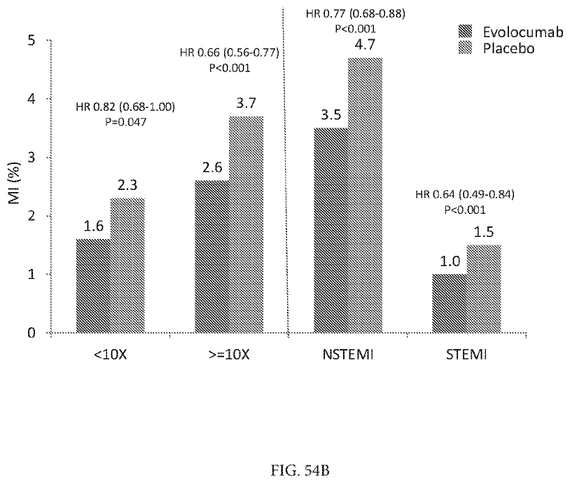

A total of 1107 subjects had a total of 1288 MIs. The majority (68%) of the MIs were atherothrombotic (Type 1), with 15% supply/demand mismatch MI (Type 2) and 15% PCI-related (Type 4). Sudden death MI (Type 3) and CABG-related MI (Type 5) accounted for a total of 21 MIs (<2%). See FIG. 54A. Evolocumab significantly reduced the risk of first MI by 27% (4.4 vs 6.3%, P<0.001), Type 1 MI by 32% and Type 4 MI by 35%, with no effect on Type 2 MI (FIG. 54A). Troponin values were available for 1151 MIs. Using fold elevation of Tn, the majority of MIs (689, 60%) were large with Tn≥10×ULN. One fifth of MIs (238, 18%) were STEMI. The benefit of evolocumab was highly significant and consistent regardless of the size of MI with a 34% reduction in MIs with Tn≥10×ULN and a 36% reduction in STEMI (FIG. 54B).

Conclusion:

LDL-C lowering with evolocumab was highly effective in reducing the risk of myocardial infarction. This reduction included a robust benefit across multiple subtypes of MI related to plaque rupture, smaller and larger MIs, and both STEMI and NSTEMI.

### Example 25

The present example examines the efficacy of intensive LDL-cholesterol lowering with PCSK9-inhibitor evolocumab in combination with statin treatment in patients with cerebrovascular disease.

Evolocumab is a monoclonal antibody that inhibits proprotein convertase subtilisin-kexin type 9 (PCSK9), lowers low-density lipoprotein (LDL) cholesterol levels by approximately 60% and reduced major vascular events in patients with clinically evident cardiovascular disease in a large randomized trial. The present example assists in detailing specific effects in patients with a history of ischemic stroke.

### Methods

FOURIER was a randomized, double-blind, placebo-controlled trial enrolling 27,564 patients with prior myocardial infarction, prior non-hemorrhagic stroke or symptomatic peripheral artery disease, additional atherosclerotic risk factors, and LDL cholesterol levels ≥70 mg/dl or non HDL cholesterol ≥100 mg/Dl on statin therapy. Patients were assigned to additional treatment with subcutaneous injections of evolocumab 140 mg bi-weekly or 420 mg monthly or matching placebo. The primary endpoint was the composite of cardiovascular death, myocardial infarction, stroke, hospitalization for unstable angina, or coronary revascularization.

### Results

The trial enrolled 5,337 patients with a history of ischemic stroke, representing 19% of all randomized. In these patients with a history of ischemic (non-hemorrhagic) stroke, mean age was 64, 66% were male. At 48 weeks, mean reduction in LDL cholesterol levels with evolocumab, as compared with placebo was 59% from 91 mg/dl to 29 mg/dl. Evolocumab treatment significantly reduced the primary endpoint relative to placebo (n=259, [9.6%] vs. n=300, [11.3%], hazard ratio 0.85 (95% CI 0.72-1.00); p=0.047). There was no evidence of heterogeneity of benefit for reduction of the key secondary endpoint of cardiovascular death, myocardial infarction, and ischemic or hemorrhagic stroke alone; and reductions of ischemic stroke and transient ischemic attack combined. Hemorrhagic stroke and neurocognitive adverse events were not increased. Conclusions

Inhibition of PCSK9 with evolocumab on a background of statin therapy in patients with a history of ischemic stroke lowered LDL-cholesterol levels to a median of 29 mg/dl and reduced the risk of cardiovascular events. These findings indicate that patients with ischemic stroke benefit from lowering of LDL cholesterol levels below current targets.

### Introduction

Patients who have experienced an ischemic stroke are at high risk of suffering future ischemic cerebral, cardiac, and peripheral events.1, I, ii Treatment to lower LDL-cholesterol with statins and with the combination of statins and ezetimibe have been shown to reduce the risk of non-hemorrhagic stroke in patients at risk of atherosclerotic cardiovascular disease.²⁻³ More intensive therapy to reduce plasma levels of LDL-cholesterol can be achieved with the combination of a statin and evolocumab, a monoclonal antibody that inhibits proprotein convertase subtilisin-kexin type 9 (PCSK9)⁴.

The present disclosure includes the result of a randomized clinical trial with 27 564 patients with prior myocardial infarction, prior non-hemorrhagic stroke or symptomatic peripheral arterial disease, additional atherosclerotic risk factors, and LDL-cholesterol levels of 70 mg/dl or higher while receiving high to moderate-intensity statin therapy, in which all patients were treated with either add-on evolocumab or non HDL cholesterol ≥100 mg/dL, placebo in a double-blind fashion⁵⁻⁶. The Further cardiovascular Outcomes Research with PCSK9 Inhibition in subjects with Elevated Risk (FOURIER) trial showed that evolocumab reduced the median LDL cholesterol level to a median of 30 mg/dl (inter quartile range 19-46 mg/dl) while the patient treated with statins alone remained at a median level of 90 mg/dl (inter quartile range 80-109 mg/dl).⁶ After a median follow-up of 2.2 years for the entire cohort, there was a significant reduction in cardiovascular events in the group treated with evolocumab compared to the placebo group. In this report the effect of this among the subgroup of patients enrolled in the study who had a prior non-hemorrhagic stroke is examined.

### Methods

Patients were recruited at 1242 sites in 49 countries including Europe, Asia, Australia, North and South America and the South African Republic. To qualify patients had to be between 40 and 85 years of age and have clinically evident cardiovascular disease: prior myocardial infarction, prior non-hemorrhagic stroke, or symptomatic peripheral artery disease. Furthermore, the patients needed to have at least one additional major or at least additional minor atherosclerotic risk factors. Major risk factors were: 1. diabetes, 2. age ≥65 years, 3. MI or stroke <6 months before screening, 4. Additional diagnosis of myocardial infarction or non-hemorrhagic stroke excluding the qualifying event, 5. Current daily cigarette smoking, 6. History of symptomatic peripheral artery disease if eligible by myocardial infarction or stroke. Minor risk factors were: 1. History of non-myocardial infarction related coronary revascularization, 2. Residual coronary artery disease with ≥40% stenosis in ≥2 large vessels, 3. Most recent HDL-cholesterol <40 mg/dl for men and <50 mg/dl for women, 4. Most recent high-sensitive C-reactive protein (hsCRP) >2.0 mg/L, 5. Most recent LDL-cholesterol ≥130 mg/dl or non-HDL cholesterol ≥160 mg/dl. After ≥2 weeks of stable statin therapy LDL cholesterol had to be ≥70 mg/dl or non-HDL cholesterol had to be ≥100 mg/dl. Furthermore, fasting triglycerides had to be ≤400 mg/dl, all lipid measurements had to be performed at a central laboratory.

Leading exclusion criteria were qualifying event occurring within 4 weeks, previous hemorrhagic stroke, severe heart failure, severe renal failure, malignancy within the past 10 years, active liver disease or hepatic dysfunction, untreated or inadequately treated hyperthyroidism or hypothyroidism and severe concomitant non-cardiovascular disease.

Patients were randomly assigned (1:1) subcutaneous evolocumab (either 140 mg every 2 weeks or 420 mg every month according to patient preference) or matching placebo. Study visits were scheduled at 2, 4, and 12 weeks and every 12 weeks thereafter.

The primary study endpoint was the composite of cardiovascular death, myocardial infarction, ischemic or hemorrhagic stroke, hospitalization for unstable angina, or coronary revascularization. The key secondary endpoint was the composite of cardiovascular death, myocardial infarction, or ischemic or hemorrhagic stroke. All events were adjudicated by an independent endpoint committee blinded to treatment allocation and on-treatment lipid levels. The modified Rankin Score was determined ≥30 days after the event in patients suffering a stroke. The subgroup analysis of results obtained in the stroke population was predefined in the statistical analysis plan.

### Results

Of the 27,564 patients randomized between February 2013 through June 2015, 19% (n=5337) had a history of non-hemorrhagic stroke. The median time from the most recent ischemic stroke to randomization was 3.2 years and 27% of these patients were randomized less than 1 year after the stroke. Of the patients randomized with a history of ischemic stroke, 30.1% and 31.3% of patients in the evolocumab and placebo group respectively also had a history of myocardial infarction. The main baseline characteristics in patients with a history of ischemic stroke are shown in Table 25.1. Among patients with a history of ischemic stroke there were no major differences between the two treatment groups. Compared with patients enrolled without a prior ischemic stroke, patients with a prior ischemic stroke were older, more often female, more frequently had a history of hypertension, diabetes, atrial fibrillation, and transient ischemic attack, and were less often Caucasian and more often Asian, were less frequently current smokers (Table 25.3).

At the time of randomization, the median LDL-cholesterol level was 91 mg/dl (interquartile range 79.0-108.5) in the evolocumab group and 92 mg/dl (interquartile range 80-110) in the placebo group. After 4 weeks the median LDL-cholesterol level had dropped to 31 mg/dl (interquartile range 21-46 mg/dl) in the evolocumab group. In the evolocumab group, 20% of patients reached LDL cholesterol levels of <19 mg/dl at 4 weeks. At 48 weeks the median level in the evolocumab was 29 mg/dl (interquartile range 18-48 mg/dl) while in the placebo group the median LDL level was 89 mg/dl (interquartile range 74-110). HDL cholesterol levels remained relatively stable during the trial with median levels in both treatment groups of 46 mg/dl (interquartile range 38-55 mg/dl) at baseline, rising to 49 mg/dl in the evolocumab group and 46 mg/dl in the placebo group at 48 weeks. In patients coming back for lipid measurement the effect on LDL-cholesterol remained stable in the two groups with a 56% mean reduction in the evolocumab group at 48 weeks compared to the placebo group.

### Efficacy

Among the patients enrolled with a history of non-hemorrhagic stroke, evolocumab significantly reduced the primary composite endpoint of cardiovascular death, myocardial infarction, ischemic and hemorrhagic stroke, hospitalization for unstable angina, or coronary revascularization. This endpoint occurred in 259 patients in the evolocumab group and 300 patients in the placebo group (hazard ratio 0.85, 95% confidence interval 0.72-1.00, (p=0.047). The result is similar to that observed for the entire study population. The secondary endpoints were consistent in direction and magnitude with that observed in the entire trial cohort. In particular, the key secondary endpoint of the composite of cardiovascular death, myocardial infarction or stroke had a hazard ratio 0.80 (95% confidence interval 0.73-0.88, (p<0.00001)); for myocardial infarction, (hazard ratio 0.74, (95% CI 0.55-1.19); and ischemic or hemorrhagic stroke, hazard ratio 0.90 (95% CI 0.68-1.19).

Considering subtypes of cerebrovascular outcome events, hazard ratios were nominally lower for recurrent cerebral ischemic events than for cerebral hemorrhagic events.

The benefits of evolocumab with regard to the risk of the primary and key secondary composite end points were largely consistent across major subgroups of patients with prior ischemic stroke, including those based on age, sex, and entry LDL level

The type of primary endpoint events accruing over the course of the trial differed among patients enrolled with a history of ischemic stroke and those without. In the control arm, patients with versus without a history of ischemic stroke had a substantially higher rate of recurrent ischemic and hemorrhagic stroke and a higher rate of cardiovascular death, and a lower rate of myocardial infarction.

### Safety

The study treatment was well tolerated among patients enrolled with a history of ischemic stroke and there were no differences for any specific adverse event category between the treatment groups (Table 25.2). The pattern of adverse events was similar for patients qualifying for the study with a history of stroke as for those without. Neurocogntive adverse events were not increased among evolocumab vs placebo patients (2.0% vs 2.1%) and were also not increased in the subset of among patients achieving very low levels (<30 mg/dl) of LDL cholesterol.

### Discussion

Among patients with a history of ischemic stroke, further lowering of LDL cholesterol by addition of evolocumab to statin therapy significantly reduced the risk of cardiovascular events, with a 15% reduction in the risk of the primary composite endpoint of cardiovascular death, myocardial infarction, ischemic and hemorrhagic stroke, hospitalization for unstable angina, or coronary vascularization. These effects, and those for all secondary endpoints were consonant with those among the entire study population, indicating that patients with a prior ischemic stroke benefitted from evolocumab the same as patients with other types of atherosclerotic cardiovascular disease. The ischemic stroke patients allocated to evolocumab reached unprecendented low levels of LDL cholesterol with one-fifth of patients having LDL cholesterol levels less than 19 mg/dl within one month of randomization.

These results extend insights regarding the benefit of moderate and intensive reductions of LDL cholesterol level among patients with an ischemic stroke and atherosclerotic risk factors. In FOURIER, it was found that additional reductions in cardiovascular event rates among patients with ischemic stroke when LDL cholesterol levels were further lowered to a median of 29 mg/dl. These observations accord well with observational studies demonstrating an association of PCSK9 gene polymorphsims and plasma levels of LDL cholesterol with development and progression of carotid artery intima-media thickness and atherosclerosis.viii, ix, x

Achievement of very low LDL-cholesterol levels with evolocumab was not associated with an increase in adverse effects among ischemic stroke patients compared to the placebo group. In particular, there was no trend of increased rate of hemorrhagic stroke associated with extremely low levels of LDL-cholesterol, even among this subgroup of patients entering the trial with past ischemic stroke and, by definition, damaged cerebral vessels. This finding is reassuring given signal from observational studies and randomized trials of other LDL-cholesterol lowering therapies that raised concern that low LDL-cholesterol levels might be associated with an increased risk of hemorrhagic stroke. In meta-analyses, statin therapy was associated with non-significant increased risk of hemorrhagic stroke across 21 primary and secondary prevention trials (RR 1.15, 95% CI 0.87-1.51)⁷ and across 2 trials of secondary prevention specifically in patients with prior symptomatic cerebrovascular disease (RR 1.71, 95% CI 1.19-2.50).XI Similarly, in a large trial of the cholesterol absorption inhibitor ezetimibe there was a non-significant trend for increased risk of hemorrhagic stroke (HR 1.38, 95% ci 0.89-2.04).³ The lack of association between hemorrhagic stroke and the more extreme lowering of LDL-cholesterol in the current trial suggests that cholesterol-lowering per se may not increase hemorrhagic stroke risk, and any hemorrhagic tendencies of statins and ezetimibe may be mediated by other mechanisms such as those agents' known pleiotropic, off-target, antiplatelet and antithrombotic effect, which may differ quantitatively and qualitatively from the pleiotropic antithrombotic profile of PCSK9 inhibitors.xii, xiii, xiv

The FOURIER trial was powered based on all eligible patients, so the sample size of patients specifically qualifying with ischemic stroke was modest, and power to explore subgroup effects among patients enrolled with ischemic stroke was moderate. The duration of follow-up in the FOURIER trial was relatively short compared to most statin trials which were on average 5 years in duration. The trial was originally planned to be approximately 4 years, but the event rate in the control group was approximately 50% higher than projected, so the prespecified number of events were accrued more quickly. Information was not collected regarding mechanistic subtypes of ischemic stroke such as large artery atherosclerosis, small artery atherosclerosis, cardioembolic, and other. But the requirement for presence of atherosclerotic risk factors and for ischemic rather than hemorrhagic stroke would strongly select for patients with ischemic stroke of atherosclerotic origin.

In conclusion, among patients with prior ischemic stroke and additional atherosclerotic risk factors, inhibition of PCSK9 with evolocumab on a background of statin therapy lowered LDL cholesterol levels to a median of 29 mg/dl, was safe and reduced the risk of further cardiovascular events, including stroke. These findings indicate that patients with ischemic stroke and additional atherosclerotic risk factors benefit from lowering LDL cholesterol levels below current targets.

### REFERENCES FOR EXAMPLE 25

1. Kernan W N, Ovbiagle B, Black B R, Bravata D M, Chimowitz M I, Ezekoviwitz M D, et al. Guidelines for the prevention of stroke in patients with stroke and transient ischemic attack. A guideline for healthcare professionals from the American heart Association/American Stroke Association. Stroke 2014; 45:2160-236.
2. Cholesterol Treatment Trialists' (CTT) Collaboration. Efficacy and safety of LDL-lowering therapy among men and women: meta-analysis of individual data from 174 000 participants in 27 randomized trials. Lancet 2015; 385:1397-405.
3. Cannon C P, Blazing M A, Giugliano R P, McCagg A, White J A, Theroux P, et al. Ezetimibe added to statin therapy after acute coronary syndromes. N Engl J Med 2015; 372:2387-97.
4. Sabatine M S, Giugliano R P, Wiviott S D, Raal F J, Blom D J, Robinson J, et al. Efficacy and safety of evolocumab in reducing lipids and cardiovascular events. N Engl J Med 2015; 372:1500-09.
5. Sabatine M S, Giugliano R P, Keech A, Honarpour N, Wang H, Liu T, et al. Rationale and design of the Further cardiovascular Outcomes Research with PCSK9 Inhibition in subjects with Elevated Risk trial. Am heart J 2016; 173:94-101.
6. Sabatine M S, Giugliano R P, Keech A C, Honarpour N, Wiviott S D, Murphy S M, et al. Evolcumab and clinical outcomes in patients with cardiovascular disease. N Engl J Med 2017; 376:1713-1722.
7. Giugliano R P, Pedersen T R, Park J-G, De Ferrari G M, Giaciong Z A, Ceska R, et al. Clinical efficacy and safety of achieving very low LDL-cholesterol concentrations with the PCSK9 inhibitor evolocumab: a prespecified secondary analysis of the FOURIER trial. Lancet 2017; http “colon”//dx.doi “dot” org/10.1016/50140-6736(17)32290-0.
8. Cholesterol Treatment Trialists' (CTT) Collaboration. Efficacy and safety of more intensive lowering of LDL cholesterol: a meta-analysis of data from 170 000 participants in 26 randomised trials. Lancet 2010; 376:1670-81.

### ADDITIONAL REFERENCES

I Dhamoon M S, Sciacca R R, Rundek T, Sacco R L, Elkind M S: Recurrent stroke and cardiac risks after first ischemic stroke: the Northern Manhattan Study. Neurology 2006; 66:641-646.
II Steg P G, Bhatt D L, Wilson P W, D'Agostino R Sr, Oilman E M, Rother J, Liau C S, Hirsch A T, Mas J L, Ikeda Y, Pencina M J, Goto S: One-year cardiovascular event rates in outpatients with atherothrombosis. JAMA 2007; 297:1197-1206
III Amarenco P, Bogousslaysky J, Callahan A 3rd, Goldstein L B, Hennerici M, Rudolph A E, Sillesen H, Simunovic L, Szarek M, Welch K M, Zivin J A; Stroke Prevention by Aggressive Reduction in Cholesterol Levels (SPARCL) Investigators. High-dose atorvastatin after stroke or transient ischemic attack. N Engl J Med. 2006 Aug. 10; 355(6):549-59.
IV Collins R, Armitage J, Parish S, Sleight P, Peto R; Heart Protection Study Collaborative Group. Effects of cholesterol-lowering with simvastatin on stroke and other major vascular events in 20536 people with cerebrovascular disease or other high-risk conditions. Lancet. 2004 Mar. 6; 363(9411):757-67.
V The Long-Term Intervention with Pravastatin in Ischaemic Disease (LIPID) Study Group. Prevention of Cardiovascular Events and Death with Pravastatin in Patients with Coronary Heart Disease and a Broad Range of Initial Cholesterol Levels. N Engl J Med 1998; 339:1349-1357
VI Sacks F M, Pfeff er MA, Moyé L A, et al. The effect of pravastatin on coronary events after myocardial infarction in patients with average cholesterol levels. N Engl J Med 1996; 336: 1001-09.
VII Cannon C P, Blazing M A, Giugliano R P, et al. Ezetimibe added to statin therapy after acute coronary syndromes. N Engl J Med 2015; 372: 2387-97.
VIII Norata G D1, Garlaschelli K, Grigore L, Raselli S, Tramontana S, Meneghetti F, Artali R, Noto D, Cefalù A B, Buccianti G, Averna M, Catapano A L. Effects of PCSK9 variants on common carotid artery intima media thickness and relation to ApoE alleles. Atherosclerosis. 2010 January; 208(1):177-82.
IX Chan D C, Pang J, McQuillan B M, Hung J, Beilby J P, Barrett P H, Watts G F. Plasma Proprotein Convertase Subtilisin Kexin Type 9 as a Predictor of Carotid Atherosclerosis in Asymptomatic Adults. Heart Lung Circ. 2016 May; 25(5):520-5.
X Xie W, Liu J, Wang W, Wang M, Qi Y, Zhao F, Sun J, Liu J, Li Y, Zhao D. Association between plasma PCSK9 levels and 10-year progression of carotid atherosclerosis beyond LDL-C: A cohort study. Int J Cardiol. 2016 Jul. 15; 215:293-8.
XI Amarenco P, Labreuche J. Lipid management in the prevention of stroke: review and updated meta-analysis of statins for stroke prevention. Lancet Neurology 2009:8:453-463.
XII Oesterle A, Laufs U, Liao J K. Mitsios J V, Papathanasiou A I, Goudevenos J A, Tselepis A D. The antiplatelet and antithrombotic actions of statins. Pleiotropic Effects of Statins on the Cardiovascular System. Circ Res. 2017 Jan. 6; 120(1):229-24.
XIII Pesaro A E, Serrano C V Jr, Fernandes J L, et al. Pleiotropic effects of ezetimibe/simvastatin vs. high dose simvastatin. Int J Cardiol 2012; 158:400-404
XIV Navarese E P, Kolodziejczak M, Kereiakes D J, Tantry U S, O'Connor C, Gurbel P A. Proprotein Convertase Subtilisin/Kexin Type 9 Monoclonal Antibodies for Acute Coronary Syndrome: A Narrative Review. Ann Intern Med. 2016 May 3; 164(9):600-7.

TABLE 25.1
Baseline characteristics of patients with a history of ischemic stroke

| | Evolocumab | Placebo |
|---|---|---|
| | N = 2686 | N = 2651 |
| Characteristic | Mean (SD) | Mean (SD) |
| Age (years) | 64.0 (8.9) | 64.1 (8.7) |
| Weight (kg) | 83.2 (18.2) | 83.9 (18.3) |
| Race, n (%) | | |
| Time from most recent stroke (yrs) | 5.1 (5.6) | 5.3 (5.8) |
| History of myocardial infarction (n) | 809 (30.1) | 830 (30.3) |
| Time from most recent MI (yrs) | 7.3 (7.3) | 7.4 (7.5) |
| Systolic BP (mmHg) | 134.6 (16.1) | 134.6 (15.6) |
| Statin Use | | |
| High intensity | 1684 [62.7%] | 1646 [62.1%] |
| Moderate intensity | 991 [36.9%] | 1000 [37.7%] |
| Lipid measures | | |
| LDL cholesterol (mg/dl) | 97.0 (29.7) | 98.0 (26.9) |
| HDL cholesterol (mg/dl) | 47.8 (13.4) | 47.8 (13.6) |
| Triglycerides (mg/dl) | 145.9 (66.4) | 147.2 (71.4) |
| Total cholesterol (mg/dl) | 173.9 (33.9) | 175.0 (32.3) |
| High sensitivity CRP (mg/L) | 3.7 (6.1) | 3.8 (7.7) |

TABLE 25.2
Adverse Events

| | Evolocumab | Placebo |
|---|---|---|
| | (N = 2686) | (N = 2651) |
| Outcome | Number (%) | Number (%) |
| Any treatment-emergent adverse event | 2103 (78.4) | 2059 (77.8) |
| Serious treatment-emergent adverse event | 741 (27.6) | 738 (27.9) |
| Adverse event leading to discontinuation | 159 (5.9) | 131 (5.0) |
| of study drug | | |
| Injection site reactions | 54 (2.0) | 46 (1.7) |
| Allergic reactions | 89 (3.3) | 81 (3.1) |
| Neurocognitive event | 54 (2.0) | 55 (2.1) |
| Headache | 93 (3.5) | 115 (4.3) |
| Fatigue | 65 (2.4) | 52 (2.0) |
| New onset diabetes | 114 (7.6) | 106 (7.2) |
| Muscle-related event | 112 (4.2) | 110 (4.2) |
| Rhabdomyolysis | 1 | 5 (0.2) |

TABLE 25.3
Primary and Secondary End Points.

| | Evolocumab | Placebo | |
|---|---|---|---|
| | (N = 2686) | (N = 2651) | |
| | Number | Number | Hazard Ratio* |
| Outcome | (%) | (%) | (95% CI) |
| Primary endpoint: | 259 (9.6) | 300 (11.3) | 0.85 (0.72-1.00) |
| cardiovascular death, | | | |
| myocardial infarction, | | | |
| stroke, hospitalization | | | |
| for unstable angina, or | | | |
| coronary | | | |
| revascularization | | | |
| Key secondary endpoint: | 202 (7.5) | 224 (8.4) | 0.89 (0.74-1.08) |
| cardiovascular death, | | | |
| myocardial infarction or | | | |
| stroke | | | |
| Cardiovascular death | 73 (2.7) | 65 (2.5) | 1.11 (0.80-1.56) |
| Acute myocardial | 75 (2.8) | 100 (3.8) | 0.74 (0.55-1.00) |
| infarction | | | |
| Stroke (ischemic and | 95 (3.5) | 105 (4.0) | 0.90 (0.68-1.19) |
| hemorrhagic) | | | |
| Coronary | 89 (3.3) | 128 (4.8) | 0.68 (0.52-0.90) |
| revascularization | | | |
| All-cause death | 120 (4.5) | 111 (4.2) | 1.07 (x.xx-y.yy) |

*These effects in the ischemic stroke subgroup were homogenous with those in the overall trial for all endpoint (Cochran's Q heterogeneity p value > 0.10 for all).

### Example 26

The present example provides a method of reducing a relative risk of a cardiovascular event by at least 10%. A subject that is on at least a moderate intensity of a statin therapy receives a PCSK9 neutralizing antibody in an amount sufficient to lower a LDL-C level of the subject by about 20 mg/dL. This reduces the relative risk of a cardiovascular event by at least 10% in the subject.

### Example 27

The present example provides a method of decreasing percent atheroma volume (PAV). A subject is identified who has received at least a moderate level of treatment by a non-PCSK9 LDL-C lowering agent. The subject then receives evolocumab in an amount sufficient and time sufficient to lower the LDL-C level to less than 100 mg/dL, thereby decreasing a percent atheroma volume (PAV) in the subject.

### Example 28

The present example provides a method of decreasing total atheroma volume (TAV). A subject is identified who has received at least a moderate level of treatment by a non-PCSK9 LDL-C lowering agent. The subject then receives evolocumab in an amount sufficient and time sufficient to lower the LDL-C level to less than 100 mg/dL, thereby decreasing a total atheroma volume (TAV) in the subject.

### Example 29

The present example provides a method of treating coronary atherosclerosis. One first identifies a statin-intolerant subject. One then administers at least a low intensity statin treatment to the statin-intolerant subject. One then administers an effective amount of evolocumab to the subject. This is continued to thereby treat coronary atherosclerosis.

### Example 30

The present example provides a method of combining a PCSK9 inhibitor therapy and a non-PCSK9 LDL-C lowering therapy to produce greater LDL-C lowering and regression of coronary atherosclerosis at a dose that is well tolerated. One first administers at least a moderate intensity of a non-PCSK9 LDL-C lowering therapy to a subject. One then administers an adequate amount of evolocumab to the subject such that the subject's LDL-C levels drop to no more than 40 mg/dL. One then maintains the subject's LDL-C levels at no more than 40 mg/dL for at least one year to provide the noted result.

### Example 31

The present example provides a method of treating a subject that is unable to tolerate a full therapeutic dose of a non-PCSK9 LDL-C lowering agent. One identifies the subject and then administers a PCSK9 inhibitor to the subject until a LDL cholesterol level of the subject decreases beneath 60 mg/dL.

### Example 32

The present example provides a method of treating coronary atherosclerosis. One identifies a subject that has a LDL-C level of less than 70 mg/dL and administers a non-PCSK9 LDL-C lowering agent to the subject, in an amount sufficient and time sufficient to lower the LDL-C level to less than 60 mg/dL.

### Example 33

The present example provides a method of treating coronary atherosclerosis. One identifies a subject that has a LDL-C level of less than 70 mg/dL and administers a PCSK9 LDL-C lowering agent to the subject, in an amount sufficient and time sufficient to lower the LDL-C level to less than 40 mg/dL.

### Example 34

The present example provides a method of lowering LDL-C levels in a subject. One administers a first therapy to a subject. The first therapy comprises a statin. One then administers a second therapy to the subject. The second therapy comprises a PCSK9 inhibitor. Both the first and second therapies are administered to the subject for at least five years, and the subject's LDL-C level is maintained beneath 50 mg/dL. This thereby reduces the LDL-C of the subject.

### Example 35

The present example provides a method of lowering non-HDL-C levels in a subject. One administers a first therapy to a subject. The first therapy comprises a statin. One then administers a second therapy to the subject. The second therapy comprises a PCSK9 inhibitor. Both the first and second therapies are administered to the subject for at least five years, and the subject's non-HDL-C level is maintained beneath 80 mg/dL. This thereby reduces the non-HDL-C of the subject.

### Example 36

The present example provides a method of treating a subject. One first identifies a subject with peripheral artery disease and then reduces the level of PCSK9 activity in the subject by using evolocumab in an amount and for a duration adequate to reduce the risk or PAD.

### Example 37

The present example provides a method of reducing a risk of an adverse limb event in a subject. One reduces a level of PCSK9 activity in a subject by administering evolocumab to the subject. The subject has peripheral artery disease. Following the therapy, the subject will have a reduce risk of an adverse limb event.

### Example 38

The present example provides a method of reducing a risk of a major adverse limb event (“MALE”). One first administers a non-statin LDL-C lowering agent to a subject, and then administers a statin to the subject. The subject has peripheral artery disease (“PAD”). Following the therapy, the subject will have a reduce risk of MALE.

### Example 39

The present example provides a method of reducing a risk of a major cardiovascular adverse event (“MACE”). One first administers a non-statin LDL-C lowering agent to a subject, and then administers a statin to the subject. The subject has PAD. Following the therapy, the subject will have a reduce risk of MACE.

### Example 40

The present example provides a method of reducing a risk of a cardiovascular event. One first provides a first therapy to a subject, wherein the first therapy comprises a non-PCSK9 LDL-C lowering therapy. One also provides a second therapy to the subject, wherein the second therapy comprises a PCSK9 inhibitor. The subject has a Lp(a) level of 11.8 mg/dL to 40.

### Example 41

The present example provides a method of reducing a risk of a major vascular event in a subject. One identifies a subject that has at least one of: (a) a recent MI, (b) multiple prior MIs, or (c) multivessel disease. One then provides a first therapy to a subject, wherein the first therapy comprises a non-PCSK9 LDL-C lowering therapy. One then provides a second therapy to the subject, wherein the second therapy comprises a PCSK9 inhibitor. This reduces a risk that the subject will have a major vascular event.

### Example 42

The present example provides a method of treating coronary atherosclerosis. One identifies a subject that has a LDL-C level of greater than 70 mg/dL. One administers an anti-PCSK9 neutralizing antibody to the subject in an amount sufficient and time sufficient to lower the LDL-C level to less than 40 mg/dL, or in the alternative, less than 30 or in the alternative, less than 20 mg/dL.

### INCORPORATION BY REFERENCE

All references cited herein, including patents, patent applications, papers, text books, and the like, and the references cited therein, to the extent that they are not already, are hereby incorporated herein by reference in their entirety. To the extent that any of the definitions or terms provided in the references incorporated by reference differ from the terms and discussion provided herein, the present terms and definitions control.

### EQUIVALENTS

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method of treating coronary atherosclerosis, the method comprising:
a. identifying a subject that is on a first therapy, wherein the first therapy comprises a non-PCSK9 LDL-C lowering therapy; and
b. administering a second therapy to the subject, wherein the second therapy comprises a PCSK9 inhibitor therapy, wherein both the first and second therapies are administered to the subject in an amount and time sufficient to reverse coronary atherosclerosis in the subject, and wherein the first therapy is not the same as the second therapy.

2. A method of treating coronary atherosclerosis, the method comprising:
a. identifying a subject that has a LDL-C level of less than 70 mg/dL; and
b. administering an anti-PCSK9 neutralizing antibody to the subject, in an amount sufficient and time sufficient to lower the LDL-C level to less than 60 mg/dL.

3. A method of decreasing percent atheroma volume (PAV) in a subject, the method comprising:
identifying a subject that has received at least a moderate level of treatment by a statin; and
administering an anti-PCSK9 neutralizing antibody to the subject in an amount sufficient and time sufficient to lower the LDL-C level to less than 90 mg/dL, thereby decreasing a percent atheroma volume (PAV) in the subject.

4. A method of decreasing total atheroma volume (TAV) in a subject, the method comprising:
a. identifying a subject that has received at least a moderate level of treatment by a statin; and
b. administering an anti-PCSK9 neutralizing antibody to the subject in an amount sufficient and time sufficient to lower the LDL-C level to less than 90 mg/dL, thereby decreasing a total atheroma volume in the subject.

5. A method of treating coronary atherosclerosis, the method comprising:
a. identifying a statin-intolerant subject;
b. administering at least a low dose statin treatment to the statin-intolerant subject; and
c. administering an amount of an anti-PCSK9 neutralizing antibody to the subject, thereby treating coronary atherosclerosis.

6. A method of reducing an amount of atherosclerotic plaque in a subject, the method comprising administering to a subject having atherosclerotic plaque a monoclonal antibody to human PCSK9, wherein the subject is receiving optimized statin therapy, thereby reducing the amount of atherosclerotic plaque in the subject.

7. A method of combining evolocumab and a statin therapy to produce greater LDL-C lowering and regression of coronary atherosclerosis at a dose that is well tolerated, the method comprising:
administering at least a moderate intensity of a statin therapy to a subject;
administering an adequate amount of evolocumab to the subject such that the subject's LDL-C levels drop to no more than 40 mg/dL; and
maintaining the subject's LDL-C levels at no more than 40 mg/dL for at least one year.

8. A method of treating coronary atherosclerosis, the method comprising:
identifying a subject that has a LDL-C level of less than 70 mg/dL; and
administering a PCSK9 inhibitor to the subject, in an amount sufficient and time sufficient to lower the LDL-C level to less than 60 mg/dL.

9. A method of decreasing percent atheroma volume (PAV) in a subject, the method comprising:
identifying a subject that has received at least a moderate level of treatment by a non-PCSK9 LDL-C lowering agent; and
administering a PCSK9 inhibitor to the subject in an amount sufficient and time sufficient to lower the LDL-C level to less than 90 mg/dL, thereby decreasing a percent atheroma volume (PAV) in the subject.

10. A method of decreasing total atheroma volume (TAV) in a subject, the method comprising:
identifying a subject that has received at least a moderate level of treatment by a non-PCSK9 LDL-C lowering agent; and
administering a PCSK9 inhibitor to the subject in an amount sufficient and time sufficient to lower the LDL-C level to less than 90 mg/dL, thereby decreasing a total atheroma volume in the subject.

11. A method of reducing disease progression, the method comprising:
identifying a subject with a LDL-C level of no more than 60 mg/dL;
administering at least a moderate intensity of a non-PCSK9 LDL-C lowering therapy to the subject; and
administering a PCSK9 inhibitor at a level sufficient to decrease the LDL-C level of the subject to 30 mg/dL, thereby reducing disease progression.

12. A method of combining a PCSK9 inhibitor therapy and a non-PCSK9 LDL-C lowering therapy to produce greater LDL-C lowering and regression of coronary atherosclerosis at a dose that is well tolerated, the method comprising:
administering at least a moderate intensity of a non-PCSK9 LDL-C lowering therapy to a subject;
administering an adequate amount of a PCSK9 inhibitor to the subject such that the subject's LDL-C levels drop to no more than 40 mg/dL; and
maintaining the subject's LDL-C levels at no more than 40 mg/dL for at least one year.

13. A method of reducing a risk of a cardiovascular event, the method comprising:
identifying a subject that is on a first therapy, wherein the first therapy comprises a non-PCSK9 LDL-C lowering therapy; and
administering a second therapy to the subject, wherein the second therapy comprises a PCSK9 inhibitor, wherein both the first and second therapies are administered to the subject in an amount and time sufficient to reduce a risk of a cardiovascular event in the subject, and wherein the first therapy is not the same as the second therapy, and wherein the risk is a) a composite for cardiovascular death, myocardial infarction, stroke, hospitalization for unstable angina, or coronary revascularization or b) a composite for cardiovascular death, myocardial infarction, or stroke.

14. A method of reducing a risk of a cardiovascular event, the method comprising:
identifying a subject that is on a first therapy, wherein the first therapy comprises a non-PCSK9 LDL-C lowering therapy; and
administering a second therapy to the subject, wherein the second therapy comprises a PCSK9 inhibitor, wherein both the first and second therapies are administered to the subject in an amount and time sufficient to reduce a risk of a cardiovascular event in the subject, and wherein the first therapy is not the same as the second therapy, and wherein the risk is the composite of fatal MI and/or non-fatal MI and fatal and/or non-fatal coronary revascularization.

15. A method of reducing a risk of a major adverse limb event (“MALE”), said method comprising:
administering a non-statin LDL-C lowering agent to a subject; and
administering a statin to the subject, wherein the subject has peripheral artery disease (“PAD”).

16. A method of reducing a risk of a major cardiovascular adverse event (“MACE”), said method comprising:
administering a non-statin LDL-C lowering agent to a subject; and
administering a statin to the subject, wherein the subject has PAD.

17. A method of reducing a risk of a cardiovascular event, the method comprising:
providing a first therapy to a subject, wherein the first therapy comprises a non-PCSK9 LDL-C lowering therapy; and
providing a second therapy to the subject, wherein the second therapy comprises a PCSK9 inhibitor, wherein both the first and second therapies are administered to the subject, and wherein the subject has a Lp(a) level of 11.8 mg/dL to 50.

18. A method of reducing a risk of a major vascular event in a subject, the method comprising:
1) identifying a subject that has at least one of: (a) a recent MI, (b) multiple prior MIs, or (c) multivessel disease;
2) providing a first therapy to a subject, wherein the first therapy comprises a non-PCSK9 LDL-C lowering therapy; and
3) providing a second therapy to the subject, wherein the second therapy comprises a PCSK9 inhibitor,
thereby reducing a risk that the subject will have a major vascular event.

19. A method of reducing a risk of a cardiovascular event, comprising administering, to a subject that has a LDL-C level of greater than 70 mg/dL, a PCSK9 inhibitor in an amount sufficient and time sufficient to lower the LDL-C level to less than 40 mg/dL.
